(19) United States
(12) Patent Application Publication
(10) Pub. No.: US 2025/0275469 A1
(43) Pub. Date: Aug. 28, 2025

(54) Light-Emitting Device, Organic Compound, Light-Emitting Apparatus, Light-Emitting And Light-Receiving Apparatus, Electronic Appliance, and Lighting Device

(71) Applicant: Semiconductor Energy Laboratory Co., Ltd., Kanagawa-ken (JP)

(72) Inventors: Sachiko KAWAKAMI, Atsugi, Kanagawa (JP); Anna TADA, Isehara, Kanagawa (JP); Naoaki HASHIMOTO, Sagamihara, Kanagawa (JP); Takuya ISHIMOTO, Isehara, Kanagawa (JP)

(73) Assignee: Semiconductor Energy Laboratory Co., Ltd., Kanagawa-ken (JP)

(21) Appl. No.: 18/857,060

(22) PCT Filed: Apr. 12, 2023
(86) PCT No.: PCT/IB2023/053726

§ 371 Date: Oct. 15, 2024

(30) Foreign Application Priority Data
Apr. 22, 2022 (JP) 2022-070538

(51) Int. Cl. H10K 85/60; C07D 307/92; H10K 50/15; H10K 50/17; H10K 101/30

(52) U.S. Cl. CPC H10K 85/636; C07D 307/92; H10K 85/633; H10K 50/15; H10K 50/17; H10K 85/615; H10K 85/6574; H10K 2101/30

(57) ABSTRACT

The driving voltage of a light-emitting device is lowered to improve the emission efficiency. The light-emitting device includes a first electrode, a second electrode, a light-emitting layer, and a first layer. The light-emitting layer is positioned between the first electrode and the second electrode. The first layer is positioned between the first electrode and the light-emitting layer. The light-emitting layer contains a light-emitting substance. The first layer contains a first organic compound. The HOMO level of the first organic compound is lower than or equal to −5.40 eV. The first organic compound provides a light-emitting device represented by General Formula (G1) below (Note that Q is O or S in General Formula (G1). One of A and B represents a group represented by General Formula (g1) above, and the other of A and B and R1 to R31 each independently represent any of H, D, an alkyl group, a cyclic saturated hydrocarbon group, an alkoxy group, a cyano group, halogen, a haloalkyl group, and an aromatic hydrocarbon group. Note that R9 and R10 may be bonded to each other to form a spirocyclic structure).

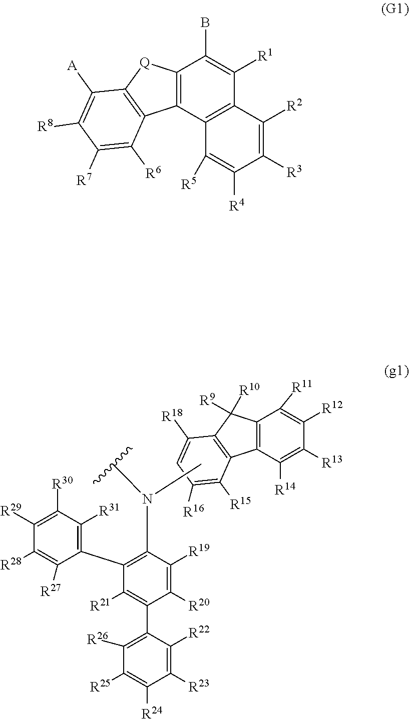

### TECHNICAL FIELD

One embodiment of the present invention relates to an organic compound, a light-emitting element, a light-emitting device, a display module, a lighting module, a display apparatus, a light-emitting apparatus, a light-emitting and light-receiving apparatus, an electronic appliance, a lighting device, and an electronic device. Note that one embodiment of the present invention is not limited to the above technical field. The technical field of one embodiment of the invention disclosed in this specification and the like relates to an object, a method, or a manufacturing method. Alternatively, one embodiment of the present invention relates to a process, a machine, manufacture, or a composition of matter. Accordingly, more specific examples of the technical field of one embodiment of the present invention disclosed in this specification include a semiconductor device, a display apparatus, a liquid crystal display apparatus, a light-emitting apparatus, a lighting device, a power storage device, a memory device, an imaging device, a driving method thereof, and a manufacturing method thereof.

### BACKGROUND ART

In recent years, research and development have been extensively conducted on light-emitting devices (also referred to as light-emitting elements) utilizing electroluminescence (EL). In the basic structure of such light-emitting devices, a layer containing a light-emitting substance is sandwiched between a pair of electrodes. By application of a voltage to the device, light emission from the light-emitting substance can be obtained.

Such light-emitting devices are self-emission type and thus have advantages over a liquid crystal display, such as high visibility of a pixel and no need of backlight, and are considered to be suitable as a flat panel display element. The light-emitting devices are also highly advantageous in that they can be thin and lightweight. Another feature is an extremely fast response speed.

Since such light-emitting devices can be formed in a film form and thus planar light emission can be obtained. Thus, a large-area element utilizing planar light emission can be easily formed. This feature is difficult to obtain with point light sources typified by incandescent lamps and LEDs or linear light sources typified by fluorescent lamps; thus, the light-emitting devices also have great potential as planar light sources, which can be used for lighting and the like.

Such light-emitting devices utilizing electroluminescence can be broadly classified according to whether a light-emitting substance is an organic compound or an inorganic compound. In the case of an organic EL device (also referred to as organic EL element) in which an organic compound is used as a light-emitting substance and a layer containing the organic compound is provided between a pair of electrodes, application of a voltage to the light-emitting device causes injection of electrons from a cathode and holes from an anode into the layer containing the organic compound and thus a current flows. The injected electrons and holes then lead the organic compound to its excited state, whereby light emission is obtained from the excited organic compound.

The types of excited states formed by an organic compound can be a singlet excited state and a triplet excited state; light emission from the singlet excited state (S*) is referred to as fluorescence, and light emission from the triplet excited state (T*) is referred to as phosphorescence.

In improving device characteristics of the light-emitting device, there are many problems which depend on a substance, and in order to solve the problems, improvement of a device structure, development of a substance, and the like have been carried out. For example, Patent Document 1 discloses a hole-transport material that can be used for forming a light-emitting device with high emission efficiency.

### REFERENCE

### Patent Document

[Patent Document 1] Japanese Published Patent Application No. 2009-298767

### SUMMARY OF THE INVENTION

### Problems to be Solved by the Invention

An object of one embodiment of the present invention is to provide a light-emitting device with low driving voltage and low power consumption. Another object of one embodiment of the present invention is to provide a light-emitting device with high emission efficiency. Another object of one embodiment of the present invention is to provide a light-emitting device with high reliability. Another object of one embodiment of the present invention is to provide a light-emitting device with favorable carrier balance.

Another object of one embodiment of the present invention is to provide a hole-transport material. Another object of one embodiment of the present invention is to provide an organic compound with a low HOMO level.

Another object of one embodiment of the present invention is to provide a light-emitting and light-receiving apparatus including a highly reliable light-emitting device and a highly reliable light-receiving device. Another object of one embodiment of the present invention is to provide a light-emitting and light-receiving apparatus including a light-receiving device with high light sensitivity.

Another object of one embodiment of the present invention is to provide a light-emitting device, a light-emitting apparatus, a light-emitting and light-receiving apparatus, an electronic appliance, and a lighting device each having low power consumption. Another object of one embodiment of the present invention is to provide a novel light-emitting device, a novel organic compound, a novel light-emitting apparatus, a novel light-emitting and light-receiving apparatus, a novel electronic appliance, and a novel lighting device.

Note that the description of these objects does not preclude the existence of other objects. Note that one embodiment of the present invention does not necessarily need to achieve all of these objects. Note that other objects will be apparent from the description of the specification, the drawings, the claims, and the like, and other objects can be derived from the description of the specification, the drawings, the claims, and the like.

### Means for Solving the Problems

One embodiment of the present invention is a light-emitting device including a first electrode, a second electrode, a light-emitting layer, and a first layer. The light-emitting layer is positioned between the first electrode and the second electrode. The first layer is positioned between the first electrode and the light-emitting layer. The light-emitting layer includes a light-emitting substance. The first layer includes a first organic compound. The HOMO level of the first organic compound is lower than or equal to −5.40 eV. The first organic compound is an organic compound represented by General Formula (G1) below.

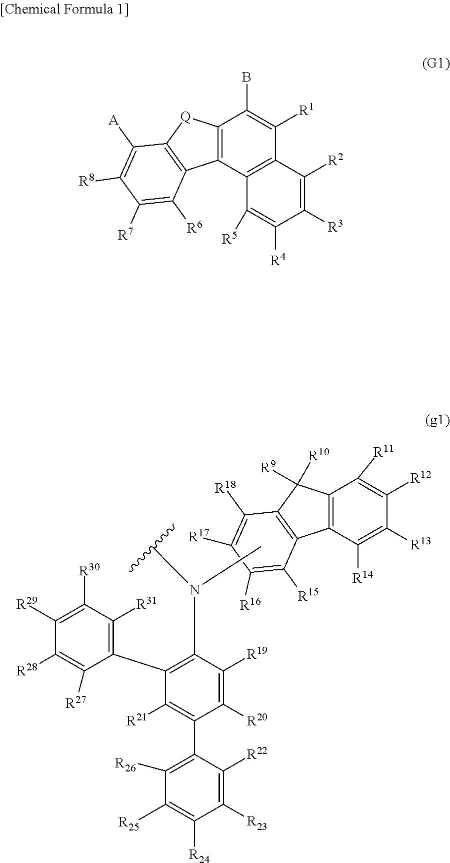

Note that in General Formula (G1) above, Q represents an oxygen atom or a sulfur atom. In addition, one of A and B is a group represented by General Formula (g1) above, and the other represents any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted aromatic hydrocarbon group having 6 to 60 carbon atoms. R¹ to R⁸ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted aromatic hydrocarbon group having 6 to 60 carbon atoms. In addition, in Formula (g1), R⁹ and R¹⁰ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted phenyl group. Note that R⁹ and R¹⁰ may be bonded to each other to form a spiro ring structure. R¹¹ to R³¹ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted aromatic hydrocarbon group having 6 to 60 carbon atoms. Note that any one of R¹⁵ to R¹⁸ represents a bond with a nitrogen atom.

Another embodiment of the present invention is a light-emitting device including a first electrode, a second electrode, a light-emitting layer, and a first layer. The light-emitting layer is positioned between the first electrode and the second electrode. The first layer is positioned between the first electrode and the light-emitting layer. The light-emitting layer includes a light-emitting substance and a host material. The first layer includes a first organic compound. A difference between the HOMO level of the first organic compound and the HOMO level of the host material is less than or equal to 0.60 eV. The first organic compound is an organic compound represented by General Formula (G1) below.

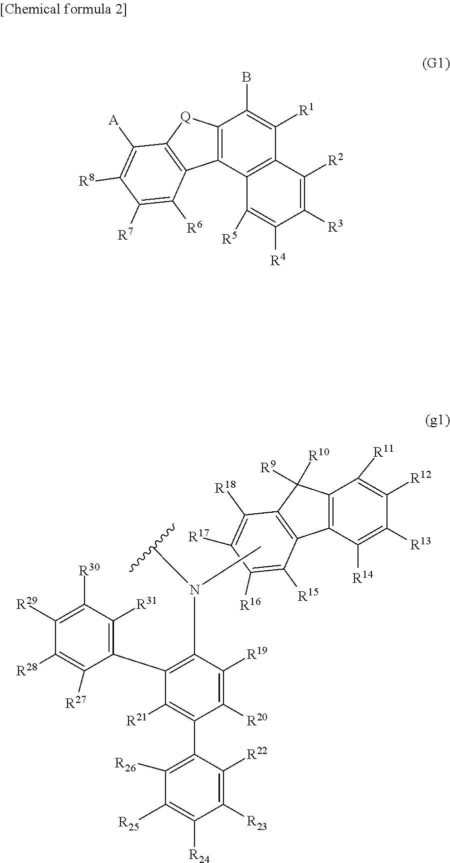

Note that in General Formula (G1) above, Q represents an oxygen atom or a sulfur atom. In addition, one of A and B is a group represented by General Formula (g1) above, and the other represents any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted aromatic hydrocarbon group having 6 to 60 carbon atoms. R¹ to R⁸ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted aromatic hydrocarbon group having 6 to 60 carbon atoms. In addition, in Formula (g1), R⁹ and R¹⁰ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted phenyl group. Note that R⁹ and R¹⁰ may be bonded to each other to form a spiro ring structure. R¹¹ to R³¹ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted aromatic hydrocarbon group having 6 to 60 carbon atoms. Note that any one of R¹⁵ to R¹⁸ represents a bond with a nitrogen atom.

Another embodiment of the present invention is a light-emitting device including a first electrode, a second electrode, a light-emitting layer, a first layer, and a second layer. The light-emitting layer is positioned between the first electrode and the second electrode. The first layer is positioned between the first electrode and the light-emitting layer. The second layer is positioned between the light-emitting layer and the first layer. The light-emitting layer includes a light-emitting substance and a host material. The first layer includes a first organic compound. The second layer includes a second organic compound. A difference between the HOMO level of the first organic compound and the HOMO level of the host material is less than or equal to 0.60 eV. A difference between the HOMO level of the first organic compound and the HOMO level of the second organic compound is less than or equal to 0.30 eV. The first organic compound is an organic compound represented by General Formula (G1) below. The second organic compound is a material having a hole-transport property.

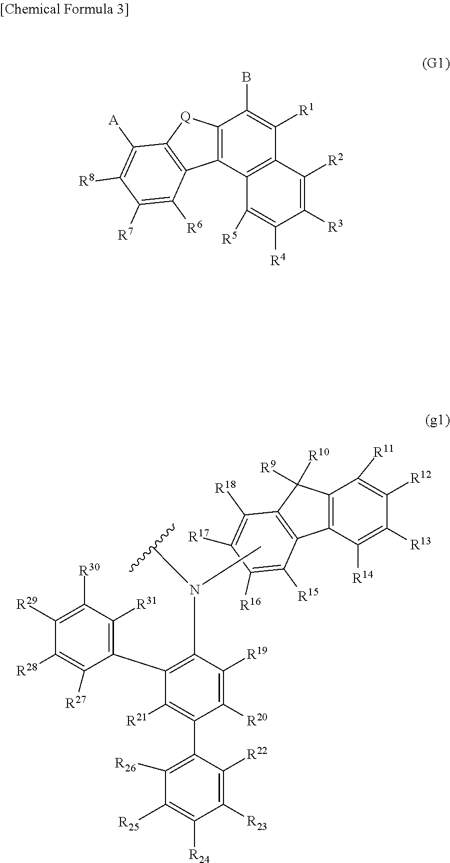

Note that in General Formula (G1) above, Q represents an oxygen atom or a sulfur atom. In addition, one of A and B is a group represented by General Formula (g1) above, and the other represents any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted aromatic hydrocarbon group having 6 to 60 carbon atoms. R¹ to R⁸ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted aromatic hydrocarbon group having 6 to 60 carbon atoms. In addition, in Formula (g1), R⁹ and R¹⁰ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted phenyl group. Note that R⁹ and R¹⁰ may be bonded to each other to form a spiro ring structure. R¹¹ to R³¹ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted aromatic hydrocarbon group having 6 to 60 carbon atoms. Note that any one of R¹⁵ to R¹⁸ represents a bond with a nitrogen atom.

Another embodiment of the present invention is an organic compound represented by General Formula (G5) below.

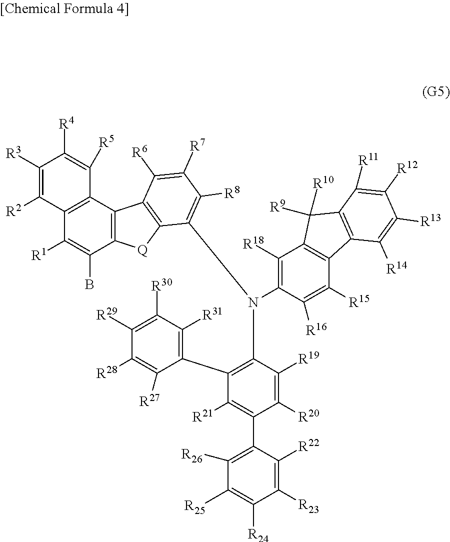

Note that in General Formula (G5) above, Q represents an oxygen atom or a sulfur atom. In addition, B, R¹ to R⁸, R¹¹ to R¹⁶, and R¹⁸ to R³¹ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted aromatic hydrocarbon group having 6 to 60 carbon atoms. In addition, R⁹ and R¹⁰ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted phenyl group. Note that R⁹ and R¹⁰ may be bonded to each other to form a spiro ring structure.

Another embodiment of the present invention is an organic compound represented by Structural Formula (300) below.

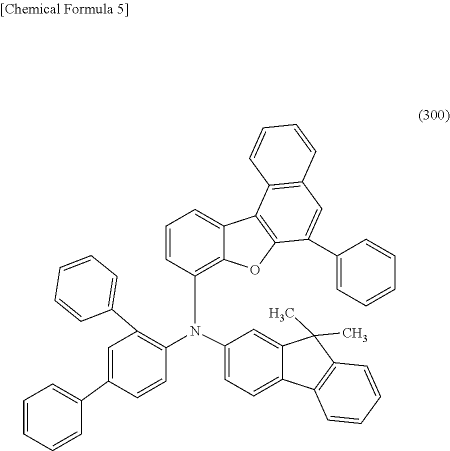 [SMILES: CC1(C)c2ccccc2-c2ccc(N(c3ccc(-c4ccccc4)cc3-c3ccccc3)c3cccc4c3oc3c(-c5ccccc5)cc5ccccc5c34)cc21]

Another embodiment of the present invention is a light-emitting and light-receiving apparatus including a light-emitting device and a light-receiving device. The light-emitting device and the light-receiving device are positioned over the same substrate. The light-emitting device includes a first electrode, a second electrode, a light-emitting layer, and a first layer. The light-emitting layer is positioned between the first electrode and the second electrode. The first layer is positioned between the first layer and the light-emitting layer. The first layer includes a first organic compound. The light-receiving device includes a third electrode, a fourth electrode, a light-receiving layer, and a second layer. The light-receiving layer is positioned between the third electrode and the fourth electrode. The second layer is positioned between the third electrode and the light-receiving layer. The second layer includes the first organic compound. The HOMO level of the first organic compound is lower than or equal to −5.40 eV. The first organic compound is an organic compound represented by General Formula (G10) below.

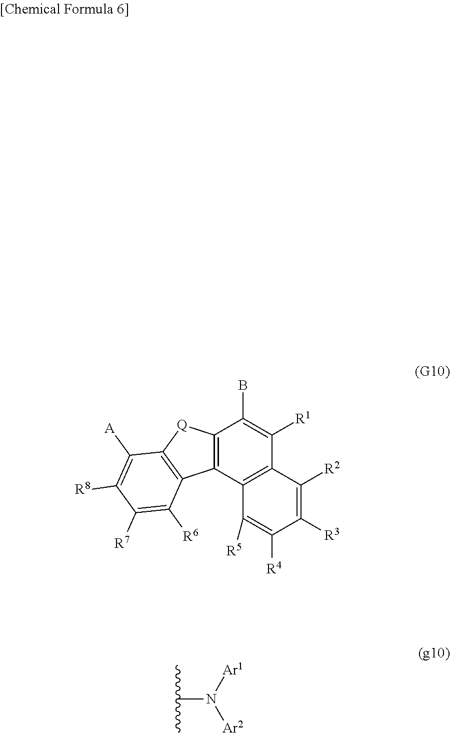

Note that in General Formula (G10) above, Q represents an oxygen atom or a sulfur atom. In addition, one of A and B is a group represented by General Formula (g10) above, and the other represents any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted aromatic hydrocarbon group having 6 to 60 carbon atoms. R¹ to R⁸ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted aromatic hydrocarbon group having 6 to 60 carbon atoms. In addition, in Formula (g1), Ar¹ and Ar² each independently represent a substituted or unsubstituted aromatic hydrocarbon group having 6 to 60 carbon atoms.

Note that the light-emitting apparatus in this specification includes, in its category, an image display device that uses a light-emitting device. The light-emitting apparatus may also include a module in which a light-emitting device over a substrate is provided with a connector such as an anisotropic conductive film or a TCP (Tape Carrier Package), a module in which a printed wiring board is provided at the end of a TCP, and a module in which an IC (integrated circuit) is directly mounted on a light-emitting device by a COG (Chip On Glass) method. Furthermore, in some cases, lighting equipment or the like includes the light-emitting apparatus.

### Effect of the Invention

According to one embodiment of the present invention, a light-emitting device with low driving voltage and low power consumption can be provided. According to another embodiment of the present invention, a light-emitting device with high emission efficiency can be provided. According to another embodiment of the present invention, a light-emitting device with high reliability can be provided. According to another embodiment of the present invention, a light-emitting device with favorable carrier balance can be provided.

According to another embodiment of the present invention, a hole-transport material can be provided. According to another embodiment of the present invention, an organic compound with a low HOMO level can be provided.

According to another embodiment of the present invention, a light-emitting and light-receiving apparatus including a highly reliable light-emitting device and a highly reliable light-receiving device can be provided. According to another embodiment of the present invention, a light-emitting and light-receiving apparatus including a light-receiving device with high light sensitivity.

According to another embodiment of the present invention, a light-emitting device, a light-emitting apparatus, a light-emitting and light-receiving apparatus, an electronic appliance, and a lighting device each with low power consumption can be provided. According to another embodiment of the present invention, a novel light-emitting device, a novel organic compound, a novel light-emitting apparatus, a novel light-emitting and light-receiving apparatus, a novel electronic appliance, and a novel lighting device can each be provided.

Note that the description of these effects does not preclude the existence of other effects. Note that one embodiment of the present invention does not necessarily need to have all these effects. Note that other effects will be apparent from the description of the specification, the drawings, the claims, and the like and other effects can be derived from the description of the specification, the drawings, the claims, and the like.

### BRIEF DESCRIPTION OF THE DRAWINGS

FIG. 1A to FIG. 1F are diagrams illustrating structures of light-emitting devices according to one embodiment.

FIG. 2 is a diagram showing energy levels of a light-emitting device according to one embodiment.

FIG. 3A to FIG. 3C are diagrams illustrating light-emitting devices and light-receiving devices according to one embodiment.

FIG. 4A to FIG. 4C are diagrams illustrating light-emitting devices and light-receiving devices according to one embodiment.

FIG. 5A to FIG. 5D are diagrams illustrating light-emitting apparatuses according to one embodiment.

FIG. 6A to FIG. 6C are diagrams illustrating a method for manufacturing a light-emitting apparatus according to one embodiment.

FIG. 7A to FIG. 7C are diagrams illustrating a method for manufacturing a light-emitting apparatus according to one embodiment.

FIG. 8A to FIG. 8C are diagrams illustrating a method for manufacturing a light-emitting apparatus according to one embodiment.

FIG. 9A to FIG. 9D are diagrams illustrating a method for manufacturing a light-emitting apparatus according to one embodiment.

FIG. 10A to FIG. 10D are diagrams illustrating a method for manufacturing a light-emitting apparatus according to one embodiment.

FIG. 11A to FIG. 11C are diagrams illustrating light-emitting apparatuses according to one embodiment.

FIG. 12A to FIG. 12F are diagrams illustrating an apparatus and pixel arrangements according to one embodiment.

FIG. 13A to FIG. 13C are diagrams and a cross-sectional view illustrating pixel circuits according to one embodiment.

FIG. 14 is a diagram illustrating a light-emitting apparatus according to one embodiment.

FIG. 15A to FIG. 15E are diagrams illustrating electronic appliances according to one embodiment.

FIG. 16A to FIG. 16E are diagrams illustrating electronic appliances according to one embodiment.

FIG. 17A and FIG. 17B are diagrams illustrating electronic appliances according to one embodiment.

FIG. 18A and FIG. 18B are diagrams illustrating a lighting device according to one embodiment.

FIG. 19 is a diagram illustrating lighting devices according to one embodiment.

FIG. 20A and FIG. 20B show ¹H NMR spectra of N-(9,9-dimethyl-9H-fluoren-2-yl)-6-phenyl-benzo[b]naphtho[1,2-d]furan-8-amine.

FIG. 21A and FIG. 21B show ¹H NMR spectra of FopTPBnf.

FIG. 22 shows an absorption spectrum and an emission spectrum in a toluene solution of FopTPBnf.

FIG. 23 shows an absorption spectrum and an emission spectrum of a solid thin film of FopTPBnf.

FIG. 24A to FIG. 24D are diagrams showing the distributions of HOMO and LUMO of FopTPBnf and a comparative compound.

FIG. 25 is a diagram illustrating a structure of a light-emitting device 1.

FIG. 26 shows the luminance-current density characteristics of the light-emitting device 1 to a light-emitting device 3 and a comparative light-emitting device 4.

FIG. 27 shows the current efficiency-luminance characteristics of the light-emitting device 1 to the light-emitting device 3.

FIG. 28 shows the current-voltage characteristics of the light-emitting device 1 to the light-emitting device 3 and the comparative light-emitting device 4.

FIG. 29 shows the external quantum efficiency-luminance characteristics of the light-emitting device 1 to the light-emitting device 3.

FIG. 30 shows the emission spectra of the light-emitting device 1 to the light-emitting device 3 and the comparative light-emitting device 4.

FIG. 31 is a diagram showing luminance changes over driving time of the light-emitting device 2, the light-emitting device 3, and the comparative light-emitting device 4.

FIG. 32 shows the luminance-current density characteristics of a light-emitting device 5 to a light-emitting device 7 and a comparative light-emitting device 8.

FIG. 33 shows the current efficiency-luminance characteristics of the light-emitting device 5 to the light-emitting device 7.

FIG. 34 shows the current-voltage characteristics of the light-emitting device 5 to the light-emitting device 7 and the comparative light-emitting device 8.

FIG. 35 shows the external quantum efficiency-luminance characteristics of the light-emitting device 5 to the light-emitting device 7.

FIG. 36 shows the emission spectra of the light-emitting device 5 to the light-emitting device 7 and the comparative light-emitting device 8.

FIG. 37 is a diagram showing luminance changes over driving time of the light-emitting device 5 to the light-emitting device 7 and the comparative light-emitting device 8.

### MODE FOR CARRYING OUT THE INVENTION

Embodiments of the present invention will be described in detail below with reference to the drawings. Note that the present invention is not limited to the following description, and it will be readily appreciated by those skilled in the art that modes and details of the present invention can be modified in various ways without departing from the spirit and scope of the present invention. Therefore, the present invention should not be construed as being limited to the description in the following embodiments.

### Embodiment 1

In this embodiment, a light-emitting device of one embodiment of the present invention and an organic compound of one embodiment of the present invention that can be used in a light-emitting device of one embodiment of the present invention will be described.

### <<Basic Structure of Light-Emitting Device>>

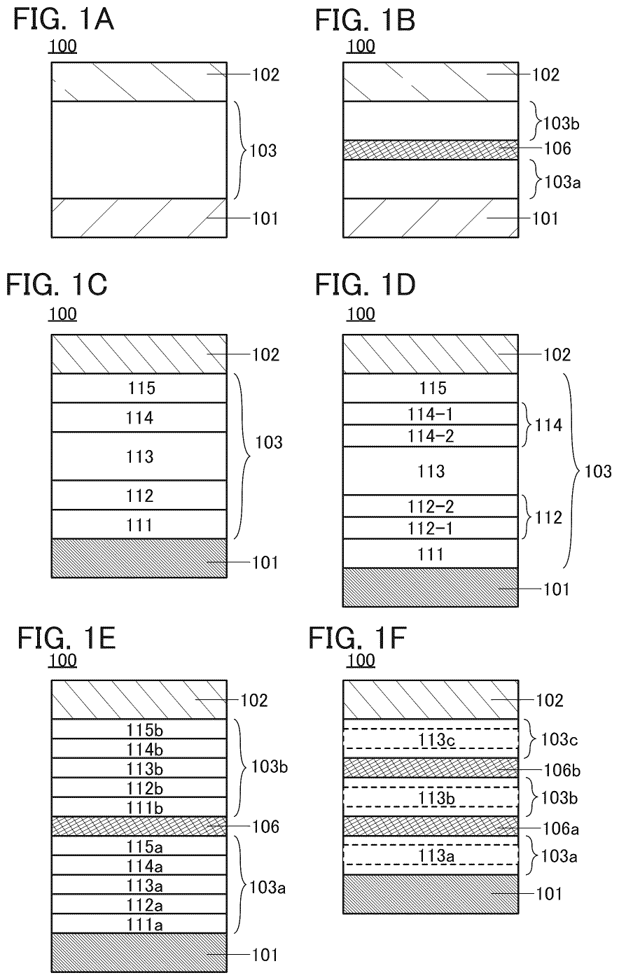

First, a basic structure of a light-emitting device of one embodiment of the present invention will be described. FIG. 1A illustrates a light-emitting device 100 including, between a pair of electrodes, an EL layer including a light-emitting layer. Specifically, the light-emitting device has a structure in which an EL layer 103 is sandwiched between a first electrode 101 and a second electrode 102. The EL layer 103 preferably contains at least a light-emitting substance and a first organic compound. Thus, a light-emitting device with low driving voltage and low power consumption can be provided. A light-emitting device with high emission efficiency can be provided. A light-emitting device with high reliability can be obtained. A light-emitting device with favorable carrier balance can be provided.

FIG. 1B illustrates the light-emitting device 100 with a stacked-layer structure (tandem structure) in which a plurality of (two layers, in FIG. 1B) EL layers (103a and 103b) are provided between a pair of electrodes and a charge-generation layer 106 is provided between the EL layers. With a light-emitting device having a tandem structure, a light-emitting apparatus with high efficiency can be achieved without changing the current amount. Each of the EL layers (103a and 103b) contains at least the light-emitting substance and the first organic compound. Note that at least one of the EL layer 103a and the EL layer 103b preferably contains the first organic compound. Thus, a tandem light-emitting device with low driving voltage and low power consumption can be provided. A tandem light-emitting device with high emission efficiency can be provided. In addition, a highly reliable light-emitting device having a tandem structure can be obtained. A tandem light-emitting device with favorable carrier balance can be provided.

FIG. 1C illustrates a stacked-layer structure of the EL layer 103 in the light-emitting device 100 of one embodiment of the present invention. Note that in this case, the first electrode 101 functions as an anode and the second electrode 102 functions as a cathode. The EL layer 103 has a structure in which a hole-injection layer 111, a hole-transport layer 112, a light-emitting layer 113, an electron-transport layer 114, and an electron-injection layer 115 are stacked in this order over the first electrode 101. Note that in the EL layer 103, it can be said that the light-emitting layer 113 is positioned between the first electrode 101 and the second electrode 102, the hole-transport layer 112 is positioned between the first electrode 101 and the light-emitting layer 113, the electron-transport layer 114 is positioned between the light-emitting layer 113 and the second electrode 102, the hole-injection layer 111 is positioned between the first electrode 101 and the hole-transport layer 112, and the electron-injection layer 115 is positioned between the electron-transport layer 114 and the second electrode 102. In the case where the EL layer 103 has such a stacked-layer structure, one or more of the hole-injection layer 111, the hole-transport layer 112, and the light-emitting layer 113 preferably contain the first organic compound. It is further preferable that one or more of the hole-injection layer 111 and the hole-transport layer 112 contain the first organic compound. Thus, a light-emitting device with low driving voltage and low power consumption can be provided. A light-emitting device with high emission efficiency can be provided. A light-emitting device with high reliability can be provided.

FIG. 1D illustrates a modification example of the stacked-layer structure illustrated in FIG. 1C. Also in this case, the first electrode 101 functions as an anode and the second electrode 102 functions as a cathode. In this modification example, the hole-transport layer 112 and the electron-transport layer 114 each have a stacked-layer structure of two layers. That is, the EL layer 103 has a structure in which the hole-injection layer 111, a first hole-transport layer 112-1, a second hole-transport layer 112-2, the light-emitting layer 113, a second electron-transport layer 114-2, a first electron-transport layer 114-1, and the electron-injection layer 115 are stacked in this order over the first electrode 101. Note that the light-emitting layer 113 is positioned between the first electrode 101 and the second electrode 102. The first hole-transport layer 112-1 is positioned between the first electrode 101 and the light-emitting layer 113. The first electron-transport layer 114-1 is positioned between the light-emitting layer 113 and the second electrode 102. The hole-injection layer 111 is positioned between the first electrode 101 and the hole-transport layer 112. The electron-injection layer 115 is positioned between the electron-transport layer 114 and the second electrode 102. The second hole-transport layer 112-2 is positioned between the first hole-transport layer and the light-emitting layer 113. It can be said that the second electron-transport layer 114-2 is positioned between the light-emitting layer 113 and the first electron-transport layer 114-1. In the case where the EL layer 103 has such a stacked-layer structure, one or more of the hole-injection layer 111, the hole-transport layer 112, and the light-emitting layer 113 preferably contain the first organic compound. In the stacked-layer structure of the hole-transport layer 112, it is preferable that the first hole-transport layer 112-1 contain the first organic compound. Accordingly, a light-emitting device with low driving voltage and low power consumption can be provided. A light-emitting device with high emission efficiency can be provided. A light-emitting device with high reliability can be provided.

The second hole-transport layer 112-2 is provided to prevent passing of electrons from the light-emitting layer 113 to the first electrode 101 side, for example. Thus, the second hole-transport layer 112-2 can also be referred to as an electron-blocking layer. The second electron-transport layer 114-2 is provided to prevent passing of holes from the light-emitting layer 113 to the second electrode 102 side, for example. Thus, the second electron-transport layer 114-2 can also be referred to as a hole-blocking layer.

The light-emitting device 100 illustrated in FIG. 1E is a light-emitting device having a tandem structure. Owing to a microcavity structure of the light-emitting device, light (monochromatic light) with different wavelengths from the EL layers (103a and 103b) can be extracted. Thus, side-by-side patterning for obtaining different emission colors (e.g., RGB) is not necessary. Therefore, higher resolution can be easily achieved. In addition, a combination with coloring layers (color filters) is also possible. Furthermore, the emission intensity of light with a specific wavelength in the front direction can be increased, whereby power consumption can be reduced. The EL layer 103a has a structure in which a hole-injection layer 111a, a hole-transport layer 112a, a light-emitting layer 113a, an electron-transport layer 114a, and an electron-injection layer 115a are stacked in this order, and the EL layer 103b has a structure in which a hole-injection layer 111b, a hole-transport layer 112b, a light-emitting layer 113b, an electron-transport layer 114b, and an electron-injection layer 115b are stacked in this order. In the case of a light-emitting device having a tandem structure in which the EL layers (103a and 103b) each have such a stacked-layer structure, any one or more of the hole-injection layers (111a and 111b), the hole-transport layers (112a and 112b), and the light-emitting layers (113a and 113b) contain the first organic compound. It is further preferable that one or more of the hole-injection layers (111a and 111b) and the hole-transport layers (112a and 112b) contain the first organic compound. Thus, a light-emitting device with low driving voltage and low power consumption can be provided. A light-emitting device with high emission efficiency can be provided. A light-emitting device with high reliability can be provided. Moreover, a change in voltage with respect to the initial voltage in the driving test can be inhibited.

The light-emitting device 100 illustrated in FIG. 1F is an example of the light-emitting device with the tandem structure illustrated in FIG. 1B, and includes three EL layers (103a, 103b, and 103c) stacked with charge-generation layers (106a and 106b) therebetween, as illustrated in the drawing. Note that the three EL layers (103a, 103b, and 103c) include respective light-emitting layers (113a, 113b, and 113c) and the emission colors of the respective light-emitting layers can be combined freely. For example, the light-emitting layer 113a can emit blue light, the light-emitting layer 113b can emit red, green, or yellow light, and the light-emitting layer 113c can emit blue light; for another example, the light-emitting layer 113a can emit red light, the light-emitting layer 113b can emit blue, green, or yellow light, and the light-emitting layer 113c can emit red light. Each of the EL layers (103a, 103b, and 103c) contains at least the light-emitting substance and the first organic compound. Note that at least one of the EL layers (103a, 103b, and 103c) contains the first organic compound.

Next, in the light-emitting device 100, the hole-injection layers (111, 111a, and 111b), the hole-transport layers (112, 112a, and 112b), and the light-emitting layers (113, 113a, 113b, and 113c) for which the first organic compound can be used are described.

The hole-injection layers (111, 111a, and 111b) are layers which inject holes from the first electrode 101 serving as the anode and the charge-generation layers (106, 106a, and 106b) to the EL layers (103, 103a, and 103b) and contain an organic acceptor material (electron-accepting material) or a material having a high hole-injection property. As the material having a high hole-injection property, a mixed material containing an organic acceptor material and a hole-transport material can also be used. For example, the first organic compound can be used as the hole-transport material in the hole-injection layers (111, 111a, and 111b). When the first organic compound having a high hole-transport property is used for the hole-injection layers (111, 111a, and 111b), a light-emitting device with low driving voltage and low power consumption can be provided. A light-emitting device with high emission efficiency can be provided. A light-emitting device with high reliability can be provided.

The hole-transport layers (112, 112a, and 112b) are each a layer that transports the holes, which are injected from the first electrode 101 by the hole-injection layers (111, 111a, and 111b), to the light-emitting layers (113, 113a, and 113b). Note that the hole-transport layers (112, 112a, and 112b) are each a layer containing a hole-transport material. Thus, for the hole-transport layers (112, 112a, and 112b), a hole-transport material that can be used for the hole-injection layers (111, 111a, and 111b) can be used. For example, the first organic compound can be used as the hole-transport material in the hole-transport layers (112, 112a, and 112b). When the first organic compound having a high hole-transport property is used for the hole-transport layers (112, 112a, and 112b), a light-emitting device with low driving voltage and low power consumption can be provided. A light-emitting device with high emission efficiency can be provided. A light-emitting device with high reliability can be provided.

It is further preferable that the first organic compound be used for both the hole-injection layers (111, 111a, and 111b) and the hole-transport layers (112, 112a, and 112b). Such a structure reduces a difference between the HOMO level of the hole-injection layers (111, 111a, and 111b) and the HOMO level of the hole-transport layers (112, 112a, and 112b). Thus, holes are injected smoothly from the hole-injection layers (111, 111a, and 111b) to the hole-transport layers (112, 112a, and 112b), whereby the driving voltage of the light-emitting device can be further reduced. Accordingly, a light-emitting device with low power consumption can be provided. A light-emitting device with high emission efficiency can be provided. A light-emitting device with high reliability can be provided.

The light-emitting layers (113, 113a, 113b, and 113c) each contain a light-emitting substance (guest material) and another or other substances (host material and the like) in appropriate combination, so that fluorescence or phosphorescence of a desired emission color can be obtained.

Examples of the organic compound used as the host material include an organic compound that satisfies requirements for the host material used for the light-emitting layer, such as a hole-transport material and an electron-transport material. The first organic compound may be used as the host material in the light-emitting layer 113. Thus, a light-emitting device with favorable carrier balance can be provided.

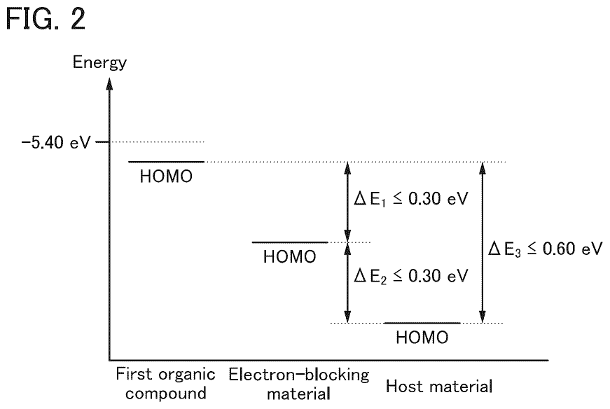

When the first organic compound is used for one or both of the hole-injection layers (111, 111a, and 111b) and the hole-transport layers (112, 112a, and 112b) and the highest occupied molecular orbital (HOMO) level of the first organic compound is high, the driving voltage of the light-emitting device easily increases. Thus, the HOMO level of the first organic compound is preferably lower than or equal to −5.40 eV (see FIG. 2). This enables smooth hole transport from the layer containing the first organic compound to the light-emitting layer. Accordingly, the driving voltage of a light-emitting device can be reduced and the emission efficiency can be improved.

In the case where the first organic compound is used for one or both of the hole-injection layers (111, 111a, and 111b) and the hole-transport layers (112, 112a, and 112b), the HOMO level of the first organic compound is preferably higher than the HOMO level of the host material in the light-emitting layer 113 (see FIG. 2). In the same case, a difference between the HOMO level of the first organic compound and the HOMO level of the host material contained in the light-emitting layer 113 (hereinafter, simply referred to as a host material) is preferably less than or equal to 0.60 eV. This reduces a barrier due to a step in the HOMO level between the layers, which enables smooth hole transport to the light-emitting layer. Accordingly, the driving voltage of the light-emitting device can be reduced, and the emission efficiency of the light-emitting device can be improved. In the same case, the host material is assumed to be an organic compound different from the first organic compound.

In the case where the hole-transport layer 112 illustrated in FIG. 1D has a stacked-layer structure of the first hole-transport layer 112-1 and the second hole-transport layer 112-2, and the first organic compound is used for one or both of the hole-injection layer 111 and the first hole-transport layer 112-1, the HOMO level of the first organic compound is preferably higher than the HOMO level of a material used for the second hole-transport layer 112-2 (hereinafter referred to as an electron-blocking material), and the HOMO level of the electron-blocking material is preferably higher than the HOMO level of the host material (see FIG. 2). In the same case, a difference (ΔE₁) between the HOMO level of the first organic compound and the HOMO level of the electron-blocking material is preferably less than or equal to 0.30 eV, further preferably less than or equal to 0.10 eV. In the same case, a difference (ΔE₂) between the HOMO level of the electron-blocking material and the HOMO level of the host material is preferably less than or equal to 0.30 eV. In the same case, a difference (ΔE₃) between the HOMO level of the first organic compound and the HOMO level of the host material is preferably less than or equal to 0.60 eV. In the same case, the HOMO level of the electron-blocking material is preferably lower than or equal to −5.50 eV, and the thickness of the second hole-transport layer 112-2 is preferably less than or equal to 200 nm in terms of driving voltage. With the above structure, a barrier due to a step in the HOMO level between the layers is reduced, so that hole transport to the light-emitting layer can be smooth. Accordingly, the driving voltage of the light-emitting device can be reduced, and the light-emitting device with low power consumption can be provided. Since the emission efficiency can be improved, a light-emitting device with high reliability can be obtained. Moreover, a change in voltage with respect to the initial voltage in the driving test can be inhibited.

Note that the values of a HOMO level and a LUMO (lowest unoccupied molecular orbital) level used in this specification can be obtained by electrochemical measurement. Typical examples of the electrochemical measurement include cyclic voltammetry (CV) measurement and differential pulse voltammetry (DPV) measurement.

In the cyclic voltammetry (CV) measurement, the values (E) of HOMO and LUMO levels can be calculated on the basis of an oxidation peak potential (Eₚₐ) and a reduction peak potential (E_{pc}), which are obtained by changing the potential of a working electrode with respect to a reference electrode. In the measurement, a HOMO level and a LUMO level are obtained by potential scanning in positive direction and potential scanning in negative direction, respectively. The scanning speed in the measurement is 0.1 V/s.

Specific calculation steps of a HOMO level and a LUMO level are described. A standard oxidation-reduction potential (Eₒ) (=(Eₚₐ+E_{pc})/2) is calculated from an oxidation peak potential (Eₚₐ) and a reduction peak potential (E_{pc}), which are obtained by the cyclic voltammogram of a material. Then, the standard oxidation-reduction potential (Eₒ) is subtracted from the potential energy (Eₓ) of the reference electrode with respect to a vacuum level, whereby the value (E) (=Eₓ−Eₒ) of each of a HOMO level and a LUMO level can be obtained.

Note that the reversible oxidation-reduction wave is obtained in the above case; in the case where an irreversible oxidation-reduction wave is obtained, the HOMO level is calculated as follows: a value obtained by subtracting a predetermined value (0.1 eV) from an oxidation peak potential (Eₚₐ) is assumed to be a reduction peak potential (E_{pc}), and a standard oxidation-reduction potential (Eₒ) is calculated to one decimal place. The LUMO level is calculated as follows: a value obtained by adding a predetermined value (0.1 eV) to a reduction peak potential (E_{pc}) is assumed to be an oxidation peak potential (Eₚₐ), and a standard oxidation-reduction potential (Eₒ) is calculated to one decimal place.

With the above structure, a light-emitting device with low driving voltage and low power consumption can be provided. A light-emitting device with high emission efficiency can be provided. A light-emitting device with high reliability can be obtained.

With the use of the light-emitting device of one embodiment of the present invention, a light-emitting apparatus or a light-emitting and light-receiving apparatus can be provided. Note that the light-emitting and light-receiving apparatus of one embodiment of the present invention using the first organic compound will be described in detail in a later embodiment.

Next, the first organic compound, which is an organic compound of one embodiment of the present invention that can be used in the light-emitting device of one embodiment of the present invention, will be described in detail.

The first organic compound is an amine compound containing a substituted or unsubstituted benzo[b]naphtho[1,2-d]furanyl group or a substituted or unsubstituted benzo[b]naphtho[1,2-d]thiophenyl group, a substituted or unsubstituted first aryl group, and a substituted or unsubstituted second aryl group.

In such an amine compound, part of the HOMO is distributed in a benzene skeleton, which is directly bonded to a nitrogen atom, of the benzo[b]naphtho[1,2-d]furan skeleton or the benzo[b]naphtho[1,2-d]thiophene skeleton; however, the electron density of the benzene skeleton can be reduced owing to the influence of an oxygen atom in the benzo[b]naphtho[1,2-d]furan skeleton or a sulfur atom in the benzo[b]naphtho[1,2-d]thiophene skeleton. Accordingly, the HOMO level of the amine compound can be reduced.

In such an amine compound, the LUMO is likely to be distributed in the benzo[b]naphtho[1,2-d]furan skeleton or the benzo[b]naphtho[1,2-d]thiophene skeleton, and is less likely to be distributed in a nitrogen atom at the center of the amine compound, the first aryl group, and the second aryl group. This is because a benzo[b]naphtho[1,2-d]furan skeleton and a benzo[b]naphtho[1,2-d]thiophene skeleton have a naphthalene structure in the skeleton and thus have a property of easily spreading conjugation and accepting electrons. When a benzo[b]naphtho[1,2-d]furan skeleton or a benzo[b]naphtho[1,2-d]thiophene skeleton is introduced into an amine compound, an amine compound highly resistant to repeated reduction can be obtained. Thus, with the use of such a first organic compound, a highly reliable light-emitting device can be provided.

In a light-emitting device with the conventional structure, a hole-injection layer, a hole-transport layer, and the like might deteriorate because of passing electrons from the light-emitting layer to the anode side. This is because the hole-transport material used for these layers has low resistance to repeated reduction.

By contrast, the first organic compound has high resistance to repeated reduction. Thus, when the first organic compound is used as one or both of the hole-injection layer 111 and the first hole-transport layer 112-1, the layer is less likely to deteriorate even when electrons pass from the light-emitting layer to the second electrode 102 (anode) side. Thus, with the use of the first organic compound, a highly reliable light-emitting device can be obtained. Note that as illustrated in FIG. 1D, a light-emitting device including the second hole-transport layer 112-2, which is provided in order to prevent electrons from passing from the light-emitting layer 113 to the first electrode 101 side, is preferable because deterioration of the hole-injection layer 111 and the first hole-transport layer 112-1 can be inhibited more effectively.

The first organic compound is further preferably an amine compound containing the substituted or unsubstituted benzo[b]naphtho[1,2-d]furanyl group, the substituted or unsubstituted first aryl group, and the substituted or unsubstituted second aryl group. When the benzo[b]naphtho[1,2-d]furanyl group is introduced, the refractive index of the organic compound can be lowered as compared with the case where a benzo[b]naphtho[1,2-d]thiophenyl group is introduced. An organic compound with a lower refractive index is preferably used because the light extraction efficiency of the light-emitting device can be increased.

Note that in this specification and the like, benzo[b]naphtho[1,2-d]furan is simply referred to as benzonaphthofuran in some cases. In this specification and the like, a benzo[b]naphtho[1,2-d]furanyl group, which is a substituent made by removing one hydrogen from benzo[b]naphtho[1,2-d]furan, is referred to as a benzonaphthofuranyl group in some cases.

Note that in this specification and the like, benzo[b]naphtho[1,2-d]thiophene is sometimes simply referred to as benzonaphthothiophene. In this specification and the like, a benzo[b]naphtho[1,2-d]thiophenyl group, which is a substituent made by removing one hydrogen from benzo[b]naphtho[1,2-d]thiophene, is referred to as a benzonaphthothiophenyl group in some cases.

Note that examples of the first aryl group and the second aryl group that can be used in the first organic compound include a substituted or unsubstituted aromatic hydrocarbon group having 6 to 60 carbon atoms. Specific examples of the aromatic hydrocarbon group having 6 to 60 carbon atoms will be described later in this embodiment.

Specific examples of the first organic compound are described below using General Formulae (G10), (G11), and (G1) to (G5).

Specific examples of the first organic compound include an organic compound represented by General Formula (G10) below.

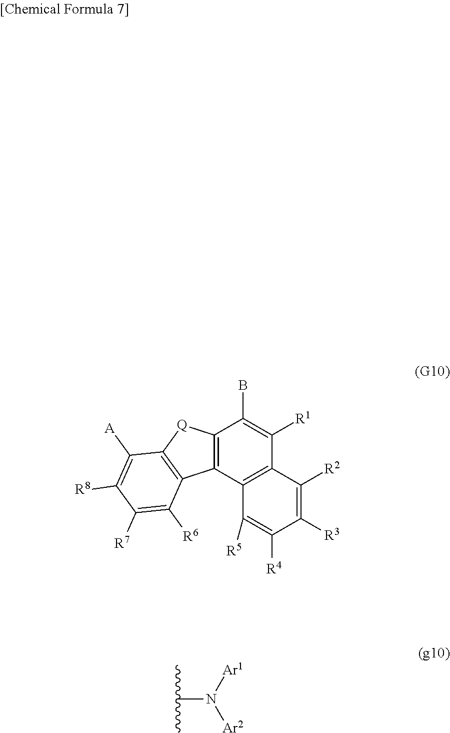

Note that in General Formula (G10) above, Q represents an oxygen atom or a sulfur atom. In addition, one of A and B is a group represented by General Formula (g10) above, and the other represents any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted aromatic hydrocarbon group having 6 to 60 carbon atoms. R¹ to R⁸ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted aromatic hydrocarbon group having 6 to 60 carbon atoms. In addition, in Formula (g10), Ar¹ and Ar² each independently represent a substituted or unsubstituted aromatic hydrocarbon group having 6 to 60 carbon atoms.

In General Formula (G10) above, carbon at the 6-position or the 8-position of the benzonaphthofuran skeleton or the benzonaphthothiophene skeleton is bonded to a nitrogen atom, whereby an arylamine compound is formed. As described above, with the structure in which carbon at the 6-position or the 8-position of the benzonaphthofuran skeleton or the benzonaphthothiophene skeleton is bonded to a nitrogen atom, the electron density of a benzene skeleton which is directly bonded to a nitrogen atom in the benzonaphthofuran skeleton or the benzonaphthothiophene skeleton is lowered owing to the influence of an oxide atom of the benzonaphthofuran skeleton or a sulfur atom of the benzonaphthothiophene skeleton; accordingly, an effect of reducing the HOMO level of the organic compound is easily obtained. Thus, a material with a desired HOMO level corresponding to the structure of the light-emitting device of the present invention can be provided. Thus, when the first organic compound is used in the light-emitting device, holes can be injected and transported smoothly in the light-emitting device, and a change in voltage with respect to the initial voltage in the driving test can be inhibited.

Specific examples of the first organic compound include an organic compound represented by General Formula (G11) below.

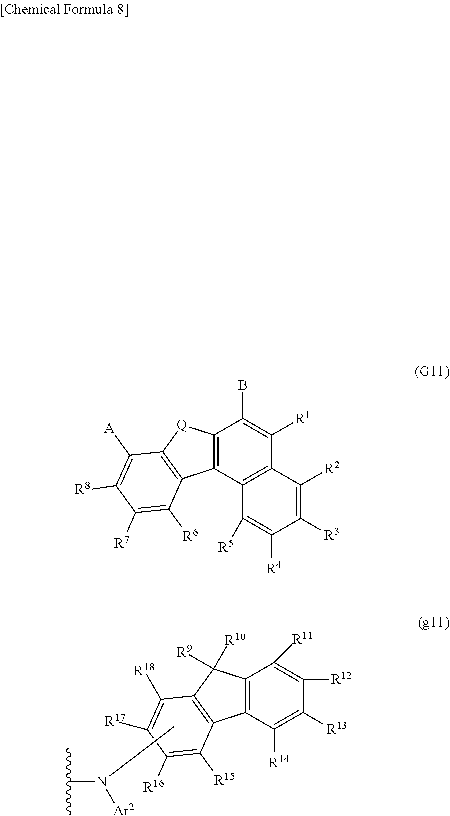

Note that in General Formula (G11) above, Q represents an oxygen atom or a sulfur atom. In addition, one of A and B is the group represented by General Formula (g11) above, and the other represents any of hydrogen, an alkyl group having 1 to 6 carbon atoms, a cyclic saturated hydrocarbon group having 3 to 6 carbon atoms, an alkoxy group having 1 to 6 carbon atoms, a cyano group, halogen, a haloalkyl group having 1 to 6 carbon atoms, and a substituted or unsubstituted aromatic hydrocarbon group having 6 to 60 carbon atoms. R¹ to R⁸ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, halogen, a haloalkyl group having 1 to 6 carbon atoms, and a substituted or unsubstituted aromatic hydrocarbon group having 6 to 60 carbon atoms. In addition, in Formula (g11), Ar² represents a substituted or unsubstituted aromatic hydrocarbon group having 6 to 60 carbon atoms, and R⁹ and R¹⁰ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, halogen, a haloalkyl group having 1 to 6 carbon atoms, and a substituted or unsubstituted phenyl group. R¹¹ to R¹⁸ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, halogen, a haloalkyl group having 1 to 6 carbon atoms, and a substituted or unsubstituted aromatic hydrocarbon group having 6 to 60 carbon atoms. Note that any one of R¹⁵ to R¹⁸ represents a bond with a nitrogen atom.

General Formula (G11) is different from General Formula (G10) above in that Ar¹ in General Formula (G10) is limited to a 9H-fluorenyl group. The introduction of the 9H-fluorenyl group can further increase the hole-transport property of the first organic compound. Furthermore, this is preferable in terms of improving the heat resistance.

Note that as an organic compound used in the light-emitting device of one embodiment of the present invention, organic compounds represented by General Formulae (G1) to (G5) below are further preferable.

Specific examples of the first organic compound include an organic compound represented by General Formula (G1) below.

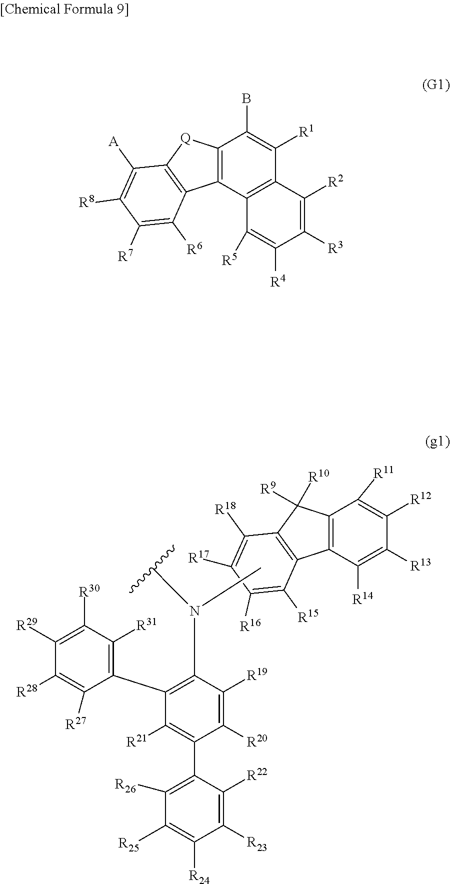

Note that in General Formula (G1) above, Q represents an oxygen atom or a sulfur atom. In addition, one of A and B is a group represented by General Formula (g1) above, and the other represents any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted aromatic hydrocarbon group having 6 to 60 carbon atoms. R¹ to R⁸ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted aromatic hydrocarbon group having 6 to 60 carbon atoms. In addition, in Formula (g1), R⁹ and R¹⁰ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted phenyl group. Note that R⁹ and R¹⁰ may be bonded to each other to form a spiro ring structure. R¹¹ to R³¹ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted aromatic hydrocarbon group having 6 to 60 carbon atoms. Note that any one of R¹⁵ to R¹⁸ represents a bond with a nitrogen atom.

General Formula (G1) is different from General Formula (G1) in that Ar² in General Formula (G11) above is limited to an m-terphenyl-4′-yl group in order to make Ar² specific. The m-terphenyl-4′-yl group has a larger steric hindrance than the p-terphenyl group and thus is effective in inhibiting intramolecular motion. The introduction of the m-terphenyl-4′-yl group can increase the glass transition point of the first organic compound. Accordingly, the heat resistance of the light-emitting device using the first organic compound can be improved, whereby the reliability can be increased.

Specific examples of the first organic compound include an organic compound represented by General Formula (G2) below.

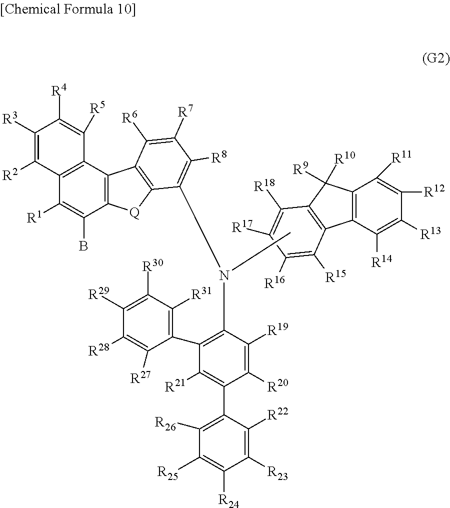

Note that in General Formula (G2) above, Q represents an oxygen atom or a sulfur atom. In addition, R¹ to R⁸, R¹¹ to R³¹, and B each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted aromatic hydrocarbon group having 6 to 60 carbon atoms. Note that any one of R¹⁵ to R¹⁸ represents a bond with a nitrogen atom. In addition, R⁹ and R¹⁰ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted phenyl group. Note that R9 and R10 may be bonded to each other to form a spiro ring structure.

General Formula (G2) above is different from General Formula (G1) above in that the bonding position between a nitrogen atom and a benzonaphthofuran skeleton or a benzonaphthothiophene skeleton is limited to the 8-position of the benzonaphthofuran skeleton or the benzonaphthothiophene skeleton. With such a structure in which carbon at the 8-position of the benzonaphthofuran skeleton or the benzonaphthothiophene skeleton is bonded to a nitrogen atom, a compound with a high phosphorescent level can be obtained, and high emission efficiency can be achieved when the compound is used in a phosphorescent light-emitting device.

Specific examples of the first organic compound include an organic compound represented by General Formula (G3) below.

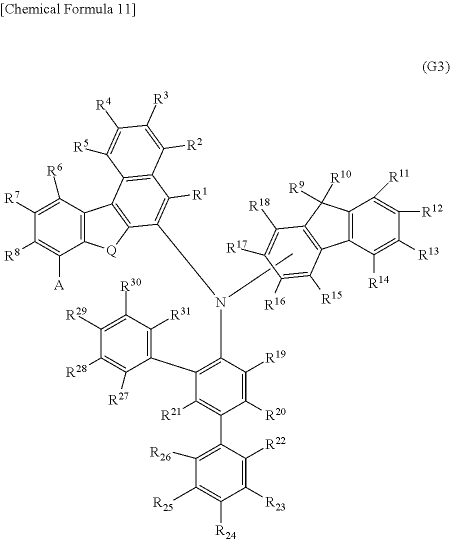

Note that in General Formula (G3) above, Q represents an oxygen atom or a sulfur atom. In addition, R¹ to R⁸, R¹¹ to R³¹, and A each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted aromatic hydrocarbon group having 6 to 60 carbon atoms. Note that any one of R¹⁵ to R¹⁸ represents a bond with a nitrogen atom. In addition, R⁹ and R¹⁰ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted phenyl group. Note that R⁹ and R¹⁰ may be bonded to each other to form a spiro ring structure.

General Formula (G2) above is different from General Formula (G1) above in that the bonding position between a nitrogen atom and a benzonaphthofuran skeleton or a benzonaphthothiophene skeleton is limited to the 6-position of the benzonaphthofuran skeleton or the benzonaphthothiophene skeleton.

Specific examples of the first organic compound include an organic compound represented by General Formula (G4) below.

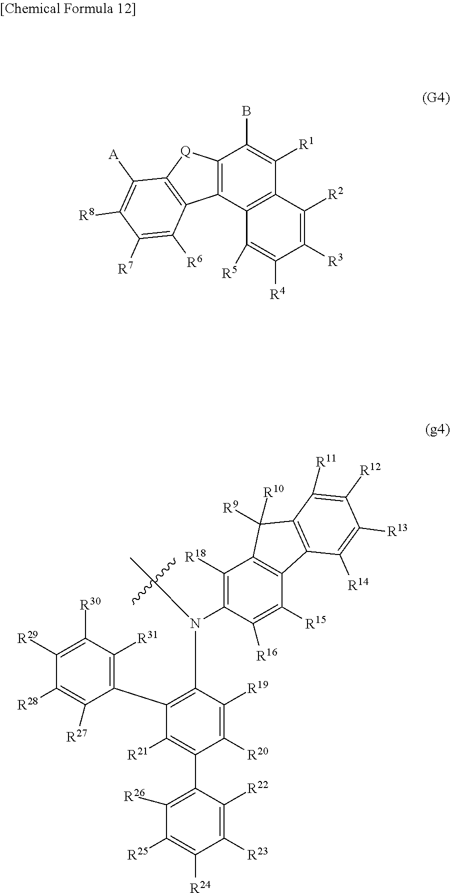

Note that in General Formula (G4) above, Q represents an oxygen atom or a sulfur atom. In addition, one of A and B is the group represented by General Formula (g4) above, and the other represents any of hydrogen, an alkyl group having 1 to 6 carbon atoms, a cyclic saturated hydrocarbon group having 3 to 6 carbon atoms, an alkoxy group having 1 to 6 carbon atoms, a cyano group, halogen, a haloalkyl group having 1 to 6 carbon atoms, and a substituted or unsubstituted aromatic hydrocarbon group having 6 to 60 carbon atoms. R¹ to R⁸ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, halogen, a haloalkyl group having 1 to 6 carbon atoms, and a substituted or unsubstituted aromatic hydrocarbon group having 6 to 60 carbon atoms. In addition, in Formula (g6), R⁹ and R¹⁰ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, halogen, a haloalkyl group having 1 to 6 carbon atoms, and a substituted or unsubstituted phenyl group, and R⁹ and R¹⁰ may be bonded to each other to form a spiro ring structure. R¹¹ to R¹⁶ and R¹⁸ to R³¹ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, halogen, a haloalkyl group having 1 to 6 carbon atoms, and a substituted or unsubstituted aromatic hydrocarbon group having 6 to 60 carbon atoms.

General Formula (G4) is different from General Formula (G1) in that the bond position between a 9H-fluorenyl group and a nitrogen atom is limited to the 2-position. When the bonding position of a 9H-fluorenyl group and a nitrogen atom is the 2-position, the HOMO level of (G4) can be adjusted to be more appropriate.

Note that the organic compound represented by General Formula (G5) below is further preferable as the organic compound used in the light-emitting device of one embodiment of the present invention.

Specific examples of the first organic compound include an organic compound represented by General Formula (G5) below.

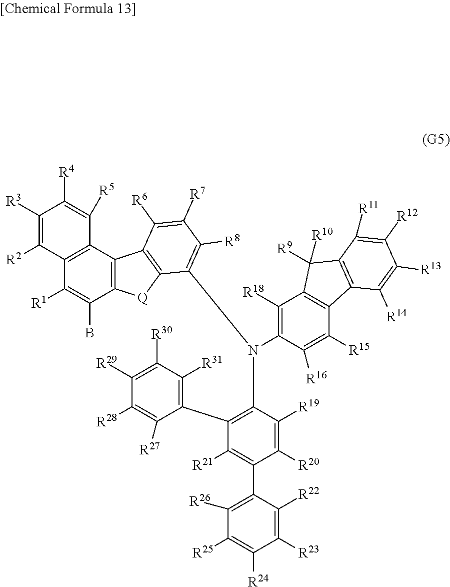

Note that in General Formula (G5) above, Q represents an oxygen atom or a sulfur atom. In addition, B, R¹ to R⁸, R¹¹ to R¹⁶, and R¹⁸ to R¹¹ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted aromatic hydrocarbon group having 6 to 60 carbon atoms. In addition, R⁹ and R¹⁰ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted phenyl group. Note that R⁹ and R¹⁰ may be bonded to each other to form a spiro ring structure.

General Formula (G5) above is different from General Formula (G4) above in that the bonding position between a nitrogen atom and a benzonaphthofuran skeleton or a benzonaphthothiophene skeleton is limited to the 8-position of the benzonaphthofuran skeleton or the benzonaphthothiophene skeleton. With such a structure in which carbon at the 8-position of the benzonaphthofuran skeleton or the benzonaphthothiophene skeleton is bonded to a nitrogen atom, a compound with a high phosphorescent level can be obtained, and high emission efficiency can be achieved when the compound is used in a phosphorescent light-emitting device.

Note that when Q is an oxygen atom in the organic compounds represented by General Formulae (G10), (G11), and (G1) to (G5) above, the organic compounds can have a lower refractive index than the compound containing a sulfur atom. When an organic compound with a lower refractive index is used, it is preferable that the light extraction efficiency of the light-emitting device can be increased.

Next, specific examples of an alkyl group having 1 to 6 carbon atoms, a cyclic saturated hydrocarbon group having 3 to 6 carbon atoms, an alkoxy group having 1 to 6 carbon atoms, halogen, a haloalkyl group having 1 to 6 carbon atoms, and an aromatic hydrocarbon group having 6 to 60 carbon atoms, which can be used in the first organic compound, will be described. Note that the group that can be used in the first organic compound is not limited to the following specific examples. Some or all of hydrogen atoms in the alkyl group having 1 to 6 carbon atoms, the cyclic saturated hydrocarbon group having 3 to 6 carbon atoms, the alkoxy group having 1 to 6 carbon atoms, the halogen, the haloalkyl group having 1 to 6 carbon atoms, and the aromatic hydrocarbon group having 6 to 60 carbon atoms may be deuterium.

### <<Specific Examples of Alkyl Group Having 1 to 6 Carbon Atoms>>

Specific examples of the alkyl group having 1 to 6 carbon atoms include a methyl group, an ethyl group, a propyl group, an isopropyl group, a butyl group, a sec-butyl group, an isobutyl group, a tert-butyl group, a pentyl group, an isopentyl group, a sec-pentyl group, a tert-pentyl group, a neopentyl group, a hexyl group, an isohexyl group, a sec-hexyl group, a tert-hexyl group, a neohexyl group, a 3-methylpentyl group, a 2-methylpentyl group, a 2-ethylbutyl group, a 1,2-dimethylbutyl group, and a 2,3-dimethylbutyl group.

### <<Specific Examples of Cyclic Saturated Hydrocarbon Group Having 3 to 6 Carbon Atoms>>

Specific examples of the cyclic saturated hydrocarbon group having 3 to 6 carbon atoms include a cyclopropyl group, a cyclobutyl group, a cyclopentyl group, and a cyclohexyl group.

### <<Specific Examples of Alkoxy Group Having 1 to 6 Carbon Atoms>>

Specific examples of the alkoxyl group having 1 to 6 carbon atoms include a methoxy group, an ethoxy group, an n-propoxy group, an isopropoxy group, an n-butoxy group, a sec-butoxy group, an isobutoxy group, a tert-butoxy group, an n-pentyloxy group, an isopentyloxy group, a sec-pentyloxy group, a tert-pentyloxy group, a neopentyloxy group, an n-hexyloxy group, an isohexyloxy group, a sec-hexyloxy group, a tert-hexyloxy group, a neohexyloxy group, and a cyclohexyloxy group.

### <<Specific Examples of Halogen>>

Specific examples of halogen include fluorine, chlorine, bromine, and iodine.

### <<Specific Examples of Haloalkyl Group Having 1 to 6 Carbon Atoms>>

Specific examples of the haloalkyl group having 1 to 6 carbon atoms include a group in which one or more of hydrogen atoms contained in the alkyl group having 1 to 6 carbon atoms are substituted with halogen described above. More specifically, a fluoromethyl group, a difluoromethyl group, a difluorochloromethyl group, a trifluoromethyl group, a chloromethyl group, a dichloromethyl group, a bromomethyl group, a 1,1-difluoroethyl group, a 2,2,2-trifluoroethyl group, a 1,1,2,2-tetrafluoroethyl group, a pentafluoroethyl group, a 3,3,3-trifluoropropyl group, a 1,1,1,3,3,3-hexafluoroisopropyl group, a 1,1,2,2,3,3,3-heptafluoropropyl group, and the like can be given.

### <<Aromatic Hydrocarbon Group Having 6 to 60 Carbon Atoms>>

Specific examples of the aromatic hydrocarbon group having 6 to 60 carbon atoms include substituents represented by Structural Formulae (Ar-1) to (Ar-21) below and the like. Note that there is no limitation on the substitution position in Structural Formulae (Ar-1) to (Ar-21) below.

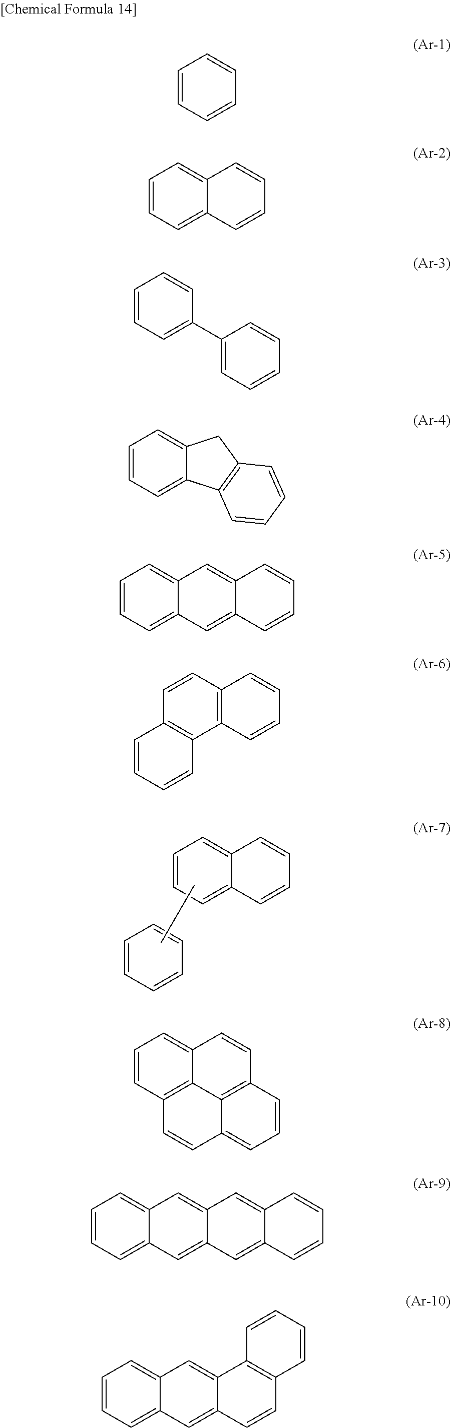

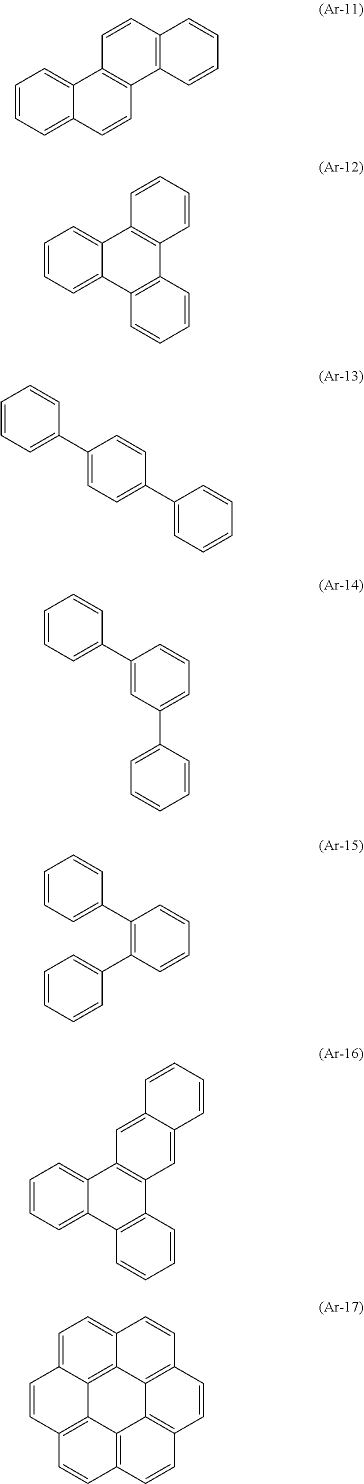

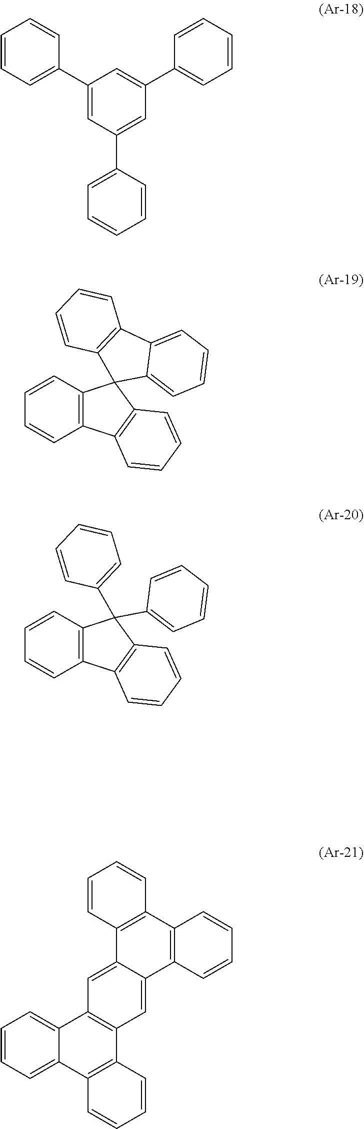

In the case where the aromatic hydrocarbon group having 6 to 60 carbon atoms further includes a substituent, the substituent can be a cyano group or the above-described alkyl group having 1 to 6 carbon atoms, cyclic saturated hydrocarbon group having 3 to 6 carbon atoms, alkoxy group having 1 to 6 carbon atoms, halogen, haloalkyl group having 1 to 6 carbon atoms, or aromatic hydrocarbon having 6 to 60 carbon atoms. The above-described specific examples can be referred to for specific examples of the alkyl group having 1 to 6 carbon atoms, the cyclic saturated hydrocarbon group having 3 to 6 carbon atoms, the alkoxy group having 1 to 6 carbon atoms, the halogen, the haloalkyl group having 1 to 6 carbon atoms, and the aromatic hydrocarbon having 6 to 60 carbon atoms.

Next, specific examples of the first organic compound are represented by structural formulae. First, specific examples of the organic compounds represented by General Formulae (G10) and (G11) are shown below.

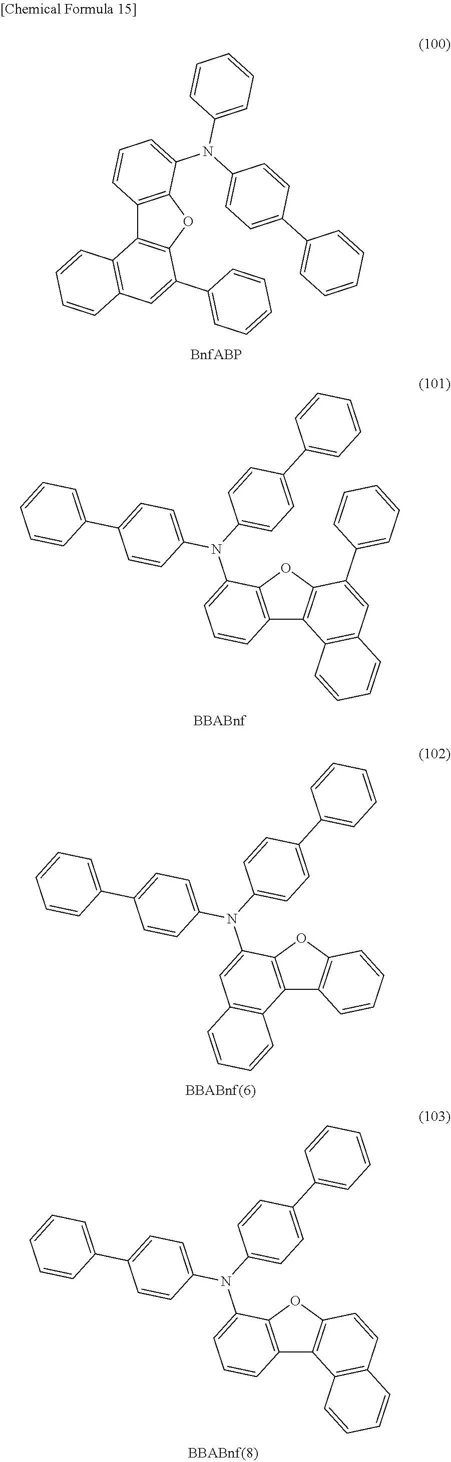

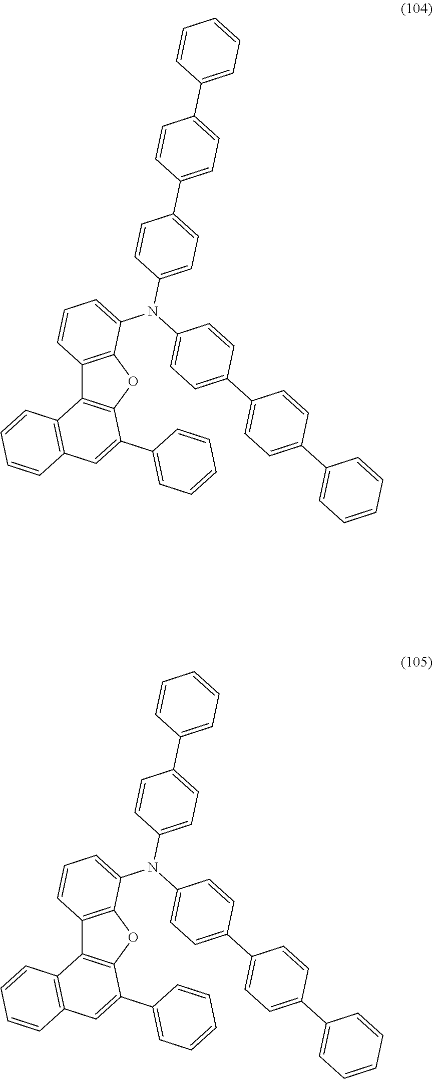

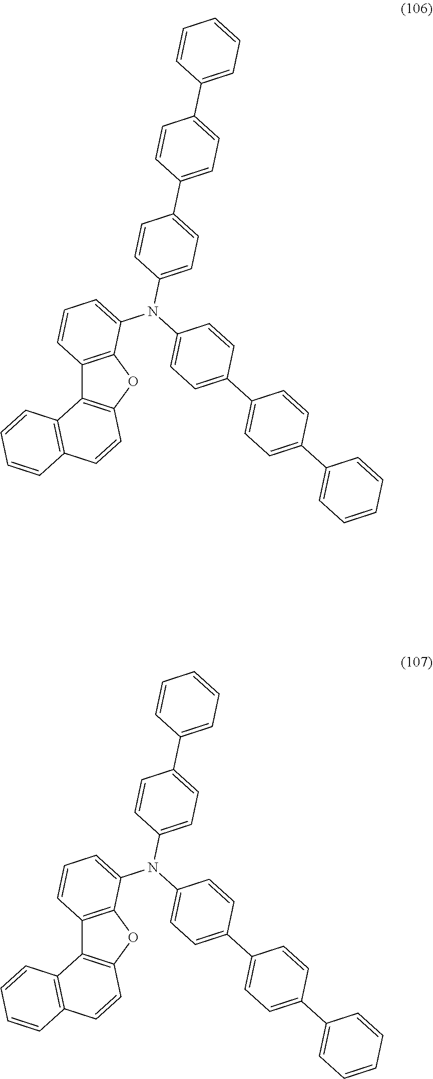

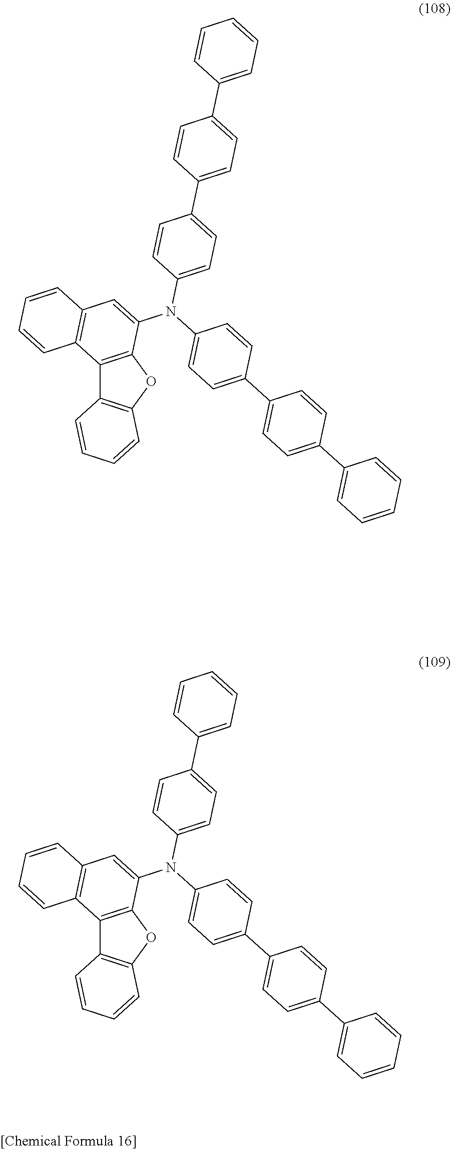

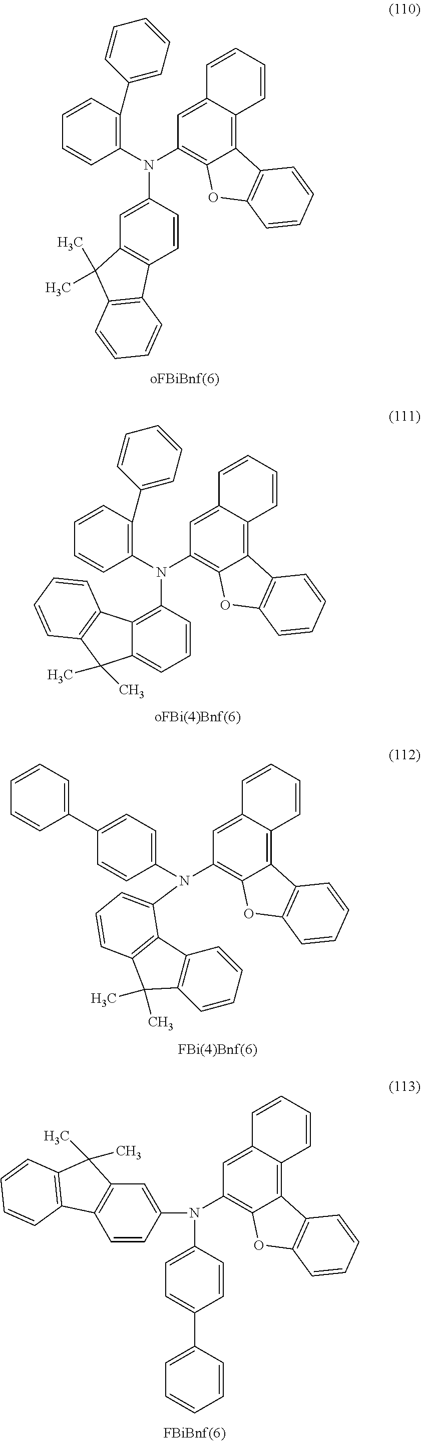

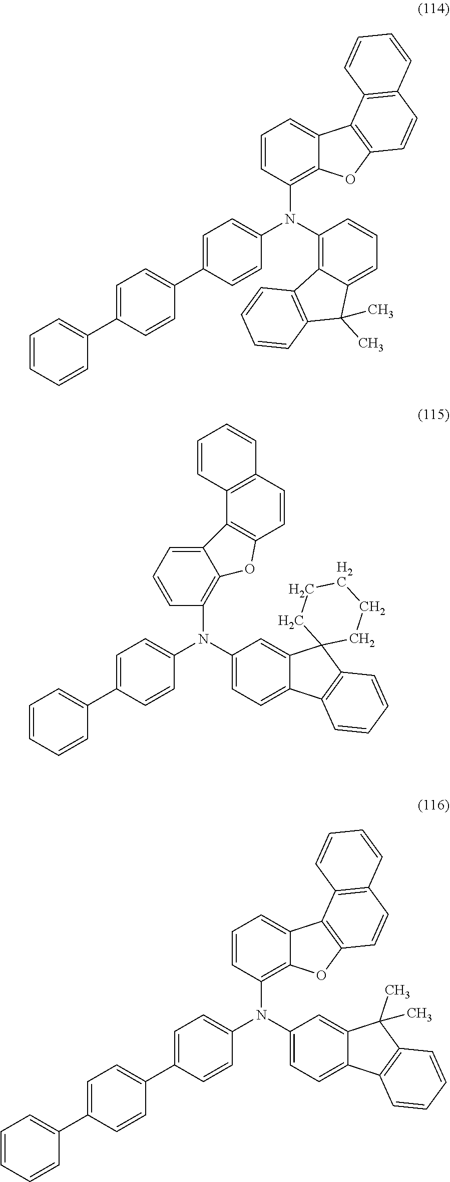

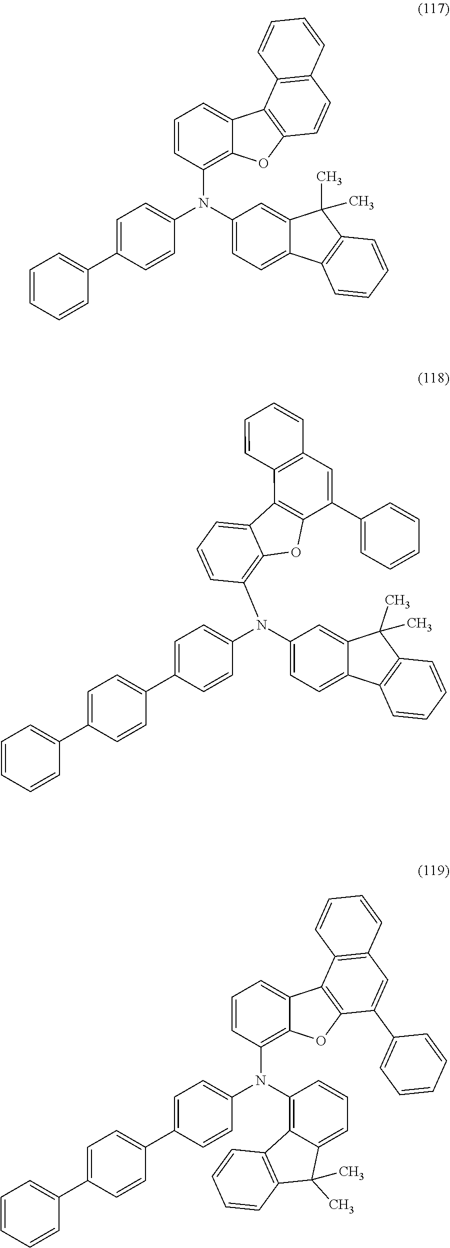

Next, specific examples of the organic compounds represented by General Formula (G1) to (G4) above are shown below. Note that since General Formulae (G1) to (G4) above are each a general formula made by specifically limiting part of structures of General Formulae (G10) and (G11) above; thus, the following specific examples can also be referred to as specific examples of the organic compounds represented by General Formulae (G10), (G11), and (G1) to (G4) above.

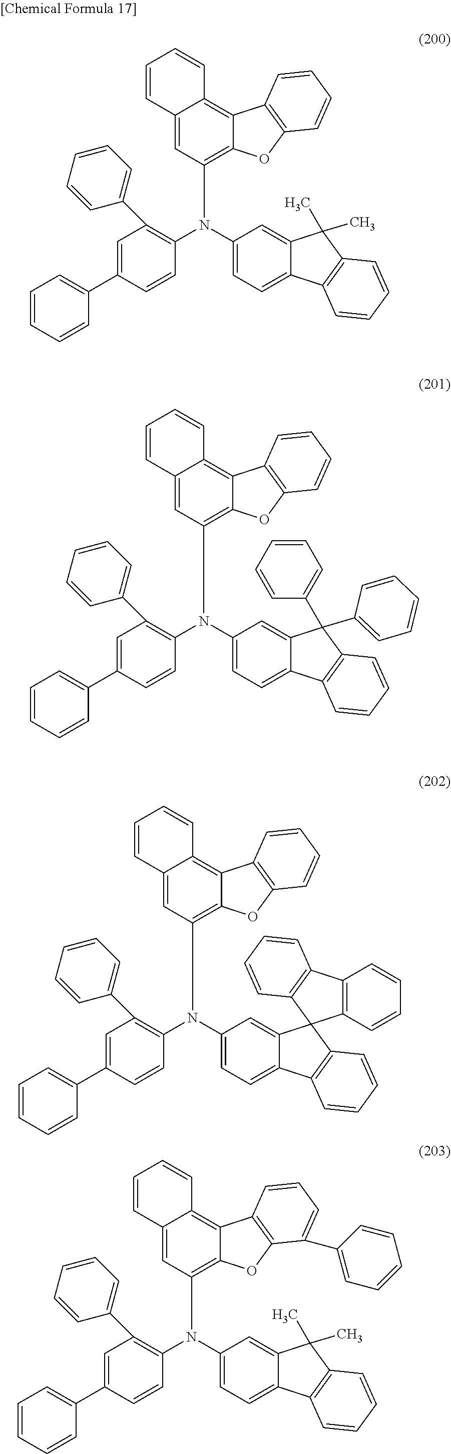

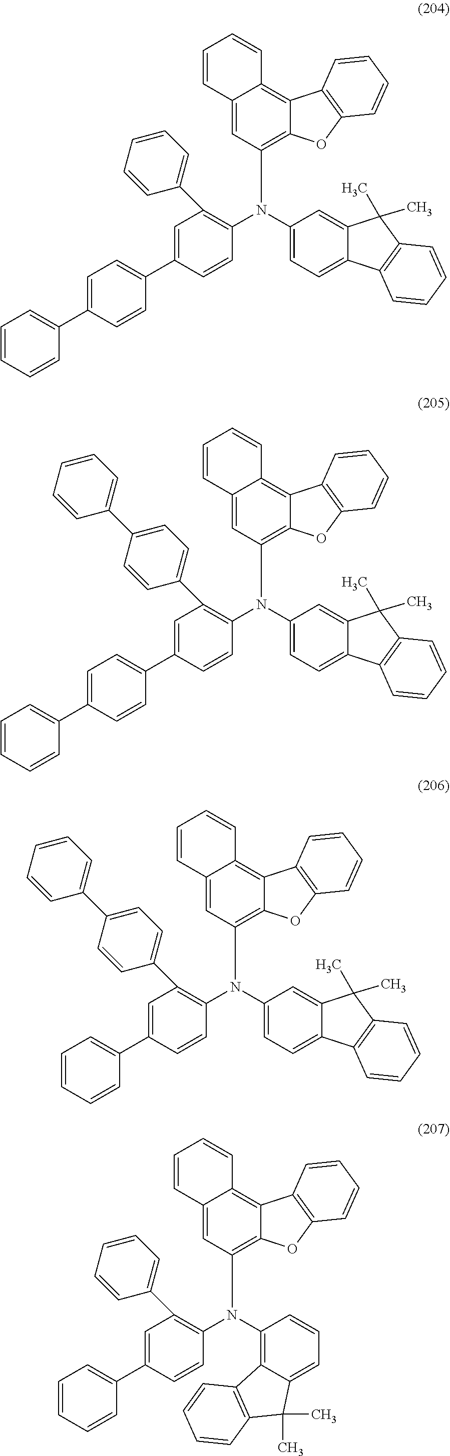

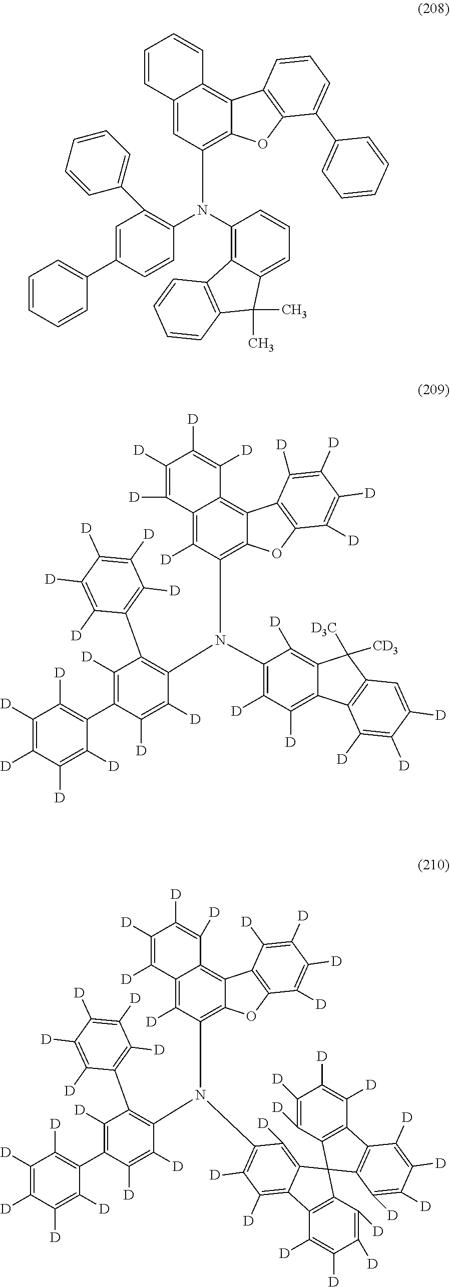

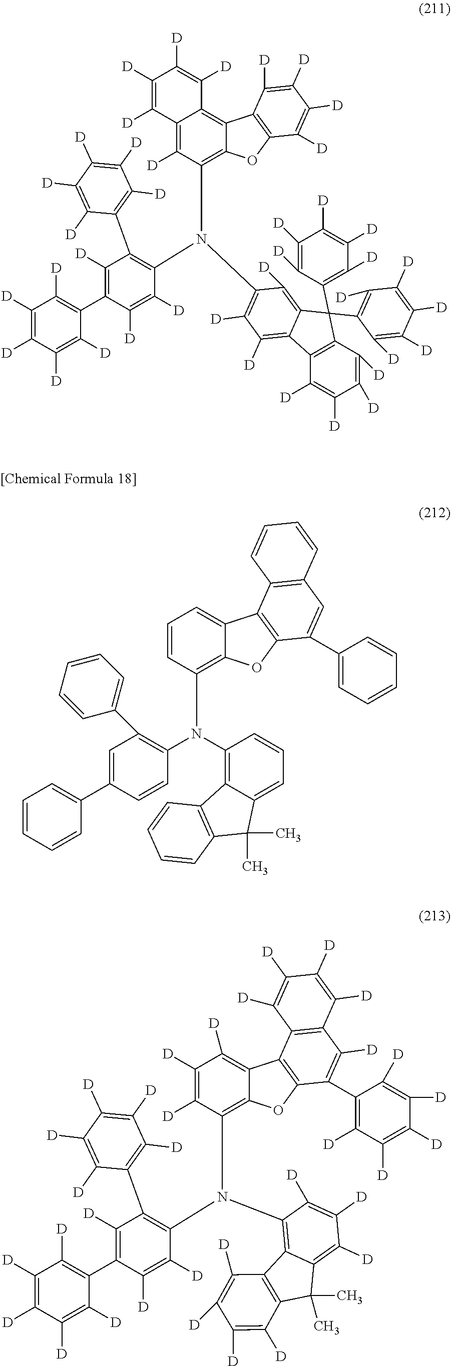

Next, specific examples of the organic compound represented by General Formula (G5) above are shown below. Note that General Formula (G5) above is a general formula made by specifically limiting part of the structures of General Formulae (G10), (G11), and (G1) to (G4) above; thus, the following specific examples can be regarded as specific examples of the organic compounds represented by General Formulae (G10), (G11), and (G1) to (G5) above.

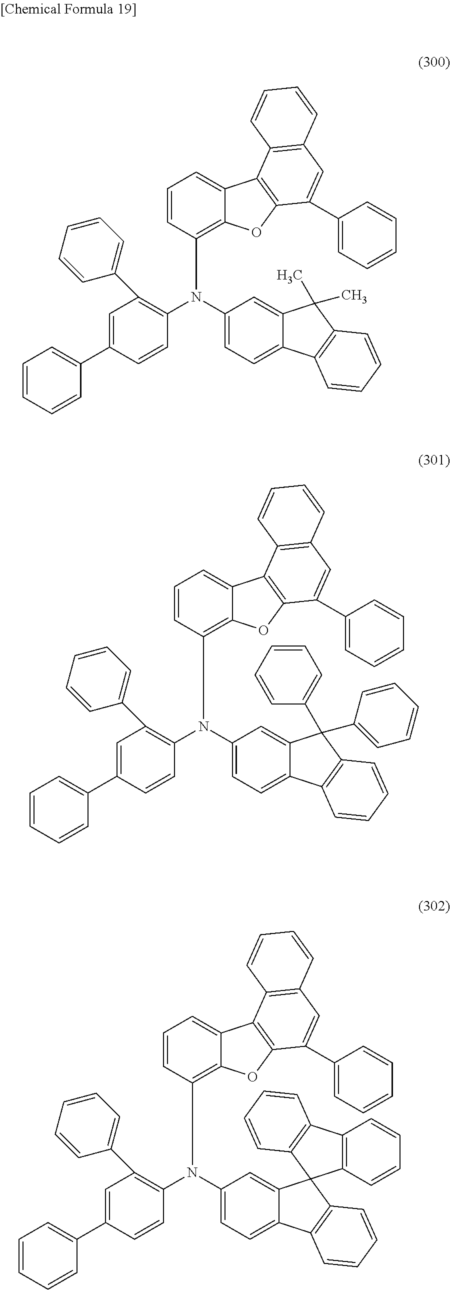

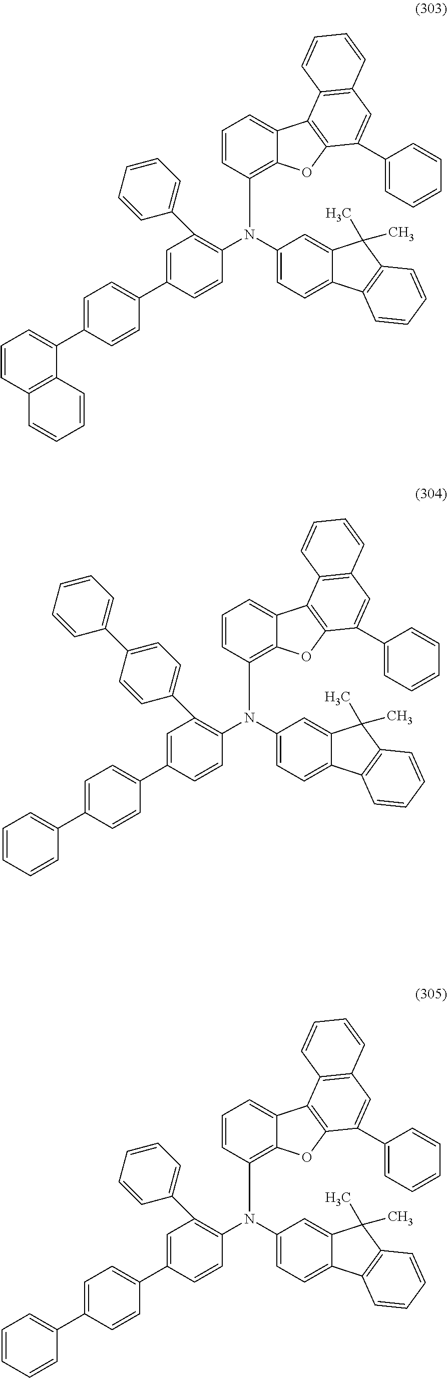

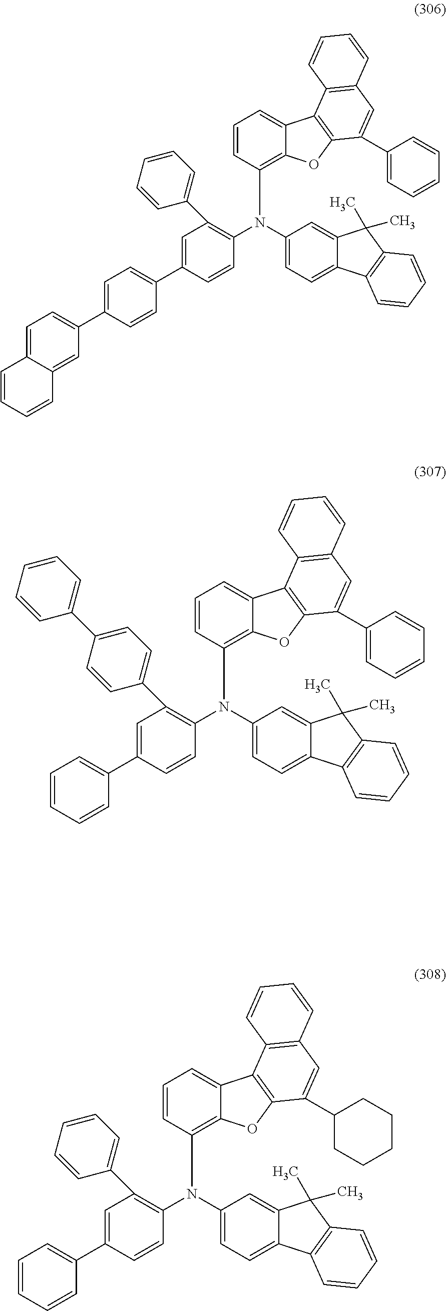

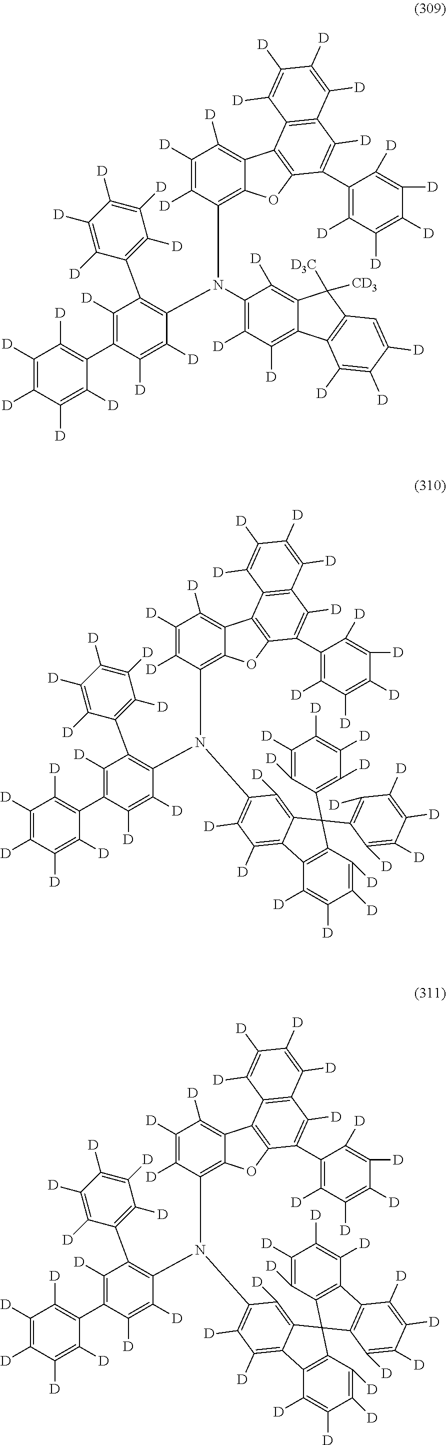

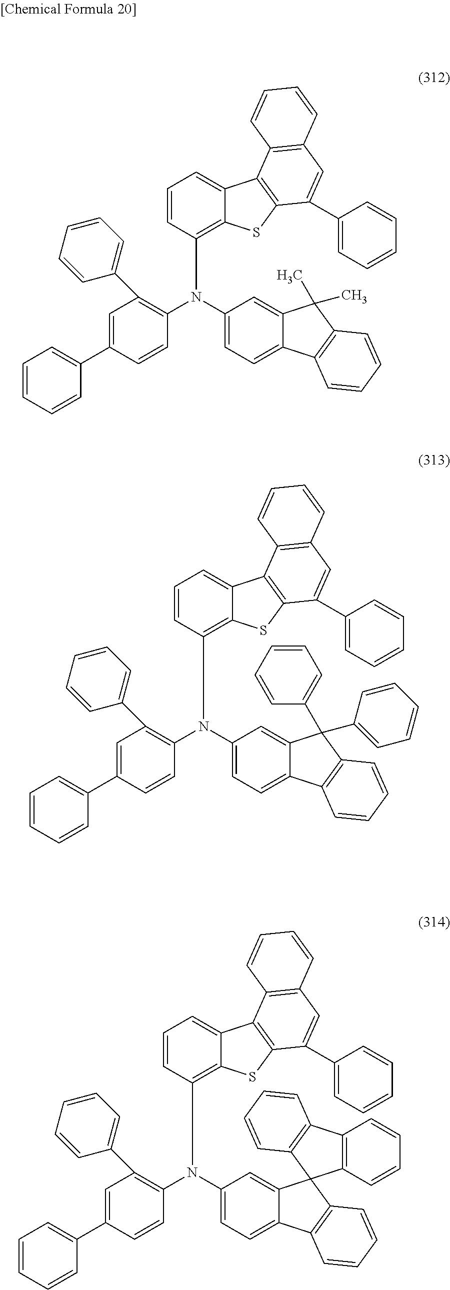

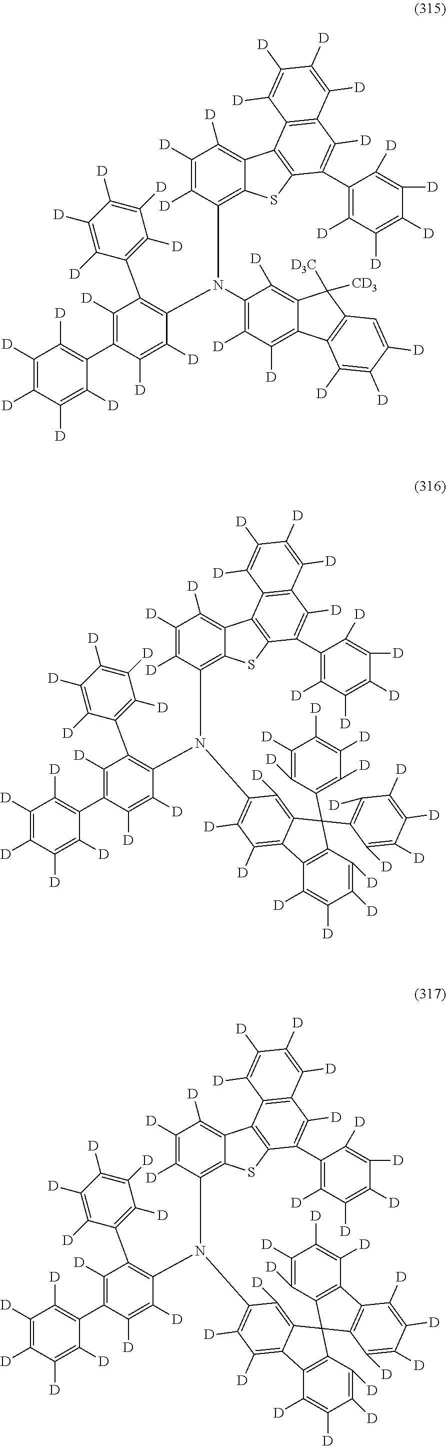

The organic compounds represented by Structural Formulae (100) to (119), (200) to (213), and (300) to (311) above are examples of the organic compounds represented by General Formulae (G10), (G11), (G1) to (G4), and (G5) above; however, the organic compounds that can be used as the first organic compounds are not limited thereto.

Next, a method for synthesizing the organic compound represented by General Formula (G5) below is described as an example of the organic compound that can be used as the first organic compound. Note that the method for synthesizing the organic compound represented by General Formula (G5) can employ a variety of reactions and is not limited to the following methods for synthesizing.

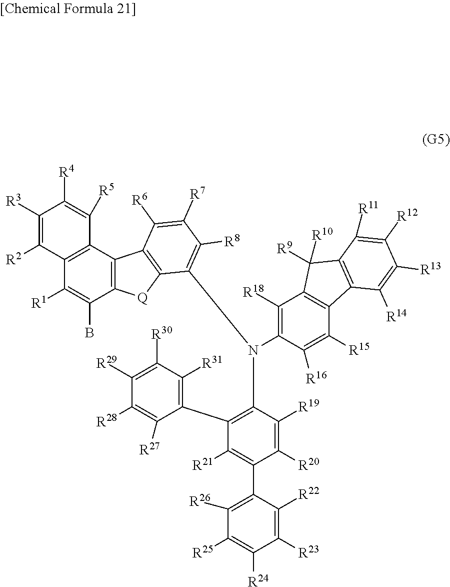

Note that in General Formula (G5) above, Q represents an oxygen atom or a sulfur atom. In addition, B, R¹ to R⁸, R¹¹ to R¹⁶, and R⁸ to R³¹ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted aromatic hydrocarbon group having 6 to 60 carbon atoms. In addition, R⁹ and R¹⁰ each independently represent any of hydrogen (including deuterium), an alkyl group having 1 to 6 carbon atoms, a cyclic saturated hydrocarbon group having 3 to 6 carbon atoms, an alkoxy group having 1 to 6 carbon atoms, a cyano group, a halogen, a haloalkyl group having 1 to 6 carbon atoms, and a substituted or unsubstituted phenyl group. Note that R⁹ and R¹⁰ may be bonded to each other to form a spiro ring structure.

### <<Method for Synthesizing Organic Compound Represented by General Formula (G5)>>

The organic compound represented by General Formula (G5) of the present invention can be synthesized by Synthesis Schemes (A-1) and (A-2) below.

First, Synthesis Scheme (A-1a), which is an example of Synthesis Scheme (A-1), is described. Specifically, an aryl compound (Compound 1) and an arylamine compound (Compound 2) are coupled, whereby an arylamine compound (Compound 3) can be obtained. Synthesis Scheme (A-1a) is shown below.

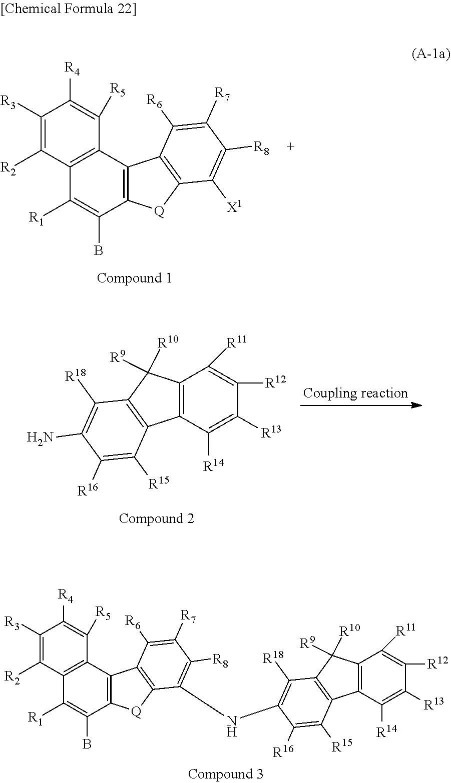

The arylamine compound (Compound 3) can also be obtained in Synthesis Scheme (A-1b), which is another example of Synthesis Scheme (A-1). That is, an arylamine compound (Compound 4) and an aryl compound (Compound 5) are coupled, whereby the arylamine compound (Compound 3) can be obtained. Synthesis Scheme (A-1b) is shown below.

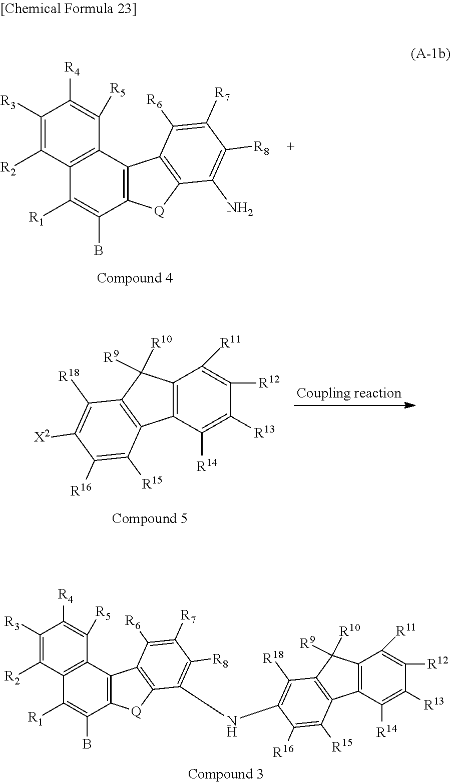

Next, Synthesis Scheme (A-2) is described. That is, the arylamine compound (Compound 3) and an aryl compound (Compound 6) are coupled, whereby an arylamine compound (G5), which is a target substance, can be obtained. Synthesis Scheme (A-2) is shown below.

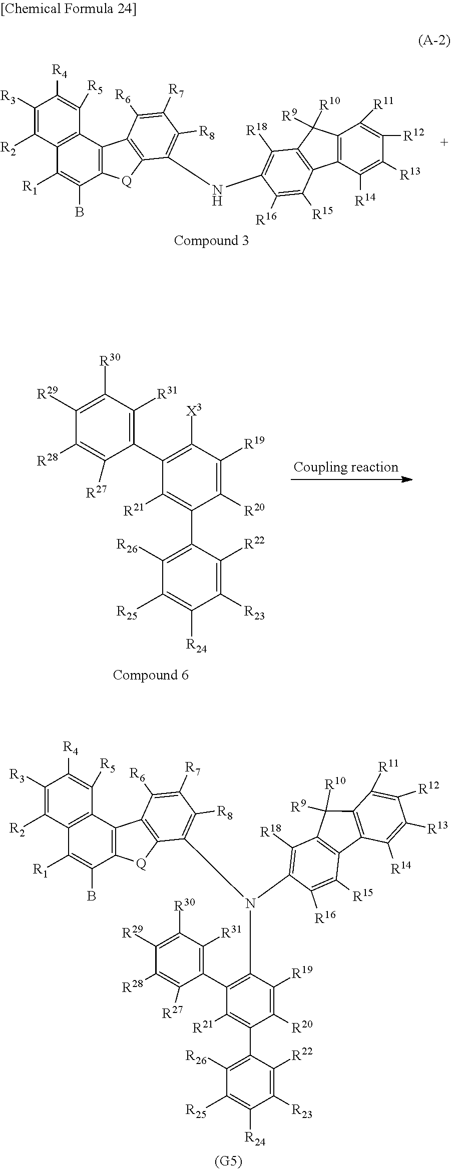

In addition, (G5), which is the target substance, can be obtained by Synthesis Scheme (A-3) and Synthesis Scheme (A-4).

First, Synthesis Scheme (A-3a), which is an example of Synthesis Scheme (A-3), is described. Specifically, the aryl compound (Compound 6) and the arylamine compound (Compound 2) are coupled, whereby an arylamine compound (Compound 7) can be obtained. Synthesis Scheme (A-3a) is shown below.

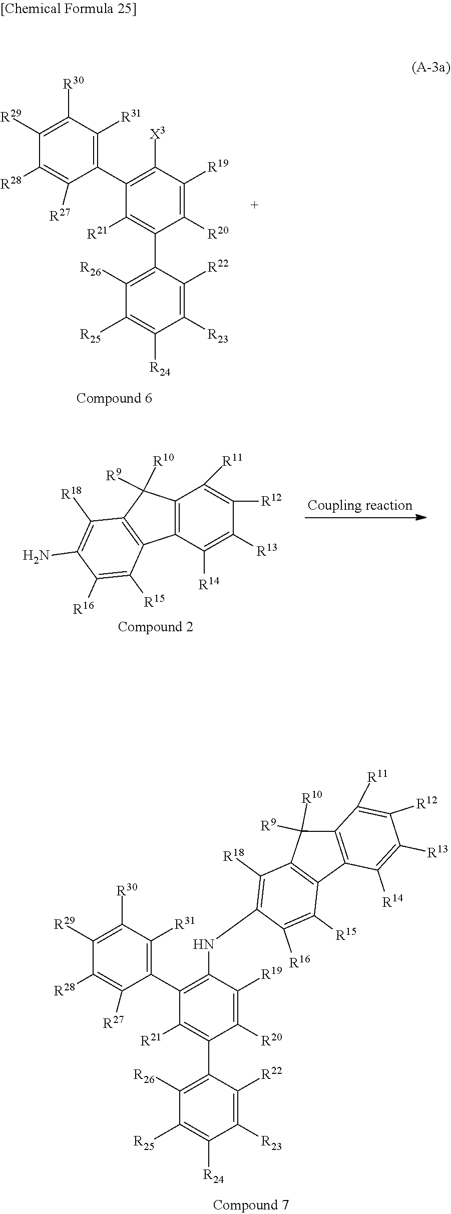

The arylamine compound (Compound 7) can also be obtained in Synthesis Scheme (A-3b), which is another example of Synthesis Scheme (A-3). That is, an arylamine compound (Compound 8) and the aryl compound (Compound 5) are coupled, whereby the arylamine compound (Compound 7) can be obtained. Synthesis Scheme (A-3b) is shown below.

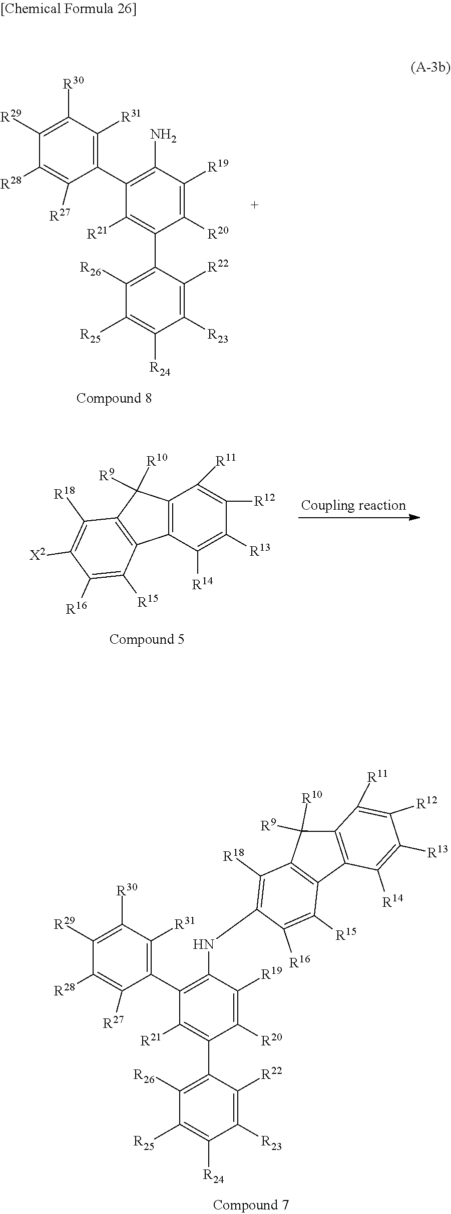

Next, Synthesis Scheme (A-4) is described. That is, the aryl compound (Compound 1) and the arylamine compound (Compound 7) are coupled, whereby the target arylamine compound (G5) can be obtained. The synthesis scheme (A-4) is shown below.

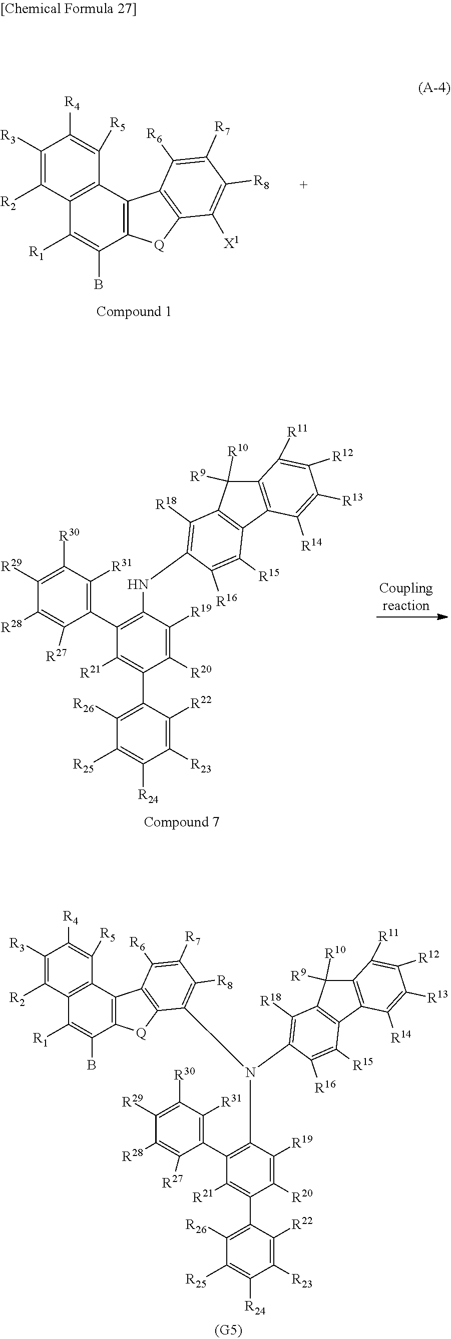

In addition, (G5), which is the target substance, can be obtained by Synthesis Scheme (A-5) and Synthesis Scheme (A-6).

First, Synthesis Scheme (A-5a), which is an example of Synthesis Scheme (A-5), is described. That is, the arylamine compound (Compound 4) and the aryl compound (Compound 6) are coupled, whereby an arylamine compound (Compound 9) can be obtained. Synthesis Scheme (A-5a) is shown below.

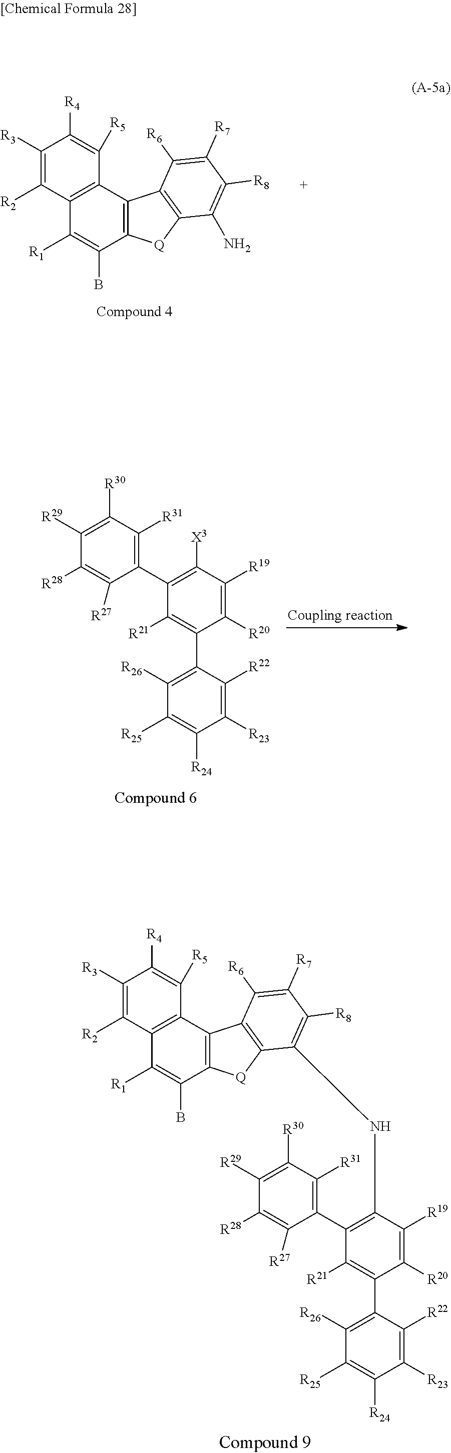

The arylamine compound (Compound 9) can also be obtained in Synthesis Scheme (A-5b), which is another example of Synthesis Scheme (A-5). That is, the aryl compound (Compound 1) and the arylamine compound (Compound 8) are coupled, whereby the arylamine compound (Compound 9) can be obtained. Synthesis Scheme (A-5b) is shown below.

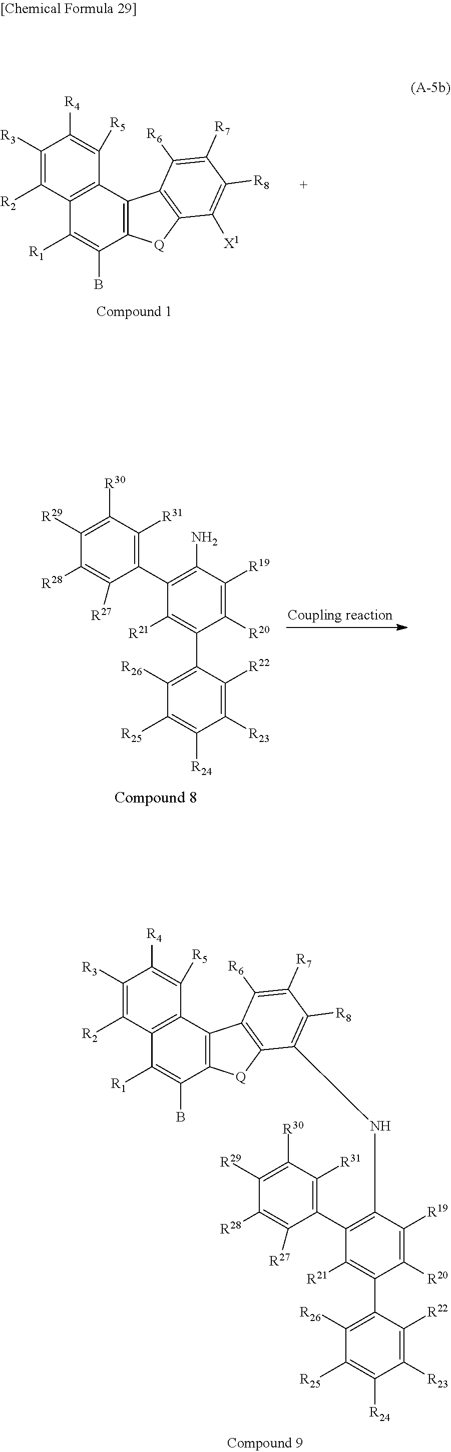

Next, Synthesis Scheme (A-6) is described. That is, the arylamine compound (Compound 9) and the aryl compound (Compound 5) are coupled, whereby the arylamine compound (G5), which is the target substance, can be obtained. Synthesis Scheme (A-6) is shown below.

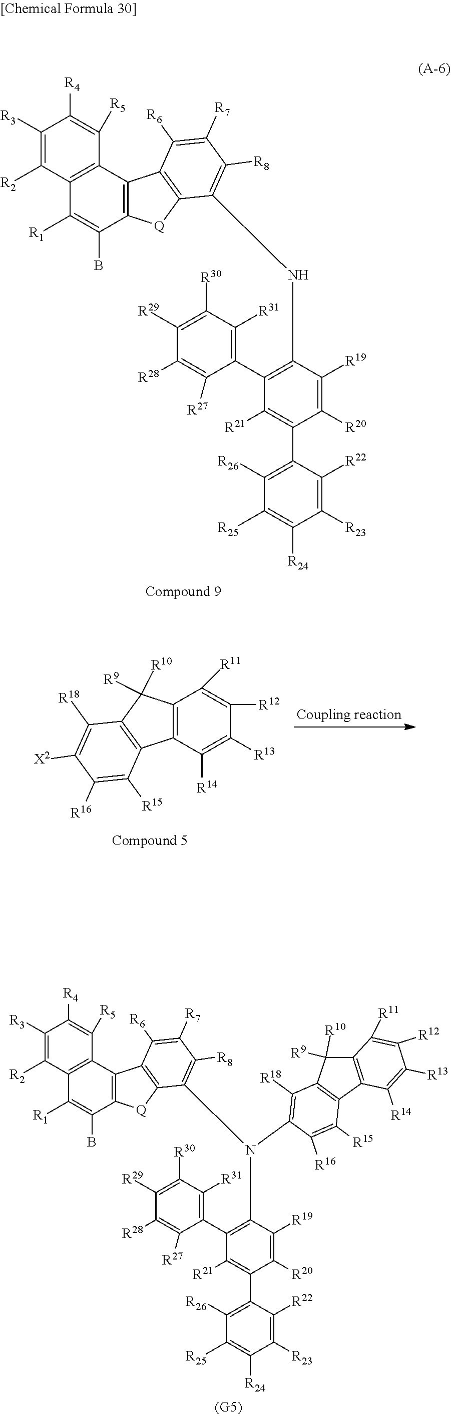

In Synthesis Schemes (A-1) to (A-6) above, X¹ to X³ each independently represent chlorine, bromine, iodine, or a triflate group, and halogen is preferably chlorine, bromine, or iodine, further preferably bromine or iodine in consideration of reactivity, and still further preferably chlorine or bromine in consideration of cost.

In the case where the Buchwald-Hartwig reaction is performed using a palladium catalyst in Synthesis Schemes (A-1) and (A-6), a palladium compound such as bis(dibenzylideneacetone)palladium(0), palladium(II) acetate, [1,1-bis(diphenylphosphino)ferrocene]palladium(II) dichloride, tetrakis(triphenylphosphine)palladium(0), or allylpalladium(II) chloride (dimer) and a ligand such as tri(tert-butyl)phosphine, tri(n-hexyl)phosphine, tricyclohexylphosphine, di(1-adamantyl)-n-butylphosphine, 2-dicyclohexylphosphino-2′,6′-dimethoxybiphenyl, tri(ortho-tolyl)phosphine, or (S)-(6,6′-dimethoxybiphenyl-2,2′-diyl)bis(diisopropylphosphine) (abbreviation: cBRIDP) can be used. In the reaction, an organic base such as sodium tert-butoxide, an inorganic base such as potassium carbonate, cesium carbonate, or sodium carbonate, or the like can be used. In the reaction, toluene, xylene, benzene, tetrahydrofuran, dioxane, or the like can be used as a solvent. Reagents that can be used in the reaction are not limited to the above-described reagents.

Alternatively, in Synthesis Schemes (A-1) to (A-6), the Ullmann reaction using copper or a copper compound can be performed. Copper or a copper compound can be used in the reaction. As the base to be used, an inorganic base such as potassium carbonate can be given. As the solvent that can be used in the reaction, 1,3-dimethyl-3,4,5,6-tetrahydro-2(1H)pyrimidinone (DMPU), toluene, xylene, benzene, and the like can be given. In the Ullmann reaction, when the reaction temperature is 100° C. or higher, a target substance can be obtained in a shorter time in a higher yield; therefore, it is preferable to use DMPU or xylene having a high boiling point. A reaction temperature of 150° C. or higher is further preferred, and accordingly, DMPU is further preferably used. Reagents that can be used in the reaction are not limited to the above-described reagents.

The method of synthesizing the organic compound represented by General Formula (G5) is described above; however, a method for synthesizing the organic compound is not limited to Synthesis Schemes (A-1) to (A-6).

The structures described in this embodiment can be used in an appropriate combination with any of the structures described in the other embodiments.

### Embodiment 2

In this embodiment, a light-emitting device and a light-receiving device that can be used in a light-emitting and light-receiving apparatus of one embodiment of the present invention will be described.

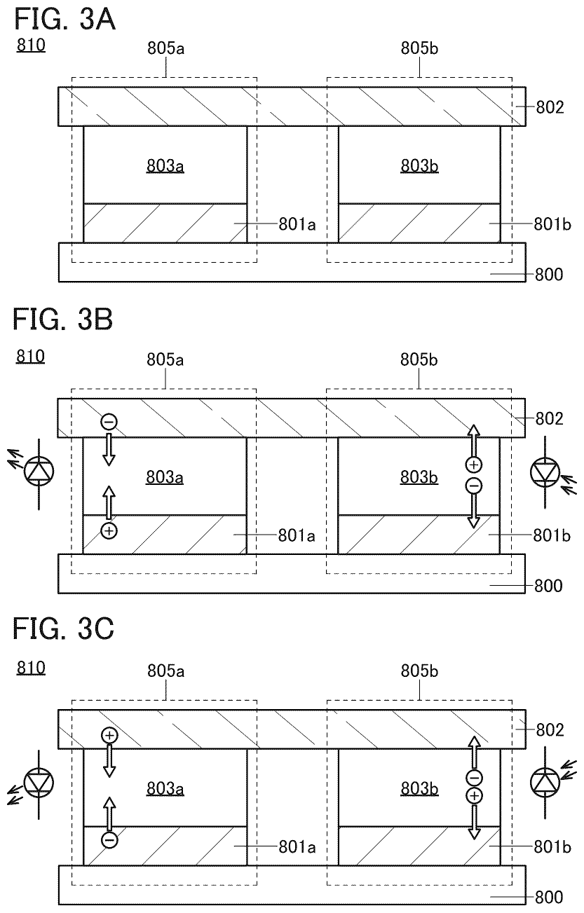

FIG. 3A is a schematic cross-sectional view illustrating a light-emitting device 805a and a light-receiving device 805b included in a light-emitting and light-receiving apparatus 810 of one embodiment of the present invention.

The light-emitting device 805a has a function of emitting light (hereinafter also referred to as a light-emitting function). The light-emitting device 805a includes an electrode 801a, an EL layer 803a, and an electrode 802. The light-emitting device 805a is preferably a light-emitting device utilizing organic EL (an organic EL device) described in Embodiment 1. The EL layer 803a interposed between the electrode 801a and the electrode 802 includes at least a light-emitting layer. The light-emitting layer contains a light-emitting substance. The EL layer 803a emits light when voltage is applied between the electrode 801a and the electrode 802. The EL layer 803a contains the first organic compound described in Embodiment 1. The EL layer 803a may include, in addition to the light-emitting layer, any of a variety of layers such as a hole-injection layer, a hole-transport layer, an electron-transport layer, an electron-injection layer, a carrier-blocking (hole-blocking or electron-blocking) layer, and a charge-generation layer; the first organic compound described in Embodiment 1 can be used for one or both of the hole-injection layer and the hole-transport layer, for example.

The light-receiving device 805b has a function of detecting light (hereinafter also referred to as a light-receiving function). Specific examples of light detected by the light-receiving device 805b include visible light and infrared light. For example, a pn or pin photodiode can be used as the light-receiving device 805b. The light-receiving device 805b includes an electrode 801b, a light-receiving layer 803b, and the electrode 802. The light-receiving layer 803b interposed between the electrode 801b and the electrode 802 includes at least an active layer. The light-receiving layer 803b contains the first organic compound described in Embodiment 1. Note that for the light-receiving layer 803b, any of materials which are used for a variety of layers (e.g., a hole-injection layer, a hole-transport layer, a light-emitting layer, an electron-transport layer, an electron-injection layer, a carrier-blocking (hole-blocking or electron-blocking) layer, and a charge-generation layer) included in the above-described EL layer 803a can be used; the first organic compound described in Embodiment 1 can be used as the materials for the hole-injection layer and the hole-transport layer included in the EL layer 803a, for example.

The light-receiving device 805b functions as a photoelectric conversion device and generates charge on the basis of incident light on the light-receiving layer 803b, and the charge can be extracted as a current. At this time, voltage may be applied between the electrode 801b and the electrode 802. The amount of generated charge is determined depending on the amount of light incident on the light-receiving layer 803b.

In this specification and the like, a blue (B) wavelength range is greater than or equal to 400 nm and less than 490 nm, and blue (B) light has at least one emission spectrum peak in the wavelength range. A green (G) wavelength range is greater than or equal to 490 nm and less than 580 nm, and green (G) light has at least one emission spectrum peak in the wavelength range. A red (R) wavelength range is greater than or equal to 580 nm and less than 700 nm, and red (R) light has at least one emission spectrum peak in the wavelength range. In this specification and the like, a visible light wavelength range is greater than or equal to 400 nm and less than 700 nm, and visible light has at least one emission spectrum peak in the wavelength range. An infrared (IR) wavelength range is greater than or equal to 700 nm and less than 900 nm, and infrared (IR) light has at least one emission spectrum peak in the wavelength range.

The active layer of the light-receiving device 805b contains a semiconductor. Examples of the semiconductor include an inorganic semiconductor such as silicon and an organic semiconductor including an organic compound. As the light-receiving device 805b, an organic semiconductor device (or an organic photodiode) including an organic semiconductor in the active layer is preferably used. An organic photodiode, which is easily made thin, lightweight, and large in area and has high flexibility in shape and design, can be employed for a variety of display apparatuses. With use of an organic semiconductor, the EL layer 803a included in the light-emitting device 805a and the light-receiving layer 803b included in the light-receiving device 805b can be formed by the same method (e.g., a vacuum evaporation method) with the same manufacturing apparatus, which is preferable. The first organic compound described in Embodiment 1 may be used for the active layer.

In the light-emitting and light-receiving apparatus of one embodiment of the present invention, an organic EL device can be used as the light-emitting device 805a and an organic photodiode can be suitably used as the light-receiving device 805b. The organic EL device and the organic photodiode can be formed over the same substrate. Thus, the organic photodiode can be incorporated in the display apparatus using the organic EL device. The light-emitting and light-receiving apparatus of one embodiment of the present invention has an image displaying function and one or both of an image capturing function and a sensing function.

The electrode 801a and the electrode 801b are provided on the same plane. FIG. 3A illustrates a structure where the electrode 801a and the electrode 801b are provided over a substrate 800. The electrode 801a and the electrode 801b can be formed by processing a conductive film formed over the substrate 800 into island-like shapes, for example. In other words, the electrode 801a and the electrode 801b can be formed through the same process.

As the substrate 800, a substrate having heat resistance high enough to withstand the formation of the light-emitting device 805a and the light-receiving device 805b can be used. When an insulating substrate is used as the substrate 800, a glass substrate, a quartz substrate, a sapphire substrate, a ceramic substrate, an organic resin substrate, or the like can be used. Alternatively, a single crystal semiconductor substrate or a polycrystalline semiconductor substrate using silicon or silicon carbide as a material, a compound semiconductor substrate of silicon germanium or the like, or a semiconductor substrate such as an SOI substrate can be used.

As the substrate 800, it is particularly preferable to use the above-described insulating substrate or semiconductor substrate where a semiconductor circuit including a semiconductor element such as a transistor is formed. The semiconductor circuit preferably forms a pixel circuit, a gate line driver circuit (a gate driver), a source line driver circuit (a source driver), or the like. In addition to the above, an arithmetic circuit, a memory circuit, or the like may be formed.

A conductive film transmitting visible light and infrared light is used as the electrode through which light exits or enters among the electrode 801a, the electrode 801b, and the electrode 802. A conductive film reflecting visible light and infrared light is preferably used as the electrode through which light neither exits nor enters.

The electrode 802 in the light-emitting and light-receiving apparatus of one embodiment of the present invention functions as one of the electrodes in each of the light-emitting device 805a and the light-receiving device 805b.

FIG. 3B illustrates the case where the electrode 801a of the light-emitting device 805a has a potential higher than that of the electrode 802. In this case, the electrode 801a functions as an anode and the electrode 802 functions as a cathode in the light-emitting device 805a. The electrode 801b of the light-receiving device 805b has a potential lower than that of the electrode 802. For easy understanding of the direction of current flow, FIG. 3B illustrates a circuit symbol of a light-emitting diode on the left of the light-emitting device 805a and a circuit symbol of a photodiode on the right of the light-receiving device 805b. The flow directions of carriers (electrons and holes) are also schematically indicated in each device by arrows.

In the structure illustrated in FIG. 3B, when a first potential is supplied to the electrode 801a through a first wiring, a second potential is supplied to the electrode 802 through a second wiring, and a third potential is supplied to the electrode 801b through a third wiring, the following relationship is satisfied: the first potential>the second potential>the third potential.

FIG. 3C illustrates the case where the electrode 801a of the light-emitting device 805a has a potential lower than that of the electrode 802. In this case, the electrode 801a functions as a cathode and the electrode 802 function as an anode in the light-emitting device 805a. The electrode 801b of the light-receiving device 805b has a potential lower than that of the electrode 802 and a potential higher than that of the electrode 801a. For easy understanding of the direction of current flow, FIG. 3B illustrates a circuit symbol of a light-emitting diode on the left of the light-emitting device 805a and a circuit symbol of a photodiode on the right of the light-receiving device 805b. The flow directions of carriers (electrons and holes) are also schematically indicated in each device by arrows.

In the structure illustrated in FIG. 3C, when the first potential is supplied to the electrode 801a through the first wiring, the second potential is supplied to the electrode 802 through the second wiring, and the third potential is supplied to the electrode 801b through the third wiring, the following relationship is satisfied: the second potential>the third potential>the first potential.

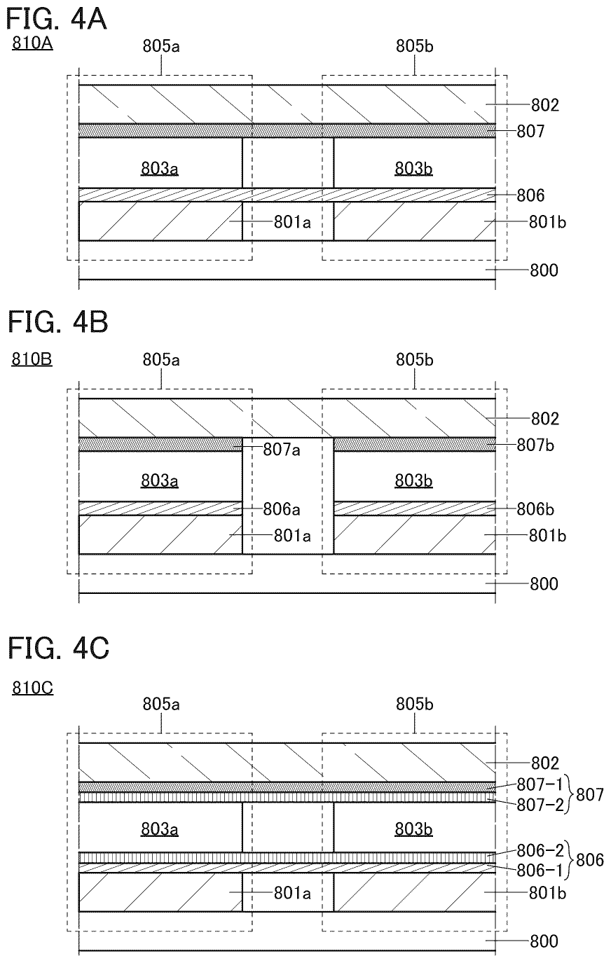

FIG. 4A illustrates a light-emitting and light-receiving apparatus 810A that is a modification example of the light-emitting and light-receiving apparatus 810. The light-emitting and light-receiving apparatus 810A is different from the light-emitting and light-receiving apparatus 810 in that the EL layer 803a and the light-receiving layer 803b include a common layer 806 and a common layer 807. In the light-emitting device 805a, the common layer 806 and the common layer 807 function as part of the EL layer 803a. In the light-receiving device 805b, the common layer 806 and the common layer 807 function as part of the light-receiving layer 803b. The common layer 806 includes a hole-injection layer and a hole-transport layer, for example. The common layer 807 includes an electron-transport layer and an electron-injection layer, for example.

The first organic compound described in Embodiment 1 is preferably used for the common layer 806. Thus, a light-emitting device with low driving voltage and low power consumption can be provided. A light-emitting device with high emission efficiency can be provided. A light-emitting device and a light-receiving device with high reliability can be obtained.

With the common layer 806 and the common layer 807, a light-receiving device can be incorporated without a significant increase in the number of times of separate formation of devices, whereby the light-emitting and light-receiving apparatus 810A can be manufactured with a high throughput.

FIG. 4B illustrates a light-emitting and light-receiving apparatus 810B that is a modification example of the light-emitting and light-receiving apparatus 810. The light-emitting and light-receiving apparatus 810A is different from the light-emitting and light-receiving apparatus 810A in that the EL layer 803a includes a layer 806a and a layer 807a and the light-receiving layer 803b includes a layer 806b and a layer 807b. The layer 806a and the layer 806b are formed using different materials, and each include a hole-injection layer and a hole-transport layer, for example. Note that the layer 806a and the layer 806b may be formed using the same material. The layer 807a and the layer 807b are formed using different materials, and each include an electron-transport layer and an electron-injection layer, for example. Note that the layer 807a and the layer 807b may be formed using the same material.

The first organic compound described in Embodiment 1 is preferably used for the layer 806a and the layer 806b. Thus, a light-emitting device with a low driving voltage and low power consumption can be provided. A light-emitting device with high emission efficiency can be provided. A light-emitting device and a light-receiving device with high reliability can be obtained.

For example, an optimum material for forming the light-emitting device 805a is selected for the layer 806a and the layer 807a from the first organic compounds described in Embodiment 1 and a different optimum material for forming the light-receiving device 805b is selected for the layer 806b and the layer 807b from the first organic compounds described in Embodiment 1, whereby performance of the light-emitting device 805a and the light-receiving device 805b in the light-emitting and light-receiving apparatus 810B can be improved. Specifically, as the light-emitting device 805a, a light-emitting device with a low driving voltage and low power consumption can be provided. A light-emitting device with high emission efficiency can be provided. A light-emitting device with high reliability can be provided. The light-receiving device 805b can be a light-receiving device with high light-receiving sensitivity and high efficiency.

FIG. 4C illustrates a light-emitting and light-receiving apparatus 810C that is a modification example of the light-emitting and light-receiving apparatus 810A. The light-emitting and light-receiving apparatus 810C is different from the light-emitting and light-receiving apparatus 810A in that the common layer 806 has a stacked-layer structure of a common layer 806-1 and a common layer 806-2 and the common layer 807 has a stacked-layer structure of a common layer 807-1 and a common layer 807-2. For example, the common layer 806-1 includes a first hole-transport layer, the common layer 806-2 includes a second hole-transport layer, the common layer 807-1 includes a first electron-transport layer, and the common layer 807-2 includes a second electron-transport layer.

The first organic compound is preferably used for any of the layers included in the common layer 806, and the first organic compound is further preferably used for the common layer 806-1. Accordingly, a light-emitting device and a light-receiving device with a low driving voltage, low power consumption, high efficiency, and high reliability can be obtained.

In the case where the first organic compound is used for any of the common layer 806 (see FIG. 4A and FIG. 4C), the layer 806a, and the layer 806b (see FIG. 4B), the HOMO level of the first organic compound is preferably lower than or equal to −5.40 eV. Accordingly, a light-emitting device and a light-receiving device with a low driving voltage, low power consumption, high efficiency, and high reliability can be obtained.

Note that the light-receiving device 805b described in this embodiment can be arranged at a resolution higher than or equal to 100 ppi, preferably higher than or equal to 200 ppi, more preferably higher than or equal to 300 ppi, further preferably higher than or equal to 400 ppi, still further preferably higher than or equal to 500 ppi and lower than or equal to 2000 ppi, lower than or equal to 1000 ppi, or lower than or equal to 600 ppi, for example. In particular, when the light-receiving device 805b is arranged at a resolution higher than or equal to 200 ppi and lower than or equal to 600 ppi, preferably higher than or equal to 300 ppi and lower than or equal to 600 ppi, the light-receiving devices can be suitably used for image capturing of a fingerprint. In the case where fingerprint authentication is performed with the light-emitting and light-receiving apparatus of one embodiment of the present invention, the increased resolution of the light-receiving device 805b enables, for example, highly accurate extraction of the minutiae of fingerprints; thus, the accuracy of the fingerprint authentication can be increased. The resolution is preferably higher than or equal to 500 ppi, in which case the authentication conforms to the standard by the National Institute of Standards and Technology (NIST) or the like. On the assumption that the resolution at which the light-receiving devices are arranged is 500 ppi, the size of each pixel is 50.8 μm, which indicates that the resolution is adequate for image capturing of a fingerprint ridge distance (typically, greater than or equal to 300 μm and less than or equal to 500 μm).

The structures described in this embodiment can be used in an appropriate combination with any of the structures described in the other embodiments.

### Embodiment 3

In this embodiment, the light-emitting device of one embodiment of the present invention will be described in more detail.

### <<Specific Structure of Light-Emitting Device>>

Next, a specific structure of the light-emitting device of one embodiment of the present invention will be described. Here, description is made using FIG. 1E illustrating the tandem structure. Note that the structure of the EL layer applies also to the light-emitting devices having a single structure in FIG. 1A, FIG. 1C, and FIG. 1D. In the case where the light-emitting device illustrated in FIG. 1E has a microcavity structure, the first electrode 101 is formed as a reflective electrode and the second electrode 102 is formed as a transflective electrode. Thus, a single-layer structure or a stacked-layer structure can be formed using one or more kinds of desired electrode materials. Note that the second electrode 102 is formed after formation of the EL layer 103b, with the use of a material selected as appropriate.

### <First Electrode and Second Electrode>

As materials for forming the first electrode 101 and the second electrode 102, any of the following materials can be used in an appropriate combination as long as the functions of the both electrodes described above can be fulfilled. For example, a metal, an alloy, an electrically conductive compound, and a mixture of these can be used as appropriate. Specifically, an In—Sn oxide (also referred to as ITO), an In—Si—Sn oxide (also referred to as ITSO), an In—Zn oxide, and an In—W—Zn oxide are given. In addition, it is also possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals. It is also possible to use an element belonging to Group 1 or Group 2 in the periodic table, which is not listed above as an example (for example, lithium (Li), cesium (Cs), calcium (Ca), or strontium (Sr)), a rare earth metal such as europium (Eu) or ytterbium (Yb), an alloy containing an appropriate combination of any of these elements, graphene, or the like.

In the light-emitting device illustrated in FIG. 1E, when the first electrode 101 is an anode, a hole-injection layer 111a and a hole-transport layer 112a of the EL layer 103a are sequentially stacked over the first electrode 101 by a vacuum evaporation method. After the EL layer 103a and the charge-generation layer 106 are formed, the hole-injection layer 111b and the hole-transport layer 112b of the EL layer 103b are sequentially stacked over the charge-generation layer 106 in a similar manner.

In addition, the light-emitting device illustrated in FIG. 1E can have a micro optical resonator (microcavity) structure with the first electrode 101 being a reflective electrode and the second electrode 102 being a transflective electrode, and light emitted from the light-emitting layer 113 included in the EL layer 103 can be resonated between the electrodes and light emitted through the second electrode 102 can be intensified.

Note that when the first electrode 101 of the light-emitting device is a reflective electrode having a stacked-layer structure of a reflective conductive material and a light-transmitting conductive material (a transparent conductive film), optical adjustment can be performed by adjusting the thickness of the transparent conductive film. Specifically, when the wavelength of light obtained from the light-emitting layer 113 is λ, the optical path length (the product of the film thickness and the refractive index) between the first electrode 101 and the second electrode 102 is preferably adjusted to mλ/2 (m is an integer of 1 or larger) or the vicinity thereof.

To amplify desired light (wavelength: λ) obtained from the light-emitting layer 113, the optical path length from the first electrode 101 to a region where the desired light is obtained in the light-emitting layer 113 (a light-emitting region) and the optical path length from the second electrode 102 to the region where the desired light is obtained in the light-emitting layer 113 (the light-emitting region) are preferably adjusted to (2m′+1)λ/4 (m′ is an integer of 1 or larger) or the vicinity thereof. Here, the light-emitting region refers to a region where holes and electrons are recombined in the light-emitting layer 113.

By performing such optical adjustment, the spectrum of specific monochromatic light obtained from the light-emitting layer 113 can be narrowed and light emission with high color purity can be obtained.

Note that in the above case, the optical path length between the first electrode 101 and the second electrode 102 is, to be exact, the total thickness from a reflective region in the first electrode 101 to a reflective region in the second electrode 102. However, it is difficult to precisely determine the reflective regions in the first electrode 101 and the second electrode 102; thus, it is assumed that the above effect can be sufficiently obtained with given positions in the first electrode 101 and the second electrode 102 being supposed to be reflective regions. Furthermore, the optical path length between the first electrode 101 and the light-emitting layer from which the desired light is obtained is, to be exact, the optical path length between the reflective region in the first electrode 101 and the light-emitting region in the light-emitting layer from which the desired light is obtained. However, it is difficult to precisely determine the reflective region in the first electrode 101 and the light-emitting region in the light-emitting layer from which the desired light is obtained; thus, it is assumed that the above effect can be sufficiently obtained with a given position in the first electrode 101 being supposed to be the reflective region and a given position in the light-emitting layer from which the desired light is obtained being supposed to be the light-emitting region.

In the above light-emitting device of one embodiment of the present invention, at least one of the first electrode 101 and the second electrode 102 is a light-transmitting electrode (a transparent electrode, a transflective electrode, or the like). In the case where the light-transmitting electrode is a transparent electrode, the visible light transmittance of the transparent electrode is 40% or higher. In the case where the light-transmitting electrode is a transflective electrode, the visible light reflectance of the transflective electrode is higher than or equal to 20% and lower than or equal to 80%, preferably higher than or equal to 40% and lower than or equal to 70%. The resistivity of these electrodes is preferably 1×10⁻² Ωcm or lower.

In the case where one of the first electrode 101 and the second electrode 102 is an electrode having a reflecting property (a reflective electrode) in the above light-emitting device of one embodiment of the present invention, the visible light reflectance of the electrode having a reflecting property is higher than or equal to 40% and lower than or equal to 100%, preferably higher than or equal to 70% and lower than or equal to 100%. The resistivity of this electrode is preferably 1×10⁻² Ωcm or lower.

### <Hole Injection Layer>

The hole-injection layers (111, 111a, and 111b) are each a layer that injects holes from the first electrode 101 which is an anode or from the charge-generation layers (106, 106a, and 106b) to the EL layers (103, 103a, and 103b) and contains an organic acceptor material and a material with a high hole-injection property.

The organic acceptor material is a material that allows holes to be generated in another organic compound whose HOMO level value is close to the LUMO level value of the organic acceptor material when charge separation is caused between the organic acceptor material and the organic compound. Thus, as the organic acceptor material, a compound having an electron-withdrawing group (a halogen group or a cyano group), such as a quinodimethane derivative, a chloranil derivative, or a hexaazatriphenylene derivative, can be used. For example, it is possible to use 7,7,8,8-tetracyano-2,3,5,6-tetrafluoroquinodimethane (abbreviation: F₄-TCNQ), 3,6-difluoro-2,5,7,7,8,8-hexacyanoquinodimethane, chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), or 2-(7-dicyanomethylen-1,3,4,5,6,8,9,10-octafluoro-7H-pyren-2-ylidene)malononitrile. Note that among organic acceptor materials, a compound in which electron-withdrawing groups are bonded to condensed aromatic rings each having a plurality of heteroatoms, such as HAT-CN, is particularly preferred because it has a high acceptor property and stable film quality against heat. Alternatively, a [3]radialene derivative having an electron-withdrawing group (in particular, a cyano group or a halogen group such as a fluoro group) has a very high electron-accepting property and thus is preferable. Specifically, α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], α,α′,α″-1,2,3-cyclopropanetriylidenetris[2,3,4,5,6-pentafluorobenzeneacetonitrile], or the like can be used.

As the material having a high hole-injection property, an oxide of a metal belonging to Group 4 to Group 8 in the periodic table (e.g., a transition metal oxide such as a molybdenum oxide, a vanadium oxide, a ruthenium oxide, a tungsten oxide, or a manganese oxide) can be used. As specific examples, a molybdenum oxide, a vanadium oxide, a niobium oxide, a tantalum oxide, a chromium oxide, a tungsten oxide, a manganese oxide, and a rhenium oxide are given. In particular, a molybdenum oxide is preferable because it is stable in the air, has a low hygroscopic property, and is easily handled. It is also possible to use phthalocyanine (abbreviation: H₂Pc), a phthalocyanine-based compound such as copper phthalocyanine (abbreviation: CuPc), or the like.

In addition to the above materials, it is also possible to use an aromatic amine compound, which is a low molecular compound, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), N,N-bis[4-bis(3-methylphenyl)aminophenyl]-N,N-diphenyl-4,4′-diaminobiphenyl (abbreviation: DNTPD), 1,3,5-tris[N-(4-diphenylaminophenyl)-N-phenylamino]benzene (abbreviation: DPA3B), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), or 3-[N-(1-naphthyl)-N-(9-phenylcarbazol-3-yl)amino]-9-phenylcarbazole (abbreviation: PCzPCN1).

It is also possible to use a high molecular compound (an oligomer, a dendrimer, a polymer, or the like) such as poly(N-vinylcarbazole) (abbreviation: PVK), poly(4-vinyltriphenylamine) (abbreviation: PVTPA), poly[N-(4-{N-[4-(4-diphenylamino)phenyl]phenyl-N′-phenylamino}phenyl)methacrylamide](abbreviation: PTPDMA), or poly[N,N′-bis(4-butylphenyl)-N,N′-bis(phenyl)benzidine](abbreviation: Poly-TPD). It is also possible to use a high molecular compound to which acid such as poly(3,4-ethylenedioxythiophene)/polystyrenesulfonic acid (abbreviation: PEDOT/PSS) or polyaniline/polystyrenesulfonic acid (abbreviation: PAni/PSS) is added.

As the material with a high hole-injection property, a mixed material containing a hole-transport material and the above-described organic acceptor material (an electron-accepting material) can be used. In that case, the organic acceptor material extracts electrons from the hole-transport material, so that holes are generated in the hole-injection layer 111 and the holes are injected into the light-emitting layer 113 through the hole-transport layer 112. Note that the hole-injection layer 111 may be formed as a single layer of a mixed material containing the hole-transport material and the organic acceptor material (an electron-accepting material), or may be formed by stacking a layer containing the hole-transport material and a layer containing the organic acceptor material (the electron-accepting material).

The hole-transport material is preferably a substance having a hole mobility higher than or equal to 1×10⁻⁶ cm²/Vs in the case where the square root of the electric field strength [V/cm] is 600. Note that other substances can be used as long as they have a property of transporting more holes than electrons.

As the hole-transport material, a material having a high hole-transport property such as a compound having a π-electron rich heteroaromatic ring (e.g., a carbazole derivative, a furan derivative, or a thiophene derivative) or an aromatic amine (an organic compound having an aromatic amine skeleton), is preferable. The compound described in Embodiment 1 has a hole-transport property and thus can be used as a hole-transport material.

Examples of the above carbazole derivative (an organic compound having a carbazole ring) include a bicarbazole derivative (e.g., a 3,3′-bicarbazole derivative) and an aromatic amine having a carbazolyl group.

Specific examples of the above bicarbazole derivative (e.g., a 3,3′-bicarbazole derivative) include 3,3′-bis(9-phenyl-9H-carbazole) (abbreviation: PCCP), 9,9′-bis(biphenyl-4-yl)-3,3′-bi-9H-carbazole (abbreviation: BisBPCz), 9,9′-bis(1,1′-biphenyl-3-yl)-3,3′-bi-9H-carbazole (abbreviation: BismBPCz), 9-(biphenyl-3-yl)-9′-(biphenyl-4-yl)-9H,9′H-3,3′-bicarbazole (abbreviation: mBPCCBP), and 9-(2-naphthyl)-9′-phenyl-9H,9′H-3,3′-bicarbazole (abbreviation: PNCCP).

Specific examples of the above aromatic amine having a carbazolyl group include 4-phenyl-4′-(9-phenyl-9H-carbazol-3-yl)triphenylamine (abbreviation: PCBA1BP), N-(4-biphenyl)-N-(9,9-dimethyl-9H-fluoren-2-yl)-9-phenyl-9H-carbazol-3-amine (abbreviation: PCBiF), N-(biphenyl-4-yl)-N-[4-(9-phenyl-9H-carbazol-3-yl)phenyl]-9,9-dimethyl-9H-fluoren-2-amine (abbreviation: PCBBiF), N-[4-(9-phenyl-9H-carbazol-3-yl)phenyl]-bis(9,9-dimethyl-9H-fluoren-2-yl)amine (abbreviation: PCBFF), N-(1,1′-biphenyl-4-yl)-N-[4-(9-phenyl-9H-carbazol-3-yl)phenyl]-9,9-dimethyl-9H-fluoren-4-amine, N-[4-(9-phenyl-9H-carbazol-3-yl)phenyl]-(9,9-dimethyl-9H-fluoren-2-yl)-9,9-dimethyl-9H-fluoren-4-amine, N-(1,1′-biphenyl-4-yl)-N-[4-(9-phenyl-9H-carbazol-3-yl)phenyl]-9,9-diphenyl-9H-fluoren-2-amine, N-(1,1′-biphenyl-4-yl)-N-[4-(9-phenyl-9H-carbazol-3-yl)phenyl]-9,9-diphenyl-9H-fluoren-4-amine, N-(1,1′-biphenyl-4-yl)-N-[4-(9-phenyl-9H-carbazol-3-yl)phenyl]-9,9′-spirobi(9H-fluoren)-2-amine, N-(1,1′-biphenyl-4-yl)-N-[4-(9-phenyl-9H-carbazol-3-yl)phenyl]-9,9′-spirobi(9H-fluoren)-4-amine, N-[4-(9-phenyl-9H-carbazol-3-yl)phenyl]-N-(1,1′:3′,1″-terphenyl-4-yl)-9,9-dimethyl-9H-fluoren-2-amine, N-[4-(9-phenyl-9H-carbazol-3-yl)phenyl]-N-(1,1′:4′,1″-terphenyl-4-yl)-9,9-dimethyl-9H-fluoren-2-amine, N-[4-(9-phenyl-9H-carbazol-3-yl)phenyl]-N-(1,1′: 3′, 1″-terphenyl-4-yl)-9,9-dimethyl-9H-fluoren-4-amine, N-[4-(9-phenyl-9H-carbazol-3-yl)phenyl]-N-(1,1′:4′,1″-terphenyl-4-yl)-9,9-dimethyl-9H-fluoren-4-amine, 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 4-phenyldiphenyl-(9-phenyl-9H-carbazol-3-yl)amine (abbreviation: PCA1BP), N,N-bis(9-phenylcarbazol-3-yl)-N,N-diphenylbenzene-1,3-diamine (abbreviation: PCA2B), N,N,N′-triphenyl-N,N′,N″-tris(9-phenylcarbazol-3-yl)benzene-1,3,5-triamine (abbreviation: PCA3B), 9,9-dimethyl-N-phenyl-N-[4-(9-phenyl-9H-carbazol-3-yl)phenyl]fluoren-2-amine (abbreviation: PCBAF), N-phenyl-N-[4-(9-phenyl-9H-carbazol-3-yl)phenyl]-9,9′-spirobi[9H-fluoren]-2-amine (abbreviation: PCBASF), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 3-[N-(4-diphenylaminophenyl)-N-phenylamino]-9-phenylcarbazole (abbreviation: PCzDPA1), 3,6-bis[N-(4-diphenylaminophenyl)-N-phenylamino]-9-phenylcarbazole (abbreviation: PCzDPA2), 3,6-bis[N-(4-diphenylaminophenyl)-N-(1-naphthyl)amino]-9-phenylcarbazole (abbreviation: PCzTPN2), 2-[N-(9-phenylcarbazol-3-yl)-N-phenylamino]spiro-9,9′-bifluorene (abbreviation: PCASF), N-[4-(9H-carbazol-9-yl)phenyl]-N-(4-phenyl)phenylaniline (abbreviation: YGA1BP), N,N′-bis[4-(carbazol-9-yl)phenyl]-N,N-diphenyl-9,9-dimethylfluorene-2,7-diamine (abbreviation: YGA2F), and 4,4′,4″-tris(carbazol-9-yl)triphenylamine (abbreviation: TCTA).

In addition to the above, other examples of the carbazole derivative include 9-[4-(9-phenyl-9H-carbazol-3-yl)-phenyl]phenanthren (abbreviation: PCPPn), 3-[4-(1-naphthyl)-phenyl]-9-phenyl-9H-carbazole (abbreviation: PCPN), 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 1,3,5-tris[4-(N-carbazolyl)phenyl]benzene (abbreviation: TCPB), and 9-[4-(10-phenyl-9-anthracenyl)phenyl]-9H-carbazole (abbreviation: CzPA).

Specific examples of the above furan derivative (an organic compound having a furan ring) include 4,4′,4″-(benzene-1,3,5-triyl)tri(dibenzofuran) (abbreviation: DBF3P-II), and 4-{3-[3-(9-phenyl-9H-fluoren-9-yl)phenyl]phenyl}dibenzofuran (abbreviation: mmDBFFLBi-II).

Specific examples of the above thiophene derivative (an organic compound having a thiophene ring) include an organic compound and the like containing thiophene ring such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV).

Specific examples of the above aromatic amine include 4,4′-bis[N-(1-naphthyl)-N-phenylamino]biphenyl (abbreviation: NPB or α-NPD), N,N-diphenyl-N,N-bis(3-methylphenyl)-4,4′-diaminobiphenyl (abbreviation: TPD), N,N-bis(9,9′-spirobi[9H-fluoren]-2-yl)-N,N-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), N-(9,9-dimethyl-9H-fluoren-2-yl)-N-{9,9-dimethyl-2-[N-phenyl-N-(9,9-dimethyl-9H-fluoren-2-yl)amino]-9H-fluoren-7-yl}phenylamine (abbreviation: DFLADFL), N-(9,9-dimethyl-2-diphenylamino-9H-fluoren-7-yl)diphenylamine (abbreviation: DPNF), 2-[N-(4-diphenylaminophenyl)-N-phenylamino]spiro-9,9′-bifluorene (abbreviation: DPASF), 2,7-bis[N-(4-diphenylaminophenyl)-N-phenylamino]-spiro-9,9′-bifluorene (abbreviation: DPA2SF), 4,4′,4″-tris[N-(1-naphthyl)-N-phenylamino]triphenylamine (abbreviation: 1′-TNATA), 4,4′,4″-tris(N,N-diphenylamino)triphenylamine (abbreviation: TDATA), 4,4′,4″-tris[N-(3-methylphenyl)-N-phenylamino]triphenylamine (abbreviation: m-MTDATA), N,N-di(p-tolyl)-N,N-diphenyl-p-phenylenediamine (abbreviation: DTDPPA), 4,4′-bis[N-(4-diphenylaminophenyl)-N-phenylamino]biphenyl (abbreviation: DPAB), DNTPD, 1,3,5-tris[N-(4-diphenylaminophenyl)-N-phenylamino]benzene (abbreviation: DPA3B), N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBAαNβNB), 4,4′-diphenyl-4″-(7;1′-binaphthyl-2-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)-triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis(biphenyl-4-yl)-9,9′-spirobi[9H-fluoren]-2-amine (abbreviation: BBASF), N,N-bis(biphenyl-4-yl)-9,9′-spirobi[9H-fluoren]-4-amine (abbreviation: BBASF(4)), N-(biphenyl-2-yl)-N-(9,9-dimethyl-9H-fluoren-2-yl)-9,9′-spirobi[9H-fluoren]-4-amine (abbreviation: oFBiSF), N-(biphenyl-4-yl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-[4-(9-phenylfluoren-9-yl)phenyl]triphenylamine (abbreviation: BPAFLBi), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

Alternatively, it is also possible to use, as the hole-transport material, a high molecular compound (e.g., an oligomer, a dendrimer, or a polymer) such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N-phenylamino}phenyl)methacrylamide](abbreviation: PTPDMA), poly[N,N′-bis(4-butylphenyl)-N,N-bis(phenyl)benzidine](abbreviation: Poly-TPD), or the like. It is also possible to use a high molecular compound to which acid such as poly(3,4-ethylenedioxythiophene)/polystyrenesulfonic acid (abbreviation: PEDOT/PSS) or polyaniline/polystyrenesulfonic acid (abbreviation: PAni/PSS) is added.

Note that the hole-transport material is not limited to the above, and one of or a combination of various known materials may be used as the hole-transport material.

Note that the hole-injection layers (111, 111a, and 111b) can be formed by any of various known deposition methods, and can be formed by a vacuum evaporation method, for example.

### <Hole-Transport Layer>

The hole-transport layers (112, 112a, and 112b) are each a layer that transports the holes, which are injected from the first electrode 101 by the hole-injection layers (111, 111a, and 111b), to the light-emitting layers (113, 113a, and 113b). Note that the hole-transport layers (112, 112a, and 112b) are each a layer containing a hole-transport material. Thus, for the hole-transport layers (112, 112a, and 112b), a hole-transport material that can be used for the hole-injection layers (111, 111a, and 111b) can be used.

Note that in the light-emitting device of one embodiment of the present invention, the organic compound used for the hole-transport layers (112, 112a, and 112b) can also be used for the light-emitting layers (113, 113a, and 113b). The use of the same organic compound for the hole-transport layers (112, 112a, and 112b) and the light-emitting layers (113, 113a, and 113b) is preferable, in which case holes can be efficiently transported from the hole-transport layers (112, 112a, and 112b) to the light-emitting layers (113, 113a, and 113b).

### <Light-Emitting Layer>

The light-emitting layers (113, 113a, and 113b) are each a layer containing a light-emitting substance. Note that as the light-emitting substance that can be used for the light-emitting layers (113, 113a, and 113b), a substance that exhibits an emission color of blue, violet, bluish violet, green, yellowish green, yellow, orange, red, or the like can be used as appropriate. In the case where a plurality of light-emitting layers are provided, different light-emitting substances are used for the light-emitting layers; thus, different emission colors can be exhibited (for example, complementary emission colors are combined to obtain white light emission). In the case where a plurality of light-emitting layers are included, the light-emitting layers can exhibit the same color. The structure in which a plurality of light-emitting layers that emit light of the same color are stacked can achieve higher reliability than a single-layer structure in some cases. Furthermore, one light-emitting layer may have a stacked-layer structure containing different light-emitting substances.

The light-emitting layers (113, 113a, and 113b) may each contain one or more kinds of organic compounds (a host material and the like) in addition to a light-emitting substance (a guest material).

In the case where a plurality of host materials are used in the light-emitting layers (113, 113a, and 113b), a second host material that is additionally used is preferably a substance having a larger energy gap than those of a known guest material and a first host material. Preferably, the lowest singlet excitation energy level (S1 level) of the second host material 3 is higher than the S1 level of the first host material, and the lowest triplet excitation energy level (T1 level) of the second host material is higher than the T1 level of the guest material. Furthermore, the lowest triplet excitation energy level (T1 level) of the second host material is preferably higher than the T1 level of the first host material. With such a structure, an exciplex can be formed by the two kinds of host materials. To form an exciplex efficiently, it is particularly preferable to combine a compound that easily accepts holes (a hole-transport material) and a compound that easily accepts electrons (an electron-transport material). With this structure, high efficiency, low voltage, and a long lifetime can be achieved at the same time.

As an organic compound used as the above host material (including the first host material and the second host material), organic compounds such as the hole-transport materials that can be used for the hole-transport layers (112, 112a, and 112b) described above and electron-transport materials that can be used for the electron-transport layers (114, 114a, and 114b) described later can be used as long as they satisfy requirements for the host material used in the light-emitting layer. Another example is an exciplex formed by a plurality of kinds of organic compounds (the first host material and the second host material). An exciplex (also referred to as Exciplex) whose excited state is formed by a plurality of kinds of organic compounds has an extremely small difference between the S1 level and the T1 level and functions as a TADF material capable of converting triplet excitation energy into singlet excitation energy. As a combination of the plurality of kinds of organic compounds forming an exciplex, for example, it is preferable that one have a π-electron deficient heteroaromatic ring and the other have a π-electron rich heteroaromatic ring. A phosphorescent substance such as an iridium-, rhodium-, or platinum-based organometallic complex or a metal complex may be used as one of the combination forming an exciplex. Since the organic compound described in Embodiment 1 has a hole-transport property, it can be used as the host material.

The light-emitting substance that can be used in the light-emitting layers (113, 113a, and 113b) is not particularly limited, and a light-emitting substance that converts singlet excitation energy into light emission in the visible light range or a light-emitting substance that converts triplet excitation energy into light emission in the visible light range can be used.

<<Light-Emitting Substance that Converts Singlet Excitation Energy into Light Emission>>

The following substances emitting fluorescent light (fluorescent substances) are given as the light-emitting substance that can be used for the light-emitting layers (113, 113a, and 113b) and convert singlet excitation energy into light emission. The examples include a pyrene derivative, an anthracene derivative, a triphenylene derivative, a fluorene derivative, a carbazole derivative, a dibenzothiophene derivative, a dibenzofuran derivative, a dibenzoquinoxaline derivative, a quinoxaline derivative, a pyridine derivative, a pyrimidine derivative, a phenanthrene derivative, and a naphthalene derivative. A pyrene derivative is particularly preferable because it has a high emission quantum yield. Specific examples of the pyrene derivative include N,N-bis(3-methylphenyl)-N,N-bis[3-(9-phenyl-9H-fluoren-9-yl)phenyl]pyrene-1,6-diamine (abbreviation: 1,6mMemFLPAPrn), N,N-diphenyl-N,N-bis[4-(9-phenyl-9H-fluoren-9-yl)phenyl]pyrene-1,6-diamine (abbreviation: 1,6FLPAPrn), N,N-bis(dibenzofuran-2-yl)-N,N-diphenylpyrene-1,6-diamine (abbreviation: 1,6FrAPrn), N,N-bis(dibenzothiophen-2-yl)-N,N-diphenylpyrene-1,6-diamine (abbreviation: 1,6ThAPrn), N,N-(pyrene-1,6-diyl)bis[(N-phenylbenzo[b]naphtho[1,2-d]furan)-6-amine](abbreviation: 1,6BnfAPrn), N,N-(pyrene-1,6-diyl)bis[(N-phenylbenzo[b]naphtho[1,2-d]furan)-8-amine](abbreviation: 1,6BnfAPrn-02), and N,N-(pyrene-1,6-diyl)bis[(6,N-diphenylbenzo[b]naphtho[1,2-d]furan)-8-amine](abbreviation: 1,6BnfAPrn-03).

In addition, it is possible to use 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N-bis[4-(9H-carbazol-9-yl)phenyl]-N,N-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), 4-(10-phenyl-9-anthryl)-4′-(9-phenyl-9H-carbazol-3-yl)triphenylamine (abbreviation: PCBAPA), 4-[4-(10-phenyl-9-anthryl)phenyl]-4′-(9-phenyl-9H-carbazol-3-yl)triphenylamine (abbreviation: PCBAPBA), perylene, 2,5,8,11-tetra-tert-butylperylene (abbreviation: TBP), N,N′-(2-tert-butylanthracene-9,10-diyldi-4,1-phenylene)bis(N,N,N-triphenyl-1,4-phenylenediamine) (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N,N-triphenyl-1,4-phenylenediamine (abbreviation: 2DPAPPA), or the like.

It is also possible to use, for example, N-[9,10-bis(biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(biphenyl-2-yl)-2-anthryl]-N,N,N-triphenyl-1,4-phenylenediamine (abbreviation: 2DPABPhA), 9,10-bis(biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N-diphenylquinacridone (abbreviation: DPQd), rubrene, 5,12-bis(1,1′-biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), 1,6BnfAPrn-03, 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). In particular, pyrenediamine compounds such as 1,6FLPAPrn, 1,6mMemFLPAPrn, and 1,6BnfAPrn-03 can be used, for example.

<<Light-Emitting Substance that Converts Triplet Excitation Energy into Light Emission>>

Next, as examples of the light-emitting substance that converts triplet excitation energy into light emission and can be used for the light-emitting layer 113, a substance that emits phosphorescent light (a phosphorescent substance) and a thermally activated delayed fluorescent (TADF) material that exhibits thermally activated delayed fluorescence are given.

A phosphorescent substance refers to a compound that exhibits phosphorescence but does not exhibit fluorescence at a temperature higher than or equal to low temperatures (e.g., 77 K) and lower than or equal to room temperature (i.e., higher than or equal to 77 K and lower than or equal to 313 K). The phosphorescent substance preferably contains a metal element with large spin-orbit interaction, and can be an organometallic complex, a metal complex (a platinum complex), a rare earth metal complex, or the like. Specifically, a transition metal element is preferable and it is particularly preferable that a platinum group element (ruthenium (Ru), rhodium (Rh), palladium (Pd), osmium (Os), iridium (Ir), or platinum (Pt)), especially iridium, be contained, in which case the transition probability relating to direct transition between the singlet ground state and the triplet excited state can be increased.

<<Phosphorescent Substance (from 450 nm to 570 nm: Blue or Green)>>

As a phosphorescent substance that exhibits blue or green and whose emission spectrum has a peak wavelength at greater than or equal to 450 nm and less than or equal to 570 nm, the following substances are given.

The examples include: organometallic complexes having a 4H-triazole ring, such as tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), tris[4-(3-biphenyl)-5-isopropyl-3-phenyl-4H-1,2,4-triazolato]iridium(III) (abbreviation: [Ir(iPrptz-3b)₃]), and tris[3-(5-biphenyl)-5-isopropyl-4-phenyl-4H-1,2,4-triazolato]iridium(III) (abbreviation: [Ir(iPr5btz)₃]); organometallic complexes having a 1H-triazole ring, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); organometallic complexes having an imidazole ring, such as fac-tris[1-(2,6-diisopropylphenyl)-2-phenyl-1H-imidazole]iridium(III) (abbreviation: [Ir(iPrpim)₃]) and tris[3-(2,6-dimethylphenyl)-7-methylimidazo[1,2-f]phenanthridinato]iridium(III) (abbreviation: [Ir(dmpimpt-Me)₃]); and organometallic complexes in which a ligand is a phenylpyridine derivative having an electron-withdrawing group such as bis[2-(4′,6′-difluorophenyl)pyridinato-N,C²′]iridium(III) tetrakis(1-pyrazolyl)borate (abbreviation: FIr6), bis[2-(4′,6′-difluorophenyl)pyridinato-N,C²′]iridium(III) picolinate (abbreviation: FIrpic), bis{2-[3′,5′-bis(trifluoromethyl)phenyl]pyridinato-N,C²′}iridium(III) picolinate (abbreviation: [Ir(CF₃ppy)₂(pic)]), and bis[2-(4′,6′-difluorophenyl)pyridinato-N,C²′]iridium(III) acetylacetonate (abbreviation: FIr(acac)).

<<Phosphorescent Substance (from 495 nm to 590 nm: Green or Yellow)>>

As a phosphorescent substance that exhibits green or yellow and whose emission spectrum has a peak wavelength at greater than or equal to 495 nm and less than or equal to 590 nm, the following substances are given.

The examples include: organometallic iridium complexes having a pyrimidine ring, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), (acetylacetonato)bis{4,6-dimethyl-2-[6-(2,6-dimethylphenyl)-4-pyrimidinyl-κN³]phenyl-κC}iridium(III) (abbreviation: [Ir(dmppm-dmp)₂(acac)]), and (acetylacetonato)bis(4,6-diphenylpyrimidinato)iridium(III) (abbreviation: [Ir(dppm)₂(acac)]); organometallic iridium complexes having a pyrazine ring, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine ring, such as tris(2-phenylpyridinato-N,C²′)iridium(III) (abbreviation: [Ir(ppy)₃]), bis(2-phenylpyridinato-N,C²′)iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C²′)iridium(III) (abbreviation: [Ir(pq)₃]), bis(2-phenylquinolinato-N,C²′)iridium(III) acetylacetonate (abbreviation: [Ir(pq)₂(acac)]), bis[2-(2-pyridinyl-κN)phenyl-κC][2-(4-phenyl-2-pyridinyl-κN)phenyl-κC]iridium(III) (abbreviation: [Ir(ppy)₂(4dppy)]), bis[2-(2-pyridinyl-κN)phenyl-κC][2-(4-methyl-5-phenyl-2-pyridinyl-κN)phenyl-KC], [2-d3-methyl-8-(2-pyridinyl-N)benzofuro[2,3-b]pyridine-κC]bis[2-(5-d3-methyl-2-pyridinyl-κN2)phenyl-κC]iridium(III) (abbreviation: Ir(5mppy-d3)₂(mbfpypy-d3)), {2-(methyl-d3)-8-[4-(1-methylethyl-1-d)-2-pyridinyl-κN]benzofuro[2,3-b]pyridine-7-yl-KC}bis{5-(methyl-d3)-2-[5-(methyl-d3)-2-pyridinyl-KM]phenyl-κC}iridium(III) (abbreviation: Ir(5mtpy-d6)₂(mbfpypy-iPr-d4)), [2-d3-methyl-(2-pyridinyl-κN)benzofuro[2,3-b]pyridine-κC]bis[2-(2-pyridinyl-κN)phenyl-κC]iridium(III) (abbreviation: Ir(ppy)₂(mbfpypy-d3)), and [2-(4-methyl-5-phenyl-2-pyridinyl-κN)phenyl-κC]bis[2-(2-pyridinyl-κN)phenyl-κC]iridium(III) (abbreviation: Ir(ppy)₂(mdppy)); organometallic complexes such as bis(2,4-diphenyl-1,3-oxazolato-N,C²′)iridium(III) acetylacetonate (abbreviation: [Ir(dpo)₂(acac)]), bis{2-[4′-(perfluorophenyl)phenyl]pyridinato-N,C²′}iridium(III) acetylacetonate (abbreviation: [Ir(p-PF-ph)₂(acac)]), and bis(2-phenylbenzothiazolato-N,C²′)iridium(III) acetylacetonate (abbreviation: [Ir(bt)₂(acac)]); and rare earth metal complexes such as tris(acetylacetonato) (monophenanthroline)terbium(III) (abbreviation: [Tb(acac)₃(Phen)]).

<<Phosphorescent Substance (from 570 nm to 750 nm: Yellow or Red)>>

As a phosphorescent substance that exhibits yellow or red and whose emission spectrum has a peak wavelength at greater than or equal to 570 nm and less than or equal to 750 nm, the following substances are given.

The examples include organometallic complexes having a pyrimidine ring, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and (dipivaloylmethanato)bis[4,6-di(naphthalen-1-yl)pyrimidinato]iridium(III) (abbreviation: [Ir(d1npm)₂(dpm)]); organometallic complexes having a pyrazine ring, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), bis{4,6-dimethyl-2-[3-(3,5-dimethylphenyl)-5-phenyl-2-pyrazinyl-N]phenyl-κC}(2,6-dimethyl-3,5-heptanedionato-κ²O,O′)iridium(III) (abbreviation: [Ir(dmdppr-P)₂(dibm)]), bis{4,6-dimethyl-2-[5-(4-cyano-2,6-dimethylphenyl)-3-(3,5-dimethylphenyl)-2-pyrazinyl-κN]phenyl-κC}(2,2,6,6-tetramethyl-3,5-heptanedionato-k²O,O′)iridium(III) (abbreviation: [Ir(dmdppr-dmCP)₂(dpm)]), bis{2-[5-(2,6-dimethylphenyl)-3-(3,5-dimethylphenyl)-2-pyrazinyl-κN]-4,6-dimethylphenyl-κC}(2,2′,6,6′-tetramethyl-3,5-heptadionato-κ²O,O′)iridium(III) (abbreviation: [Ir(dmdppr-dmp)₂(dpm)]), (acetylacetonato)bis[2-methyl-3-phenylquinoxalinato-N,C²′]iridium(III) (abbreviation: [Ir(mpq)₂(acac)]), (acetylacetonato)bis(2,3-diphenylquinoxalinato-N,C²′)iridium(III) (abbreviation: [Ir(dpq)₂(acac)]), and (acetylacetonato)bis[2,3-bis(4-fluorophenyl)quinoxalinato]iridium(III) (abbreviation: [Ir(Fdpq)₂(acac)]); organometallic complexes having a pyridine ring, such as tris(1-phenylisoquinolinato-N,C²′)iridium(III) (abbreviation: [Ir(piq)₃]), bis(1-phenylisoquinolinato-N,C²′)iridium(III) acetylacetonate (abbreviation: [Ir(piq)₂(acac)]), and bis[4,6-dimethyl-2-(2-quinolinyl-κN)phenyl-KC](2,4-pentanedionato-κ²O,O′)iridium(III) (abbreviation: [Ir(dmpqn)₂(acac)]); platinum complexes such as 2,3,7,8,12,13,17,18-octaethyl-21H,23H-porphyrinplatinum(II) (abbreviation: [PtOEP]); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]).

### <<TADF Material>>

Any of materials shown below can be used as the TADF material. The TADF material refers to a material that has a small difference (preferably, less than or equal to 0.20 eV) between the S1 level and the T1 level, can up-convert a triplet excited state into a singlet excited state (reverse intersystem crossing) using a little thermal energy, and efficiently exhibits light emission (fluorescence) from the singlet excited state. The thermally activated delayed fluorescence is efficiently obtained under the condition where the difference in energy between the triplet excited energy level and the singlet excited energy level is greater than or equal to 0.00 eV and less than or equal to 0.20 eV, preferably greater than or equal to 0.00 eV and less than or equal to 0.10 eV. Delayed fluorescence by the TADF material refers to light emission having a spectrum similar to that of normal fluorescence and an extremely long lifetime. The lifetime is 1×10⁻⁶ seconds or longer, or 1×10⁻³ seconds or longer.

Note that the TADF material can be also used as an electron-transport material, a hole-transport material, or a host material.

Examples of the TADF material include fullerene, a derivative thereof, an acridine derivative such as proflavine, and eosin. Other examples include a metal-containing porphyrin such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (abbreviation: SnF₂(Proto IX)), a mesoporphyrin-tin fluoride complex (abbreviation: SnF₂(Meso IX)), a hematoporphyrin-tin fluoride complex (abbreviation: SnF₂(Hemato IX)), a coproporphyrin tetramethyl ester-tin fluoride complex (abbreviation: SnF₂(Copro III-4Me)), an octaethylporphyrin-tin fluoride complex (abbreviation: SnF₂(OEP)), an etioporphyrin-tin fluoride complex (abbreviation: SnF₂(Etio I)), and an octaethylporphyrin-platinum chloride complex (abbreviation: PtCl₂OEP).

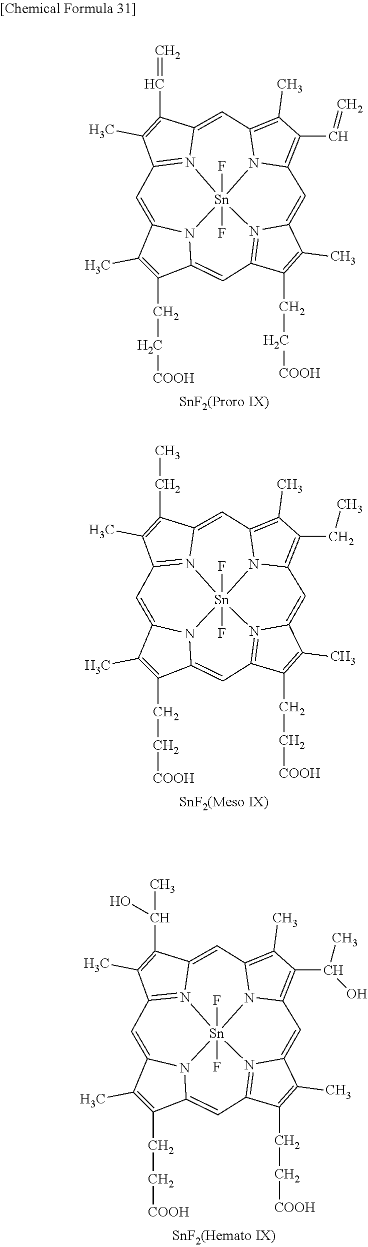

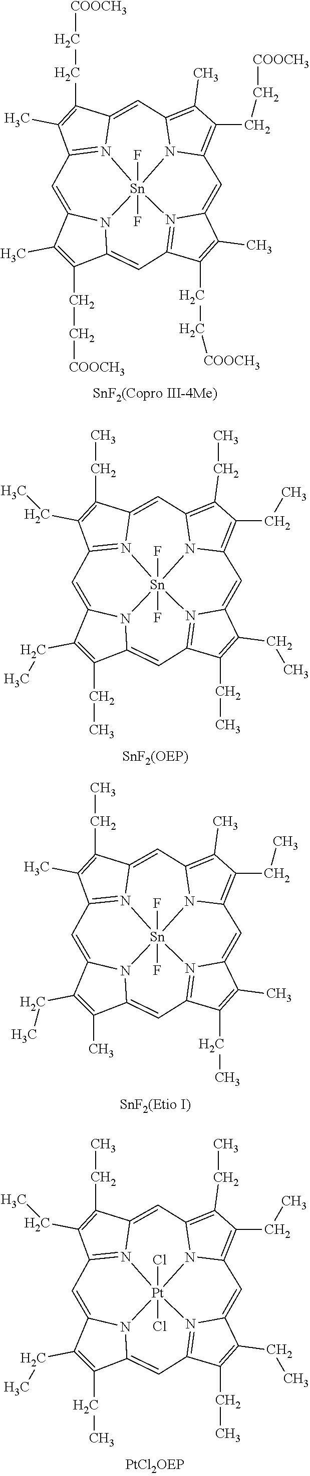

Alternatively, a heteroaromatic compound having a π-electron rich heteroaromatic compound and a π-electron deficient heteroaromatic compound, such as 2-(biphenyl-4-yl)-4,6-bis(12-phenylindolo[2,3-a]carbazol-11-yl)-1,3,5-triazine (abbreviation: PIC-TRZ), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), 10-phenyl-10H,10′H-spiro[acridin-9,9′-anthracen]-10′-one (abbreviation: ACRSA), 4-(9′-phenyl-3,3′-bi-9H-carbazol-9-yl)benzofuro[3,2-d]pyrimidine (abbreviation: 4PCCzBfpm), 4-[4-(9′-phenyl-3,3′-bi-9H-carbazol-9-yl)phenyl]benzofuro[3,2-d]pyrimidine (abbreviation: 4PCCzPBfpm), and 9-[3-(4,6-diphenyl-1,3,5-triazin-2-yl)phenyl]-9′-phenyl-2,3′-bi-9H-carbazole (abbreviation: mPCCzPTzn-02), may be used.

Note that a substance in which a π-electron rich heteroaromatic compound is directly bonded to a π-electron deficient heteroaromatic compound is particularly preferable because both the donor property of the π-electron rich heteroaromatic compound and the acceptor property of the π-electron deficient heteroaromatic compound are improved and the energy difference between the singlet excited state and the triplet excited state becomes small. As the TADF material, a TADF material (TADF 100) in which a singlet excited state and a triplet excited state is in a thermal equilibrium state may be used. Since such a TADF material enables a short emission lifetime (excitation lifetime), a decrease in efficiency of a light-emitting device in a high-luminance region can be inhibited.

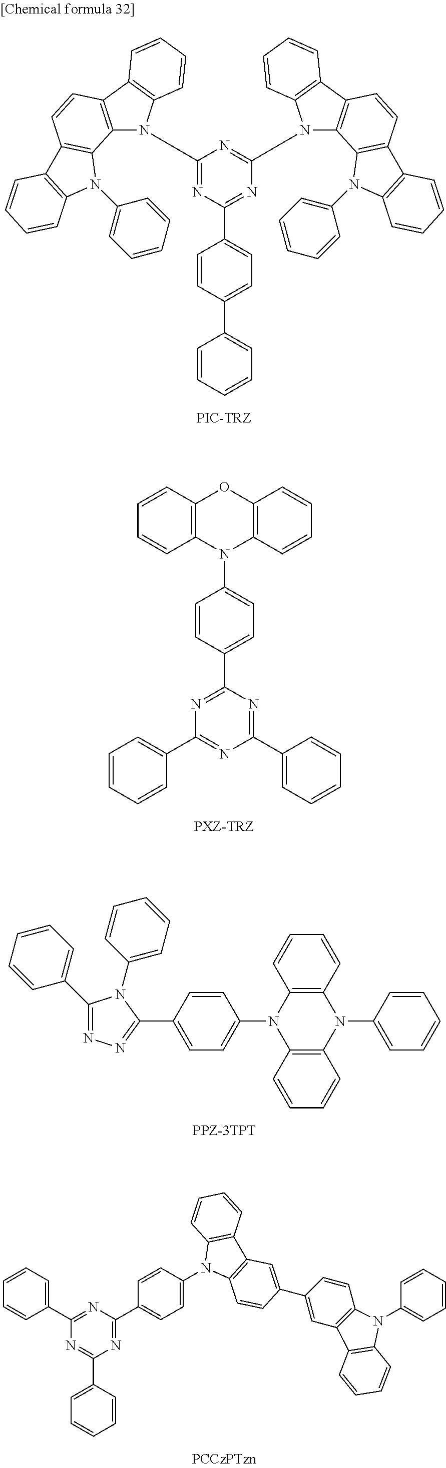 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-c3ccc(N4c5ccccc5Oc5ccccc54)cc3)n2)cc1 ; c1ccc(-c2nnc(-c3ccc(N4c5ccccc5N(c5ccccc5)c5ccccc54)cc3)n2-c2ccccc2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1]

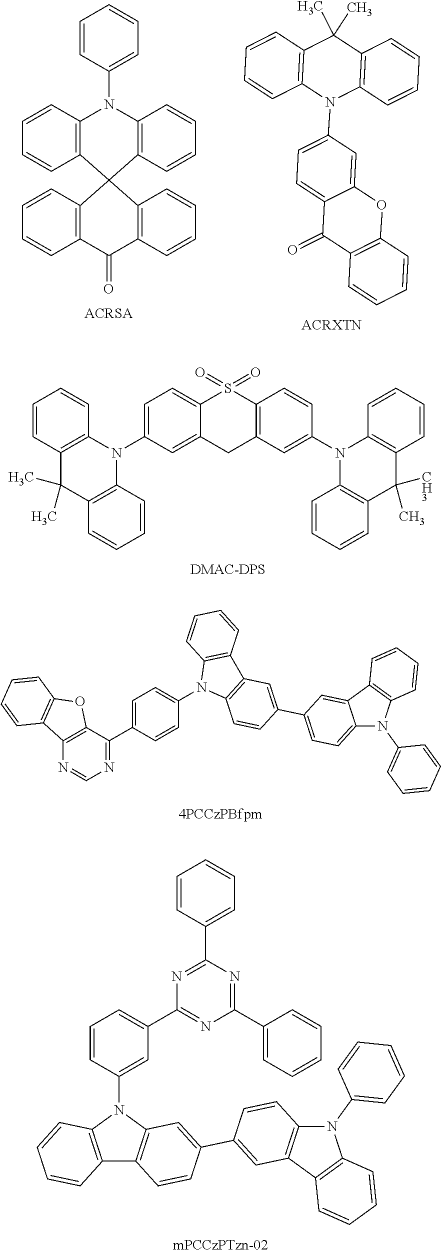

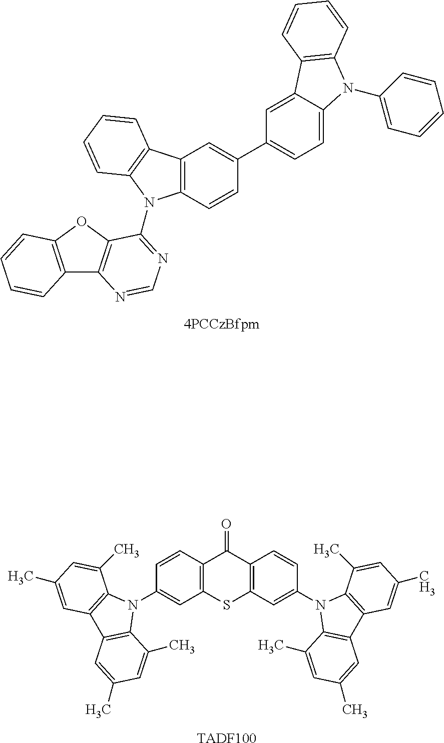

As the material having a function of converting triplet excitation energy into light emission, a nanostructure of a transition metal compound having a perovskite structure is also given in addition to the above. In particular, a nanostructure of a metal-halide perovskite material is preferable. The nanostructure is preferably a nanoparticle or a nanorod.

As the organic compounds (the host material and the like) used in combination with the above-described light-emitting substance (the guest material) in the light-emitting layers (113, 113a, 113b, and 113c), one or more kinds of substances having a larger energy gap than the light-emitting substance (the guest material) may be selected to be used.

### <<Host Material for Fluorescent Light Emission>>

In the case where the light-emitting substance used in the light-emitting layers (113, 113a, 113b, and 113c) is a fluorescent substance, an organic compound (a host material) used in combination with the light-emitting substance is preferably an organic compound that has a high energy level in a singlet excited state and has a low energy level in a triplet excited state or an organic compound having a high fluorescence quantum yield. Therefore, the hole-transport material (described above) and the electron-transport material (described below) in this embodiment, for example, can be used as long as it is an organic compound that satisfies such a condition. The organic compounds described in Embodiment 1 can also be used.

In terms of a preferable combination with the light-emitting substance (the fluorescent substance), examples of the organic compound (the host material) include condensed polycyclic aromatic compounds such as an anthracene derivative, a tetracene derivative, a phenanthrene derivative, a pyrene derivative, a chrysene derivative, and a dibenzo[g,p]chrysene derivative, although some of them overlap with the above specific examples.

Note that specific examples of the organic compound (the host material) preferably used in combination with the fluorescent substance include 9-phenyl-3-[4-(10-phenyl-9-anthryl)phenyl]-9H-carbazole (abbreviation: PCzPA), 3,6-diphenyl-9-[4-(10-phenyl-9-anthryl)phenyl]-9H-carbazole (abbreviation: DPCzPA), 3-[4-(1-naphthyl)-phenyl]-9-phenyl-9H-carbazole (abbreviation: PCPN), 9,10-diphenylanthracene (abbreviation: DPAnth), N,N-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), YGAPA, PCAPA, N,9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N,9-diphenyl-9H-carbazol-3-amine (abbreviation: 2PCAPA), 6,12-dimethoxy-5,11-diphenylchrysene, N,N,N,N,N′,N′,N″,N″-octaphenyldibenzo[g,p]chrysene-2,7,10,15-tetraamine (abbreviation: DBC1), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]-benzo[b]naphtho[1,2-d]furan (abbreviation: 2mBnfPPA), 9-phenyl-10-[4′-(9-phenyl-9H-fluoren-9-yl)-biphenyl-4-yl]anthracene (abbreviation: FLPPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9-(1-naphthyl)-10-(2-naphthyl)anthracene (abbreviation: α,β-ADN), 2-(10-phenylanthracen-9-yl)dibenzofuran, 2-(10-phenyl-9-anthracenyl)benzo[b]naphtho[2,3-d]furan (abbreviation: Bnf(II)PhA), 9-(1-naphthyl)-10-[4-(2-naphthyl)phenyl]anthracene (abbreviation: αN-βNPAnth), 2,9-di(1-naphthyl)-10-phenylanthracene (abbreviation: 2αN-αNPhA), 9-(1-naphthyl)-10-[3-(1-naphthyl)phenyl]anthracene (abbreviation: αN-mαNPAnth), 9-(2-naphthyl)-10-[3-(1-naphthyl)phenyl]anthracene (abbreviation: βN-mαNPAnth), 9-(1-naphthyl)-10-[4-(1-naphthyl)phenyl]anthracene (abbreviation: αN-αNPAnth), 9-(2-naphthyl)-10-[4-(2-naphthyl)phenyl]anthracene (abbreviation: βN-βNPAnth), 2-(1-naphthyl)-9-(2-naphthyl)-10-phenylanthracene (abbreviation: 2αN-βNPhA), 9-(2-naphthyl)-10-[3-(2-naphthyl)phenyl]anthracene (abbreviation: βN-mβNPAnth), 1-[4-(10-(biphenyl-4-yl)-9-anthracenyl)phenyl]-2-ethyl-1H-benzimidazole (abbreviation: EtBImPBPhA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 1,3,5-tri(1-pyrenyl)benzene (abbreviation: TPB3), 5,12-diphenyltetracene, 5,12-bis(biphenyl-2-yl)tetracene, and the like.

### <<Host Material for Phosphorescent Light Emission>>

In the case where the light-emitting substance used for the light-emitting layers (113, 113a, 113b, and 113c) is a phosphorescent substance, an organic compound having triplet excitation energy (energy difference between a ground state and a triplet excited state) which is higher than that of the light-emitting substance is preferably selected as the organic compound (the host material) used in combination with the light-emitting substance. Note that in the case where a plurality of organic compounds (e.g., a first host material and a second host material (or an assist material)) are used in combination with a light-emitting substance in order to form an exciplex, the plurality of organic compounds are preferably mixed with a phosphorescent substance. The organic compounds described in Embodiment 1 can also be used.

Such a structure makes it possible to efficiently obtain light emission utilizing ExTET (Exciplex-Triplet Energy Transfer), which is energy transfer from an exciplex to a light-emitting substance. Note that a combination of the plurality of organic compounds that easily forms an exciplex is preferably employed, and it is particularly preferable to combine a compound that easily accepts holes (a hole-transport material) and a compound that easily accepts electrons (an electron-transport material).

In terms of a preferable combination with the light-emitting substance (phosphorescent substance), examples of the organic compounds (the host material and the assist material) include an aromatic amine (an organic compound having an aromatic amine skeleton), a carbazole derivative (an organic compound having a carbazole ring), a dibenzothiophene derivative (an organic compound having a dibenzothiophene ring), a dibenzofuran derivative (an organic compound having a dibenzofuran ring), an oxadiazole derivative (an organic compound having an oxadiazole ring), a triazole derivative (an organic compound having a triazole ring), a benzimidazole derivative (an organic compound having a benzimidazole ring), a quinoxaline (an organic compound having a quinoxaline ring) derivative, a dibenzoquinoxaline derivative (an organic compound having a dibenzoquinoxaline ring), a pyrimidine derivative (an organic compound having a pyrimidine ring), a triazine derivative (an organic compound having a triazine ring), a pyridine derivative (an organic compound having a pyridine ring), a bipyridine derivative (an organic compound having a bipyridine ring), a phenanthroline derivative (an organic compound having a phenanthroline ring), a furodiazine derivative (an organic compound having a furodiazine ring), and zinc- or aluminum-based metal complexes, although some of them overlap with the above specific examples.

Among the above organic compounds, specific examples of the aromatic amine and the carbazole derivative, which are organic compounds having a high hole-transport property, are the same as the specific examples of the hole-transport materials described above. Any of these is preferable as the host material.

Among the above organic compounds, specific examples of the dibenzothiophene derivative and the dibenzofuran derivative, which are organic compounds having a high hole-transport property, include 4-{3-[3-(9-phenyl-9H-fluoren-9-yl)phenyl]phenyl}dibenzofuran (abbreviation: mmDBFFLBi-II), 4,4′,4″-(benzene-1,3,5-triyl)tri(dibenzofuran) (abbreviation: DBF3P-II), DBT3P-II, 2,8-diphenyl-4-[4-(9-phenyl-9H-fluoren-9-yl)phenyl]dibenzothiophene (abbreviation: DBTFLP-III), 4-[4-(9-phenyl-9H-fluoren-9-yl)phenyl]-6-phenyldibenzothiophene (abbreviation: DBTFLP-IV), and 4-[3-(triphenylen-2-yl)phenyl]dibenzothiophene (abbreviation: mDBTPTp-II). Any of these is preferable as the host material.

In addition, a metal complex having an oxazole-based or thiazole-based ligand, such as bis[2-(2-benzoxazolyl)phenolato]zinc(II) (abbreviation: ZnPBO) or bis[2-(2-benzothiazolyl)phenolato]zinc(II) (abbreviation: ZnBTZ), or the like is given as a preferable example of the host material.

Specific examples of the oxadiazole derivative, the triazole derivative, the benzimidazole derivative, the quinoxaline derivative, the dibenzoquinoxaline derivative, the quinazoline derivative, the phenanthroline derivative, and the like, which are organic compounds having a high electron-transport property among the above organic compounds, include an organic compound including a heteroaromatic ring having a polyazole ring, such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), and 4,4′-bis(5-methylbenzoxazol-2-yl)stilbene (abbreviation: BzOs); an organic compound including a heteroaromatic ring having a phenanthroline ring, such as bathophenanthroline (abbreviation: Bphen), bathocuproine (abbreviation: BCP), 2,9-di(naphthalen-2-yl)-4,7-diphenyl-1,10-phenanthroline (abbreviation: NBphen), and 2,2-(1,3-phenylene)bis[9-phenyl-1,10-phenanthroline](abbreviation: mPPhen2P); and an organic compound including a heteroaromatic ring having a dibenzoquinoxaline ring, such as 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 2-{4-[9,10-di(2-naphthyl)-2-anthryl]phenyl}-1-phenyl-1H-benzimidazole (abbreviation: ZADN), and 2-[4′-(9-phenyl-9H-carbazol-3-yl)-3,1′-biphenyl-1-yl]dibenzo[f,h]quinoxaline (abbreviation: 2mpPCBPDBq). Any of these is preferable as the host material.

Among the above organic compounds, specific examples of the pyridine derivative, the diazine derivative (including the pyrimidine derivative, the pyrazine derivative, and the pyridazine derivative), the triazine derivative, and the furodiazine derivative, which are organic compounds having a high electron-transport property, include an organic compound including a heteroaromatic ring having a diazine ring such as 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), 9,9′-[pyrimidine-4,6-diylbis(biphenyl-3,3′-diyl)]bis(9H-carbazole) (abbreviation: 4,6mCzBP2Pm), 2-[3′-(9,9-dimethyl-9H-fluoren-2-yl)biphenyl-3-yl]-4,6-diphenyl-1,3,5-triazine (abbreviation: mFBPTzn), 8-(1,1′-biphenyl-4-yl)-4-[3-(dibenzothiophen-4-yl)phenyl]-[1]benzofuro[3,2-d]pyrimidine (abbreviation: 8BP-4mDBtPBfpm), 9-[3′-(dibenzothiophen-4-yl)biphenyl-3-yl]naphtho[1′,2′:4,5]furo[2,3-b]pyrazine (abbreviation: 9mDBtBPNfpr), 9-[(3′-dibenzothiophen-4-yl)biphenyl-4-yl]naphtho[1′,2′:4,5]furo[2,3-b]pyrazine (abbreviation: 9pmDBtBPNfpr), 11-[(3′-(dibenzothiophen-4-yl)biphenyl-3-yl]phenanthro[9′,10′:4,5]furo[2,3-b]pyrazine (abbreviation: 11mDBtBPPnfpr), 11-[(3′-dibenzothiophen-4-yl)biphenyl-4-yl]phenanthro[9′,10′:4,5]furo[2,3-b]pyrazine, 11-[(3′-(9H-carbazol-9-yl)biphenyl-3-yl]phenanthro[9′,10′:4,5]furo[2,3-b]pyrazine, 12-(9′-phenyl-3,3′-bi-9H-carbazol-9-yl)phenanth[9′,10′:4,5]furo[2,3-b]pyrazine (abbreviation: 12PCCzPnfpr), 9-[(3′-9-phenyl-9H-carbazol-3-yl)biphenyl-4-yl]naphtho[1′,2′:4,5]furo[2,3-b]pyrazine (abbreviation: 9pmPCBPNfpr), 9-(9′-phenyl-3,3′-bi-9H-carbazol-9-yl)naphtho[1′,2′:4,5]furo[2,3-b]pyrazine (abbreviation: 9PCCzNfpr), 10-(9′-phenyl-3,3′-bi-9H-carbazol-9-yl)naphtho[1′,2′:4,5]furo[2,3-b]pyrazine (abbreviation: 1OPCCzNfpr), 9-[3′-(6-phenylbenzo[b]naphtho[1,2-d]furan-8-yl)biphenyl-3-yl]naphtho[1′,2′:4,5]furo[2,3-b]pyrazine (abbreviation: 9mBnfBPNfpr), 9-{3-[6-(9,9-dimethylfluoren-2-yl)dibenzothiophen-4-yl]phenyl}naphtho[1′,2′:4,5]furo[2,3-b]pyrazine (abbreviation: 9mFDBtPNfpr), 9-[3′-(6-phenyldibenzothiophen-4-yl)biphenyl-3-yl]naphtho[1′,2′:4,5]furo[2,3-b]pyrazine (abbreviation: 9mDBtBPNfpr-02), 9-[3-(9′-phenyl-3,3′-bi-9H-carbazol-9-yl)phenyl]naphtho[1′,2′:4,5]furo[2,3-b]pyrazine (abbreviation: 9mPCCzPNfpr), 9-{(3′-[2,8-diphenyldibenzothiophen-4-yl)biphenyl-3-yl}naphtho[1′,2′:4,5]furo[2,3-b]pyrazine, 11-{(3′-[2,8-diphenyldibenzothiophen-4-yl]biphenyl-3-yl}phenanthro[9′,10′:4,5]furo[2,3-b]pyrazine, 5-[3-(4,6-diphenyl-1,3,5-triazin-2-yl)phenyl]-7,7-dimethyl-5H,7H-indeno[2,1-b]carbazole (abbreviation: mINc(II)PTzn), 2-[3′-(triphenylen-2-yl)biphenyl-3-yl]-4,6-diphenyl-1,3,5-triazine (abbreviation: mTpBPTzn), 2-(-biphenyl-4-yl)-4-phenyl-6-(9,9′-spirobi[9H-fluoren]-2-yl)-1,3,5-triazine (abbreviation: BP-SFTzn), 2,6-bis(4-naphthalen-1-ylphenyl)-4-[4-(3-pyridyl)phenyl]pyrimidine (abbreviation: 2,4NP-6PyPPm), 3-[9-(4,6-diphenyl-1,3,5-triazin-2-yl)-2-dibenzofuranyl]-9-phenyl-9H-carbazole (abbreviation: PCDBfTzn), 2-(biphenyl-3-yl)-4-phenyl-6-{8-[(1,1′:4′,1″-terphenyl)-4-yl]-1-dibenzofuranyl}-1,3,5-triazine (abbreviation: mBP-TPDBfTzn), 6-(biphenyl-3-yl)-4-[3,5-bis(9H-carbazol-9-yl)phenyl]-2-phenylpyrimidine (abbreviation: 6mBP-4Cz2PPm), and 4-[3,5-bis(9H-carbazol-9-yl)phenyl]-2-phenyl-6-(biphenyl-4-yl)pyrimidine (abbreviation: 6BP-4Cz2PPm). Any of these examples is preferred as the host material.

Among the above organic compounds, specific examples of the metal complexes, which are organic compounds having a high electron-transport property, include zinc- and aluminum-based metal complexes, such as tris(8-quinolinolato)aluminum(III) (abbreviation: Alq), tris(4-methyl-8-quinolinolato)aluminum(III) (abbreviation: Almq₃), bis(10-hydroxybenzo[h]quinolinato)beryllium(II) (abbreviation: BeBq₂), bis(2-methyl-8-quinolinolato) (4-phenylphenolato)aluminum(III) (abbreviation: BAlq), and bis(8-quinolinolato)zinc(II) (abbreviation: Znq), and metal complexes having a quinoline ring or a benzoquinoline ring. Any of these is preferable as the host material.

In addition, a high molecular compound such as poly(2,5-pyridinediyl) (abbreviation: PPy), poly[(9,9-dihexylfluorene-2,7-diyl)-co-(pyridine-3,5-diyl)](abbreviation: PF-Py), or poly[(9,9-dioctylfluorene-2,7-diyl)-co-(2,2′-bipyridine-6,6′-diyl)](abbreviation: PF-BPy) or the like is also preferable as the host material.

Furthermore, an organic compound having a bipolar property, i.e., both a high hole-transport property and a high electron-transport property, and including a diazine ring such as 9-phenyl-9′-(4-phenyl-2-quinazolinyl)-3,3′-bi-9H-carbazole (abbreviation: PCCzQz), 2-[4′-(9-phenyl-9H-carbazol-3-yl)-3,1′-biphenyl-1-yl]dibenzo[f,h]quinoxaline (abbreviation: 2mpPCBPDBq), 5-[3-(4,6-diphenyl-1,3,5-triazin-2-yl)phenyl]-7,7-dimethyl-5H,7H-indeno[2,1-b]carbazole (abbreviation: mINc(II)PTzn), 11-[4-(biphenyl-4-yl)-6-phenyl-1,3,5-triazin-2-yl]-11,12-dihydro-12-phenyl-indolo[2,3-a]carbazole (abbreviation: BP-Icz(II)Tzn), or 7-[4-(9-phenyl-9H-carbazol-2-yl)quinazolin-2-yl]-7H-dibenzo[c,g]carbazole (abbreviation: PC-cgDBCzQz) can be used as the host material.

### <Electron-Transport Layer>

The electron-transport layers (114, 114a, and 114b) are each a layer that transports the electrons, which are injected from the second electrode 102 and charge-generation layers (106, 106a, and 106b) by the electron-injection layers (115, 115a, and 115b) to be described later, to the light-emitting layers (113, 113a, and 113b). Note that the heat resistance of the light-emitting device of one embodiment of the present invention can be improved by including the stacked electron-transport layers. As an electron-transport material used for the electron-transport layers (114, 114a, and 114b), a substance having an electron mobility higher than or equal to 1×10⁻⁶ cm²/Vs in the case where the square root of the electric field strength [V/cm] is 600 is preferable. Note that other substances can also be used as long as they have an electron-transport property higher than a hole-transport property. Each of the electron-transport layers (114, 114a, and 114b) functions even in the form of a single layer but may have a stacked-layer structure of two or more layers. Note that since the above-described mixed material has heat resistance, performing a photolithography step over the electron-transport layer including such a material can inhibit the influence of a thermal process on the device characteristics.

### <<Electron-Transport Material>>

As the electron-transport material that can be used for the electron-transport layers (114, 114a, and 114b), an organic compound with a high electron-transport property can be used; for example, a heteroaromatic compound can be used. The heteroaromatic compound refers to a cyclic compound containing at least two different kinds of elements in a ring. Examples of cyclic structures include a three-membered ring, a four-membered ring, a five-membered ring, a six-membered ring, and the like, among which a five-membered ring and a six-membered ring are particularly preferable; the elements contained in the heteroaromatic compound are preferably one or more of nitrogen, oxygen, sulfur, and the like, as well as carbon. In particular, a heteroaromatic compound containing nitrogen (a nitrogen-containing heteroaromatic compound) is preferable, and any of materials having a high electron-transport property (electron-transport materials), such as a nitrogen-containing heteroaromatic compound and a π-electron deficient heteroaromatic compound including the nitrogen-containing heteroaromatic compound, is preferably used.

Note that the electron-transport material can be different from the materials used for the light-emitting layer. Not all excitons formed by recombination of carriers in the light-emitting layer can contribute to light emission and some excitons might be diffused into a layer in contact with the light-emitting layer or a layer in the vicinity of the light-emitting layer. In order to avoid this phenomenon, the energy level (the lowest singlet excitation energy level or the lowest triplet excitation energy level) of a material used for the layer in contact with the light-emitting layer or the layer in the vicinity of the light-emitting layer is preferably higher than that of a material used for the light-emitting layer. Therefore, when a material different from the material of the light-emitting layer is used as the electron-transport material, a light-emitting device with high efficiency can be obtained.

The heteroaromatic compound is an organic compound including at least one heteroaromatic ring.

Note that the heteroaromatic ring includes any one of a pyridine ring, a diazine ring, a triazine ring, a polyazole ring, an oxazole ring, a thiazole ring, and the like. A heteroaromatic ring having a diazine ring includes a heteroaromatic ring having a pyrimidine ring, a pyrazine ring, a pyridazine ring, or the like. A heteroaromatic ring having a polyazole ring includes a heteroaromatic ring having an imidazole ring, a triazole ring, or an oxadiazole ring.

The heteroaromatic ring includes a fused heteroaromatic ring having a fused ring structure. Examples of the fused heteroaromatic ring include a quinoline ring, a benzoquinoline ring, a quinoxaline ring, a dibenzoquinoxaline ring, a quinazoline ring, a benzoquinazoline ring, a dibenzoquinazoline ring, a phenanthroline ring, a furodiazine ring, and a benzimidazole ring.

Examples of a heteroaromatic compound including carbon and one or more of nitrogen, oxygen, sulfur, and the like and having a five-membered ring structure include a heteroaromatic compound having an imidazole ring, a heteroaromatic compound having a triazole ring, a heteroaromatic compound having an oxazole ring, a heteroaromatic compound having an oxadiazole ring, a heteroaromatic compound having a thiazole ring, and a heteroaromatic compound having a benzimidazole ring.

Examples of a heteroaromatic compound including carbon and one or more of nitrogen, oxygen, sulfur, and the like and having a six-membered ring structure include a heteroaromatic compound having a heteroaromatic ring such as a pyridine ring, a diazine ring (a pyrimidine ring, a pyrazine ring, a pyridazine ring, or the like), a triazine ring, or a polyazole ring. Other examples include a heteroaromatic compound having a bipyridine structure and a heteroaromatic compound having a terpyridine structure, which are included in heteroaromatic compounds in which pyridine rings are connected.

Examples of the heteroaromatic compound having a fused ring structure including the above six-membered ring structure as a part include a heteroaromatic compound having a fused heteroaromatic ring such as a quinoline ring, a benzoquinoline ring, a quinoxaline ring, a dibenzoquinoxaline ring, a phenanthroline ring, a furodiazine ring (including a structure in which an aromatic ring is fused to the furan ring of a furodiazine ring), or a benzimidazole ring.

Specific examples of the above-described heteroaromatic compound having a five-membered ring structure (e.g., a polyazole ring (including an imidazole ring, a triazole ring, an oxadiazole ring), an oxazole ring, a thiazole ring, or a benzimidazole ring) include 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-4-phenyl-5-(4-tert-butylphenyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), and 4,4′-bis(5-methylbenzoxazol-2-yl)stilbene (abbreviation: BzOs).

Specific examples of the above-described heteroaromatic compound having a six-membered ring structure (including a heteroaromatic ring having a pyridine ring, a diazine ring, a triazine ring, or the like) include a heteroaromatic compound including a heteroaromatic ring having a pyridine ring, such as 3,5-bis[3-(9H-carbazol-9-yl)phenyl]pyridine (abbreviation: 35DCzPPy) or 1,3,5-tri[3-(3-pyridyl)phenyl]benzene (abbreviation: TmPyPB), a heteroaromatic compound including a heteroaromatic ring having a triazine ring, such as 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-[3′-(triphenylen-2-yl)biphenyl-3-yl]-4,6-diphenyl-1,3,5-triazine (abbreviation: mTpBPTzn), 2-(biphenyl-4-yl)-4-phenyl-6-(9,9′-spirobi[9H-fluoren]-2-yl)-1,3,5-triazine (abbreviation: BP-SFTzn), 2,6-bis(4-naphthalen-1-ylphenyl)-4-[4-(3-pyridyl)phenyl]pyrimidine (abbreviation: 2,4NP-6PyPPm), 3-[9-(4,6-diphenyl-1,3,5-triazin-2-yl)-2-dibenzofuranyl]-9-phenyl-9H-carbazole (abbreviation: PCDBfTzn), 2-(biphenyl-3-yl)-4-phenyl-6-{8-[(1,1′:4′,1″-terphenyl)-4-yl]-1-dibenzofuranyl}-1,3,5-triazine (abbreviation: mBP-TPDBfTzn), 2-{3-[3-(dibenzothiophen-4-yl)phenyl]phenyl}-4,6-diphenyl-1,3,5-triazine (abbreviation: mDBtBPTzn), or mFBPTzn, and a heteroaromatic compound including a heteroaromatic ring having a diazine (pyrimidine) ring, such as 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 4,6mCzBP2Pm, 6-(biphenyl-3-yl)-4-[3,5-bis(9H-carbazol-9-yl)phenyl]-2-phenylpyrimidine (abbreviation: 6mBP-4Cz2PPm), 4-[3,5-bis(9H-carbazol-9-yl)phenyl]-2-phenyl-6-(biphenyl-4-yl)pyrimidine (abbreviation: 6BP-4Cz2PPm), 4-[3-(dibenzothiophen-4-yl)phenyl]-8-(naphthalen-2-yl)-[1]benzofuro[3,2-d]pyrimidine (abbreviation: 8PN-4mDBtPBfpm), 8BP-4mDBtPBfpm, 9mDBtBPNfpr, 9pmDBtBPNfpr, 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfpm), 8-[3′-(dibenzothiophen-4-yl)biphenyl-3-yl]naphtho[1′,2′:4,5]furo[3,2-d]pyrimidine (abbreviation: 8mDBtBPNfpm), or 8-[(2,2′-binaphthalen)-6-yl]-4-[3-(dibenzothiophen-4-yl)phenyl]-[1]benzofuro[3,2-d]pyrimidine (abbreviation: 8(PN2)-4mDBtPBfpm). Note that the above aromatic compounds including a heteroaromatic ring include a heteroaromatic compound having a fused heteroaromatic ring.

Other examples include a heteroaromatic compound including a heteroaromatic ring having a diazine (pyrimidine) ring, such as 2,2′-(pyridine-2,6-diyl)bis(4-phenylbenzo[h]quinazoline) (abbreviation: 2,6(P-Bqn)2Py), 2,2′-(2,2′-bipyridine-6,6′-diyl)bis(4-phenylbenzo[h]quinazoline) (abbreviation: 6,6′(P-Bqn)2BPy), 2,2′-(pyridine-2,6-diyl)bis{4-[4-(2-naphthyl)phenyl]-6-phenylpyrimidine}(abbreviation: 2,6(NP-PPm)2Py), or 6-(biphenyl-3-yl)-4-[3,5-bis(9H-carbazol-9-yl)phenyl]-2-phenylpyrimidine (abbreviation: 6mBP-4Cz2PPm), and a heteroaromatic compound including a heteroaromatic ring having a triazine ring, such as 2,4,6-tris[3′-(pyridin-3-yl)biphenyl-3-yl]-1,3,5-triazine (abbreviation: TmPPPyTz), 2,4,6-tris(2-pyridyl)-1,3,5-triazine (abbreviation: 2Py3Tz), or 2-[3-(2,6-dimethyl-3-pyridyl)-5-(9-phenanthryl)phenyl]-4,6-diphenyl-1,3,5-triazine (abbreviation: mPn-mDMePyPTzn).

Specific examples of the above-described heteroaromatic compound having a fused ring structure including the six-membered ring structure as a part (a heteroaromatic compound having a fused ring structure) include a heteroaromatic compound having a quinoxaline ring, such as bathophenanthroline (abbreviation: Bphen), bathocuproine (abbreviation: BCP), 2,9-di(naphthalen-2-yl)-4,7-diphenyl-1,10-phenanthroline (abbreviation: NBphen), 2,2′-(1,3-phenylene)bis(9-phenyl-1,10-phenanthroline) (abbreviation: mPPhen2P), 2,2′-(pyridine-2,6-diyl)bis(4-phenylbenzo[h]quinazoline) (abbreviation: 2,6(P-Bqn)2Py), 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), or 2mpPCBPDBq.

For the electron-transport layers (114, 114a, and 114b), any of the metal complexes given below as well as the heteroaromatic compounds given above can be used. Examples of the metal complexes include a metal complex having a quinoline ring or a benzoquinoline ring, such as tris(8-quinolinolato)aluminum(III) (abbreviation: Alq₃), Almq₃, 8-quinolinolato-lithium (abbreviation: Liq), BeBq₂, bis(2-methyl-8-quinolinolato)(4-phenylphenolato)aluminum(III) (abbreviation: BAlq), or bis(8-quinolinolato)zinc(II) (abbreviation: Znq), and a metal complex having an oxazole ring or a thiazole ring, such as bis[2-(2-benzoxazolyl)phenolato]zinc(II) (abbreviation: ZnPBO) or bis[2-(2-benzothiazolyl)phenolato]zinc(II) (abbreviation: ZnBTZ).

A high molecular compound such as poly(2,5-pyridinediyl) (abbreviation: PPy), poly[(9,9-dihexylfluorene-2,7-diyl)-co-(pyridine-3,5-diyl)](abbreviation: PF-Py), or poly[(9,9-dioctylfluorene-2,7-diyl)-co-(2,2′-bipyridine-6,6′-diyl)](abbreviation: PF-BPy) can also be used as an electron-transport material.

The electron-transport layer (114, 114a, or 114b) is not limited to a single layer, and may be a stack of two or more layers each made of any of the above substances.

### <Electron-Injection Layer>

The electron-injection layers (115, 115a, and 115b) are each a layer containing a substance having a high electron-injection property. The electron-injection layers (115, 115a, and 115b) are each a layer for increasing the efficiency of electron injection from the second electrode 102 and are each preferably formed using a material whose LUMO level value has a small difference (0.50 eV or less) from the work function value of the material used for the second electrode 102. Thus, the electron-injection layer 115 can be formed using an alkali metal, an alkaline earth metal, or a compound thereof, such as lithium, cesium, lithium fluoride (LiF), cesium fluoride (CsF), calcium fluoride (CaF₂), 8-quinolinolato-lithium (abbreviation: Liq), 2-(2-pyridyl)phenolatolithium (abbreviation: LiPP), 2-(2-pyridyl)-3-pyridinolatolithium (abbreviation: LiPPy), 4-phenyl-2-(2-pyridyl)phenolatolithium (abbreviation: LiPPP), lithium oxide (LiOₓ), or cesium carbonate. A rare earth metal compound such as erbium fluoride (ErF₃) or ytterbium (Yb) can also be used. It is also possible to use a compound including a 1,3,4,6,7,8-tetrahydro-2H-pyrimido[1,2-a]pyrimidine skeleton, such as 1-(9,9′-spirobi[9H-fluoren]-2-yl)-1,3,4,6,7,8-hexahydro-2H-pyrimido[1,2-a]pyrimidine (abbreviation: 2hppSF), 1,1′-(9,9′-spirobi[9H-fluorene]-2,7-diyl)bis(1,3,4,6,7,8-hexahydro-2H-pyrimido[1,2-a]pyrimidine) (abbreviation: 2,7hpp2SF), or 1,1′-pyridine-2,6-diyl-bis(1,3,4,6,7,8-hexahydro-2H-pyrimido[1,2-a]pyrimidine) (abbreviation: hpp2Py). Note that to form the electron-injection layers (115, 115a, and 115b), a plurality of kinds of the above-described materials may be mixed or a plurality of kinds of the above-described materials may be stacked. Electride may also be used for the electron-injection layers (115, 115a, and 115b). Examples of the electride include a substance in which electrons are added at high concentration to a mixed oxide of calcium and aluminum. Note that any of the substances used in the electron-transport layers (114, 114a, and 114b), which are given above, can also be used.

A mixed material in which an organic compound and an electron donor (donor) are mixed may also be used in the electron-injection layers (115, 115a, and 115b). Such a mixed material is excellent in an electron-injection property and an electron-transport property because electrons are generated in the organic compound by the electron donor. In this case, the organic compound is preferably a material excellent in transporting the generated electrons; specifically, for example, the above-mentioned electron-transport materials (metal complexes, heteroaromatic compounds, and the like) used in the electron-transport layers (114, 114a, and 114b) can be used. Any substance showing an electron-donating property with respect to the organic compound can serve as an electron donor. Specifically, an alkali metal, an alkaline earth metal, and a rare earth metal are preferable, and lithium, cesium, magnesium, calcium, erbium, ytterbium, and the like are given. In addition, an alkali metal oxide and an alkaline earth metal oxide are preferable, and lithium oxide, calcium oxide, barium oxide, and the like are given. A Lewis base such as magnesium oxide can also be used. An organic compound such as tetrathiafulvalene (abbreviation: TTF) can also be used. Alternatively, a stack of these materials may be used.

Moreover, a mixed material in which an organic compound and a metal are mixed may also be used in the electron-injection layers (115, 115a, and 115b). The organic compound used here preferably has a LUMO level higher than or equal to −3.60 eV and lower than or equal to −2.30 eV. Moreover, a material having an unshared electron pair is preferable.

Thus, as the organic compound used in the above mixed material, a mixed material obtained by mixing a metal and the heteroaromatic compound given above as the material that can be used for the electron-transport layer may be used. Preferable examples of the heteroaromatic compound include materials having an unshared electron pair, such as a heteroaromatic compound having a five-membered ring structure (e.g., an imidazole ring, a triazole ring, an oxazole ring, an oxadiazole ring, a thiazole ring, or a benzimidazole ring), a heteroaromatic compound having a six-membered ring structure (e.g., a pyridine ring, a diazine ring (including a pyrimidine ring, a pyrazine ring, a pyridazine ring, and the like), a triazine ring, a bipyridine ring, or a terpyridine ring), and a heteroaromatic compound having a fused ring structure including a six-membered ring structure as a part (e.g., a quinoline ring, a benzoquinoline ring, a quinoxaline ring, a dibenzoquinoxaline ring, or a phenanthroline ring). Since the materials are specifically described above, description thereof is omitted here.

As the metal used for the above mixed material, a transition metal that belongs to Group 5, Group 7, Group 9, or Group 11 in the periodic table or a material that belongs to Group 13 is preferably used, and Ag, Cu, Al, In, and the like can be given as examples. In this case, the organic compound forms a singly occupied molecular orbital (SOMO) with the transition metal.

Note that to amplify light obtained from the light-emitting layer 113b, for example, the optical path length between the second electrode 102 and the light-emitting layer 113b is preferably less than one fourth of the wavelength k of light emitted from the light-emitting layer 113b. In that case, the optical path length can be adjusted by changing the thickness of the electron-transport layer 114b or the electron-injection layer 115b.

When the charge-generation layer 106 is provided between the two EL layers (103a and 103b) as in the light-emitting device in FIG. 1E, a structure in which a plurality of EL layers are stacked between a pair of electrodes (the structure is also referred to as a tandem structure) can be obtained.

### <Charge-Generation Layer 106>

The charge-generation layer 106 has a function of injecting electrons into the EL layer 103a and injecting holes into the EL layer 103b when a voltage is applied between the first electrode (anode) 101 and the second electrode (cathode) 102. The charge-generation layer 106 may have either a structure in which an electron acceptor (acceptor) is added to a hole-transport material (also referred to as a P-type layer) or a structure in which an electron donor (donor) is added to an electron-transport material (also referred to as an electron-injection buffer layer). Alternatively, both of these structures may be stacked. Furthermore, an electron-relay layer may be provided between the P-type layer and the electron-injection buffer layer. Note that forming the charge-generation layer 106 with the use of any of the above materials can inhibit an increase in driving voltage caused by the stack of the EL layers.

In the case where the charge-generation layer 106 has a structure in which an electron acceptor is added to a hole-transport material that is an organic compound (P-type layer), any of the materials described in this embodiment can be used as the hole-transport material. As examples of the electron acceptor, 7,7,8,8-tetracyano-2,3,5,6-tetrafluoroquinodimethane (abbreviation: F₄-TCNQ), chloranil, and the like can be given. Other examples include oxides of metals that belong to Group 4 to Group 8 of the periodic table. Specific examples include vanadium oxide, niobium oxide, tantalum oxide, chromium oxide, molybdenum oxide, tungsten oxide, manganese oxide, and rhenium oxide. Any of the above-described acceptor materials may be used. Furthermore, a mixed film obtained by mixing materials of the P-type layer or a stack of single films containing the respective materials may be used.

In the case where the charge-generation layer 106 has a structure in which an electron donor is added to an electron-transport material (electron-injection buffer layer), any of the materials described in this embodiment can be used as the electron-transport material. As the electron donor, it is possible to use an alkali metal, an alkaline earth metal, a rare earth metal, a metal belonging to Group 2 or Group 13 of the periodic table, or an oxide or a carbonate thereof. Specifically, lithium (Li), cesium (Cs), magnesium (Mg), calcium (Ca), ytterbium (Yb), indium (In), lithium oxide (Li₂O), cesium carbonate, or the like is preferably used. An alkali metal compound such as Liq may be used. An organic compound such as tetrathianaphthacene may be used as the electron donor. An organic compound including a 1,3,4,6,7,8-tetrahydro-2H-pyrimido[1,2-a]pyrimidine skeleton, such as 2hppSF, 2,7hpp2SF, or hpp2Py may be used as the electron donor. When any of these organic compounds is used as the electron donor, the electron-transport material to be combined with the electron donor is preferably an organic compound including a heteroaromatic ring having a phenanthroline ring, such as bathophenanthroline (abbreviation: Bphen), bathocuproine (abbreviation: BCP), 2,9-di(naphthalen-2-yl)-4,7-diphenyl-1,10-phenanthroline (abbreviation: NBphen), or 2,2′-(1,3-phenylene)bis(9-phenyl-1,10-phenanthroline) (abbreviation: mPPhen2P), in which case driving voltage of the light-emitting device can be reduced.

When an electron-relay layer is provided between a P-type layer and an electron-injection buffer layer in the charge-generation layer 106, the electron-relay layer contains at least a substance having an electron-transport property and has a function of preventing an interaction between the electron-injection buffer layer and the P-type layer and transferring electrons smoothly. The LUMO level of the substance having an electron-transport property in the electron-relay layer is preferably between the LUMO level of the acceptor substance in the P-type layer and the LUMO level of the substance having an electron-transport property in the electron-transport layer in contact with the charge-generation layer 106. A specific energy level of the LUMO level of the substance having an electron-transport property in the electron-relay layer is preferably higher than or equal to −5.00 eV, further preferably higher than or equal to −5.00 eV and lower than or equal to −3.00 eV. Note that as the substance having an electron-transport property in the electron-relay layer, a phthalocyanine-based material or a metal complex having a metal-oxygen bond and an aromatic ligand is preferably used.

Note that in terms of light extraction efficiency, it is preferable that the charge-generation layer 106 have a light-transmitting property with respect to visible light (specifically, the visible light transmittance with respect to the charge-generation layer 106 is preferably 40% or higher). Furthermore, the charge-generation layer 106 functions even when having lower conductivity than the first electrode 101 or the second electrode 102.

Although FIG. 1E illustrates the structure in which two EL layers 103 are stacked, three or more EL layers may be stacked with charge-generation layers each provided between different EL layers.

### <Cap Layer>

Although not illustrated in FIG. 1A to FIG. 1E, a cap layer may be provided over the second electrode 102 of the light-emitting device. For example, a material with a high refractive index can be used for the cap layer. When the cap layer is provided over the second electrode 102, extraction efficiency of light emitted from the second electrode 102 can be improved.

Specific examples of a material that can be used for the cap layer include 5,5′-diphenyl-2,2′-di-5H-[1]benzothieno[3,2-c]carbazole (abbreviation: BisBTc) and 4,4′,4″-(benzene-1,3,5-triyl)tri(dibenzothiophene) (abbreviation: DBT3P-II).

### <Substrate>

The light-emitting device described in this embodiment can be formed over a variety of substrates. Note that the type of substrate is not limited to a certain type. Examples of the substrate include semiconductor substrates (e.g., a single crystal substrate and a silicon substrate), an SOI substrate, a glass substrate, a quartz substrate, a plastic substrate, a metal substrate, a stainless steel substrate, a substrate including stainless steel foil, a tungsten substrate, a substrate including tungsten foil, a flexible substrate, an attachment film, paper including a fibrous material, and a base material film including a fibrous material.

Examples of the glass substrate include barium borosilicate glass, aluminoborosilicate glass, and soda lime glass. Examples of the flexible substrate, the attachment film, and the base material film include plastics typified by polyethylene terephthalate (PET), polyethylene naphthalate (PEN), and polyether sulfone (PES), a synthetic resin such as acrylic resin, polypropylene, polyester, polyvinyl fluoride, polyvinyl chloride, polyamide, polyimide, aramid, epoxy resin, an inorganic vapor deposition film, and paper.

For fabrication of the light-emitting device described in this embodiment, a vapor phase method such as an evaporation method or a liquid phase method such as a spin coating method and an ink-jet method can be used. In the case of using an evaporation method, a physical vapor deposition method (PVD method) such as a sputtering method, an ion plating method, an ion beam evaporation method, a molecular beam evaporation method, or a vacuum evaporation method, a chemical vapor deposition method (CVD method), or the like can be used. Specifically, layers having a variety of functions (the hole-injection layer 111, the hole-transport layer 112, the light-emitting layer 113, the electron-transport layer 114, and the electron-injection layer 115) included in the EL layer of the light-emitting device can be formed by an evaporation method (e.g., a vacuum evaporation method), a coating method (e.g., a dip coating method, a die coating method, a bar coating method, a spin coating method, or a spray coating method), a printing method (e.g., an ink-jet method, a screen printing (stencil) method, an offset printing (planography) method, a flexography (relief printing) method, a gravure printing method, or a micro-contact printing method), or the like.

In the case where a film formation method such as the coating method or the printing method described above is employed, a high molecular compound (e.g., an oligomer, a dendrimer, or a polymer), a middle molecular compound (a compound between a low molecular compound and a high molecular compound with a molecular weight of greater than or equal to 400 and less than or equal to 4000), an inorganic compound (e.g., a quantum dot material), or the like can be used. The quantum dot material can be a colloidal quantum dot material, an alloyed quantum dot material, a core-shell quantum dot material, a core quantum dot material, or the like.

Materials that can be used for the layers (the hole-injection layer 111, the hole-transport layer 112, the light-emitting layer 113, the electron-transport layer 114, and the electron-injection layer 115) included in the EL layer 103 of the light-emitting device described in this embodiment are not limited to the materials described in this embodiment, and other materials can be used in combination as long as the functions of the layers are fulfilled.

Note that in this specification and the like, the term “layer” and the term “film” can be interchanged with each other as appropriate.

The structures described in this embodiment can be used in an appropriate combination with any of the structures described in the other embodiments.

### Embodiment 4

In this embodiment, specific structure examples and an example of a manufacturing method of a light-emitting and light-receiving apparatus of one embodiment of the present invention are described.

### <Structure Example of Light-Emitting and Light-Receiving Apparatus 700>

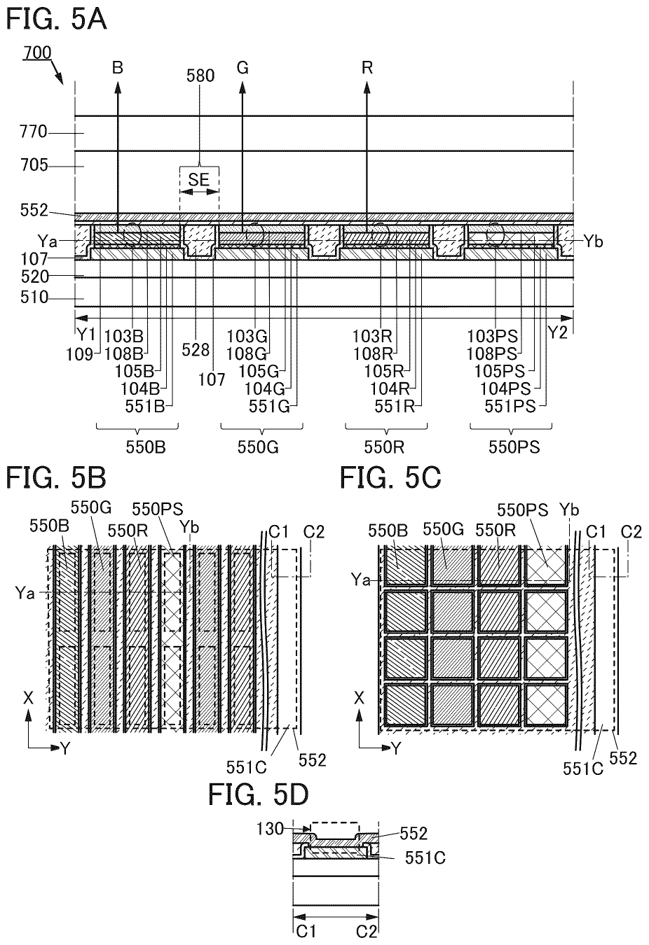

A light-emitting and light-receiving apparatus 700 illustrated in FIG. 5A includes a light-emitting device 550B, a light-emitting device 550G, a light-emitting device 550R, and a light-receiving device 550PS that are formed over a functional layer 520 over a first substrate 510. The functional layer 520 includes driver circuits such as a gate driver and a source driver that are composed of a plurality of transistors and wirings that electrically connect these circuits. Note that these driver circuits are electrically connected to the light-emitting device 550B, the light-emitting device 550G, the light-emitting device 550R, and the light-receiving device 550PS, for example, and can drive them. The light-emitting and light-receiving apparatus 700 includes an insulating layer 705 over the functional layer 520 and the devices (the light-emitting devices and the light-receiving device), and the insulating layer 705 has a function of bonding a second substrate 770 and the functional layer 520.

The light-emitting device 550B, the light-emitting device 550G, and the light-emitting device 550R each have the device structure described in Embodiment 1. In addition, the structure of the EL layer 103 (see FIG. 1A) differs between the devices; for example, a light-emitting layer 105B of an EL layer 103B can emit blue light, a light-emitting layer 105G of an EL layer 103G can emit green light, and a light-emitting layer 105R of the EL layer 103G can emit red light.

Note that although in this embodiment, the case where the devices (a plurality of light-emitting devices and the light-receiving device) are formed separately is described, part of an EL layer of the light-emitting device (a hole-injection layer, a hole-transport layer, and an electron-transport layer) and part of an active layer of the light-receiving device (a hole-injection layer, a hole-transport layer, and an electron-transport layer) may be formed using the same material at the same time in the manufacturing process. The details will be described in Embodiment 8.

In this specification and the like, a structure in which light-emitting layers in light-emitting devices of different colors (e.g., blue (B), green (G), and red (R)) and a light-receiving layer in light-receiving device are separately formed or separately patterned may be referred to as an SBS (Side By Side) structure. Note that in the light-emitting and light-receiving apparatus 700 illustrated in FIG. 5A, the light-emitting device 550B, the light-emitting device 550G, the light-emitting device 550R, and the light-receiving device 550PS are arranged in this order; however, one embodiment of the present invention is not limited thereto. For example, in the light-emitting and light-receiving apparatus 700, the light-emitting device 550R, the light-emitting device 550G, the light-emitting device 550B, and the light-receiving device 550PS may be arranged in this order.

In FIG. 5A, the light-emitting device 550B includes an electrode 551B, an electrode 552, and the EL layer 103B interposed between the electrode 551B and the electrode 552. The light-emitting device 550G includes an electrode 551G, the electrode 552, and the EL layer 103G interposed between the electrode 551G and the electrode 552. The light-emitting device 550R includes an electrode 551R, the electrode 552, and an EL layer 103R interposed between the electrode 551R and the electrode 552. The EL layers (103B, 103G, and 103R) each have a stacked-layer structure of layers having different functions including the light-emitting layers (105B, 105G, and 105R). A specific structure of each layer of the light-emitting device is as described in Embodiment 1 to 3.

In FIG. 5A, the light-receiving device 550PS includes an electrode 551PS, the electrode 552, and a light-receiving layer 103PS interposed between the electrode 551PS and the electrode 552. The light-receiving layer 103PS has a stacked-layer structure of layers having different functions including an active layer 105PS. Note that a specific structure of each layer of the light-receiving device is as described in Embodiment 8.

FIG. 5A illustrates a case where the EL layer 103B includes a hole-injection/transport layer 104B, the light-emitting layer 105B, an electron-transport layer 108B, and an electron-injection layer 109; the EL layer 103G includes a hole-injection/transport layer 104G, the light-emitting layer 105G, an electron-transport layer 108G, and the electron-injection layer 109; the EL layer 103R includes a hole-injection/transport layer 104R, the light-emitting layer 105R, an electron-transport layer 108R, and the electron-injection layer 109; and the light-receiving layer 103PS includes a hole-injection/transport layer 104PS, the active layer 105PS, an electron-transport layer 108PS, and the electron-injection layer 109. However, the present invention is not limited to the case.

Note that the electron-transport layers (108B, 108G, and 108R) may have a function of blocking holes moving from the anode side to the cathode side through the light-emitting layers (103B, 103G, and 103R). The electron-injection layer 109 may have a stacked-layer structure in which some or all of layers are formed using different materials.

As illustrated in FIG. 5A, an insulating layer 107 may be formed on side surfaces (or end portions) of the hole-injection/transport layers (104B, 104G, and 104R), the light-emitting layers (105B, 105G, and 105R), and the electron-transport layers (108B, 108G, and 108R) included in the EL layers (103B, 103G, and 103R), and side surfaces (or end portions) of the hole-injection/transport layer 104PS, the active layer 105PS, and the electron-transport layer 108PS included in the light-receiving layer 103PS. The insulating layer 107 is formed in contact with the side surfaces (or the end portions) of the EL layers (103B, 103G, and 103R) and the light-receiving layer 103PS. This can inhibit entry of oxygen, moisture, or constituent elements thereof into the inside through the side surfaces of the EL layers (103B, 103G, and 103R) and the light-receiving layer 103PS. For the insulating layer 107, aluminum oxide, magnesium oxide, hafnium oxide, gallium oxide, indium gallium zinc oxide, silicon nitride, silicon nitride oxide, or the like can be used, for example. The above-described materials may be stacked to form the insulating layer 107. The insulating layer 107 can be formed by a sputtering method, a CVD method, an MBE method, a PLD method, an ALD method, or the like and is formed preferably by an ALD method, which enables favorable coverage. Note that the insulating layer 107 continuously covers the side surfaces (or the end portions) of parts of the EL layers (103B, 103G, and 103R) and parts of the light-receiving layer 103PS of adjacent light-emitting devices. For example, in FIG. 5A, the side surfaces of part of the EL layer 103G of the light-emitting device 550B and part of the EL layer 103G of the light-emitting device 550G are covered with the insulating layer 107. In a region covered with the insulating layer 107, a partition wall 528 formed using an insulating material is preferably formed, as illustrated in FIG. 5A.

In FIG. 5A, the electron-injection layer 109 and the electrode 552 are layers (common layers) shared by the devices (550B, 550G, 550R, and 550PS). Note that the electron-injection layer 109 may have a stacked-layer structure of two or more layers (for example, stacked layers having different electric resistances).

The partition walls 528 are provided between the electrodes (551i, 551G, 551R, and 551PS), parts of the EL layers (103B, 103G, and 103R), and part of the light-receiving layer 103PS. As illustrated in FIG. 5A, the partition walls 528 are in contact with the side surfaces (or the end portions) of the electrodes (551B, 551G, 551R, and 551PS) and parts of the EL layers (103B, 103G, and 103R) and part of the light-receiving layer 103PS of the devices through the insulating layer 107.

In each of the EL layers and the light-receiving layer, particularly the hole-injection layer, which is included in the hole-transport region between the anode and the light-emitting layer and the hole-transport region between the anode and the active layer, often has high conductivity; therefore, a hole-injection layer formed as a layer shared by adjacent devices might cause crosstalk. Thus, as described in this structure example, the partition walls 528 formed of an insulating material are provided between the EL layers and between the EL layer and the light-receiving layer, which can inhibit occurrence of crosstalk between adjacent devices (between the light-receiving device and the light-emitting device, between the light-emitting device and the light-emitting device, or between the light-receiving device and the light-receiving device).

In the manufacturing method described in this embodiment, side surfaces (or end portions) of the EL layer and the light-receiving layer are exposed in the patterning step. This may promote deterioration of the EL layer and the light-receiving layer by allowing the entry of oxygen, water, or the like through the side surfaces (or the end portions) of the EL layer and the light-receiving layer. Therefore, providing the partition wall 528 can inhibit the deterioration of the EL layer and the light-receiving layer in the manufacturing process.

Providing the partition wall 528 can flatten a depressed portion formed between adjacent devices (between the light-receiving device and the light-emitting device, between the light-emitting device and the light-emitting device, or between the light-receiving device and the light-receiving device). When the depressed portion is reduced, disconnection of the electrode 552 formed over the EL layers and the light-receiving layer can be inhibited. As an insulating material used for forming the partition wall 528, an organic material such as an acrylic resin, a polyimide resin, an epoxy resin, an imide resin, a polyamide resin, a polyimide-amide resin, a silicone resin, a siloxane resin, a benzocyclobutene-based resin, a phenol resin, or precursors of these resins can be used, for example. An organic material such as polyvinyl alcohol (PVA), polyvinyl butyral, polyvinylpyrrolidone, polyethylene glycol, polyglycerin, pullulan, water-soluble cellulose, or an alcohol-soluble polyamide resin can also be used. A photosensitive resin such as a photoresist can also be used. Note that as the photosensitive resin, a positive material or a negative material can be used.

With the photosensitive resin, the partition wall 528 can be fabricated only by light exposure and development steps. The partition wall 528 may be formed using a negative photosensitive resin (e.g., a resist material). In the case where an insulating layer containing an organic material is used as the partition wall 528, a material absorbing visible light is suitably used. When a material that absorbs visible light is used for the partition wall 528, light emitted from the EL layer can be absorbed by the partition wall 528, so that light that might leak to the adjacent EL layer and the adjacent light-receiving layer (stray light) can be inhibited. Thus, a display panel having high display quality can be provided.

For example, the difference between the top-surface level of the partition wall 528 and the top-surface level of any of the EL layers (103B, 103G, and 103R) and the light-receiving layer 103PS is preferably 0.5 times or less, further preferably 0.3 times or less the thickness of the partition wall 528. The partition wall 528 may be provided such that the top-surface level of any of the EL layer 103B, the EL layer 103G, the EL layer 103R, and the light-receiving layer 103PS is higher than the top-surface level of the partition wall 528, for example. Alternatively, the partition wall 528 may be provided such that the top-surface level of the partition wall 528 is higher than the top-surface level of the light-emitting layer included in each of the EL layer 103B, the EL layer 103G, the EL layer 103R, and the light-receiving layer 103PS, for example.

When electrical continuity is established between the EL layer 103B, the EL layer 103G, the EL layer 103R, and the light-receiving layer 103PS in a light-emitting and light-receiving apparatus (display panel) with a high resolution exceeding 1000 ppi, a crosstalk phenomenon occurs, resulting in a narrower color gamut of the light-emitting and light-receiving apparatus. Providing the partition wall 528 in a high-resolution display panel with more than 1000 ppi, preferably more than 2000 ppi, or further preferably in an ultrahigh-resolution display panel with more than 5000 ppi allows the display panel to express vivid colors.

FIG. 5B and FIG. 5C are each a schematic top view of the light-emitting and light-receiving apparatus 700 taken along the dashed-dotted line Ya-Yb in the cross-sectional view of FIG. 5A. Each of the light-emitting devices (550B, 550G, and 550R) are arranged in a matrix. FIG. 5B illustrates what is called stripe arrangement, in which the light-emitting devices of the same color are arranged in X-direction. FIG. 5C illustrates a structure in which the light-emitting devices of the same color are arranged in the X-direction and separated by patterning for each pixel. Note that the arrangement method of the light-emitting devices is not limited thereto; another arrangement method such as delta arrangement, zigzag arrangement, PenTile arrangement, or diamond arrangement may also be used.

Each of the EL layers (103B, 103G, and 103R) and the light-receiving layer 103PS are processed to be separated by patterning using a photolithography method; hence, a high-resolution light-emitting and light-receiving apparatus (display panel) can be fabricated. End portions (side surfaces) of the EL layer processed by patterning using a photolithography method have substantially one surface (or are positioned on substantially the same plane). In this case, the width (SE) of a space 580 between the EL layers and between the EL layer and the light-receiving layer is preferably 5 μm or less, further preferably 1 μm or less.

In the EL layer, particularly the hole-injection layer, which is included in the hole-transport region positioned between the anode and the light-emitting layer, often has high conductivity; therefore, a hole-injection layer formed as a layer shared by adjacent light-emitting devices might cause crosstalk. Therefore, processing the EL layers to be separated by patterning using a photolithography method as shown in this structure example can suppress occurrence of crosstalk between adjacent light-emitting devices.

FIG. 5D is a schematic cross-sectional view taken along the dashed-dotted line C1-C2 in FIG. 5B and FIG. 5C. FIG. 5D illustrates a connection portion 130 where a connection electrode 551C and the electrode 552 are electrically connected. In the connection portion 130, the electrode 552 is provided over and in contact with the connection electrode 551C. In addition, the partition wall 528 is provided to cover the end portion of the connection electrode 551C.

### <Example of Manufacturing Method of Light-Emitting and Light-Receiving Apparatus>

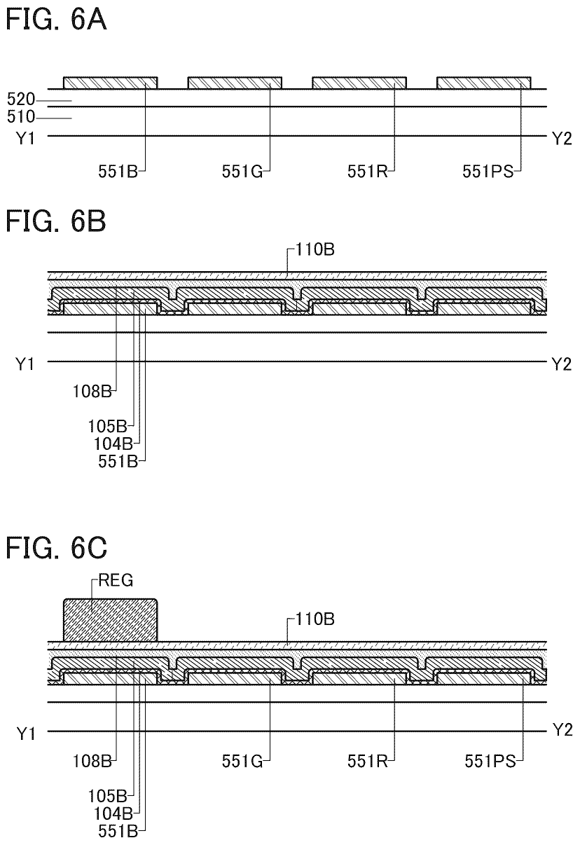

The electrode 551B, the electrode 551G, the electrode 551R, and the electrode 551PS are formed as illustrated in FIG. 6A. For example, a conductive film is formed over the functional layer 520 over the first substrate 510 and processed into predetermined shapes by a photolithography method.

The conductive film can be formed by a sputtering method, a chemical vapor deposition (CVD) method, a molecular beam epitaxy (MBE) method, a vacuum evaporation method, a pulsed laser deposition (PLD) method, an atomic layer deposition (ALD) method, or the like. Examples of the CVD method include a plasma-enhanced chemical vapor deposition (PECVD: Plasma Enhanced CVD) method and a thermal CVD method. As an example of the thermal CVD method, a metal organic chemical vapor deposition (MOCVD: Metal Organic CVD) method can be given.

The conductive film may be processed by a nanoimprinting method, a sandblasting method, a lift-off method, or the like as well as a photolithography method described above. Alternatively, island-shaped thin films may be directly formed by a film formation method using a shielding mask such as a metal mask.

There are two typical processing methods using a photolithography method. In one of the methods, a resist mask is formed over a thin film that is to be processed, the thin film is processed by etching or the like, and then the resist mask is removed. In the other method, a photosensitive thin film is formed and then processed into a desired shape by light exposure and development. The former method involves heat treatment steps such as heating after resist application (PAB: Pre Applied Bake) and heating after light exposure (PEB: Post Exposure Bake). In one embodiment of the present invention, a lithography method is used not only for processing of a conductive film but also for processing of a thin film used for formation of an EL layer (a film made of an organic compound or a film partly including an organic compound).

As light for light exposure in a photolithography method, it is possible to use the i-line (wavelength: 365 nm), the g-line (wavelength: 436 nm), the h-line (wavelength: 405 nm), or light in which the i-line, the g-line, and the h-line are mixed. Alternatively, ultraviolet light, KrF laser light, ArF laser light, or the like can be used. Light exposure may be performed by liquid immersion light exposure technique. As the light for light exposure, extreme ultraviolet (EUV) light or X-rays may also be used. Instead of the light for light exposure, an electron beam can be used. It is preferable to use extreme ultraviolet light, X-rays, or an electron beam because extremely minute processing can be performed. Note that a photomask is not needed when light exposure is performed by scanning with a beam such as an electron beam.

For etching of a thin film using a resist mask, a dry etching method, a wet etching method, a sandblast method, or the like can be used.

Subsequently, as illustrated in FIG. 6B, the hole-injection/transport layer 104B, the light-emitting layer 105B, and the electron-transport layer 108B are formed over the electrode 551B, the electrode 551G, the electrode 551R, and the electrode 551PS. Note that the hole-injection/transport layer 104B, the light-emitting layer 105B, and the electron-transport layer 108B can be formed using a vacuum evaporation method, for example. Furthermore, a sacrificial layer 110B is formed over the electron-transport layer 108B. For the formation of the hole-injection/transport layer 104B, the light-emitting layer 105B, and the electron-transport layer 108B, any of the materials described in Embodiment 1 to 3 can be used.

For the sacrificial layer 110B, it is preferable to use a film highly resistant to etching treatment performed on the hole-injection/transport layer 104B, the light-emitting layer 105B, and the electron-transport layer 108B, i.e., a film having high etching selectivity. The sacrificial layer 110B preferably has a stacked-layer structure of a first sacrificial layer and a second sacrificial layer which have different etching selectivities. Moreover, for the sacrificial layer 110B, it is possible to use a film that can be removed by a wet etching method less likely to cause damage to the EL layer 103B. In wet etching, oxalic acid or the like can be used as an etching material. Note that in this specification and the like, a sacrificial layer may be referred to as a mask layer.

The sacrificial layer 110B can be formed using an inorganic film such as a metal film, an alloy film, a metal oxide film, a semiconductor film, or an inorganic insulating film, for example. The sacrificial layer 110B can be formed by any of a variety of film formation methods such as a sputtering method, an evaporation method, a CVD method, and an ALD method.

For the sacrificial layer 110B, a metal material such as gold, silver, platinum, magnesium, nickel, tungsten, chromium, molybdenum, iron, cobalt, copper, palladium, titanium, aluminum, yttrium, zirconium, or tantalum or an alloy material containing the metal material can be used. It is particularly preferable to use a low-melting-point material such as aluminum or silver.

The sacrificial layer 110B can be formed using a metal oxide such as indium gallium zinc oxide (In—Ga—Zn oxide, also referred to as IGZO). It is also possible to use indium oxide, indium zinc oxide (In—Zn oxide), indium tin oxide (In—Sn oxide), indium titanium oxide (In—Ti oxide), indium tin zinc oxide (In—Sn—Zn oxide), indium titanium zinc oxide (In—Ti—Zn oxide), indium gallium tin zinc oxide (In—Ga—Sn—Zn oxide), or the like. Alternatively, indium tin oxide containing silicon can also be used, for example.

Note that an element M (M is one or more kinds selected from aluminum, silicon, boron, yttrium, copper, vanadium, beryllium, titanium, iron, nickel, germanium, zirconium, molybdenum, lanthanum, cerium, neodymium, hafnium, tantalum, tungsten, and magnesium) may be used instead of gallium described above. In particular, M is preferably one or more kinds selected from gallium, aluminum, and yttrium.

For the sacrificial layer 110B, an inorganic insulating material such as aluminum oxide, hafnium oxide, or silicon oxide can be used.

The sacrificial layer 110B is preferably formed using a material that can be dissolved in a solvent chemically stable with respect to the electron-transport layer 108B, which is at least the uppermost layer. In particular, a material that will be dissolved in water or alcohol can be suitably used for the sacrificial layer 110B. In formation of the sacrificial layer 110B, it is preferable that application of such a material dissolved in a solvent such as water or alcohol be performed by a wet film formation method and followed by heat treatment for evaporating the solvent. At this time, the heat treatment is preferably performed in a reduced-pressure atmosphere, in which case the solvent can be removed at a low temperature in a short time and thermal damage to the hole-injection/transport layer 104B, the light-emitting layer 105B, and the electron-transport layer 108B can be reduced accordingly.

In the case where the sacrificial layer 110B having a stacked-layer structure is formed, the stacked-layer structure can include the first sacrificial layer formed using any of the above-described materials and the second sacrificial layer thereunder.

The second sacrificial layer in that case is a film used as a hard mask for etching of the first sacrificial layer. In processing the second sacrificial layer, the first sacrificial layer is exposed. Thus, a combination of films having high etching selectivity therebetween is selected for the first sacrificial layer and the second sacrificial layer. Thus, a film that can be used for the second sacrificial layer can be selected in accordance with the etching conditions of the first sacrificial layer and the etching conditions of the second sacrificial layer.

For example, in the case where dry etching using a gas containing fluorine (also referred to as a fluorine-based gas) is performed for the etching of the second sacrificial layer, silicon, silicon nitride, silicon oxide, tungsten, titanium, molybdenum, tantalum, tantalum nitride, an alloy containing molybdenum and niobium, an alloy containing molybdenum and tungsten, or the like can be used for the second sacrificial layer. Here, a metal oxide film of IGZO, ITO, or the like is given as an example of a film having high etching selectivity (that is, enabling low etching rate) in dry etching using the fluorine-based gas above, and such a film can be used as the first sacrificial layer.

Note that the material for the second sacrificial layer is not limited to the above and can be selected from a variety of materials in accordance with the etching conditions of the first sacrificial layer and the etching conditions of the second sacrificial layer. For example, any of the films that can be used for the first sacrificial layer above can be selected.

As the second sacrificial layer, a nitride film can be used, for example. Specifically, it is possible to use a nitride such as silicon nitride, aluminum nitride, hafnium nitride, titanium nitride, tantalum nitride, tungsten nitride, gallium nitride, or germanium nitride.

Alternatively, an oxide film can be used as the second sacrificial layer. Typically, a film of an oxide or an oxynitride such as silicon oxide, silicon oxynitride, aluminum oxide, aluminum oxynitride, hafnium oxide, or hafnium oxynitride can be used.

Next, as illustrated in FIG. 6C, a resist is applied onto the sacrificial layer 110B, and the resist having a desired shape (resist mask: REG) is formed by a photolithography method. Such a method involves heat treatment steps such as heating after resist application (PAB: Pre Applied Bake) and heating after light exposure (PEB: Post Exposure Bake). The PAB temperature reaches approximately 100° C. and the PEB temperature reaches approximately 120° C., for example. Therefore, the light-emitting device needs to be resistant to such treatment temperatures.

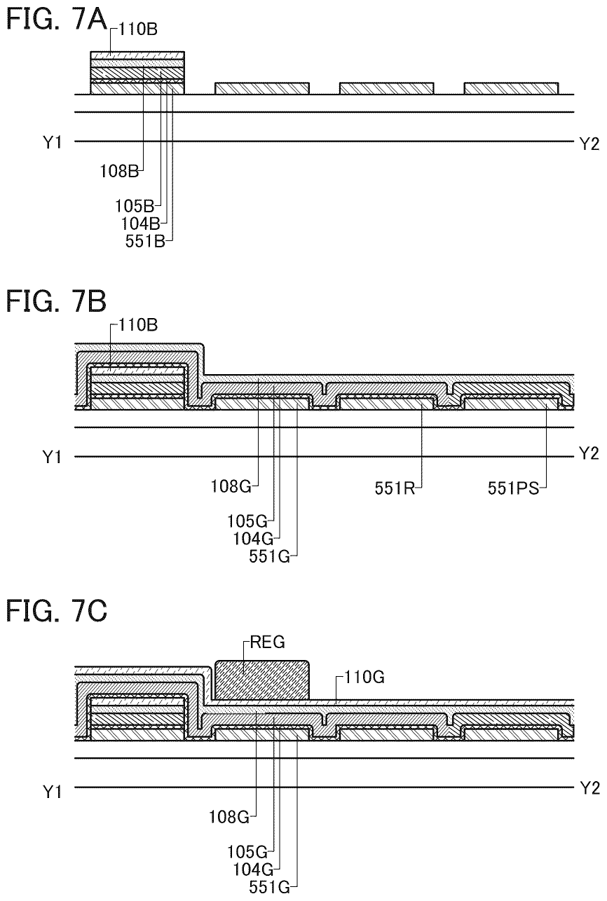

Next, part of the sacrificial layer 110B that is not covered with the resist mask REG is removed by etching using the obtained resist mask REG, the resist mask REG is removed, and then the hole-injection/transport layer 104B, the light-emitting layer 105B, and the electron-transport layer 108B that are not covered with the sacrificial layer are removed by etching, so that the hole-injection/transport layer 104B, the light-emitting layer 105B, and the electron-transport layer 108B are processed to have side surfaces (or have their side surfaces exposed) over the electrode 551B or have belt-like shapes extending in the direction intersecting with the sheet. Note that dry etching is preferably employed for the etching. In the case where the sacrificial layer 110B has the aforementioned stacked-layer structure of the first sacrificial layer and the second sacrificial layer, the hole-injection/transport layer 104B, the light-emitting layer 105B, and the electron-transport layer 108B may be processed into predetermined shapes in the following manner: part of the second sacrificial layer is etched with use of the resist mask REG, the resist mask REG is then removed, and part of the first sacrificial layer is etched with use of the second sacrificial layer as a mask. The shape illustrated in FIG. 7A is obtained through these etching treatment.

Subsequently, as illustrated in FIG. 7B, the hole-injection/transport layer 104G, the light-emitting layer 105G, and the electron-transport layer 108G are formed over the sacrificial layer 110B, the electrode 551G, the electrode 551R, and the electrode 551PS. As the materials for forming the hole-injection/transport layer 104G, the light-emitting layer 105G, and the electron-transport layer 108G, any of the materials described in Embodiment 1 to 3 can be used. Note that the hole-injection/transport layer 104G, the light-emitting layer 105G, and the electron-transport layer 108G can be formed by a vacuum evaporation method, for example.

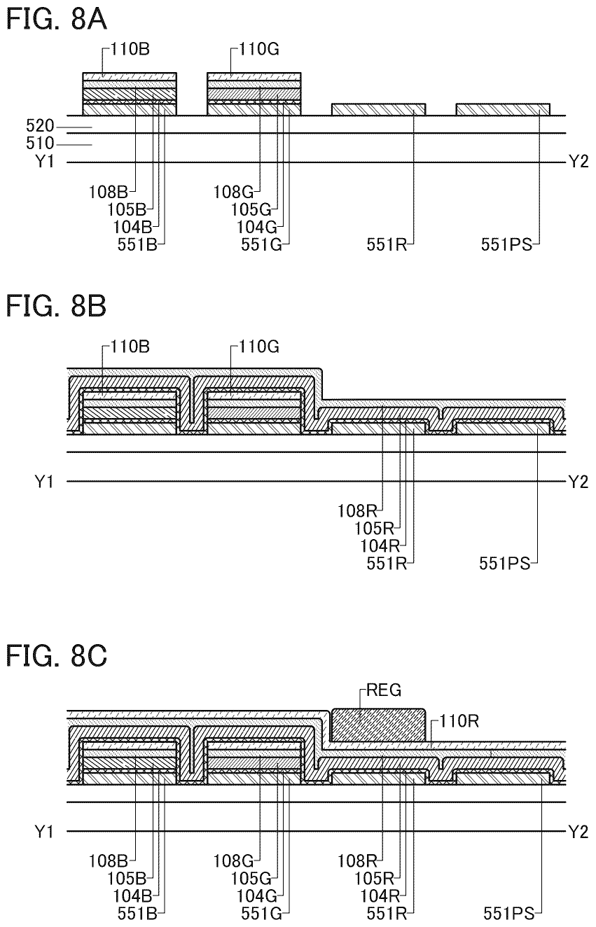

Next, as illustrated in FIG. 7C, a sacrificial layer 110G is formed over the electron-transport layer 108G, a resist is applied onto the sacrificial layer 110G, and the resist having a desired shape (resist mask: REG) is formed by a photolithography method. Part of the sacrificial layer 110G that is not covered with the obtained resist mask is removed by etching, the resist mask is then removed, and then the hole-injection/transport layer 104G, the light-emitting layer 105G, and the electron-transport layer 108G that are not covered with the sacrificial layer are removed by etching. Thus, the hole-injection/transport layer 104G, the light-emitting layer 105G, and the electron-transport layer 108G are processed to have side surfaces (or have their side surfaces exposed) over the electrode 551G or have belt-like shapes extending in the direction intersecting with the paper. Note that dry etching is preferably employed for the etching. The sacrificial layer 110G can be formed using a material similar to that for the sacrificial layer 110B. In the case where the sacrificial layer 110G has the aforementioned stacked-layer structure of the first sacrificial layer and the second sacrificial layer, the hole-injection/transport layer 104G, the light-emitting layer 105G, and the electron-transport layer 108G may be processed into predetermined shapes in the following manner: part of the second sacrificial layer is etched with use of the resist mask, the resist mask is then removed, and then part of the first sacrificial layer is etched with use of the second sacrificial layer as a mask. The shape illustrated in FIG. 8A is obtained through these etching treatment.

Next, as illustrated in FIG. 8B, the hole-injection/transport layer 104R, the light-emitting layer 105R, and the electron-transport layer 108R are formed over the sacrificial layer 110B, the sacrificial layer 110G, the electrode 551R, and the electrode 551PS. For the formation of the hole-injection/transport layer 104R, the light-emitting layer 105R, and the electron-transport layer 108R, any of the materials described in Embodiment 1 to 3 can be used. Note that the hole-injection/transport layer 104R, the light-emitting layer 105R, and the electron-transport layer 108R can be formed by a vacuum evaporation method, for example.

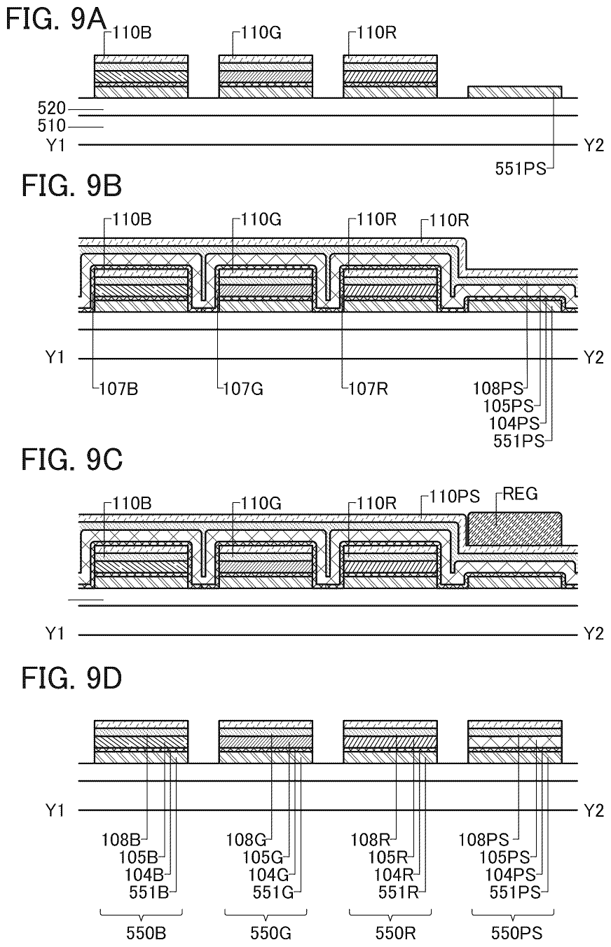

Next, as illustrated in FIG. 8C, a sacrificial layer 110R is formed over the electron-transport layer 108R, a resist is applied onto the sacrificial layer 110R, and the resist having a desired shape (resist mask: REG) is formed by a photolithography method. Part of the sacrificial layer 110R that is not covered with the obtained resist mask is removed by etching, the resist mask is then removed, and then the hole-injection/transport layer 104R, the light-emitting layer 105R, and the electron-transport layer 108R that are not covered with the sacrificial layer are removed by etching. Thus, the hole-injection/transport layer 104R, the light-emitting layer 105R, and the electron-transport layer 108R are processed to have side surfaces (or have their side surfaces exposed) over the electrode 551R or have belt-like shapes extending in the direction intersecting with the paper. Note that dry etching is preferably employed for the etching. The sacrificial layer 110R can be formed using a material similar to that for the sacrificial layer 110B. In the case where the sacrificial layer 110R has the aforementioned stacked-layer structure of the first sacrificial layer and the second sacrificial layer, the hole-injection/transport layer 104R, the light-emitting layer 105R, and the electron-transport layer 108R may be processed into predetermined shapes in the following manner: part of the second sacrificial layer is etched with use of the resist mask, the resist mask is then removed, and then part of the first sacrificial layer is etched with use of the second sacrificial layer as a mask. The shape illustrated in FIG. 9A is obtained through these etching treatment.

Next, as illustrated in FIG. 9B, the hole-injection/transport layer 104PS, the active layer 105PS, and the electron-transport layer 108PS are formed over the sacrificial layer 110B, the sacrificial layer 110G, the sacrificial layer 110R, and the electrode 551PS. As a material for forming the hole-injection/transport layer 104PS, for example, the material for the hole-injection layer and the hole-transport layer described in the description of embodiments up to Embodiment 3 can be used. As a material for the active layer 105PS, a material described in Embodiment 8 can be used. Furthermore, as a material for forming the electron-transport layer 108PS, for example, the material for the electron-transport layer and the electron-injection layer described in Embodiment 3 can be used. Note that the hole-injection/transport layer 104PS, the active layer 105PS, and the electron-transport layer 108PS can be formed by a vacuum evaporation method, for example.

Next, as illustrated in FIG. 9C, a sacrificial layer 110PS is formed over the electron-transport layer 108PS, a resist is applied onto the sacrificial layer 110PS, and the resist having a desired shape (resist mask: REG) is formed by a photolithography method. Part of the sacrificial layer 110PS that is not covered with the obtained resist mask is removed by etching, the resist mask is then removed, and the hole-injection/transport layer 104PS, the active layer 105PS, and the electron-transport layer 108PS that are not covered with the sacrificial layer are partly removed by etching. Thus, the hole-injection/transport layer 104PS, the active layer 105PS, and the electron-transport layer 108PS are processed to have side surfaces (or have their side surfaces exposed) over the electrode 551PS or have belt-like shapes extending in the direction intersecting with the paper. Note that dry etching is preferably employed for the etching. The sacrificial layer 110PS can be formed using a material similar to that for the sacrificial layer 110B. In the case where the sacrificial layer 110PS has the aforementioned stacked-layer structure of the first sacrificial layer and the second sacrificial layer, the hole-injection/transport layer 104PS, the active layer 105PS, and the electron-transport layer 108PS may be processed into a predetermined shape in the following manner: part of the second sacrificial layer is etched using the resist mask, the resist mask is then removed, and part of the first sacrificial layer is etched using the second sacrificial layer as a mask. The shape illustrated in FIG. 9D is obtained through these etching treatment.

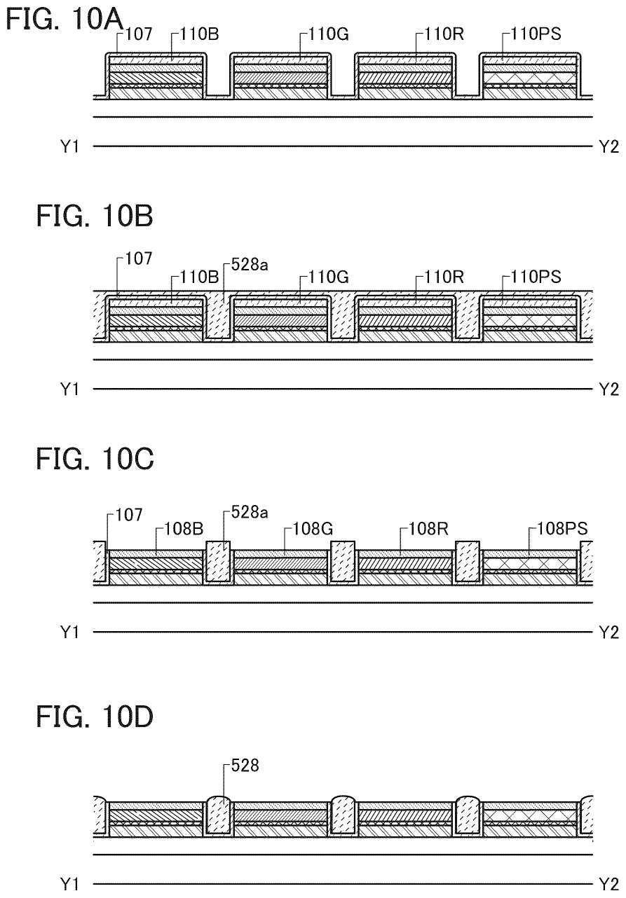

Next, as illustrated in FIG. 10A, the insulating layer 107 is formed over the sacrificial layer 110B, the sacrificial layer 110G, the sacrificial layer 110R, and the sacrificial layer 110PS.

For formation of the insulating layer 107, an ALD method can be used, for example. In this case, as illustrated in FIG. 10A, the insulating layer 107 is formed to be in contact with the side surfaces (end portions) of the hole-injection/transport layers (104B, 104G, and 104R), the light-emitting layers (103R, 103G, and 103R), and the electron-transport layers (108B, 108G, and 108R) of the light-emitting devices and the hole-injection/transport layer 104PS, the active layer 105PS, and the electron-transport layer 108PS of the light-receiving device. This can inhibit entry of oxygen, moisture, or constituent elements thereof into the inside through the side surfaces. Examples of the material used for the insulating layer 107 include aluminum oxide, magnesium oxide, hafnium oxide, gallium oxide, indium gallium zinc oxide, silicon nitride, and silicon nitride oxide.

Next, as illustrated in FIG. 10B, a resin film 528a is formed over the insulating layer 107. As the resin film 528a, for example, a negative photosensitive resin or a positive photosensitive resin can be used.

Then, as illustrated in FIG. 10C, part of the resin film 528a, part of the insulating layer 107, and the sacrificial layers (110B, 110G, 110R, and 110PS) are removed to expose the top surfaces of the electron-transport layers (108B, 108G, 108R, and 108PS).

Next, heat treatment is performed to process an upper edge portion of the resin film 528a into a curved shape, so that the partition wall 528 is formed, as illustrated in FIG. 10D. When the upper edge portion of the partition wall 528 has a curved shape, good coverage with the electron-injection layer 109 to be formed later can be obtained. For example, in the case of using a positive photosensitive acrylic resin as a material for the resin film 528a, the partition wall 528 preferably has a curved surface with a curvature radius (0.2 μm to 3 μm) at the upper end portion.

Next, the electron-injection layer 109 is formed over the insulating layer 107, the electron-transport layers (108B, 108G, 108R, and 108PS), and the partition wall 528. The electron-injection layer 109 can be formed using any of the materials described in Embodiment 3. Note that the electron-injection layer 109 is formed by a vacuum evaporation method, for example.

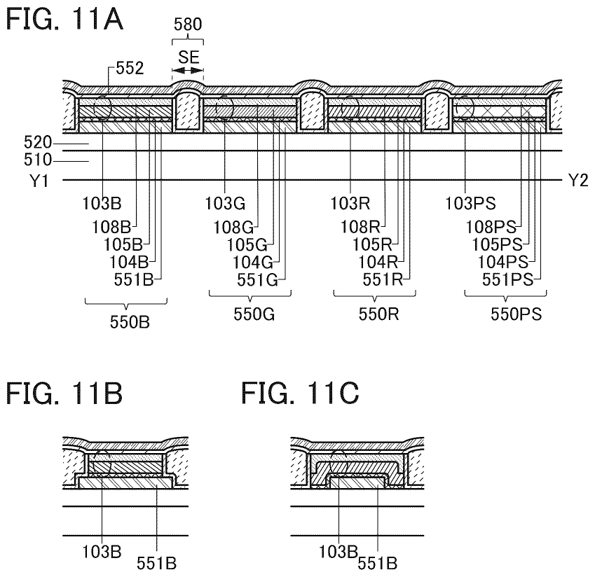

Next, as illustrated in FIG. 11A, the electrode 552 is formed over the electron-injection layer 109. The electrode 552 is formed by a vacuum evaporation method, for example.

Through the above steps, the EL layer 103B, the EL layer 103G, the EL layer 103R, and the light-receiving layer 103PS in the light-emitting device 550B, the light-emitting device 550G, the light-emitting device 550R, and the light-receiving device 550PS can be processed to be separated from each other.

The EL layers (the EL layer 103B, the EL layer 103G, and the EL layer 103R) and the light-receiving layer 103PS are processed to be separated by patterning using a photolithography method; hence, a high-resolution light-emitting and light-receiving apparatus (display panel) can be fabricated. End portions (side surfaces) of the EL layer processed by patterning using a photolithography method have substantially one surface (or are positioned on substantially the same plane).

The hole-injection/transport layers (104B, 104G, and 104R) of the EL layers and the hole-injection/transport layer 104PS of the light-receiving layer often have high conductivity, and thus might cause crosstalk when formed as layers shared by adjacent light-emitting devices. Therefore, processing the EL layers to be separated by patterning using a photolithography method as shown in this structure example can suppress occurrence of crosstalk between a light-emitting device and a light-receiving device adjacent to each other.

In this structure, the hole-injection/transport layers (104B, 104G, and 104R), the light-emitting layers (103R, 103G, and 103R), and the electron-transport layers (108B, 108G, and 108R) of the EL layers (EL layer 103B, EL layer 103G, and EL layer 103R) included in the light-emitting devices and the hole-injection/transport layer 104PS, the active layer 105PS, and the electron-transport layer 108PS of the light-receiving layer 103PS included in the light-receiving device are processed to be separated by patterning using a photolithography method; thus, the end portions (side surfaces) of the processed EL layers have substantially the same surface (or are positioned on substantially the same plane).

In addition, the hole-injection/transport layers (104B, 104G, and 104R), the light-emitting layers (103R, 103G, and 103R), and the electron-transport layers (108B, 108G, and 108R) of the EL layers (the EL layer 103B, the EL layer 103G, and the EL layer 103R) included in the light-emitting devices and the hole-injection/transport layer 104PS, the active layer 105PS, and the electron-transport layer 108PS of the light-receiving layer 103PS included in the light-receiving device are processed to be separated by patterning using a photolithography method; thus, the space 580 is provided between the processed end portions (side surfaces) of adjacent light-emitting devices. In FIG. 10C, when the space 580 is denoted by a distance SE between the EL layers in the adjacent light-emitting devices, the aperture ratio can be increased and definition can be increased as the distance SE decreases. By contrast, as the distance SE increases, the effect of the difference in the fabrication process between the adjacent light-emitting devices becomes permissible, which leads to an increase in manufacturing yield. Since the light-emitting device fabricated according to this specification is suitable for a miniaturization process, the distance SE between the EL layers in the adjacent light-emitting devices can be longer than or equal to 0.5 μm and shorter than or equal to 5 μm, preferably longer than or equal to 1 μm and shorter than or equal to 3 μm, further preferably longer than or equal to 1 μm and shorter than or equal to 2.5 μm, and still further preferably longer than or equal to 1 μm and shorter than or equal to 2 μm. Typically, the distance SE is preferably longer than or equal to 1 μm and shorter than or equal to 2 μm (e.g., 1.5 μm or a vicinity thereof).

In this specification and the like, a device formed using a metal mask or an FMM (fine metal mask, high-definition metal mask) may be referred to as a device having an MM (metal mask) structure. In this specification and the like, a device formed without using a metal mask or an FMM may be referred to as a device having an MML (metal maskless) structure. Since a light-emitting and light-receiving apparatus having the MML structure is manufactured without using a metal mask, the pixel arrangement, the pixel shape, and the like can be designed more flexibly than in a light-emitting and light-receiving apparatus having the FMM structure or the MM structure.

Note that an island-shaped EL layer of a light-emitting and light-receiving apparatus having an MML structure is formed not by patterning with use of a metal mask but by processing after formation of an EL layer. Accordingly, a light-emitting and light-receiving apparatus with a higher resolution or a higher aperture ratio than a conventional one can be achieved. Moreover, EL layers can be formed separately for the respective colors, enabling the light-emitting and light-receiving apparatus to perform extremely clear display with high contrast and high display quality. Moreover, providing the sacrificial layer over the EL layer can reduce damage to the EL layer in the fabricating process, resulting in an increase in the reliability of the light-emitting device.

In FIG. 5A and FIG. 11A, the widths of the EL layers (103B, 103G, and 103R) are substantially equal to the widths of the electrodes (551B, 551G, and 551R) in the light-emitting device 550B, the light-emitting device 550G, and the light-emitting device 550R, and the width of the light-receiving layer 103PS is substantially equal to the width of the electrode 551PS in the light-receiving device 550PS; however, one embodiment of the present invention is not limited thereto.

In the light-emitting device 550B, the light-emitting device 550G, and the light-emitting device 550R, the widths of the EL layers (103B, 103G, and 103R) may be smaller than the widths of the electrodes (551B, 551G, and 551R). In the light-receiving device 550PS, the width of the light-receiving layer 103PS may be smaller than the width of the electrode 551PS. FIG. 11B illustrates an example in which the width of the EL layer 103B is smaller than the width of the electrode 551B in the light-emitting device 550B.

In the light-emitting device 550B, the light-emitting device 550G, and the light-emitting device 550R, the widths of the EL layers (103B, 103G, and 103R) may be larger than the widths of the electrodes (551B, 551G, and 551R). In the light-receiving device 550PS, the width of the light-receiving layer 103PS may be larger than the width of the electrode 551PS. FIG. 11C illustrates an example in which the width of the EL layer 103R is larger than the width of the electrode 551R in the light-emitting device 550R.

Note that the light-emitting and light-receiving apparatus described in this embodiment includes both a light-emitting device and a light-receiving device, and can also be referred to as a light-emitting apparatus including a light-receiving device or a light-receiving apparatus including a light-emitting device. Among the structures of the light-emitting and light-receiving apparatus described in this embodiment, an apparatus that does not include a light-receiving device can also be referred to as a light-emitting apparatus. Among the structures of the light-emitting and light-receiving apparatus described in this embodiment, an apparatus that does not include a light-emitting apparatus can also be referred to as a light-receiving device.

The structures described in this embodiment can be used in an appropriate combination with any of the structures described in the other embodiments.

### Embodiment 5

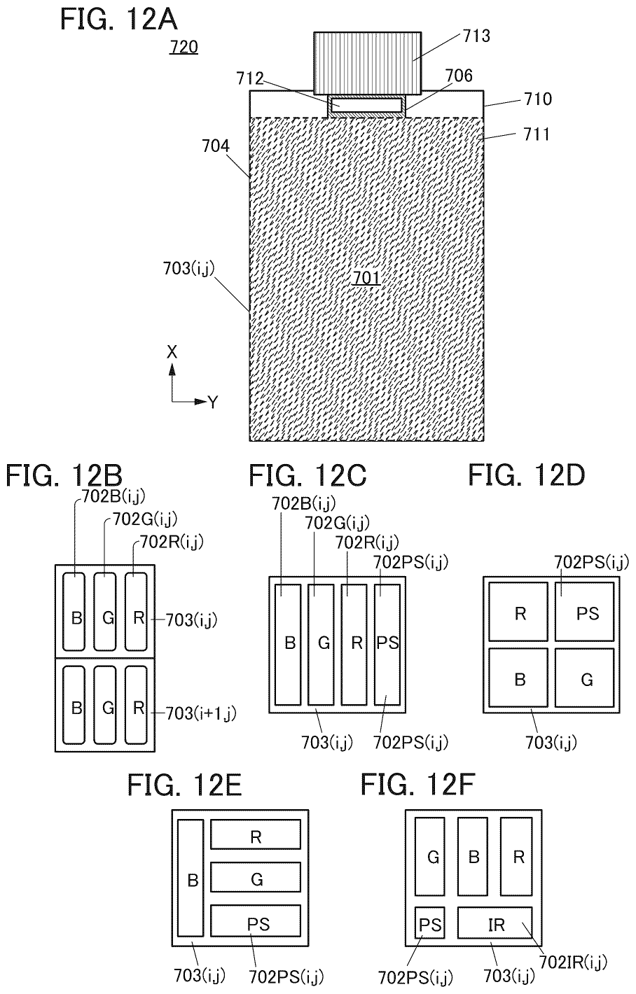

In this embodiment, an apparatus 720 will be described with reference to FIG. 12 to FIG. 14. The apparatus 720 illustrated in FIG. 12 to FIG. 14 includes any of the light-emitting devices described in Embodiment 1 and Embodiment 2 and thus is a light-emitting apparatus. Furthermore, the apparatus 720 described in this embodiment can be applied to a display portion of an electronic appliance or the like, and thus can also be referred to as a display panel or a display apparatus. Moreover, when the apparatus includes the light-emitting device as a light source and a light-receiving device that can receive light from the light-emitting device, the apparatuses can also be referred to as a light-emitting and light-receiving apparatus. Note that the light-emitting apparatus, the display panel, the display apparatus, and the light-emitting and light-receiving apparatus each include at least a light-emitting device.

Furthermore, the light-emitting apparatus, the display panel, the display apparatus, and the light-emitting and light-receiving apparatus of this embodiment can each have a high definition or a large size. Accordingly, the light-emitting apparatus, the display panel, the display apparatus, and the light-emitting and light-receiving apparatus can be used for display portions of electronic appliance such as a digital camera, a digital video camera, a digital photo frame, a mobile phone, a portable game console, a smartphone, a wristwatch terminal, a tablet terminal, a portable information terminal, and an audio reproducing device, in addition to display portions of electronic appliance with a relatively large screen, such as a television device, a desktop or laptop personal computer, a monitor of a computer or the like, digital signage, and a large game machine such as a pachinko machine.

FIG. 12A is a top view of the apparatus 720 (including the light-emitting apparatus, the display panel, the display apparatus, and the light-emitting and light-receiving apparatus).

In FIG. 12A, the apparatus 720 has a structure in which a substrate 710 and a substrate 711 are bonded to each other. In addition, the apparatus 720 includes a display region 701, a circuit 704, a wiring 706, and the like. Note that the display region 701 includes a plurality of pixels. As illustrated in FIG. 12B, a pixel 703(i,j) illustrated in FIG. 12A has a pixel 703(i+1,j) adjacent to the pixel 703(i, j).

Furthermore, as illustrated in the example of FIG. 12A, the substrate 710 is provided with an IC (integrated circuit) 712 by a COG (Chip On Glass) method, a COF (Chip On Film) method, or the like in the apparatus 720. As the IC 712, an IC including a scan line driver circuit, a signal line driver circuit, or the like can be used, for example. FIG. 12A illustrates a structure where an IC including a signal line driver circuit is used as the IC 712, and a scan line driver circuit is used as the circuit 704.

The wiring 706 has a function of supplying signals and power to the display region 701 and the circuit 704. The signals and power are input to the wiring 706 from the outside through an FPC (Flexible Printed Circuit) 713 or to the wiring 706 from the IC 712. Note that the apparatus 720 is not necessarily provided with the IC. The IC may be mounted on the FPC by a COF method or the like.

FIG. 12B illustrates the pixel 703(i,j) and the pixel 703(i+1,j) in the display region 701. The pixel 703(i,j) can have a plurality of kinds of subpixels including light-emitting devices that emit light of different colors. In addition to the above, a plurality of subpixels including light-emitting devices that emit light of the same color may be included. The pixel can include three kinds of subpixels, for example. The three subpixels can be of three colors of red (R), green (G), and blue (B) or of three colors of yellow (Y), cyan (C), and magenta (M), for example. Alternatively, the pixel can include four kinds of subpixels. As the four subpixels, subpixels of four colors of R, G, B, and white (W), subpixels of four colors of R, G, B, and Y, and the like can be given. Specifically, the pixel 703(i, j) can be composed of a subpixel 702B(i, j) displaying blue, a subpixel 702G(i, j) displaying green, and a subpixel 702R(i, j) displaying red.

The subpixel including a light-receiving device may be provided in addition to a subpixel including a light-emitting device. Note that in the case where the subpixel includes a light-receiving device, the apparatus 720 is also referred to as a light-emitting and light-receiving apparatus.

FIG. 12C to FIG. 12F illustrate various layout examples of the pixel 703(i, j) including a subpixel 702PS(i, j) including a light-receiving device. The pixel arrangement in FIG. 12C is stripe arrangement, and the pixel arrangement in FIG. 12D is matrix arrangement. In the pixel arrangement illustrated in FIG. 12E, three subpixels (the subpixel R, the subpixel G, and the subpixel S) are vertically arranged next to one subpixel (the subpixel B). In the pixel arrangement illustrated in FIG. 12F, the three vertically long subpixel G, subpixel B, and subpixel R are arranged laterally, and a subpixel PS and a horizontally long subpixel IR are arranged laterally below the three subpixels. Note that the wavelength of light detected by the subpixel 702PS(i,j) is not particularly limited; however, the light-receiving device included in the subpixel 702PS(i, j) preferably has sensitivity to light emitted from the light-emitting device included in the subpixel 702R(i, j), the subpixel 702G(i, j), the subpixel 702B(i, j), or a subpixel 702IR(i, j). The light-receiving device preferably detects one or more of light in blue, violet, bluish violet, green, yellowish green, yellow, orange, red, and infrared wavelength ranges, for example.

Furthermore, as illustrated in FIG. 12F, the subpixel 702G(i, j) that emits infrared rays may be added to any of the above-described sets of subpixels to form the pixel 703(i, j). Specifically, the subpixel that emits light including light with a wavelength greater than or equal to 650 nm and less than or equal to 1000 nm may be used in the pixel 703(i,j).

Note that the arrangement of subpixels is not limited to the structures illustrated in FIG. 12 and a variety of arrangement methods can be employed. The arrangement of subpixels may be stripe arrangement, S stripe arrangement, matrix arrangement, delta arrangement, Bayer arrangement, or PenTile arrangement, for example.

Examples of a top surface shape of the subpixel include polygons such as a triangle, a tetragon (including a rectangle and a square), and a pentagon; polygons with rounded corners; an ellipse; and a circle. Here, the top surface shape of the subpixel corresponds to a top surface shape of a light-emitting region of the light-emitting device.

In the case where a pixel includes a light-receiving device in addition to a light-emitting device, the pixel has a light-receiving function; thus, a touch or an approach of an object can be detected while an image is being displayed. For example, all the subpixels included in the light-emitting apparatus can display an image; alternatively, some of the subpixels can emit light as a light source, and the rest of the subpixels can display an image.

Note that the light-receiving area of the subpixel 702PS(i, j) is preferably smaller than the light-emitting areas of the other subpixels. A smaller light-receiving area leads to a narrower image-capturing range, inhibits a blur in a capturing result, and improves the resolution. Thus, by using the subpixel 702PS(i, j), high-definition or high-resolution image capturing is possible. For example, image capturing for personal authentication with the use of a fingerprint, a palm print, the iris, the shape of a blood vessel (including the shape of a vein and the shape of an artery), a face, or the like is possible by using the subpixel 702PS(i, j).

Moreover, the subpixel 702PS(i, j) can be used in a touch sensor (also referred to as a direct touch sensor), a near touch sensor (also referred to as a hover sensor, a hover touch sensor, a contactless sensor, or a touchless sensor), or the like. For example, the subpixel 702PS(i, j) preferably detects infrared light. Thus, a touch can be detected even in a dark place.

Here, the touch sensor or the near touch sensor can detect the approach or contact of an object (e.g., a finger, a hand, or a pen). The touch sensor can detect the object when the light-emitting and light-receiving apparatus and the object come in direct contact with each other. Furthermore, the near touch sensor can detect the object even when the object is not in contact with the light-emitting and light-receiving apparatus. For example, the display apparatus is preferably capable of detecting an object positioned in the range of 0.1 mm to 300 mm inclusive, further preferably 3 mm to 50 mm inclusive from the light-emitting and light-receiving apparatus. This structure enables the light-emitting and light-receiving apparatus to be operated without direct contact of an object, that is, enables the light-emitting and light-receiving apparatus to be operated in a contactless (touchless) manner. With the above-described structure, the light-emitting and light-receiving apparatus can be operated with a reduced risk of making the light-emitting and light-receiving apparatus dirty or damaging the light-emitting and light-receiving apparatus or without the object directly touching a dirt (e.g., dust, bacteria, or a virus) attached to the display apparatus.

For high-definition image capturing, the subpixels 702PS(i,j) are preferably provided in all pixels included in the light-emitting and light-receiving apparatus. Meanwhile, in the case where the subpixel 702PS(i, j) is used in a touch sensor, a near touch sensor, or the like, high accuracy is not required as compared to the case of capturing an image of a fingerprint or the like; accordingly, the subpixel 702PS(i, j) may be provided in some pixels in the light-emitting and light-receiving apparatus. When the number of the subpixels 702PS(i, j) included in the light-emitting and light-receiving apparatus is smaller than the number of the subpixels 702R(i,j) or the like, higher detection speed can be achieved.

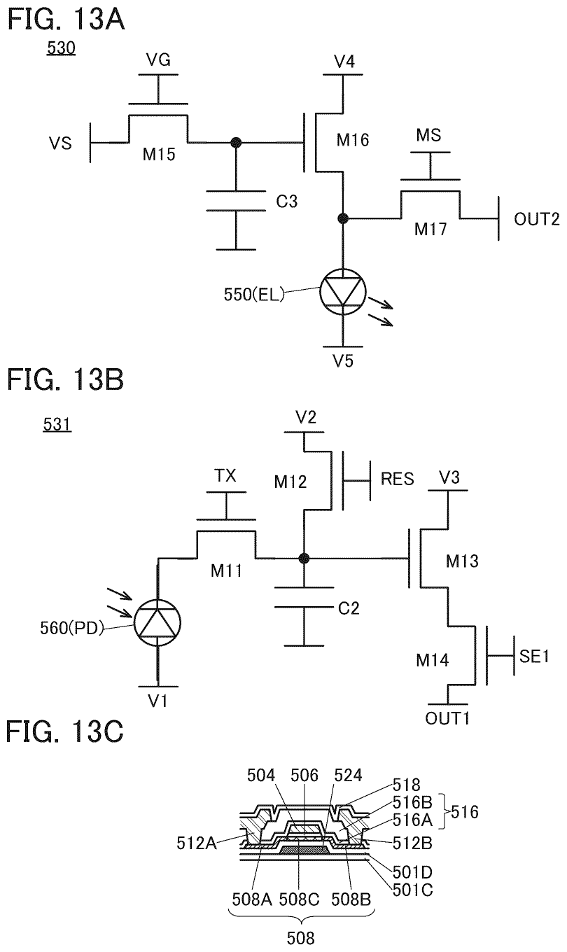

Next, an example of a pixel circuit of a subpixel including the light-emitting device is described with reference to FIG. 13A. A pixel circuit 530 illustrated in FIG. 13A includes a light-emitting device (EL) 550, a transistor M15, a transistor M16, a transistor M17, and a capacitor C3. Note that a light-emitting diode can be used as the light-emitting device 550. In particular, any of the light-emitting devices described in Embodiment 1 is preferably used as the light-emitting device 550.

In FIG. 13A, a gate of the transistor M15 is electrically connected to a wiring VG, one of a source and a drain of the transistor M15 is electrically connected to a wiring VS, and the other of the source and the drain of the transistor M15 is electrically connected to one electrode of the capacitor C3 and a gate of the transistor M16. One of a source and a drain of the transistor M16 is electrically connected to a wiring V4, and the other of the source and the drain of the transistor M16 is electrically connected to an anode of the light-emitting device 550 and one of a source and a drain of the transistor M17. A gate of the transistor M17 is electrically connected to a wiring MS, and the other of the source and the drain of the transistor M17 is electrically connected to a wiring OUT2. A cathode of the light-emitting device 550 is electrically connected to a wiring V5.

A constant potential is supplied to the wiring V4 and the wiring V5. In the light-emitting device 550, the anode side can have a high potential and the cathode side can have a lower potential than the anode side. The transistor M15 is controlled by a signal supplied to the wiring VG and functions as a selection transistor for controlling a selection state of the pixel circuit 530. The transistor M16 functions as a driving transistor that controls a current flowing through the light-emitting device 550 in accordance with a potential supplied to the gate of the transistor M16. When the transistor M15 is in a conduction state, a potential supplied to the wiring VS is supplied to the gate of the transistor M16, and the luminance of the light-emitting device 550 can be controlled in accordance with the potential. The transistor M17 is controlled by a signal supplied to the wiring MS and has a function of outputting a potential between the transistor M16 and the light-emitting device 550 to the outside through the wiring OUT2.

Here, a transistor in which a metal oxide (an oxide semiconductor) is used as a semiconductor layer where a channel is formed is preferably used as the transistor M15, the transistor M16, and the transistor M17 included in the pixel circuit 530 in FIG. 13A and a transistor M11, a transistor M12, a transistor M13, and a transistor M14 included in a pixel circuit 531 in FIG. 13B.

A transistor using a metal oxide having a wider band gap and a lower carrier density than silicon achieves an extremely low off-state current. Therefore, owing to the low off-state current, charge accumulated in a capacitor that is connected in series with the transistor can be retained for a long time. Accordingly, it is particularly preferable to use transistors containing an oxide semiconductor as the transistor M11, the transistor M12, and the transistor M15 each of which is connected in series with a capacitor C2 or the capacitor C3. When the other transistors also include an oxide semiconductor, the fabrication cost can be reduced.

Alternatively, transistors using silicon for a semiconductor in which a channel is formed can be used as the transistor M11 to the transistor M17. It is particularly preferable to use silicon with high crystallinity, such as single crystal silicon or polycrystalline silicon, because high field-effect mobility can be achieved and higher-speed operation can be performed.

Alternatively, a transistor using an oxide semiconductor may be used as one or more of the transistor M11 to the transistor M17, and transistors using silicon may be used as the other transistors.

Next, an example of a pixel circuit of a subpixel including a light-receiving device is described with reference to FIG. 13B. The pixel circuit 531 illustrated in FIG. 13B includes a light-receiving device (PD) 560, the transistor M11, the transistor M12, the transistor M13, the transistor M14, and the capacitor C2. Here, an example in which a photodiode is used as the light-receiving device (PD) 560 is illustrated.

In FIG. 13B, an anode of the light-receiving device (PD) 560 is electrically connected to a wiring V1, and a cathode of the light-receiving device (PD) 560 is electrically connected to one of a source and a drain of the transistor M11. A gate of the transistor M11 is electrically connected to a wiring TX, and the other of the source and the drain of the transistor M11 is electrically connected to one electrode of the capacitor C2, one of a source and a drain of the transistor M12, and a gate of the transistor M13. A gate of the transistor M12 is electrically connected to a wiring RES, and the other of the source and the drain of the transistor M12 is electrically connected to a wiring V2. One of a source and a drain of the transistor M13 is electrically connected to a wiring V3, and the other of the source and the drain of the transistor M13 is electrically connected to one of a source and a drain of the transistor M14. A gate of the transistor M14 is electrically connected to a wiring SE1, and the other of the source and the drain of the transistor M14 is electrically connected to a wiring OUT1.

A constant potential is supplied to each of the wiring V1, the wiring V2, and the wiring V3. When the light-receiving device (PD) 560 is driven with a reverse bias, the wiring V2 is supplied with a potential higher than the potential of the wiring V1. The transistor M12 is controlled by a signal supplied to the wiring RES and has a function of resetting the potential of a node connected to the gate of the transistor M13 to a potential supplied to the wiring V2. The transistor M11 is controlled by a signal supplied to the wiring TX and has a function of controlling the timing at which the potential of the node changes, in accordance with a current flowing through the light-receiving device (PD) 560. The transistor M13 functions as an amplifier transistor for performing output corresponding to the potential of the node. The transistor M14 is controlled by a signal supplied to the wiring SE1 and functions as a selection transistor for making an external circuit connected to the wiring OUT1 read the output corresponding to the potential of the node.

Although n-channel transistors are illustrated in FIG. 13A and FIG. 13B, p-channel transistors can alternatively be used.

The transistors included in the pixel circuit 530 and the transistors included in the pixel circuit 531 are preferably formed to be arranged over the same substrate. It is particularly preferable that the transistors included in the pixel circuit 530 and the transistors included in the pixel circuit 531 be periodically arranged in one region.

One or more layers including the transistor and/or the capacitor are preferably provided to overlap with the light-receiving device (PD) 560 or the light-emitting device (EL) 550. Thus, the effective area occupied by each pixel circuit can be reduced, and a high-resolution light-receiving portion or display portion can be achieved.

FIG. 13C illustrates an example of a specific structure of a transistor that can be used in the pixel circuit described with reference to FIG. 13A and FIG. 13B. As the transistor, a bottom-gate transistor, a top-gate transistor, or the like can be used as appropriate.

The transistor illustrated in FIG. 13C includes a semiconductor film 508, a conductive film 504, an insulating film 506, a conductive film 512A, and a conductive film 512B. The transistor is formed over an insulating film 501C, for example. The transistor also includes an insulating film 516 (an insulating film 516A and an insulating film 516B) and an insulating film 518.

The semiconductor film 508 includes a region 508A electrically connected to the conductive film 512A and a region 508B electrically connected to the conductive film 512B. The semiconductor film 508 includes a region 508C between the region 508A and the region 508B.

The conductive film 504 includes a region overlapping with the region 508C and has a function of a gate electrode.

The insulating film 506 includes a region positioned between the semiconductor film 508 and the conductive film 504. The insulating film 506 has a function of a first gate insulating film.

The conductive film 512A has one of a function of a source electrode and a function of a drain electrode, and the conductive film 512B has the other of the function of the source electrode and the function of the drain electrode.

A conductive film 524 can be used in the transistor. The conductive film 524 includes a region where the semiconductor film 508 is positioned between the conductive film 504 and the conductive film 524. The conductive film 524 has a function of a second gate electrode. An insulating film 501D is positioned between the semiconductor film 508 and the conductive film 524 and has a function of a second gate insulating film.

The insulating film 516 functions as, for example, a protective film covering the semiconductor film 508. Specifically, a film including a silicon oxide film, a silicon oxynitride film, a silicon nitride oxide film, a silicon nitride film, an aluminum oxide film, a hafnium oxide film, an yttrium oxide film, a zirconium oxide film, a gallium oxide film, a tantalum oxide film, a magnesium oxide film, a lanthanum oxide film, a cerium oxide film, or a neodymium oxide film can be used as the insulating film 516, for example.

For example, a material having a function of inhibiting diffusion of oxygen, hydrogen, water, an alkali metal, an alkaline earth metal, and the like is preferably used for the insulating film 518. Specifically, the insulating film 518 can be formed using silicon nitride, silicon oxynitride, aluminum nitride, or aluminum oxynitride, for example. In each of silicon oxynitride and aluminum oxynitride, the number of nitrogen atoms contained is preferably larger than the number of oxygen atoms contained.

Note that in a step of forming the semiconductor film used in the transistor of the pixel circuit, the semiconductor film used in the transistor of the driver circuit can be formed. A semiconductor film with the same composition as the semiconductor film used in the transistor of the pixel circuit can be used in the driver circuit, for example.

The semiconductor film 508 preferably contains indium, M (M is one or more kinds selected from gallium, aluminum, silicon, boron, yttrium, tin, copper, vanadium, beryllium, titanium, iron, nickel, germanium, zirconium, molybdenum, lanthanum, cerium, neodymium, hafnium, tantalum, tungsten, and magnesium), and zinc, for example. Specifically, M is preferably one or more kinds selected from aluminum, gallium, yttrium, and tin.

It is particularly preferable that an oxide containing indium (In), gallium (Ga), and zinc (Zn) (also referred to as IGZO) be used for the semiconductor film 508. Alternatively, it is preferable to use an oxide containing indium, tin, and zinc. Further alternatively, it is preferable to use an oxide containing indium, gallium, tin, and zinc. Alternatively, it is preferable to use an oxide containing indium (In), aluminum (Al), and zinc (Zn) (also referred to as IAZO). Further alternatively, it is preferable to use an oxide containing indium (In), aluminum (Al), gallium (Ga), and zinc (Zn) (also referred to as IAGZO).

In the case where the semiconductor film is an In-M-Zn oxide, the atomic proportion of In is preferably greater than or equal to the atomic proportion of M in the In-M-Zn oxide. Examples of the atomic ratio of the metal elements in such an In-M-Zn oxide include In:M:Zn=1:1:1 or a composition in the vicinity thereof, In:M:Zn=1:1:1.2 or a composition in the vicinity thereof, In:M:Zn=1:3:2 or a composition in the vicinity thereof, In:M:Zn=1:3:4 or a composition in the vicinity thereof, In:M:Zn=2:1:3 or a composition in the vicinity thereof, In:M:Zn=3:1:2 or a composition in the vicinity thereof, In:M:Zn=4:2:3 or a composition in the vicinity thereof, In:M:Zn=4:2:4.1 or a composition in the vicinity thereof, In:M:Zn=5:1:3 or a composition in the vicinity thereof, In:M:Zn=5:1:6 or a composition in the vicinity thereof, In:M:Zn=5:1:7 or a composition in the vicinity thereof, In:M:Zn=5:1:8 or a composition in the vicinity thereof, In:M:Zn=6:1:6 or a composition in the vicinity thereof, and In:M:Zn=5:2:5 or a composition in the vicinity thereof. Note that a composition in the vicinity includes the range of ±30% of an intended atomic ratio.

For example, when the atomic ratio is described as In:Ga:Zn=4:2:3 or a composition in the vicinity thereof, the case is included where the atomic ratio of Ga is greater than or equal to 1 and less than or equal to 3 and the atomic ratio of Zn is greater than or equal to 2 and less than or equal to 4 with the atomic ratio of In being 4. When the atomic ratio is described as In:Ga:Zn=5:1:6 or a composition in the vicinity thereof, the case is included where the atomic ratio of Ga is greater than 0.1 and less than or equal to 2 and the atomic ratio of Zn is greater than or equal to 5 and less than or equal to 7 with the atomic ratio of In being 5. When the atomic ratio is described as In:Ga:Zn=1:1:1 or a composition in the vicinity thereof, the case is included where the atomic ratio of Ga is greater than 0.1 and less than or equal to 2 and the atomic ratio of Zn is greater than 0.1 and less than or equal to 2 with the atomic ratio of In being 1.

There is no particular limitation on the crystallinity of a semiconductor material used for the transistors, and any of an amorphous semiconductor and a semiconductor having crystallinity (a microcrystalline semiconductor, a polycrystalline semiconductor, a single crystal semiconductor, or a semiconductor partly including crystal regions) may be used. A semiconductor having crystallinity is preferably used, in which case deterioration of the transistor characteristics can be inhibited.

The semiconductor layer of the transistor preferably includes a metal oxide (also referred to as an oxide semiconductor). As the oxide semiconductor having crystallinity, a CAAC (c-axis aligned crystalline)-OS, an nc (nanocrystalline)-OS, and the like are given.

Alternatively, a transistor using silicon in a channel formation region (a Si transistor) may be used. Examples of silicon include single crystal silicon (single crystal Si), polycrystalline silicon, and amorphous silicon. In particular, a transistor containing low-temperature polysilicon (LTPS) in its semiconductor layer (hereinafter also referred to as an LTPS transistor) can be used. The LTPS transistor has high field-effect mobility and favorable frequency characteristics.

With the use of a Si transistor such as an LTPS transistor, a circuit required to be driven at a high frequency (e.g., a source driver circuit) can be formed on the same substrate as the display portion. This allows simplification of an external circuit mounted on the light-emitting apparatus and a reduction in component cost and mounting cost.

An OS transistor has much higher field-effect mobility than a transistor using amorphous silicon. In addition, an OS transistor has an extremely low leakage current between a source and a drain in an off state (hereinafter also referred to as off-state current), and charge accumulated in a capacitor that is connected in series to the transistor can be retained for a long period. Furthermore, the power consumption of the light-emitting apparatus can be reduced with the OS transistor.

The off-state current value per micrometer of channel width of the OS transistor at room temperature can be lower than or equal to 1 aA (1×10⁻¹⁸ A), lower than or equal to 1 zA (1×10⁻²¹ A), or lower than or equal to 1 yA (1×10⁻²⁴ A). Note that the off-state current value per micrometer of channel width of a Si transistor at room temperature is higher than or equal to 1 fA (1×10⁻¹⁵ A) and lower than or equal to 1 pA (1×10⁻¹² A). In other words, the off-state current of an OS transistor is lower than that of a Si transistor by approximately ten orders of magnitude.

To increase the emission luminance of the light-emitting device included in a pixel circuit, it is necessary to increase the amount of current flowing through the light-emitting device. For that purpose, the source-drain voltage of the driving transistor included in the pixel circuit needs to be increased. Since an OS transistor has a higher withstand voltage between the source and the drain than a Si transistor, a high voltage can be applied between the source and the drain of the OS transistor. Thus, with use of an OS transistor as a driving transistor included in the pixel circuit, the amount of current flowing through the light-emitting device can be increased, resulting in an increase in emission luminance of the light-emitting device.

When a transistor operates in a saturation region, a change in source-drain current relative to a change in gate-source voltage can be smaller in an OS transistor than in a Si transistor. Accordingly, when an OS transistor is used as the driving transistor included in the pixel circuit, current flowing between the source and the drain can be set minutely by a change in gate-source voltage; hence, the amount of current flowing through the light-emitting device can be controlled. Accordingly, the number of gray levels in the pixel circuit can be increased.

Regarding saturation characteristics of current flowing when a transistor operates in a saturation region, even in the case where the source-drain voltage of an OS transistor increases gradually, more stable current (saturation current) can be made flow through an OS transistor than through a Si transistor. Thus, with use of an OS transistor as a driving transistor, current can be made flow stably through the light-emitting device, for example, even when a variation in current-voltage characteristics of the light-emitting device occurs. In other words, when the OS transistor operates in the saturation region, the source-drain current hardly changes with an increase in the source-drain voltage; hence, the emission luminance of the light-emitting device can be stable.

As described above, with use of an OS transistor as the driving transistor included in the pixel circuit, it is possible to achieve “inhibition of black floating”, “increase in emission luminance”, “increase in the number of gray levels”, “inhibition of variation in light-emitting devices”, and the like.

The semiconductor film used in the transistor of the driver circuit can be formed in the same step as the semiconductor film used in the transistor of the pixel circuit. The driver circuit can be formed over a substrate where the pixel circuit is formed. The number of components of an electronic appliance can be reduced.

Silicon may be used for the semiconductor film 508. Examples of silicon include single crystal silicon, polycrystalline silicon, and amorphous silicon. In particular, a transistor containing low-temperature polysilicon (LTPS) in its semiconductor layer (hereinafter also referred to as an LTPS transistor) is preferably used. The LTPS transistor has high field-effect mobility and favorable frequency characteristics.

With the use of a transistor containing silicon, such as an LTPS transistor, a circuit required to be driven at a high frequency (e.g., a source driver circuit) can be formed on the same substrate as the display portion. This allows simplification of an external circuit mounted on the light-emitting apparatus and a reduction in component cost and mounting cost.

It is preferable to use a transistor containing a metal oxide (hereinafter also referred to as an oxide semiconductor) in its semiconductor where a channel is formed (hereinafter also referred to as an OS transistor) as at least one of the transistors included in the pixel circuit. An OS transistor has much higher field-effect mobility than a transistor using amorphous silicon. In addition, an OS transistor has an extremely low leakage current between a source and a drain in an off state (hereinafter also referred to as off-state current), and charge accumulated in a capacitor that is connected in series to the transistor can be retained for a long period. Furthermore, the power consumption of the light-emitting apparatus can be reduced with the OS transistor.

When LTPS transistors are used as some of the transistors included in the pixel circuit and OS transistors are used as the rest, the light-emitting apparatus can have low power consumption and high driving capability. As a favorable example, it is preferable that an OS transistor be used as a transistor functioning as a switch for controlling electrical continuity between wirings and an LTPS transistor be used as a transistor for controlling current. Note that a structure in which an LTPS transistor and an OS transistor are combined is referred to as LTPO in some cases. LTPO enables the display panel to have low power consumption and high driving capability.

For example, one transistor provided in the pixel circuit functions as a transistor for controlling current flowing through the light-emitting device and can also be referred to as a driving transistor. One of a source and a drain of the driving transistor is electrically connected to the pixel electrode of the light-emitting device. An LTPS transistor is preferably used as the driving transistor. Thus, current flowing through the light-emitting device in the pixel circuit can be increased.

In contrast, another transistor provided in the pixel circuit functions as a switch for controlling selection and non-selection of a pixel and can also be referred to as a selection transistor. A gate of the selection transistor is electrically connected to a gate line, and one of a source and a drain thereof is electrically connected to a source line (signal line). An OS transistor is preferably used as the selection transistor. Accordingly, the gray level of the pixel can be maintained even with an extremely low frame frequency (e.g., 1 fps or less); thus, power consumption can be reduced by stopping the driver in displaying a still image.

In the case of using an oxide semiconductor in a semiconductor film, the apparatus 720 includes a light-emitting device including an oxide semiconductor in its semiconductor film and having an MML (metal maskless) structure. With this structure, the leakage current that might flow through the transistor and the leakage current that might flow between adjacent light-emitting devices (also referred to as lateral leakage current, side leakage current, or the like) can be extremely low. With the above structure, a viewer can notice any one or more of the image crispness, the image sharpness, a high chroma, and a high contrast ratio in an image displayed on the display apparatus. When the leakage current that might flow through the transistor and the lateral leakage current that might flow between light-emitting devices are extremely low, display with little leakage of light at the time of black display (what is called black floating) (such display is also referred to as deep black display) can be achieved.

In particular, in the case where a light-emitting device having an MML structure employs the above-described SBS structure, a layer provided between light-emitting devices (for example, also referred to as an organic layer or a common layer which is shared by the light-emitting devices) is divided; accordingly, display with no or extremely small lateral leakage can be achieved.

The structure of transistors used in a display panel may be selected as appropriate depending on the screen size of the display panel. For example, single crystal Si transistors can be used in the display panel with a screen diagonal greater than or equal to 0.1 inches and less than or equal to 3 inches. In addition, LTPS transistors can be used in the display panel with a screen diagonal greater than or equal to 0.1 inches and less than or equal to 30 inches, preferably greater than or equal to 1 inch and less than or equal to 30 inches. In addition, an LTPO structure (where an LTPS transistor and an OS transistor are used in combination) can be used in the display panel with a screen diagonal greater than or equal to 0.1 inches and less than or equal to 50 inches, preferably greater than or equal to 1 inch and less than or equal to 50 inches. In addition, OS transistors can be used in the display panel with a screen diagonal greater than or equal to 0.1 inches and less than or equal to 200 inches, preferably greater than or equal to 50 inches and less than or equal to 100 inches.

Note that with use of single crystal Si transistors, an increase in screen size is extremely difficult because of the size of a single crystal Si substrate. Furthermore, since a laser crystallization apparatus is used in the manufacturing process, LTPS transistors are unlikely to respond to an increase in screen size (typically to a screen diagonal greater than 30 inches). By contrast, since the manufacturing process does not necessarily require a laser crystallization apparatus or the like or can be performed at a relatively low process temperature (typically, lower than or equal to 450° C.), OS transistors can be used for a display panel with a relatively large area (typically, a screen diagonal greater than or equal to 50 inches and less than or equal to 100 inches). In addition, LTPO is applicable to a display panel with a size midway between the case of using LTPS transistors and the case of using OS transistors (typically, a diagonal size greater than or equal to 1 inch and less than or equal to 50 inches).

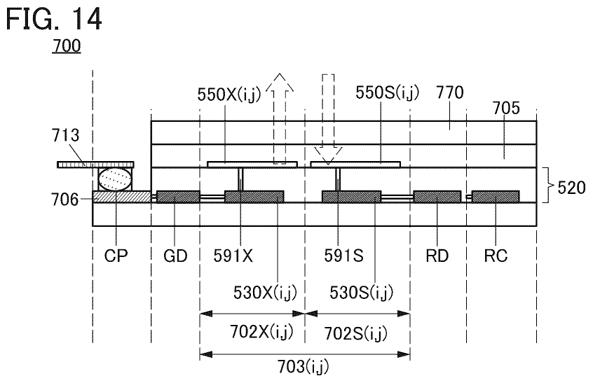

Next, a cross-sectional view of the light-emitting and light-receiving apparatus is shown. FIG. 14 is a cross-sectional view of the light-emitting and light-receiving apparatus illustrated in FIG. 12A.

FIG. 14 is a cross-sectional view of part of a region including the FPC 713 and the wiring 706 and part of the display region 701 including the pixel 703(i, j).

In FIG. 14, the light-emitting and light-receiving apparatus 700 includes the functional layer 520 between the first substrate 510 and the second substrate 770. The functional layer 520 includes, as well as the above-described transistors (M11, M12, M13, M14, M15, M16, and M17), the capacitors (C2 and C3), and the like described with reference to FIG. 13, the wirings (VS, VG, V1, V2, V3, V4, and V5) electrically connected to these components, for example. The structure of the functional layer 520 illustrated in FIG. 14 includes a pixel circuit 530X(i, j), a pixel circuit 530S(i, j), and a circuit GD; however, it is not limited thereto.

Each pixel circuit (e.g., the pixel circuit 530X(i,j) and the pixel circuit 530S(i,j) in FIG. 14) included in the functional layer 520 is electrically connected to a light-emitting device and a light receiving device (e.g., a light-emitting device 550X(i,j) and a light-receiving device 550S(i, j) in FIG. 14) formed over the functional layer 520. Specifically, the light-emitting device 550X(i, j) is electrically connected to the pixel circuit 530X(i, j) through a wiring 591X, and the light-receiving device 550S(i,j) is electrically connected to the pixel circuit 530S(i, j) through a wiring 591S. The insulating layer 705 is provided over the functional layer 520, the light-emitting device, and the light-receiving device and the insulating layer 705 has a function of bonding the second substrate 770 and the functional layer 520.

As the second substrate 770, a substrate where touch sensors are arranged in a matrix can be used. For example, a substrate provided with capacitive touch sensors or optical touch sensors can be used as the second substrate 770. Thus, the light-emitting and light receiving apparatus of one embodiment of the present invention can be used as a touch panel.

Note that the structure described in this embodiment can be used in an appropriate combination with any of the structures described in the other embodiments.

### Embodiment 6

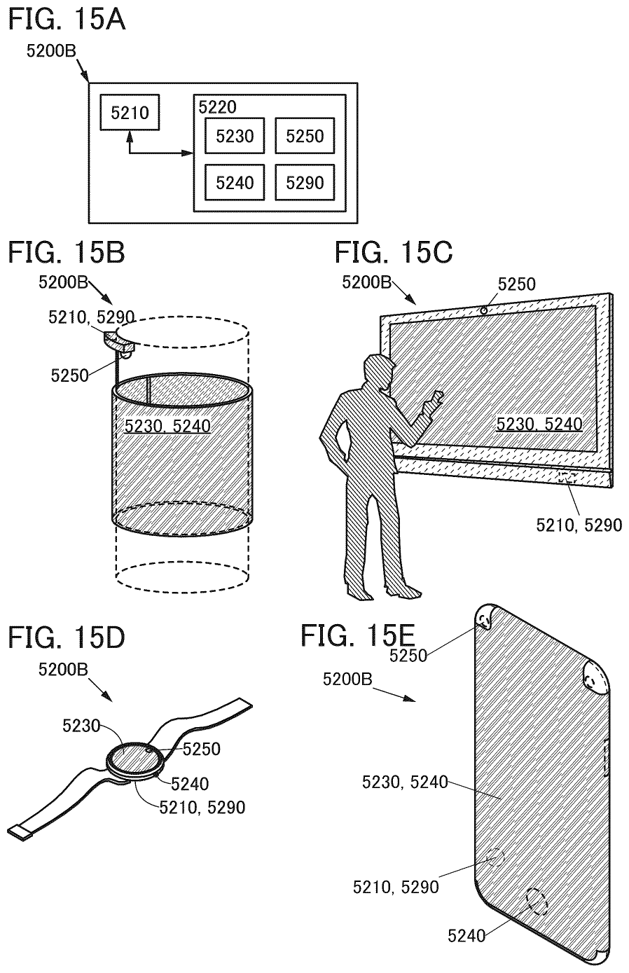

In this embodiment, electronic appliance of one embodiment of the present invention will be described with reference to FIG. 15A to FIG. 17B.

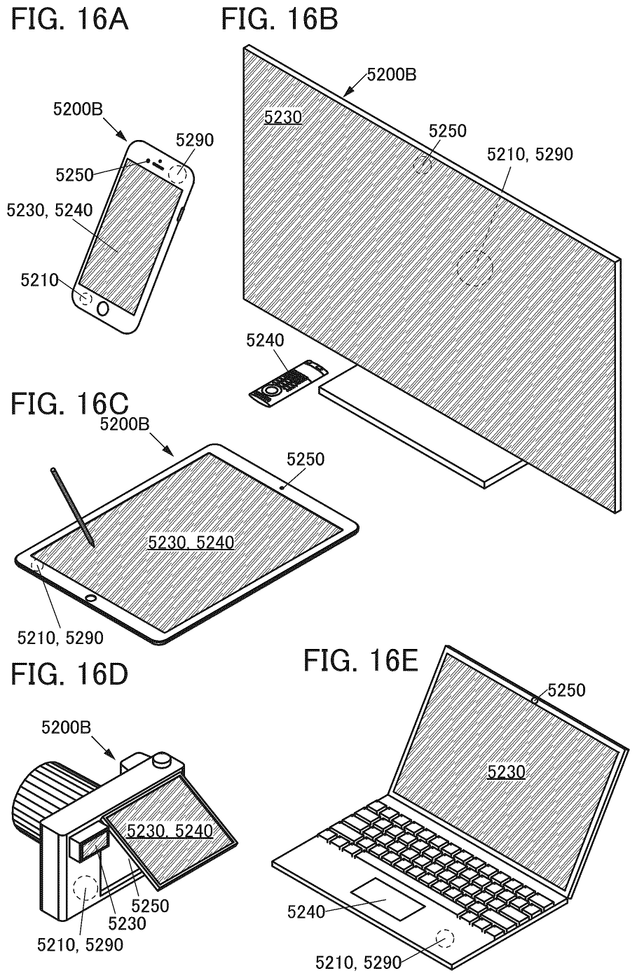

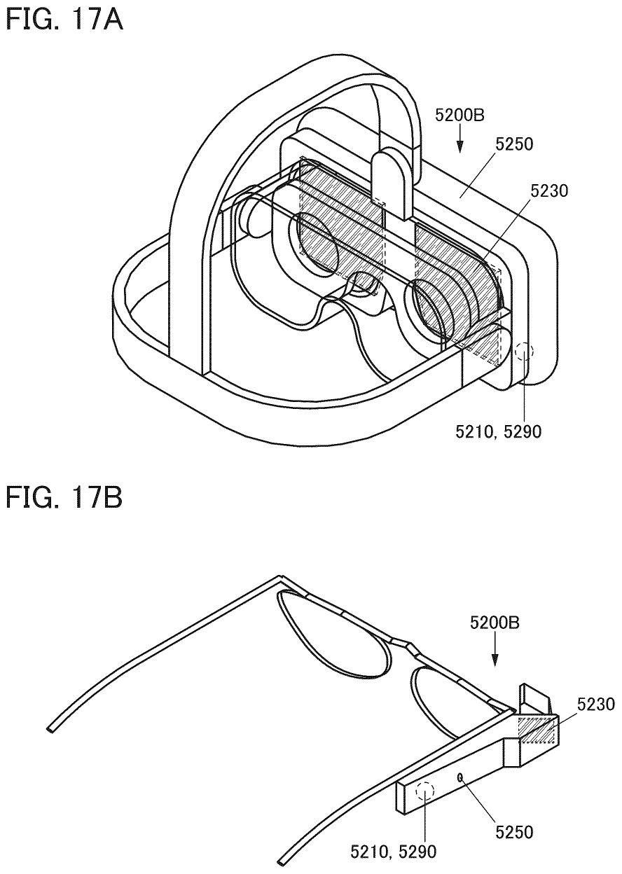

FIG. 15A to FIG. 17B are diagrams illustrating structures of electronic appliance of one embodiment of the present invention. FIG. 15A is a block diagram of the electronic appliance and FIG. 15B to FIG. 15E are perspective views illustrating structures of the electronic appliance. FIG. 16A to FIG. 16E are perspective views illustrating structures of electronic appliance. FIG. 17A and FIG. 17B are perspective views illustrating structures of electronic appliance.

An electronic appliance 5200B described in this embodiment includes an arithmetic device 5210 and an input/output device 5220 (see FIG. 15A).

The arithmetic device 5210 has a function of being supplied with operation data and has a function of supplying image data on the basis of the operation data.

The input/output device 5220 includes a display portion 5230, an input portion 5240, a detecting portion 5250, and a communication portion 5290 and has a function of supplying operation data and a function of being supplied with image data. The input/output device 5220 also has a function of supplying detection data, a function of supplying communication data, and a function of being supplied with communication data.

The input portion 5240 has a function of supplying operation data. For example, the input portion 5240 supplies operation data on the basis of operation by a user of the electronic appliance 5200B.

Specifically, a keyboard, a hardware button, a pointing device, a touch sensor, an illuminance sensor, an imaging apparatus, an audio input device, an eye-gaze input device, an attitude detection device, or the like can be used as the input portion 5240.

The display portion 5230 includes a display panel and has a function of displaying image data. For example, the display panel described in Embodiment 4 can be used for the display portion 5230.

The detecting portion 5250 has a function of supplying detection data. For example, the detecting portion 5250 has a function of detecting a surrounding environment where the electronic appliance is used and supplying detection data.

Specifically, an illuminance sensor, an imaging apparatus, an attitude detection device, a pressure sensor, a human motion sensor, or the like can be used as the detecting portion 5250.

The communication portion 5290 has a function of being supplied with communication data and a function of supplying communication data. For example, the communication portion 5290 has a function of being connected to another electronic appliance or a communication network through wireless communication or wired communication. Specifically, the communication portion 5290 has a function of wireless local area network communication, telephone communication, near field communication, or the like.

FIG. 15B illustrates an electronic appliance having an outer shape along a cylindrical column or the like. An example of such an electronic appliance is digital signage. The display panel of one embodiment of the present invention can be used for the display portion 5230. Note that the electronic appliance has a function of changing its display method in accordance with the illuminance of a usage environment. Furthermore, the electronic appliance has a function of changing displayed content in response to detected existence of a person. Thus, for example, the electronic appliance can be provided on a column of a building. The electronic appliance can display advertising, guidance, or the like.

FIG. 15C illustrates an electronic appliance having a function of generating image data on the basis of the path of a pointer used by the user. Examples of such an electronic appliance include an electronic blackboard, an electronic bulletin board, and digital signage. Specifically, the display panel with a diagonal size of 20 inches or longer, preferably 40 inches or longer, and further preferably 55 inches or longer can be used. Alternatively, a plurality of display panels can be arranged and used as one display region. Alternatively, a plurality of display panels can be arranged and used as a multiscreen.

FIG. 15D illustrates an electronic appliance that is capable of receiving data from another device and displaying the data on the display portion 5230. An example of such an electronic appliance is a wearable electronic appliance. Specifically, the electronic appliance can display several options, or allow a user to choose some from the options and send a reply to the data transmitter. Alternatively, for example, the electronic appliance has a function of changing its display method in accordance with the illuminance of a usage environment. Thus, the power consumption of the wearable electronic appliance can be reduced, for example. Alternatively, an image can be displayed on the wearable electronic appliance so that the wearable electronic appliance can be suitably used even in an environment under strong external light, e.g., outdoors in fine weather, for example.

FIG. 15E illustrates an electronic appliance including the display portion 5230 having a surface gently curved along a side surface of a housing. An example of such an electronic appliance is a mobile phone. The display portion 5230 includes a display panel, and the display panel has a function of performing display on the front surface, the side surfaces, the top surface, and the rear surface, for example. Thus, for example, a mobile phone can display data not only on the front surface but also on the side surfaces, the top surface, and the rear surface.

FIG. 16A illustrates an electronic appliance that is capable of receiving data via the Internet and displaying the data on the display portion 5230. An example of such an electronic appliance is a smartphone. For example, a created message can be checked on the display portion 5230. The created message can be sent to another device. Alternatively, for example, the electronic appliance has a function of changing its display method in accordance with the illuminance of a usage environment. Thus, the power consumption of a smartphone can be reduced. A smartphone can display an image so that the smartphone can be suitably used even in an environment under strong external light, e.g., outdoors in fine weather, for example.

FIG. 16B illustrates an electronic appliance that can use a remote controller as the input portion 5240. An example of such an electronic appliance is a television system. For example, the electronic appliance that is capable of receiving data from a broadcast station or via the Internet and performing display on the display portion 5230. An image of a user can be taken using the detecting portion 5250. The image of the user can be transmitted. The electronic appliance can acquire a viewing history of the user and provide it to a cloud service. The electronic appliance can acquire recommendation data from a cloud service and display the data on the display portion 5230. A program or a moving image can be displayed on the basis of the recommendation data. Alternatively, for example, the electronic appliance has a function of changing its display method in accordance with the illuminance of a usage environment. Accordingly, for example, the television system can display an image so that the television system can be suitably used even when irradiated with strong external light that enters a room in fine weather.

FIG. 16C illustrates an electronic appliance that is capable of receiving educational materials via the Internet and displaying them on the display portion 5230. An example of such an electronic appliance is a tablet computer. An assignment can be input with the input portion 5240 and sent via the Internet. A corrected assignment or the evaluation of the assignment can be obtained from a cloud service and displayed on the display portion 5230. Suitable educational materials can be selected on the basis of the evaluation and displayed.

For example, the display can be performed on the display portion 5230 using an image signal received from another electronic appliance. When the electronic appliance is placed on a stand or the like, the display portion 5230 can be used as a sub-display. Thus, for example, a tablet computer can display an image so that the tablet computer can be suitably used even in an environment under strong external light, e.g., outdoors in fine weather.

FIG. 16D illustrates an electronic appliance including a plurality of display portions 5230. An example of such an electronic appliance is a digital camera. For example, the display portion 5230 can display an image that the detecting portion 5250 is capturing. A captured image can be displayed on the detecting portion. A captured image can be decorated using the input portion 5240. A message can be attached to a captured image. A captured image can be transmitted via the Internet. The electronic appliance has a function of changing its shooting conditions in accordance with the illuminance of a usage environment. Accordingly, for example, the digital camera can display an object so that an image is favorably viewed even in an environment under strong external light, e.g., outdoors in fine weather.

FIG. 16E illustrates an electronic appliance in which the electronic appliance of this embodiment is used as a master to control another electronic appliance used as a slave. An example of such an electronic appliance is a portable personal computer. As an example, part of image data can be displayed on the display portion 5230 and another part of image data can be displayed on a display portion of another electronic appliance. Image signals can be supplied to another electronic appliance. With the communication portion 5290, data to be written can be obtained from an input portion of another electronic appliance. Thus, a large display region can be utilized by using the portable personal computer, for example.

FIG. 17A illustrates an electronic appliance including the detecting portion 5250 that detects an acceleration or a direction. An example of such an electronic appliance is a goggles-type electronic appliance. The detecting portion 5250 can supply data on the position of the user or the direction in which the user faces. The electronic appliance can generate image data for the right eye and image data for the left eye in accordance with the position of the user or the direction in which the user faces. The display portion 5230 includes a display region for the right eye and a display region for the left eye. Thus, a virtual reality image that gives the user a sense of immersion can be displayed on the goggles-type electronic appliance, for example.

FIG. 17B illustrates an electronic appliance including the detecting portion 5250 that detects an acceleration or a direction. An example of such an electronic appliance is a glasses-type electronic appliance. The detecting portion 5250 can supply data on the position of the user or the direction in which the user faces. The electronic appliance can generate image data in accordance with the position of the user or the direction in which the user faces. Accordingly, the data can be shown together with a real-world scene, for example. An augmented reality image can be displayed on the glasses-type electronic appliance.

Note that this embodiment can be combined with any of the other embodiments described in this specification as appropriate.

### Embodiment 7

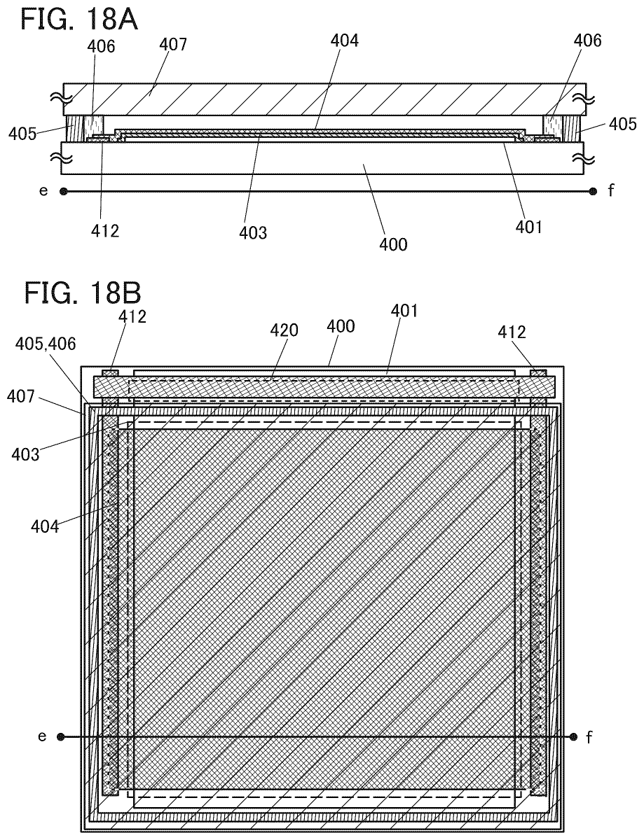

In this embodiment, a structure in which the light-emitting device described in Embodiment 1 is used for a lighting device will be described with reference to FIG. 18. FIG. 18A is a cross-sectional view taken along the line e-f in FIG. 18B which is a top view of a lighting device.

In the lighting device in this embodiment, a first electrode 401 is formed over a substrate 400 which is a support and has a light-transmitting property. The first electrode 401 corresponds to the first electrode 101 in Embodiment 1. In the case where light emission is extracted from the first electrode 401 side, the first electrode 401 is formed with a material having a light-transmitting property.

A pad 412 for supplying a voltage to a second electrode 404 is formed over the substrate 400.

An EL layer 403 is formed over the first electrode 401. The structure of the EL layer 403 corresponds to the structure of the EL layer 403 in Embodiment 3. Note that for these structures, the corresponding description can be referred to.

The second electrode 404 is formed to cover the EL layer 403. The second electrode 404 corresponds to the second electrode 102 in Embodiment 1. In the case where light-emission is extracted from the first electrode 401 side, the second electrode 404 is formed with a material having high reflectivity. The second electrode 404 is supplied with a voltage when connected to the pad 412.

As described above, the lighting device described in this embodiment includes a light-emitting device including the first electrode 401, the EL layer 403, and the second electrode 404. Since the light-emitting device is a light-emitting device with a high emission efficiency, the lighting device in this embodiment can be a lighting device with low power consumption.

The substrate 400 over which the light-emitting device having the above structure is formed is fixed to a sealing substrate 407 with sealants (405 and 406) and sealing is performed, whereby the lighting device is completed. It is possible to use only either the sealant 405 or the sealant 406. In addition, the inner sealant 406 (not illustrated in FIG. 18B) can be mixed with a desiccant, which enables moisture to be adsorbed, resulting in improved reliability.

When parts of the pad 412 and the first electrode 401 are provided to extend to the outside of the sealant 405 and the sealant 406, those can serve as external input terminals. An IC chip 420 mounted with a converter or the like may be provided over the external input terminals.

### Embodiment 8

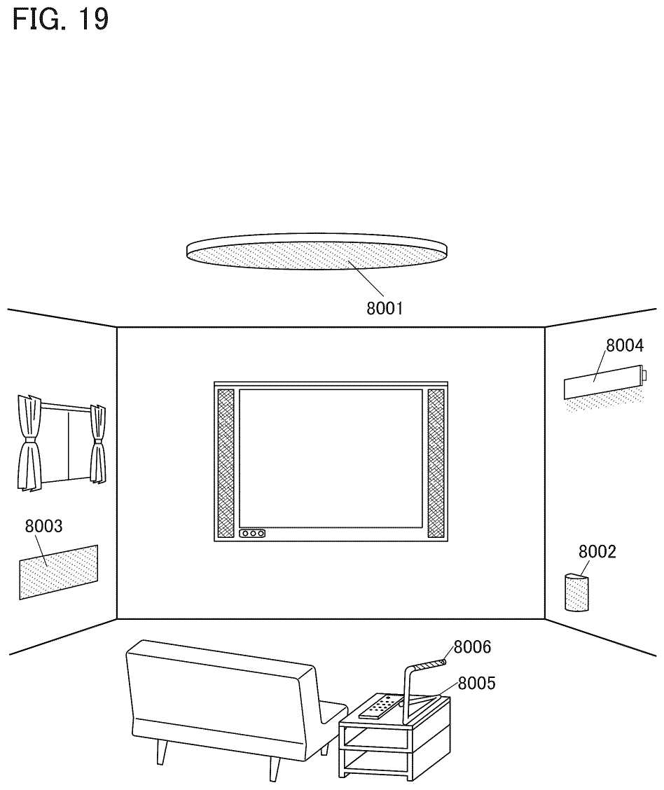

In this embodiment, application examples of lighting devices fabricated using the light-emitting apparatus of one embodiment of the present invention or the light-emitting device, which is part of the light-emitting apparatus, will be described with reference to FIG. 19.

A ceiling light 8001 can be used as an indoor lighting device. Examples of the ceiling light 8001 include a direct-mount light and an embedded light. Such lighting devices are fabricated using the light-emitting apparatus and a housing or a cover in combination. Other than that, application to a cord pendant light (light that is suspended from the ceiling by a cord) is also possible.

A foot light 8002 lights the floor so that safety on the floor can be improved. It can be effectively used in a bedroom, on a staircase, or in a passage, for example. In that case, the size or shape of the foot light can be changed in accordance with the area or structure of a room. The foot light can be a stationary lighting device made from the combination of the light-emitting apparatus and a support.

A sheet-like lighting 8003 is a thin sheet-like lighting device. The sheet-like lighting, which is attached to a wall when used, is space-saving and thus can be used for a wide variety of applications. Furthermore, the area of the sheet-like lighting can be easily increased. The sheet-like lighting can also be used on a wall or housing having a curved surface, for example.

In addition, a lighting device 8004 in which the light from a light source is controlled to be only in a desired direction can be used.

A desk lamp 8005 includes a light source 8006. As the light source 8006, the light-emitting apparatus of one embodiment of the present invention or the light-emitting device, which is part of the light-emitting apparatus, can be used.

In addition to the above examples, when the light-emitting apparatus of one embodiment of the present invention or the light-emitting device which is part of the light-emitting apparatus is used as a part of furniture in a room, a lighting device with functions of furniture can be obtained.

As described above, a variety of lighting devices that include the light-emitting apparatus can be obtained. Note that these lighting devices are also embodiments of the present invention.

The structures described in this embodiment can be used in an appropriate combination with any of the structures described in the other embodiments.

### Example 1

### Synthesis Example 1

In this example, a method for synthesizing N-(9,9-dimethyl-9H-fluoren-2-yl)-N-(m-terphenyl-4′-yl)-6-phenylbenzo[b]naphtho[1,2-d]furan-8-amine (abbreviation: FopTPBnf), which is the organic compound represented by Structural Formula (300) in Embodiment 1, will be described. The structural formula of FopTPBnf is shown below.

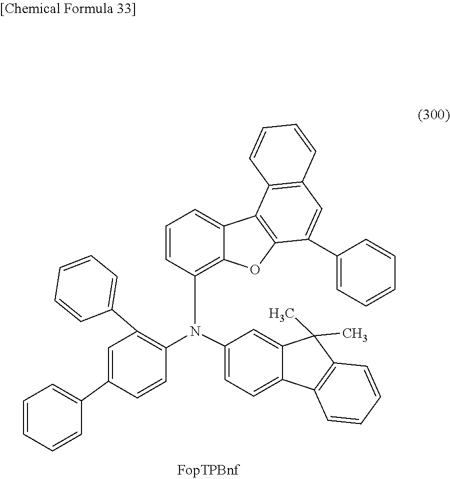 [SMILES: CC1(C)c2ccccc2-c2ccc(N(c3ccc(-c4ccccc4)cc3-c3ccccc3)c3cccc4c3oc3c(-c5ccccc5)cc5ccccc5c34)cc21]

### Step 1: Synthesis of N-(9,9-dimethyl-9H-fluoren-2-yl)-6-phenyl-benzo[b]naphtho[1,2-d]furan-8-amine

Into a 300 mL three-neck flask, 4.2 g (20 mmol) of 2-amino-9,9-dimethylfluorene and 8.4 g (20 mmol) of 8-iodo-6-phenyl-benzo[b]naphtho[1,2-d]furan were put, and the air in the system was replaced with nitrogen. Into the system, 0.12 g (0.30 mmol) of 2-dicyclohexylphosphino-2′,6′-dimethoxybiphenyl (abbreviation: SPhos), 2.9 g (30 mmol) of sodium t-butoxide (abbreviation: t-BuONa), 75 mL of xylene, and 86 mg (0.15 mmol) of bis(dibenzylideneacetone)palladium(0) (abbreviation: Pd(dba)₂) were put, and stirring was performed at 60° C. for 7 hours. After the stirring, water was added to the above mixture, and the base contained in the mixture was dissolved in the aqueous layer by ultrasonic treatment. The obtained mixture was subjected to suction filtration, and the obtained solid was washed with toluene, ethanol, and water. The obtained solid was dissolved in toluene, and an insoluble matter was removed by heat filtration. The obtained filtrate was concentrated, and the obtained solid was purified by recrystallization (solvent:toluene). The precipitated solid was collected by suction filtration and washed with toluene, acetone, and hexane. The obtained solid was dried, whereby a target white solid (6.5 g, a yield of 65%) was obtained. The synthesis scheme in Step 1 is shown in Formula (a-1) below.

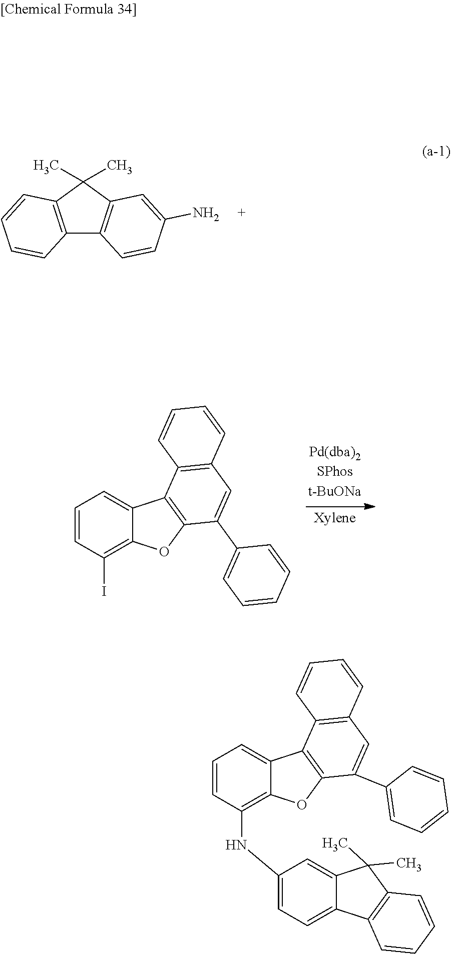 [SMILES: CC1(C)c2ccccc2-c2ccc(N)cc21 ; Ic1cccc2c1oc1c(-c3ccccc3)cc3ccccc3c12 ; CC1(C)c2ccccc2-c2ccc(Nc3cccc4c3oc3c(-c5ccccc5)cc5ccccc5c34)cc21]

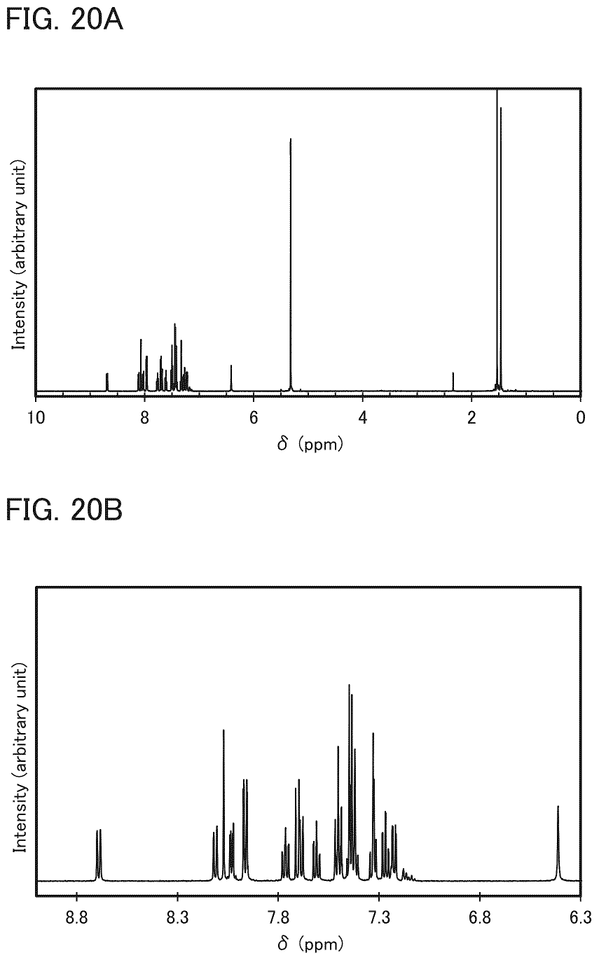

FIG. 20A and FIG. 20B show the ¹H NMR spectrum of the white solid obtained in Step 1. Results of ¹H NMR measurement are shown below. These results indicate that N-(9,9-dimethyl-9H-fluoren-2-yl)-6-phenyl-benzo[b]naphtho[1,2-d]furan-8-amine was obtained in this synthesis example.

¹H NMR (dichloromethane-d₂ 500 MHz): δ=8.69 (d, J=8.0 Hz, 1H), 8.11 (d, J=8.3 Hz, 1H), 8.07 (s, 1H), 8.03 (dd, J=6.0, 2.0 Hz, 1H), 7.97-7.95 (m, 2H), 7.76 (t, J=7.4 Hz, 1H), 7.70 (d, J=8.0 Hz, 1H), 7.68 (d, J=7.4 Hz, 1H), 7.61 (t, J=8.0 Hz, 1H), 7.52-7.49 (m, 2H), 7.46-7.40 (m, 4H), 7.35-7.31 (m, 2H), 7.27 (t, J=7.4 Hz, 1H), 7.22 (dd, J=8.0, 2.0 Hz, 1H), 6.41 (br, 1H), 1.46 (s, 6H).

### Step 2: Synthesis of FopTPBnf

Into a 200 mL three-neck flask, 1.9 g (6.2 mmol) of 4′-bromo-1,1′:3′,1″-terphenyl and 3.2 g (6.2 mmol) of N-(9,9-dimethyl-9H-fluoren-2-yl)-6-phenyl-benzo[b]naphtho[1,2-d]furan-8-amine were put, and the air in the system was replaced with nitrogen. Into the system, 51 mg (0.12 mmol) of 2-dicyclohexylphosphino-2′,6′-dimethoxybiphenyl (abbreviation: SPhos), 1.2 g (12 mmol) of sodium t-butoxide (abbreviation: t-BuONa), 45 mL of xylene, and 36 mg (62 μmol) of bis(dibenzylideneacetone)palladium(0) (abbreviation: Pd(dba)₂) were put, and the mixture was refluxed at 150° C. for 7 hours while being stirred.

After the stirring, water was added to the above mixture, the obtained mixture was separated into an organic layer and an aqueous layer, and the aqueous layer was subjected to extraction with toluene. The extracted solution and the organic layer were mixed, and the mixed solution was washed twice with water and then washed with a saturated aqueous solution of sodium chloride. The obtained organic layer was dried with magnesium sulfate, and the magnesium sulfate was removed by gravity filtration. The obtained filtrate was concentrated and the obtained solid was dried in a vacuum, whereby 5.2 g of a yellow viscous solid was obtained. The obtained yellow viscous solid was purified by silica gel chromatography (hexane and toluene were used as a development solvent; note that the ratio of hexane and toluene was hexane:toluene=10:1, and then hexane:toluene=4:1) and recrystallization (A mixed solvent of hexane and toluene was used as a solvent), and dried in a vacuum, whereby a target pale yellow solid (2.8 g, a yield of 63%) was obtained.

Next, by a train sublimation method, 2.8 g of the pale yellow solid was sublimated and purified. In the purification by sublimation, the solid was heated for 20 hours under a pressure of 4.2 Pa with an argon flow rate of 15 mL/min; the heating temperature was set to have a three-degree temperature gradient, 293° C., 290° C., and 250° C. After the purification by sublimation, a pale yellow solid (2.0 g, a collection rate of 71%) was obtained. The synthesis scheme in Step 2 is shown in Formula (a-2) below.

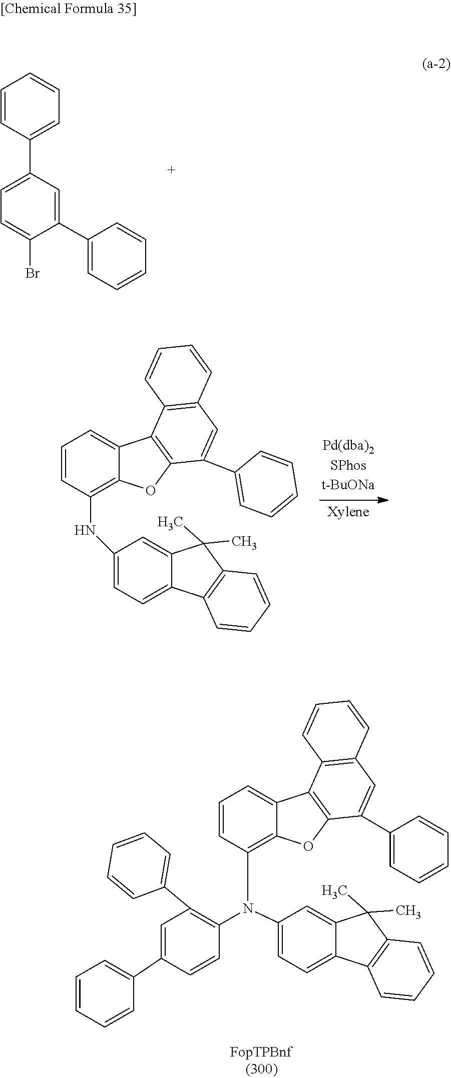 [SMILES: Brc1ccc(-c2ccccc2)cc1-c1ccccc1 ; CC1(C)c2ccccc2-c2ccc(Nc3cccc4c3oc3c(-c5ccccc5)cc5ccccc5c34)cc21 ; CC1(C)c2ccccc2-c2ccc(N(c3ccc(-c4ccccc4)cc3-c3ccccc3)c3cccc4c3oc3c(-c5ccccc5)cc5ccccc5c34)cc21]

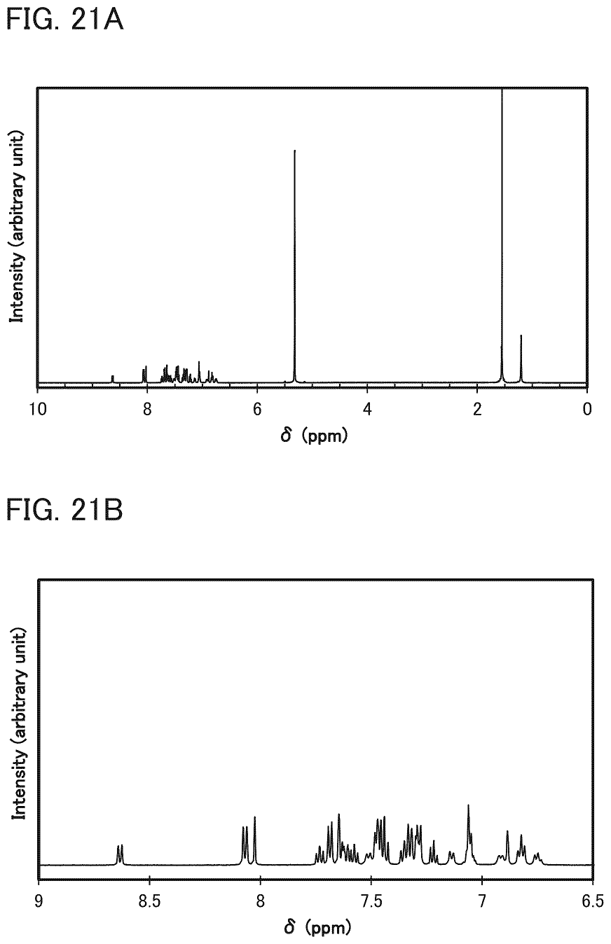

FIG. 21A and FIG. 21B show the ¹H NMR spectrum of the pale yellow solid obtained in Step 2. Results of ¹H NMR measurement are shown below. These results indicate that FopTPBnf was obtained in this synthesis example.

¹H NMR (dichloromethane-d₂ 500 MHz): δ=8.63 (d, J=8.0 Hz, 1H), 8.07 (d, J=8.0 Hz, 2H), 8.02 (s, 1H), 7.75-7.56 (m, 7H), 7.52-7.42 (m, 6H), 7.36-7.28 (m, 6H), 7.22 (t, J=7.4 Hz, 1H), 7.13 (d, J=8.0 Hz, 1H), 7.06-7.04 (m, 3H), 6.92-6.73 (m, 5H), 1.20 (s, 6H).

### <Measurement of Physical Properties>

Next, the ultraviolet-visible absorption spectra (hereinafter, simply referred to as “absorption spectra”) and emission spectra of a toluene solution and a solid thin film of FopTPBnf were measured. The solid thin film was formed over a quartz substrate by a vacuum evaporation method.

The absorption spectrum was measured with an ultraviolet-visible spectrophotometer (V-770DS, produced by JASCO Corporation). The absorption spectrum of FopTPBnf in the toluene solution was obtained by subtracting the measured absorption spectrum of only toluene put in a quartz cell from the measured absorption spectrum of the toluene solution of FopTPBnf put in a quartz cell. The absorption spectrum of the thin film was calculated using an absorbance (−log₁₀ [% T/(100−% R)] obtained from a transmittance and a reflectance of the substrate and the thin film. Note that % T represents the transmittance and % R represents the reflectance. The emission spectrum was measured with a spectrofluorometer (FP-8600DS, produced by JASCO Corporation).

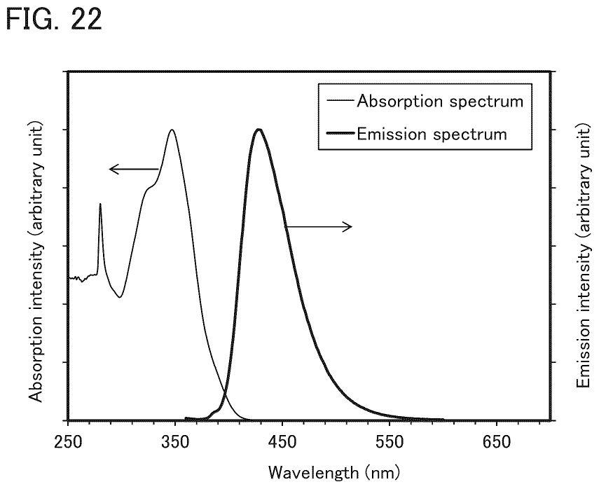

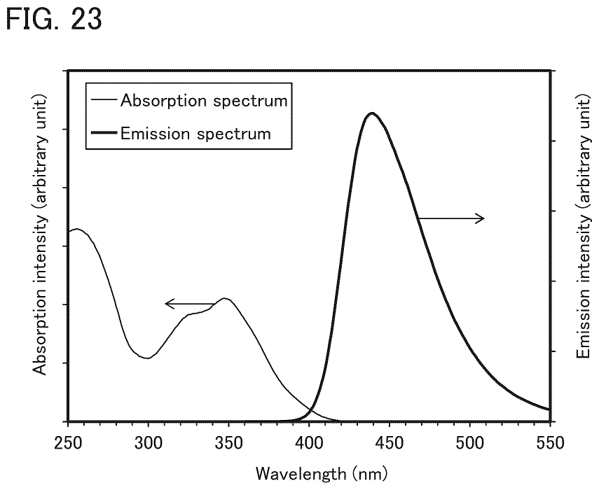

FIG. 22 shows the obtained measurement results of the absorption spectrum and the emission spectrum of the toluene solution. The horizontal axis represents the wavelength, and the vertical axes represent the absorption intensity and the emission intensity.

According to FIG. 22, the toluene solution of FopTPBnf exhibited an absorption peak at around 347 nm and an emission wavelength peak at around 427 nm (excitation wavelength: 347 nm).

FIG. 23 shows the obtained measurement results of the absorption spectrum and emission spectrum of the solid thin film. The horizontal axis represents the wavelength, and the vertical axes represent the absorption intensity and the emission intensity.

As shown in FIG. 23, the solid thin film of FopTPBnf exhibited absorption peaks at around 330 nm, around 352 nm, and around 383 nm, and an emission wavelength peak at around 439 nm (excitation wavelength: 350 nm).

Next, the HOMO level and the LUMO level of FopTPBnf calculated on the basis of cyclic voltammetry (CV) measurement are shown. The calculation method is described below.

An electrochemical analyzer (model number: ALS model 600A or 600C, produced by BAS Inc.) was used as a measurement apparatus. To prepare a solution for the CV measurement, dehydrated dimethylformamide (DMF) (produced by Sigma-Aldrich Inc., 99.8%, catalog No. 22705-6) was used as a solvent, tetra-n-butylammonium perchlorate (n-Bu₄NClO₄) (produced by Tokyo Chemical Industry Co., Ltd., catalog No. T0836) as a supporting electrolyte was dissolved at a concentration of 100 mmol/L, and the object to be measured was dissolved at a concentration of 2 mmol/L. A platinum electrode (PTE platinum electrode, produced by BAS Inc.) was used as a working electrode, another platinum electrode (Pt counter electrode for VC-3 (5 cm), produced by BAS Inc.) was used as an auxiliary electrode, and an Ag/Ag⁺ electrode (RE7 reference electrode for non-aqueous solvent, produced by BAS Inc.) was used as a reference electrode.

Note that the measurement was conducted at room temperature (20 to 25° C.). The scan speed in the CV measurement was fixed to 0.1 V/sec, and an oxidation potential Ea [V] and a reduction potential Ec [V] with respect to the reference electrode were measured. Ea is a standard oxidation potential (=(Eₚₐ+E_{pc})/2) obtained from the oxidation peak potential Eₚₐ and the reduction peak potential E_{pc} obtained by potential scanning in the positive direction, and Ec is a standard reduction potential (=(Eₚₐ+E_{pc})/2) obtained from the oxidation peak potential Eₚₐ[V] and the reduction peak potential E_{pc}[V] obtained by potential scanning in the negative direction. Here, since the potential energy of the reference electrode used in this example with respect to the vacuum level is known to be −4.94 [eV], the HOMO level and the LUMO level can be calculated by the following formulae: HOMO level [eV]−4.94−Ea and LUMO level [eV]−4.94−Ec.

CV measurement was repeated 100 times, and the oxidation-reduction wave in the hundredth cycle was compared with the oxidation-reduction wave in the first cycle to examine the electrical stability of the compound.

As a result, the HOMO level and the LUMO level of FopTPBnf were found to be −5.47 eV and −2.49 eV, respectively. In addition, in repetitive measurement of the oxidation-reduction wave, when the waveform of the first cycle was compared with that of the hundredth cycle, 81% of the peak intensity and 92% of the peak intensity were maintained in the Ea measurement and the Ec measurement, respectively, which confirmed that FopTPBnf had extremely high resistance to oxidation and reduction.

Then, thermogravimetry-differential thermal analysis (TG-DTA) of FopTPBnf was performed. The measurement was performed using a high vacuum differential type differential thermal balance (TG-DTA2410SA, manufactured by Bruker AXS K.K.). The measurement was performed under atmospheric pressure at a temperature rising rate of 10° C./min under a nitrogen stream (flow rate: 200 mL/min).

In the thermogravimetry-differential thermal analysis, the temperature (decomposition temperature) at which the weight obtained by thermogravimetry was reduced by 5% of the weight at the beginning of the measurement was found to be 427° C., which shows that FopTPBnf is a substance with high heat resistance.

Differential scanning calorimetry (DSC) of FopTPBnf was performed with DSC8500 manufactured by PerkinElmer, Inc. The differential scanning calorimetry was performed in the following manner: the temperature was raised from −10° C. to 350° C. at a temperature rising rate of 40° C./min and held at the temperature for three minute, and then the temperature was decreased to −10° C. at a temperature decreasing rate of 100° C./min. This operation was performed twice in succession.

The DSC measurement result of a second cycle showed that the glass transition point of FopTPBnf was 145° C. Since the glass transition temperature of a general hole-transport material is approximately 100° C. to 120° C., FopTPBnf was found to be a substance having high heat resistance as compared with the general hole-transport material.

### <Structural Analysis by Calculation>

Next, the HOMO and LUMO distributions of FopTPBnf were analyzed. The vibration (spin density) in the most stable structure where the singlet ground state (S₀) level of the compound is the lowest was analyzed by a calculation method. A density functional theory (DFT) method was used as the calculation method. The total energy calculated by the DFT is represented as the sum of potential energy, electrostatic energy between electrons, electronic kinetic energy, and exchange-correlation energy including all the complicated interactions between electrons. Also in the DFT, exchange-correlation interaction is approximated by a functional (a function of another function) of one electron potential represented in terms of electron density to enable high-speed calculations. Here, B3LYP which is a hybrid functional is used to specify the weight of each parameter related to exchange-correlation energy. As a basis function, 6-311G (d,p) is used. Gaussian 09 was used as a computational program.

As a comparative example, the HOMO and LUMO distributions of N-(9,9-dimethyl-9H-fluoren-2-yl)-N-(m-terphenyl-4′-yl)-6-phenyldibenzofuran-4-amine (hereinafter referred to as a comparative compound (c1)) in which a benzonaphthofuran skeleton of FopTPBnf was replaced with a dibenzofuran skeleton were analyzed. The structural formula of the comparative compound (c1) is shown below.

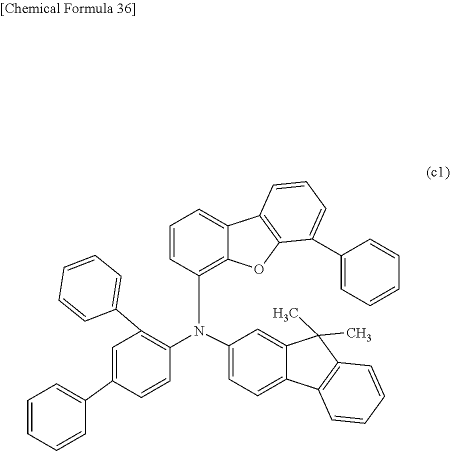 [SMILES: CC1(C)c2ccccc2-c2ccc(N(c3ccc(-c4ccccc4)cc3-c3ccccc3)c3cccc4c3oc3c(-c5ccccc5)cccc34)cc21]

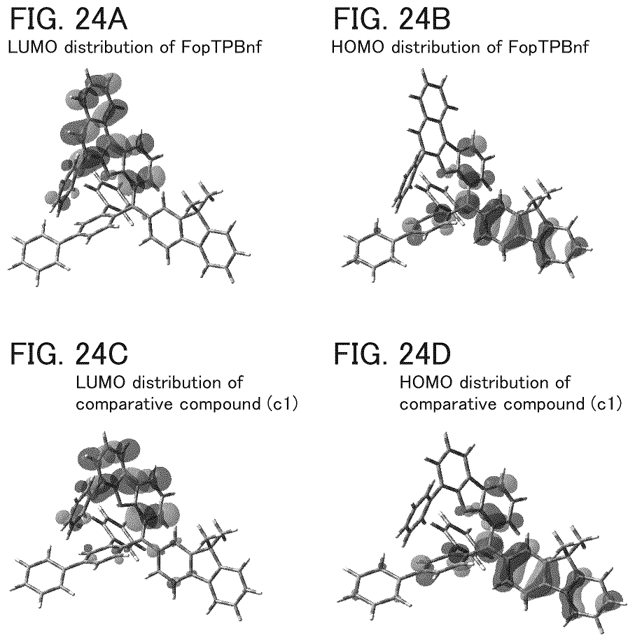

FIG. 24A to FIG. 24D show the analysis results of FopTPBnf and the comparative compound (c1). In FIG. 24A to FIG. 24D, a shadow in a molecule represents the distribution of HOMO or LUMO in the molecule. FIG. 24A shows the LUMO distribution of FopTPBnf, FIG. 24B shows the HOMO distribution of FopTPBnf, FIG. 24C shows the LUMO distribution of the comparative compound (c1), and FIG. 24D shows the HOMO distribution the comparative compound (c1).

It is found from FIG. 24A that LUMO is distributed in the benzonaphthofuran skeleton and the phenyl group bonded to the benzonaphthofuran skeleton in FopTPBnf. This is probably a factor in the property of a high resistance to reduction of FopTPBnf.

Meanwhile, it is found from FIG. 24C that in the comparative compound (c1), LUMO is distributed not only in a dibenzofuran skeleton and a phenyl group bonded to a dibenzofuran skeleton but also in a phenyl group bonded to a nitrogen atom in a fluorene skeleton and a phenyl group bonded to a nitrogen atom in a terphenyl skeleton. This indicates that the comparative compound (c1) in which the benzonaphthofuran skeleton of FopTPBnf is replaced with the dibenzofuran skeleton has lower resistance to repeated reduction than FopTPBnf.

The benzonaphthofuran skeleton has a naphthalene structure in the skeleton as described above, and conjugation is increased and electrons are easily accepted as compared with the dibenzofuran skeleton. Thus, the LUMO distribution can be placed inside the benzonaphthofuran skeleton, whereby the LUMO can be prevented from being distributed in the phenyl group bonded to the nitrogen atom in the fluorene skeleton and in the phenyl group bonded to the nitrogen atom in the terphenyl skeleton. Thus, introduction of a benzonaphthofuran skeleton can increase the resistance to reduction of the organic compound of one embodiment of the present invention.

FIG. 24B shows that, in FopTPBnf, HOMO is distributed around a fluorene skeleton, a phenyl group bonded to a nitrogen atom in a terphenyl skeleton, and a benzene skeleton directly bonded to a nitrogen atom in a benzonaphthofuran skeleton. Since the electron density of the benzene skeleton is decreased owing to the influence of an oxygen atom in the benzonaphthofuran skeleton, the HOMO level of FopTPBnf was able to be lowered.

As described above, it was found that the organic compound of one embodiment of the present invention, FopTPBnf, is a substance having favorable resistance to oxidation and reduction and extremely high heat resistance.

### Example 2

In this example, a light-emitting device 1 to a light-emitting device 3 and a comparative light-emitting device 4 were fabricated, and comparison results of the characteristics are shown. Structural formulae of organic compounds used for the light-emitting device 1 to the light-emitting device 3 and the comparative light-emitting device 4 are shown below. In addition, device structures of the light-emitting device 1 to the light-emitting device 3 and the comparative light-emitting device 4 are shown.

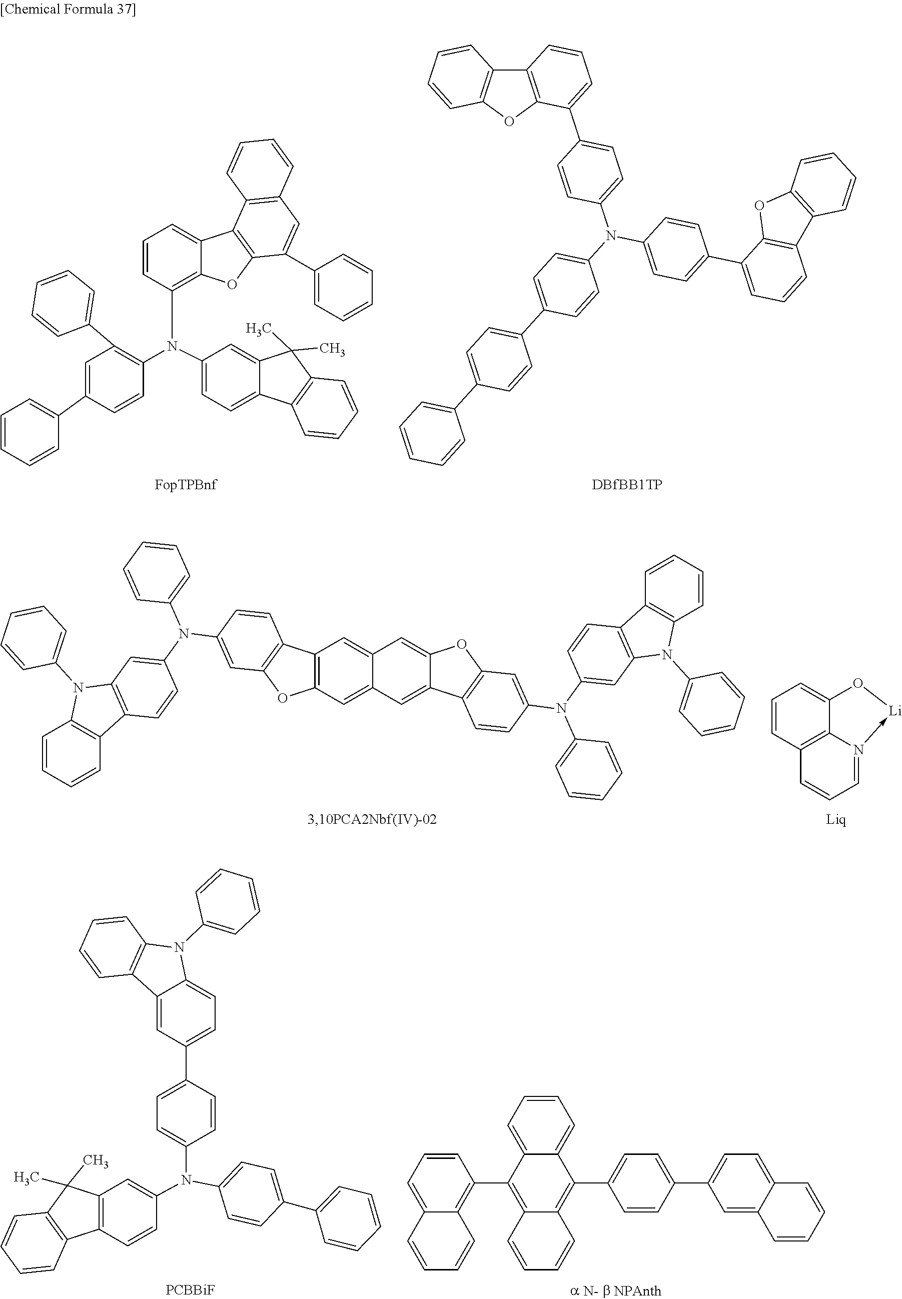

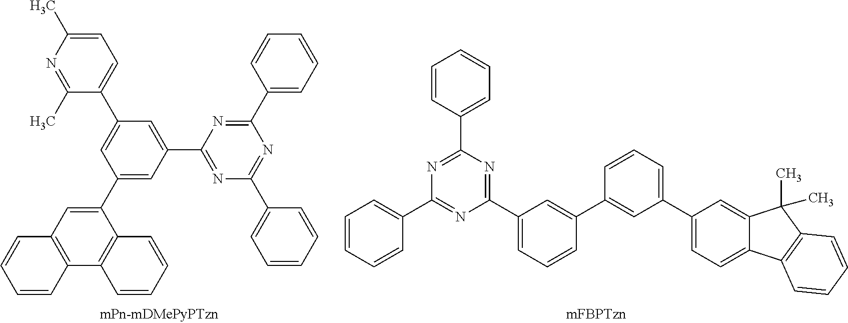

TABLE 1

| | Thickness | Light-emitting | Light-emitting | Light-emitting | Comparative |
|---|---|---|---|---|---|
| | (nm) | device 1 | device 2 | device 3 | light-emitting device 4 |
| Second electrode | 200 | Al | | | |
| Electron-injection | 1 | LiF | | | |
| layer | | | | | |
| Electron-transport layer | 15 | mPn-mDMePyPTzn:Liq (1:1) | | | |
| | 10 | mFBPTzn | | | |
| Light-emitting layer | 25 | αN-βNPAnth:3,10PCA2Nbf(IV)-02 (1:0.015) | | | |
| Hole-transport | 10 | DBfBB1TP | | | |
| layer | 90 | FopTPBnf | | | PCBBiF |
| Hole-injection | 10 | FopTPBnf:OCHD-003 | | | PCBBiF:OCHD-003 |
| layer | | (1:0.03) | (1:0.05) | (1:0.1) | (1:0.03) |

| | | |
|---|---|---|
| First electrode | 110 | ITSO |

### <<Fabrication of Light-Emitting Device 1>>

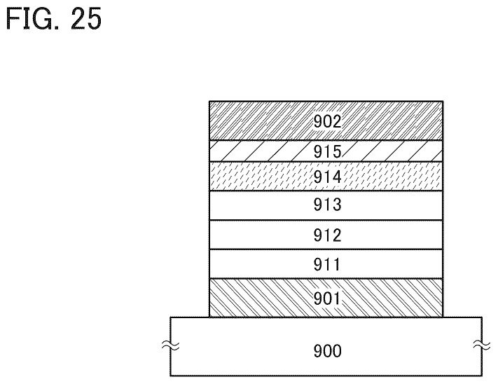

The light-emitting device 1 described in this example has a structure, as illustrated in FIG. 25, in which a hole-injection layer 911, a hole-transport layer 912, a light-emitting layer 913, an electron-transport layer 914, and an electron-injection layer 915 are stacked in this order over a first electrode 901 formed over a substrate 900, and a second electrode 902 is stacked over the electron-injection layer 915.

First, the first electrode 901 was formed over the substrate 900. The electrode area was set to 4 mm² (2 mm×2 mm). A glass substrate was used as the substrate 900. The first electrode 901 was formed to a thickness of 110 nm by a sputtering method using indium tin oxide containing silicon oxide (ITSO). Note that in this example, the first electrode 901 functioned as an anode.

As pretreatment, a surface of the substrate was washed with water, baking was performed at 200° C. for one hour, and then UV ozone treatment was performed for 370 seconds. After that, the substrate was transferred into a vacuum evaporation apparatus where the pressure was reduced to approximately 10⁻⁴ Pa, vacuum baking was performed at 170° C. for 30 minutes in a heating chamber of the vacuum evaporation apparatus, and then the substrate was cooled down for approximately 30 minutes.

Next, the hole-injection layer 911 was formed over the first electrode 901. After the pressure in the vacuum evaporation apparatus was reduced to 10⁻⁴ Pa, the hole-injection layer 911 was formed to a thickness of 10 nm by co-evaporating N-(9,9-dimethyl-9H-fluoren-2-yl)-N-(m-terphenyl-4′-yl)-6-phenylbenzo[b]naphtho[1,2-d]furan-8-amine (abbreviation: FopTPBnf), which is the organic compound of one embodiment of the present invention, and an electron acceptor material containing fluorine with a molecular weight of 672 (OCHD-003) in a weight ratio of 1:0.03 (=FopTPBnf:OCHD-003).

Then, the hole-transport layer 912 was formed over the hole-injection layer 911. The hole-transport layer 912 was formed by evaporating FopTPBnf to a thickness of 90 nm, and then evaporating N,N-bis[4-(dibenzofuran-4-yl)phenyl]-4-amino-p-terphenyl (abbreviation: DBfBB1TP) to a thickness of 10 nm.

Note that FopTPBnf is a material functioning as the first organic compound in the light-emitting device 1, and its HOMO level is −5.47 eV as described in Example 1. In addition, DBfBB1TP is a material functioning as an electron-blocking material in the light-emitting device 1, and its HOMO level is −5.49 eV. That is, the HOMO level of the first organic compound is lower than or equal to −5.40 eV. The HOMO level of the electron-blocking material is lower than the HOMO level of the first organic compound, and the difference therebetween is less than or equal to 0.30 eV.

Next, the light-emitting layer 913 was formed over the hole-transport layer 912.

The light-emitting layer 913 was formed to a thickness of 25 nm by co-evaporating 9-(1-naphthyl)-10-[4-(2-naphthyl)phenyl]anthracene (abbreviation: αN-βNPAnth) and 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02) in a weight ratio of αN-βNPAnth:3,10PCA2Nbf(IV)-02=1:0.015.

Note that α,N-βNPAnth is a material functioning as the host material in the light-emitting device 1 and its HOMO level is −5.85 eV. That is, the HOMO level of the host material is lower than the HOMO level of the first organic compound, and the difference therebetween is less than or equal to 0.60 eV. The HOMO level of the host material is lower than the HOMO level of the electron-blocking material, and the difference therebetween is less than or equal to 0.30 eV.

Next, the electron-transport layer 914 was formed over the light-emitting layer 913. The electron-transport layer 914 was formed by evaporating 2-[3′-(9,9-dimethyl-9H-fluoren-2-yl)biphenyl-3-yl]-4,6-diphenyl-1,3,5-triazine (abbreviation: mFBPTzn) to a thickness of 10 nm, and then co-evaporating 2-[3-(2,6-dimethyl-3-pyridinyl)-5-(9-phenanthrenyl)phenyl]-4,6-diphenyl-1,3,5-triazine (abbreviation: mPn-mDMePyPTzn) and 8-quinolinato-lithium (abbreviation: Liq) in a weight ratio of mPn-mDMePyPTzn:Liq=1:1 to a thickness of 15 nm.

Then, the electron-injection layer 915 was formed over the electron-transport layer 914. The electron-injection layer 915 was formed to a thickness of 1 nm by evaporation of lithium fluoride (LiF).

Next, the second electrode 902 was formed over the electron-injection layer 915. The second electrode 902 was formed by evaporating aluminum (Al) to a thickness of 200 nm. In this example, the second electrode 902 functions as a cathode.

Through the above process, the light-emitting device 1 was fabricated. Next, methods for fabricating the light-emitting device 2, the light-emitting device 3, and the comparative light-emitting device 4 will be described.

### <<Fabrication of Light-Emitting Device 2>>

The light-emitting device 2 is different from the light-emitting device 1 in that FopTPBnf and OCHD-003 used for the hole-injection layer 911 in the light-emitting device 1 were co-evaporated in a weight ratio of 1:0.05 (=FopTPBnf:OCHD-003). The other components were fabricated in a manner similar to that of the light-emitting device 1.

### <<Fabrication of Light-Emitting Device 3>>

The light-emitting device 3 is different from the light-emitting device 1 in that FopTPBnf and OCHD-003 used for the hole-injection layer 911 in the light-emitting device 1 were co-evaporated in a weight ratio of 1:0.1 (=FopTPBnf:OCHD-003). The other components were fabricated in a manner similar to that of the light-emitting device 1.

### <<Fabrication of Comparative Light-Emitting Device 4>>

The comparative light-emitting device 4 is different from the light-emitting device 1 in that FopTPBnf used for the hole-injection layer 911 and the hole-transport layer 912 in the light-emitting device 1 was replaced with N-(biphenyl-4-yl)-N-[4-(9-phenyl-9H-carbazol-3-yl)phenyl]-9,9-dimethyl-9H-fluoren-2-amine (abbreviation: PCBBiF). The other components were fabricated in a manner similar to that of the light-emitting device 1. Note that the HOMO level of PCBBiF is −5.36 eV. That is, the HOMO level of PCBBiF is higher than −5.40 eV.

The light-emitting device 1 to the light-emitting device 3 and the comparative light-emitting device 4 above were subjected to sealing with a glass substrate (a sealing material was applied to surround the devices, followed by UV treatment and one-hour heat treatment at 80° C. at the time of sealing) in a glove box containing a nitrogen atmosphere so that the light-emitting devices were not exposed to the air. Then, the initial characteristics of these light-emitting devices were measured.

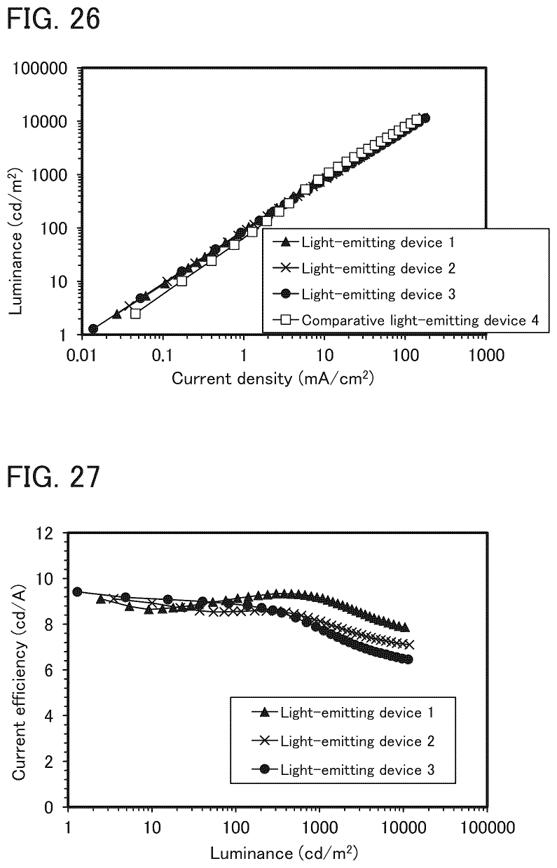

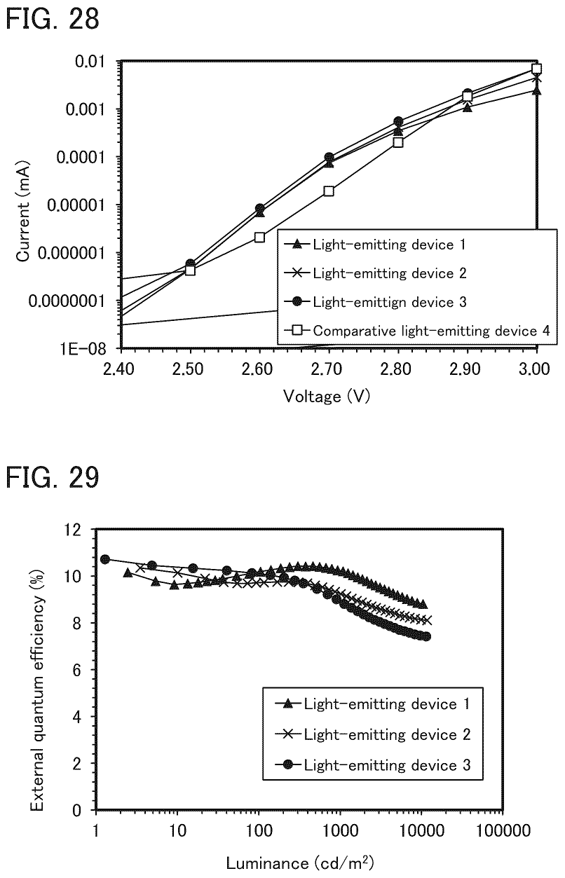

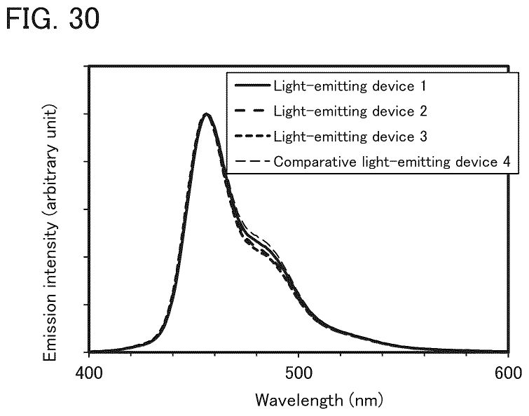

FIG. 26 shows the luminance-current density characteristics of the light-emitting device 1 to the light-emitting device 3 and the comparative light-emitting device 4. FIG. 27 shows the current efficiency-luminance characteristics thereof. FIG. 28 shows the current-voltage characteristics thereof. FIG. 29 shows the external quantum efficiency-luminance characteristics. FIG. 30 shows the emission spectra thereof. The following table shows the main characteristics of the light-emitting devices at around 1000 cd/m². The luminance, CIE chromaticity, and emission spectra were measured using a spectroradiometer (SR-UL1R, TOPCON TECHNOHOUSE CORPORATION). The external quantum efficiency was calculated from the luminance and the emission spectra measured with the spectroradiometer, on the assumption that light-distribution characteristics of light emissions from the devices were Lambertian type.

TABLE 2

| | | | | | | | External |
|---|---|---|---|---|---|---|---|
| | | | Current | | | Current | quantum |
| | Voltage | Current | density | | | efficiency | efficiency |
| | (V) | (mA) | (mA/cm²) | Chromaticity x | Chromaticity y | (cd/A) | (%) |
| Light-emitting device 1 | 6.2 | 0.41 | 10.1 | 0.138 | 0.101 | 9.19 | 10.3 |
| Light-emitting device 2 | 5.4 | 0.50 | 12.6 | 0.139 | 0.098 | 8.12 | 9.2 |
| Light-emitting device 3 | 4.2 | 0.46 | 11.4 | 0.140 | 0.097 | 7.89 | 9.0 |
| Comparative | 4.2 | 0.45 | 11.3 | 0.137 | 0.103 | 9.68 | 10.7 |
| light-emitting device 4 | | | | | | | |

It was found from FIG. 28 that the current-voltage characteristics at a voltage of 2.6 V to 2.8 V were higher in the light-emitting device 1 to the light-emitting device 3 including FopTPBnf in the hole-injection layer 911 and the hole-transport layer 912 than in the comparative light-emitting device 4. This indicates that when the light-emitting device of one embodiment of the present invention is fabricated using FopTPBnf, which is the organic compound of one embodiment of the present invention, for the hole-injection layer 911 and the hole-transport layer 912, the current-voltage characteristics of the light-emitting device can be improved.

FIG. 27 and FIG. 29 show that the light-emitting device 1 had the highest current efficiency and external quantum efficiency among the light-emitting device 1 to the light-emitting device 3 at a luminance of 100 cd/m² or more, and the light-emitting device 2 had the second highest current efficiency and external quantum efficiency. It was found from the above that in the case where FopTPBnf, which is the organic compound of one embodiment of the present invention, is used for the hole-injection layer 911, the structure in which the mixing ratio of OCHD-003 is not too high can have higher current efficiency and external quantum efficiency. Thus, the use of FopTPBnf, which is the organic compound of one embodiment of the present invention, for the hole-injection layer 911 enables the light-emitting device to have favorable characteristics even when the mixing ratio of OCHD-003 is low.

Since OCHD-003 has absorption in the visible light region, a lower mixing ratio of OCHD-003 in the hole-injection layer 911 can prevent a decrease in light extraction efficiency of the light-emitting device; thus, a light-emitting device with high emission efficiency can be provided. Specifically, the absorption edge of OCHD-003 is at around 580 nm, which is likely to affect the light extraction efficiency of a blue-light-emitting device or a green-light-emitting device. Thus, the use of FopTPBnf, which is the organic compound of one embodiment of the present invention, for the hole-injection layer 911 enables the light-emitting device to have favorable characteristics even when the mixing ratio of OCHD-003 is low; thus, the light extraction efficiency of the light-emitting device can be improved.

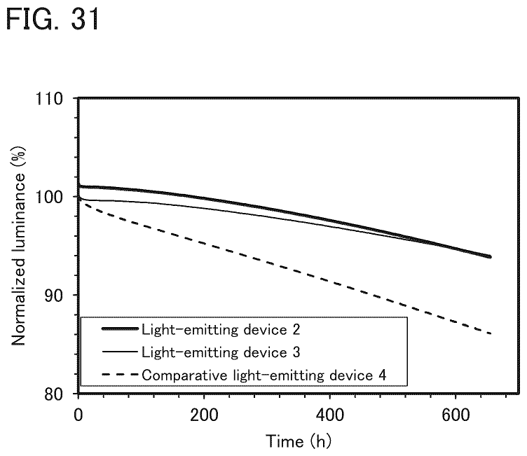

FIG. 31 shows changes in luminance over driving time when the light-emitting device 2, the light-emitting device 3, and the comparative light-emitting device 4 were driven at a constant current of 2 mA (50 mA/cm²). As shown in FIG. 31, the light-emitting device 2 and the light-emitting device 3 had a longer lifetime than the comparative light-emitting device 4. Accordingly, it was found that fabricating the light-emitting device of one embodiment of the present invention using FopTPBnf, which is the organic compound of one embodiment of the present invention, can extend the lifetime of the light-emitting device. That is, it was found that a light-emitting device having the following structure can be fabricated to be a light-emitting device having a longer lifetime than the comparative light-emitting device: the organic compound of one embodiment of the present invention is used as the first organic compound and the HOMO level of the first organic compound is −5.40 eV or lower; the HOMO level of the electron-blocking material is lower than the HOMO level of the first organic compound by 0.30 eV or lower; the HOMO level of the host material is lower than the HOMO level of the first organic compound by 0.60 eV or lower; and the HOMO level of the host material is lower than the HOMO level of the electron-blocking material by 0.30 eV or lower.

### Example 3

In this example, a light-emitting device 5 to a light-emitting device 7 of one embodiment of the present invention and a comparative light-emitting device 8 were fabricated, and comparison results of the device characteristics are shown. Structural Formulae of organic compounds used for the light-emitting device 5 to the light-emitting device 7 and the comparative light-emitting device 8 are shown below. In addition, device structures of the light-emitting device 5 to the light-emitting device 7 and the comparative light-emitting device 8 are shown.

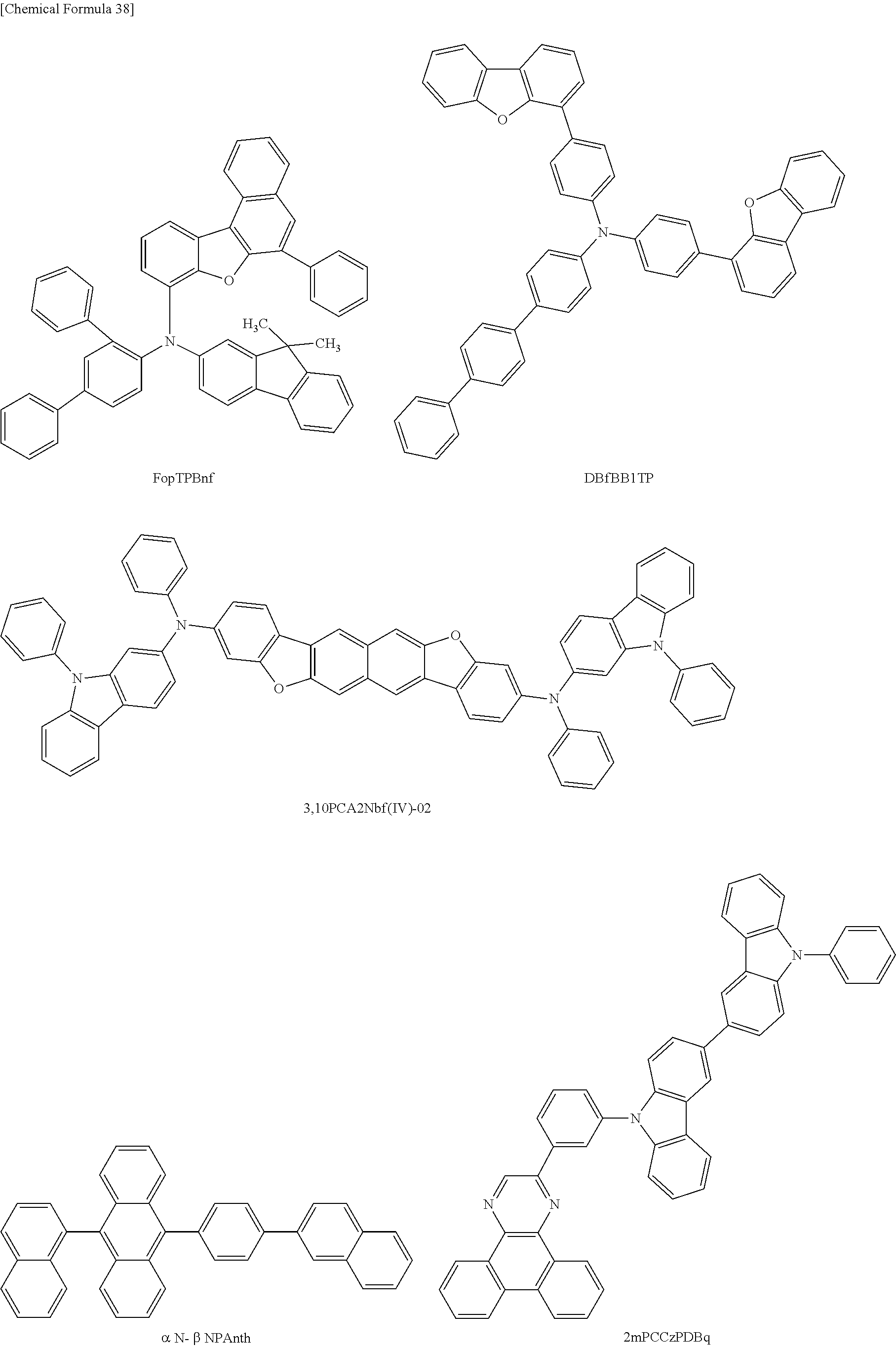

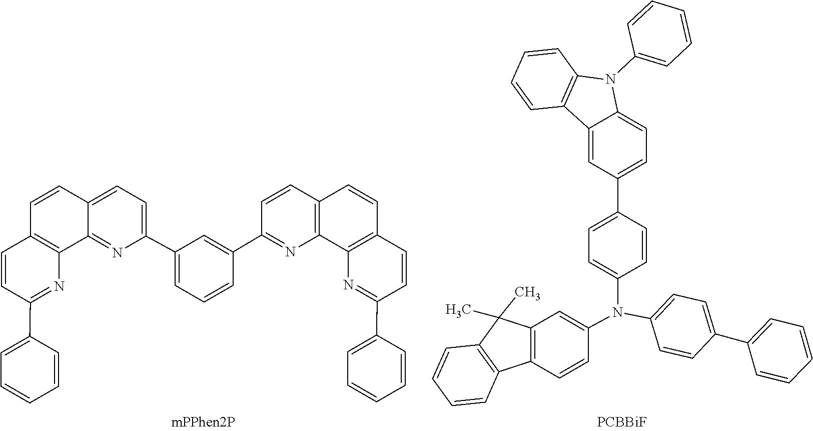

TABLE 3

| | | | | |
|---|---|---|---|---|
| Thickness | Light-emitting | Light-emitting | Light-emitting | Comparative |
| (nm) | device 5 | device 6 | device 7 | light-emitting device 8 |

| Second electrode | 200 | Al | |
|---|---|---|---|
| Electron-injection layer | 1 | LiF | |
| Electron-transport | 15 | mPPhen2P | |
| layer | 10 | 2mPCCzPDBq | |
| Light-emitting layer | 25 | αN-βNPAnth:3,10PCA2Nbf(IV)-02 (1:0.015) | |
| Hole-transport layer | 10 | DBfBB1TP | |
| | 90 | FopTPBnf | PCBBiF |
| Hole-injection layer | 10 | FopTPBnf:OCHD-003 | PCBBiF:OCHD-003 |

| | | | |
|---|---|---|---|
| (1:0.03) | (1:0.05) | (1:0.1) | (1:0.03) |

| | | |
|---|---|---|
| Fast electrode | 110 | ITSO |

### <<Fabrication of Light-Emitting Device 5>>

The light-emitting device 5 described in this example is different from the light-emitting device 1 in that mFBPTzn, which was used in the electron-transport layer 914 in the light-emitting device 1, was replaced with 2-{3-[3-(N-phenyl-9H-carbazol-3-yl)-9H-carbazol-9-yl]phenyl}dibenzo[f,h]quinoxaline (abbreviation: 2mPCCzPDBq), and evaporation of 2,2′-(1,3-phenylene)bis(9-phenyl-1,10-phenanthroline) (abbreviation: mPPhen2P) was performed instead of co-evaporation of mPn-mDMePyPTzn and Liq. The other components were fabricated in a manner similar to that of the light-emitting device 1.

### <<Fabrication of Light-Emitting Device 6>>

The light-emitting device 6 is different from the light-emitting device 5 in that FopTPBnf and OCHD-003 used for the hole-injection layer 911 in the light-emitting device 5 were co-evaporated in a weight ratio of 1:0.05 (=FopTPBnf:OCHD-003). The other components were fabricated in a manner similar to that of the light-emitting device 5.

### <<Fabrication of Light-Emitting Device 7>>

The light-emitting device 7 is different from the light-emitting device 5 in that FopTPBnf and OCHD-003 used for the hole-injection layer 911 in the light-emitting device 5 were co-evaporated in a weight ratio of 1:0.1 (=FopTPBnf:OCHD-003). The other components were fabricated in a manner similar to that of the light-emitting device 5.

### <<Fabrication of Comparative Light-Emitting Device 8>>

The comparative light-emitting device 8 is different from the light-emitting device 5 in that FopTPBnf used for the hole-injection layer 911 and the hole-transport layer 912 was replaced with PCBBiF. The other components were fabricated in a manner similar to that of the light-emitting device 5.

The light-emitting device 5 to the light-emitting device 7 and the comparative light-emitting device 8 above were subjected to sealing with a glass substrate (a sealing material was applied to surround the devices, followed by UV treatment and one-hour heat treatment at 80° C. at the time of sealing) in a glove box containing a nitrogen atmosphere so that the light-emitting devices were not exposed to the air. Then, the initial characteristics of these light-emitting devices were measured.

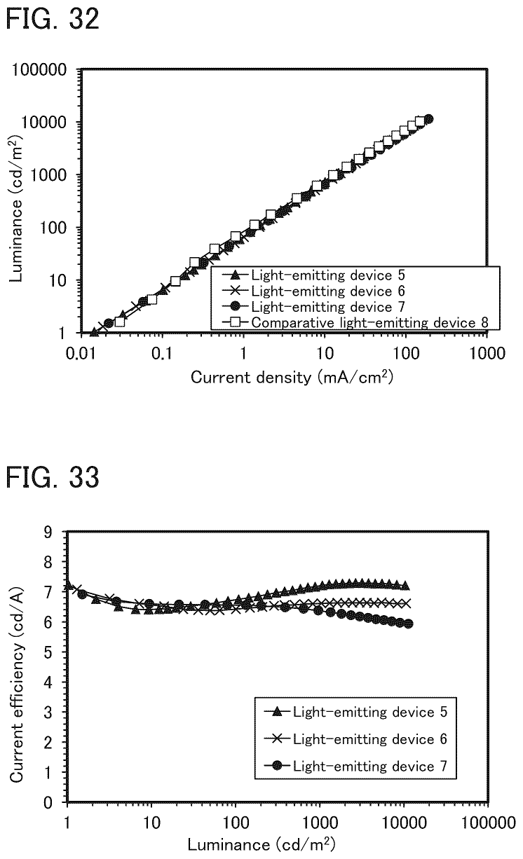

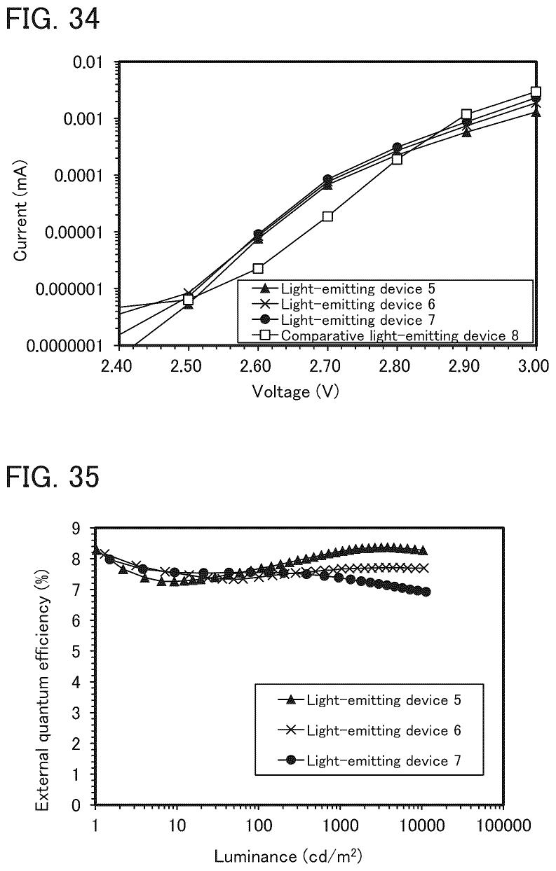

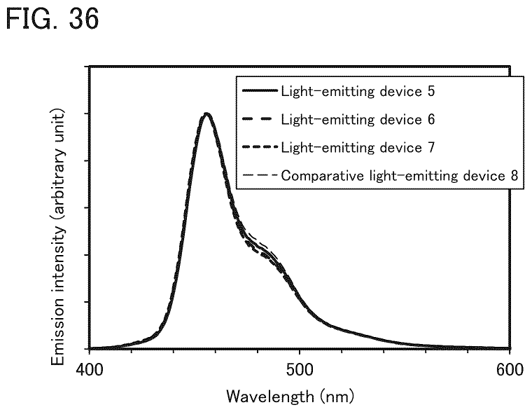

FIG. 32 shows the luminance-current density characteristics of the light-emitting device to the light-emitting device 7 and the comparative light-emitting device 8. FIG. 33 shows the current efficiency-luminance characteristics thereof. FIG. 34 shows the current-voltage characteristics thereof. FIG. 35 shows the external quantum efficiency-luminance characteristics. FIG. 36 shows the emission spectra thereof. The following table shows the main characteristics of the light-emitting devices at around 1000 cd/m². The luminance, CIE chromaticity, and emission spectra were measured using a spectroradiometer (SR-UL1R, TOPCON TECHNOHOUSE CORPORATION). The external quantum efficiency was calculated from the luminance and the emission spectra measured with the spectroradiometer, on the assumption that light-distribution characteristics of light emissions from the devices were Lambertian type.

TABLE 4

| | | | | | | | External |
|---|---|---|---|---|---|---|---|
| | | | Current | | | Current | quantum |
| | Voltage | Current | density | | | efficiency | efficiency |
| | (V) | (mA) | (mA/cm²) | Chromaticity x | Chromaticity y | (cd/A) | (%) |
| Light-emitting device 5 | 6.6 | 0.59 | 14.9 | 0.139 | 0.097 | 7.21 | 8.25 |
| Light-emitting device 6 | 5.4 | 0.63 | 15.8 | 0.140 | 0.095 | 6.62 | 7.67 |
| Light-emitting device 7 | 4.2 | 0.60 | 15.1 | 0.141 | 0.095 | 6.38 | 7.39 |
| Comparative | 4.2 | 0.51 | 12.7 | 0.138 | 0.098 | 7.58 | 8.66 |
| light-emitting device 8 | | | | | | | |

It was found from FIG. 34 that the current-voltage characteristics at a voltage of 2.6 V to 2.8 V were higher in the light-emitting device 5 to the light-emitting device 7 including FopTPBnf in the hole-injection layer 911 and the hole-transport layer 912 than in the comparative light-emitting device 8. This indicates that when the light-emitting device of one embodiment of the present invention is fabricated using FopTPBnf, which is the organic compound of one embodiment of the present invention, for the hole-injection layer 911 and the hole-transport layer 912, the current-voltage characteristics of the light-emitting device can be improved.

FIG. 33 and FIG. 35 show that the light-emitting device 5 had the highest current efficiency and external quantum efficiency among the light-emitting device 5 to the light-emitting device 7, and the light-emitting device 6 had the second highest current efficiency and external quantum efficiency. In the case where FopTPBnf, which is the organic compound of one embodiment of the present invention, is used for the hole-injection layer 911, OCHD-003 and FopTPBnf are mixed to be used; it was found that the structure in which the mixing ratio of OCHD-003 is not too high can have higher current efficiency and external quantum efficiency. Thus, the use of FopTPBnf, which is the organic compound of one embodiment of the present invention, for the hole-injection layer 911 enables the light-emitting device to have favorable characteristics even when the mixing ratio of OCHD-003 is low.

Since OCHD-003 has absorption in the visible light region, a lower mixing ratio of OCHD-003 in the hole-injection layer 911 can prevent a decrease in light extraction efficiency of the light-emitting device; thus, a light-emitting device with high emission efficiency can be provided. Specifically, the absorption edge of OCHD-003 is at around 580 nm, which is likely to affect the light extraction efficiency of a blue-light-emitting device or a green-light-emitting device. Thus, the use of FopTPBnf, which is the organic compound of one embodiment of the present invention, for the hole-injection layer 911 enables the light-emitting device to have favorable characteristics even when the mixing ratio of OCHD-003 is low; thus, the light extraction efficiency of the light-emitting device can be improved.

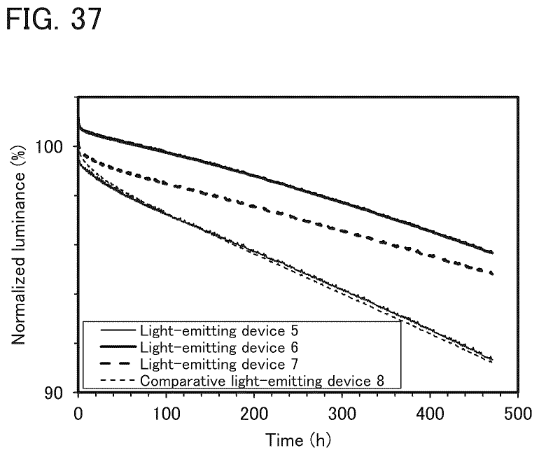

FIG. 37 shows changes in luminance over driving time when the light-emitting device 5 to the light-emitting device 7 and the comparative light-emitting device 8 were driven at a constant current of 2 mA (50 mA/cm²). As shown in FIG. 37, the light-emitting device 5 to the light-emitting device 7 had a longer lifetime than the comparative light-emitting device 8. Accordingly, it was found that the lifetime of the light-emitting device was extended with the use of FopTPBnf, which is the organic compound of one embodiment of the present invention. In addition, it was found that the light-emitting device 6 and the light-emitting device 7 each had a longer driving lifetime than the comparative light-emitting device 8. This is because the recombination region in the light-emitting layer is in the vicinity of the center of the light-emitting layer, which inhibits passing of holes or electrons toward the adjacent transport layer. Passing of carriers toward the transport layer adjacent to the light-emitting layer is considered to shorten the driving lifetime of the light-emitting device because of a decrease in emission efficiency. However, in the light-emitting device 6 and the light-emitting device 7 of the present invention, the light-emitting region is considered as a center of the light-emitting layer and passing of carriers toward an adjacent layer is less likely to occur; thus, a favorable driving lifetime can be achieved. Accordingly, the light-emitting device of one embodiment of the present invention is a light-emitting device with favorable reliability.

### REFERENCE NUMERALS

GD: circuit, IR: subpixel, M11: transistor, M12: transistor, M13: transistor, M14: transistor, M15: transistor, M16: transistor, M17: transistor, MS: wiring, PS: subpixel, REG: resist mask, RES: wiring, SE1: wiring, SE: distance, Si: single crystal, TX: wiring, VG: wiring, VS: wiring, 101: first electrode, 102: second electrode, 103: EL layer, 103a: EL layer, 103b: EL layer, 103B: EL layer, 103G: EL layer, 103R: EL layer, 103PS: light-receiving layer, 104B: hole-injection/transport layer, 104G: hole-injection/transport layer, 104R: hole-injection/transport layer, 104PS: hole-injection/transport layer, 105B: light-emitting layer, 105G: light-emitting layer, 105R: light-emitting layer, 105PS: active layer, 106: charge-generation layer, 106a: charge-generation layer, 106b: charge-generation layer, 107: insulating layer, 108B: electron-transport layer, 108G: electron-transport layer, 108R: electron-transport layer, 108PS: electron-transport layer, 109: electron-injection layer, 110B: sacrificial layer, 110G: sacrificial layer, 110R: sacrificial layer, HOPS: sacrificial layer, 111: hole-injection layer, 111a: hole-injection layer, 111b: hole-injection layer, 112: hole-transport layer, 112a: hole-transport layer, 112b: hole-transport layer, 113: light-emitting layer, 113a: light-emitting layer, 113b: light-emitting layer, 113c: light-emitting layer, 114: electron-transport layer, 114a: electron-transport layer, 114b: electron-transport layer, 115: electron-injection layer, 115a: electron-injection layer, 115b: electron-injection layer, 130: connection portion, 400: substrate, 401: first electrode, 403: EL layer, 404: second electrode, 405: sealant, 406: sealant, 407: sealing substrate, 412: pad, 420: IC chip, 501C: insulating film, 501D: insulating film, 504: conductive film, 506: insulating film, 508: semiconductor film, 508A: region, 508B: region, 508C: region, 510: first substrate, 512A: conductive film, 512B: conductive film, 516: insulating film, 516A: insulating film, 516B: insulating film, 518: insulating film, 520: functional layer, 524: conductive film, 528: partition wall, 528a: resin film, 530: pixel circuit, 530S: pixel circuit, 530X: pixel circuit, 531: pixel circuit, 550: light-emitting device, 550X: light-emitting device, 550S: light-receiving device, 550B: light-emitting device, 550G: light-emitting device, 550R: light-emitting device, 550PS: light-receiving device, 551B: electrode, 551C: connection electrode, 551G: electrode, 551R: electrode, 551PS: electrode, 552: electrode, 580: space, 591S: wiring, 591X: wiring, 700: light-emitting and light-receiving apparatus, 701: display region, 702G: subpixel, 702PS: subpixel, 702R: subpixel, 703: pixel, 704: circuit, 705: insulating layer, 706: wiring, 710: substrate, 711: substrate, 712: IC, 713: FPC, 720: apparatus, 770: substrate, 800: substrate, 801a: electrode, 801b: electrode, 802: electrode, 803a: EL layer, 803b: light-receiving layer, 805a: light-emitting device, 805b: light-receiving device, 810: light-emitting and light-receiving apparatus, 810A: light-emitting and light-receiving apparatus, 810B: light-emitting and light-receiving apparatus, 810C: light-emitting and light-receiving apparatus, 900: substrate, 901: first electrode, 902: second electrode, 911: hole-injection layer, 912: hole-transport layer, 913: light-emitting layer, 914: electron-transport layer, 915: electron-injection layer, 5200B: electronic appliance, 5210: arithmetic device, 5220: input/output device, 5230: display portion, 5240: input portion, 5250: detecting portion, 5290: communication portion, 8001: ceiling light, 8002: foot light, 8003: sheet-like lighting, 8004: lighting device, 8005: desk lamp, 8006: light source

1. A light-emitting device comprising a first electrode, a second electrode, a light-emitting layer, and a first layer,
wherein the light-emitting layer is positioned between the first electrode and the second electrode,
wherein the first layer is positioned between the first electrode and the light-emitting layer,
wherein the light-emitting layer comprises a light-emitting substance,
wherein the first layer comprises a first organic compound, and
wherein the first organic compound is an organic compound represented by General Formula (G1), 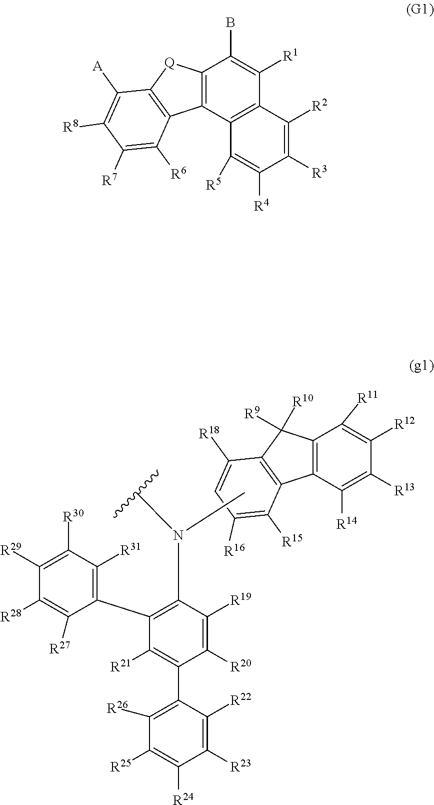
wherein:
Q represents an oxygen atom or a sulfur atom;
One of A and B is a group represented by General Formula (g1), and the other represents any of hydrogen, an alkyl group having 1 to 6 carbon atoms, a cyclic saturated hydrocarbon group having 3 to 6 carbon atoms, an alkoxy group having 1 to 6 carbon atoms, a cyano group, halogen, a haloalkyl group having 1 to 6 carbon atoms, and a substituted or unsubstituted aromatic hydrocarbon group having 6 to 60 carbon atoms;
R¹ to R⁸ each independently represent any of hydrogen, an alkyl group having 1 to 6 carbon atoms, a cyclic saturated hydrocarbon group having 3 to 6 carbon atoms, an alkoxy group having 1 to 6 carbon atoms, a cyano group, halogen, a haloalkyl group having 1 to 6 carbon atoms, and a substituted or unsubstituted aromatic hydrocarbon group having 6 to 60 carbon atoms;
R⁹ and R¹⁰ each independently represent any of hydrogen, an alkyl group having 1 to 6 carbon atoms, a cyclic saturated hydrocarbon group having 3 to 6 carbon atoms, an alkoxy group having 1 to 6 carbon atoms, a cyano group, halogen, a haloalkyl group having 1 to 6 carbon atoms, and a substituted or unsubstituted phenyl group;
R¹¹ to R³¹ each independently represent any of hydrogen, an alkyl group having 1 to 6 carbon atoms, a cyclic saturated hydrocarbon group having 3 to 6 carbon atoms, an alkoxy group having 1 to 6 carbon atoms, a cyano group, halogen, a haloalkyl group having 1 to 6 carbon atoms, and a substituted or unsubstituted aromatic hydrocarbon group having 6 to 60 carbon atoms; and
any one of R¹⁵ to R¹⁸ represents a bond with a nitrogen atom.

2. The light-emitting device according to claim 1,
wherein the light-emitting layer further comprises a host material, and
wherein a difference between a HOMO level of the first organic compound and a HOMO level of the host material is less than or equal to 0.60 eV

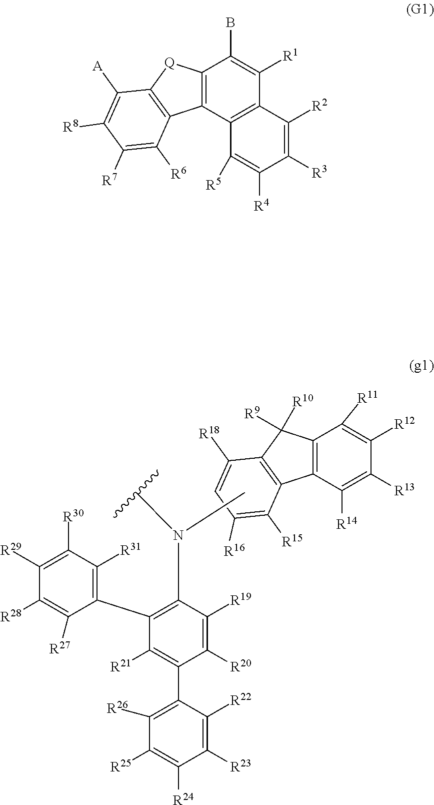

3. The light-emitting device according to claim 1, further comprising a second layer,
wherein the second layer is positioned between the light-emitting layer and the first layer,
wherein the light-emitting layer further comprises a host material,
wherein the second layer comprises a second organic compound,
wherein a difference between a HOMO level of the first organic compound and a HOMO level of the host material is less than or equal to 0.60 eV,
wherein a difference between the HOMO level of the first organic compound and a HOMO level of the second organic compound is less than or equal to 0.30 eV, and
wherein the second organic compound is a material having a hole-transport property.

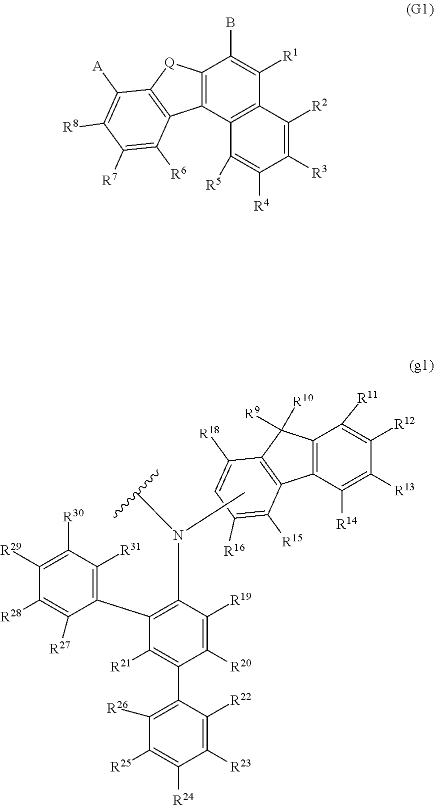

4. An organic compound represented by General Formula (G5), 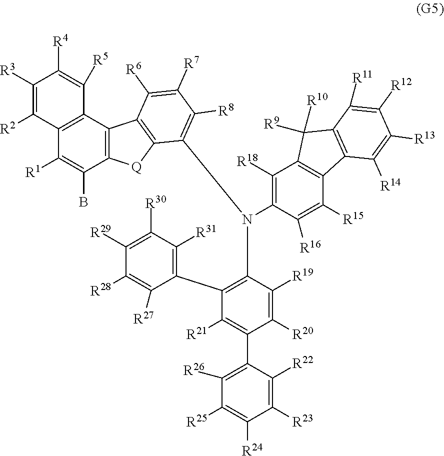
wherein:
Q represents an oxygen atom or a sulfur atom;
B, R¹ to R⁸, R¹¹ to R¹⁶, and R⁸ to R³¹ each independently represent any of hydrogen, an alkyl group having 1 to 6 carbon atoms, a cyclic saturated hydrocarbon group having 3 to 6 carbon atoms, an alkoxy group having 1 to 6 carbon atoms, a cyano group, halogen, a haloalkyl group having 1 to 6 carbon atoms, and a substituted or unsubstituted aromatic hydrocarbon group having 6 to 60 carbon atoms; and
R⁹ and R¹⁰ each independently represent any of hydrogen, an alkyl group having 1 to 6 carbon atoms, a cyclic saturated hydrocarbon group having 3 to 6 carbon atoms, an alkoxy group having 1 to 6 carbon atoms, a cyano group, halogen, a haloalkyl group having 1 to 6 carbon atoms, and a substituted or unsubstituted phenyl group.

5. The organic compound according to claim 4,
wherein the organic compound is represented by Structural Formula (300).

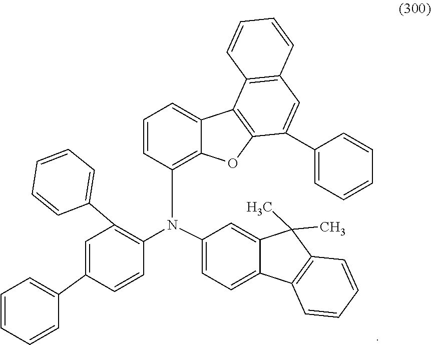 [SMILES: CC1(C)c2ccccc2-c2ccc(N(c3ccc(-c4ccccc4)cc3-c3ccccc3)c3cccc4c3oc3c(-c5ccccc5)cc5ccccc5c34)cc21]

6. A light-emitting and light-receiving apparatus comprising a light-emitting device and a light-receiving device,
wherein the light-emitting device and the light-receiving device are positioned over the same substrate,
wherein the light-emitting device comprises a first electrode, a second electrode, a light-emitting layer, and a first layer,
wherein the light-emitting layer is positioned between the first electrode and the second electrode,
wherein the first layer is positioned between the first layer and the light-emitting layer,
wherein the first layer comprises a first organic compound,
wherein the light-receiving device comprises a third electrode, a fourth electrode, a light-receiving layer, and a second layer,
wherein the light-receiving layer is positioned between the third electrode and the fourth electrode,
wherein the second layer is positioned between the third electrode and the light-receiving layer,
wherein the second layer comprises the first organic compound,
wherein a HOMO level of the first organic compound is lower than or equal to −5.40 eV, and
wherein the first organic compound is an organic compound represented by General Formula (G10), 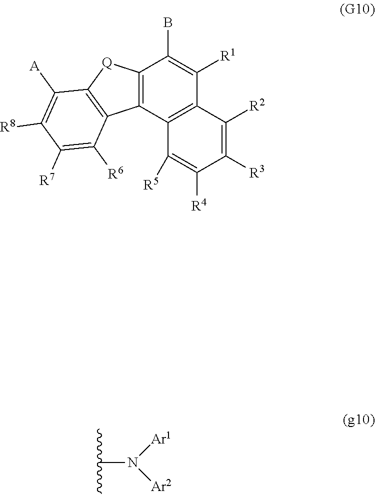
wherein:
Q represents an oxygen atom or a sulfur atom;
One of A and B is a group represented by General Formula (g10), and the other represents any of hydrogen, an alkyl group having 1 to 6 carbon atoms, a cyclic saturated hydrocarbon group having 3 to 6 carbon atoms, an alkoxy group having 1 to 6 carbon atoms, a cyano group, halogen, a haloalkyl group having 1 to 6 carbon atoms, and a substituted or unsubstituted aromatic hydrocarbon group having 6 to 60 carbon atoms;
R¹ to R⁸ each independently represent any of hydrogen, an alkyl group having 1 to 6 carbon atoms, a cyclic saturated hydrocarbon group having 3 to 6 carbon atoms, an alkoxy group having 1 to 6 carbon atoms, a cyano group, halogen, a haloalkyl group having 1 to 6 carbon atoms, and a substituted or unsubstituted aromatic hydrocarbon group having 6 to 60 carbon atoms; and
Ar¹ and Ar² each independently represent a substituted or unsubstituted aromatic hydrocarbon group having 6 to 60 carbon atoms.

7. A light-emitting apparatus comprising:
the light-emitting device according to claim 1; and
a transistor or a substrate.

8. An electronic appliance comprising:
the light-emitting apparatus according to claim 7; and
a detecting portion, an input portion, or a communication portion.

9. A lighting device comprising:
the light-emitting apparatus according to claim 7; and
a housing.

10. An electronic appliance comprising:
the light-emitting and light-receiving apparatus according to claim 6; and
a detecting portion, an input portion, or a communication portion.

11. The light-emitting device according to claim 1,
wherein a HOMO level of the first organic compound is lower than or equal to −5.40 eV.

12. The light-emitting device according to claim 1,
R⁹ and R¹⁰ are bonded to each other to form a spiro ring structure.

13. The organic compound according to claim 4,
R⁹ and R¹⁰ are bonded to each other to form a spiro ring structure.
